(19) United States
(12) Patent Application Publication
(10) Pub. No.: US 2009/0325156 A1
(43) Pub. Date: Dec. 31, 2009

(54) MATERIALS AND METHODS FOR ABCB1 POLYMORPHIC VARIANT SCREENING, DIAGNOSIS, AND TREATMENT

(75) Inventors: William D. Figg, Fairfax, VA (US); Alexander Sparreboom, Memphis, TN (US); Tristan Sissung, Annandale, VA (US); Richard L. Piekarz, Silver Spring, MD (US); Susan E. Bates, Bethesda, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: Government of the United States of America, as Represented by Secretary, Dept. of Human Services, Rockville, MD (US)

(21) Appl. No.: 12/093,225

(22) PCT Filed: Nov. 9, 2006
(86) PCT No.: PCT/US06/43656

§ 371 Date: Jan. 16, 2009

Related U.S. Application Data
(60) Provisional application No. 60/736,083, filed on Nov. 10, 2005.

(51) Int. Cl. C12Q 1/68

(52) U.S. Cl. 435/6

(57) ABSTRACT

The invention provides methods and materials for screening for polymorphic variants in ABCB1 and diagnosing altered susceptibilities for drug-induced heart rhythm irregularities based on the same. These methods allow better treatment regimens for using drugs that bind a protein encoded by the ABCB1 and/or induce heart rhythm irregularities such as the anti-cancer drug FK228.

### BACKGROUND OF THE INVENTION

Drugs that have tremendous benefits in ameliorating human suffering unfortunately can also have undesirable, and potentially dangerous, side effects. For example, treatment with FK228 (romidepsin), an anti-cancer drug, has been associated with cardiac toxicities in preclinical models, including ST/T wave flattening and asymptomatic dysrhythumias, and with reversible ECG changes. Other drugs also have negative side effects on the heart. Complicating matters, the side effects a drug has can vary between individuals. There has been and continues to be a search for ways of identifying how a drug will affect a given individual, and once that identification is made, ways of treating that individual. Accordingly, there exists a need for materials and methods for identifying individuals' susceptibility for drug induced effects on the heart and associated means of treatment.

### BRIEF SUMMARY OF THE INVENTION

The invention provides methods and materials for screening for polymorphic variants in the ABCB1 gene and diagnosing altered susceptibilities for drug-induced heart rhythm irregularities based on the same. In one aspect, a method of screening for an altered susceptibility for a drug-induced heart rhythm irregularity is provided. A sample from a subject is screened to detect the presence or absence of at least one polymorphic variant of at least one polymorphism of the ABCB1 gene, wherein the polymorphic variant is associated with an altered susceptibility for a heart rhythm irregularity induced by a drug that binds a protein encoded by the ABCB1 gene. A diagnosis for the altered susceptibility of the subject for the heart rhythm irregularity as induced by the drug is rendered based on the presence or absence of the polymorphic variant of the ABCB1 gene. In one aspect, the polymorphism comprises a polymorphism identified as rs1128503, rs2032582, rs1045642, or a combination thereof. In one aspect, the polymorphism comprises a polymorphism at position 49,910, 68,894, or 90,871 of SEQ ID NO: 1; or 1236, 2677, or 3435 of SEQ ID NO: 2; or a combination thereof. In another aspect, a method of screening for a decreased susceptibility for a depsipeptide, e.g., FK228,-induced QT interval prolongation is provided. A sample from a subject is screened to detect the presence or absence of at least one polymorphic variant of at least one polymorphism of the ABCB1 gene, wherein the polymorphic variant is associated with a decreased susceptibility for QT interval prolongation induced by the depsipeptide, and wherein the polymorphic variant comprises a thymine at position 2677 of SEQ ID NO: 2, or a thymine at position 3435 of SEQ ID NO: 2, or a combination thereof. A diagnosis of a decreased susceptibility of the subject for QT interval prolongation as induced by FK228 is rendered based on the presence or absence of the polymorphic variant of the ABCB1 gene.

Kits compatible with the methods are also provided. In one aspect, a kit is provided that includes a nucleic acid and a drug that binds a protein encoded by ABCB1. The nucleic acid is for use in screening a sample from a subject to detect the presence or absence of at least one polymorphic variant of at least one polymorphism of the ABCB1 gene, wherein the polymorphic variant is associated with an altered susceptibility for a heart rhythm irregularity induced by a drug that binds a protein encoded by the ABCB1 gene, and wherein the nucleic acid specifically binds to ABCB1 sequence comprising the at least one polymorphism or a sequence adjacent to ABCB1 sequence comprising the at least one polymorphism. In one aspect, the polymorphism comprises a polymorphism at position 49,910, 68,894, or 90,871 of SEQ ID NO: 1; or 1236, 2677, or 3435 of SEQ ID NO: 2; or a combination thereof. In another aspect, the drug is FK228.

Use of a drug such as FK228 to manufacture a medicament is also provided. In one aspect, there is a use of a drug that binds a protein encoded by the ABCB1 gene to manufacture a medicament to treat a subject that that has been screened for the presence or absence of at least one polymorphic variant in at least one polymorphism of the ABCB1 gene, wherein the polymorphic variant is associated with an altered susceptibility for a heart rhythm irregularity induced by the drug. In another aspect, the polymorphism comprises a polymorphism at position 49,910, 68,894, or 90,871 of SEQ ID NO: 1; or 1236, 2677, or 3435 of SEQ ID NO: 2, or a combination thereof.

### BRIEF DESCRIPTION OF THE SEVERAL VIEWS OF THE DRAWING(S)

FIG. 1 shows relationships between the area under the curve (AUC) of FK228 and the percentage decrease in platelet count at nadir (PLC) following FK228 treatment. Each symbol represents an individual patient. Data were fit to a sigmoidal maximum effect model (solid line) with 95% confidence intervals (dotted lines).

FIG. 2 shows relationships between ABCB1 genotypes and the baseline corrected QTc interval following FK228 treatment. FIG. 2A shows ABCB1 2677G>T/A genotypes: 1) GG genotype; 2) GT genotype; 3) TT genotype; 4) GA genotype. FIG. 2B shows ABCB1 2677G>T/A-3435C>T genotypes: 1) homozygous variant TT-TT diplotype; 2) a homozygous variant TT genotype at either the 2677G>T/A or the 3435C>T locus; 3) any other 2677G>T/A-3435C>T diplotype that does not correspond to 1) or 2). Each symbol represents an individual patient, and horizontal lines represent median values.

FIG. 3 shows clearance data related to plasma concentration versus time curves for FK228 as a function of ABCB1 2677G>T/A genotype [1) GG genotype; 2) GT genotype; 3) TT genotype; 4) GA genotype] (FIG. 3A), CYP3A4*1B genotype [1), wild-type; 2), heterozygous or homozygous variant] (FIG. 3B), and (C) CYP3A5*3C genotype [1), wild-type or heterozygous; 2), homozygous variant] (FIG. 3C). Each symbol represents an individual patient, and horizontal lines represent median values.

FIG. 4A shows the relationships between ABCB1 genotypes and the baseline corrected QTc interval following FK228 treatment for ABCB1 2677G>T/A and 3435C>T allele combination in group 1 (P=0.011).

FIG. 4B shows the relationships between ABCB1 genotypes and the baseline corrected QTc interval following FK228 treatment for ABCB1 2677G>T/A and 3435C>T allele combination in group 2 (P=0.07).

FIG. 5A shows the relationships between ABCB1 genotypes and the baseline corrected QTc interval following FK228 treatment for (B) ABCB1 3435C>T genotype in group 1 (P=0.15).

FIG. 5B shows the relationships between ABCB1 genotypes and the baseline corrected QTc interval following FK228 treatment for ABCB1 3435C>T genotype in group 2 (P=0.028).

FIG. 6A shows the relationships between ABCB1 genotypes and the baseline corrected QTc interval following FK228 treatment for ABCB1 2677G>A/T genotype in group 1 (P=0.0046).

FIG. 6B shows the relationships between ABCB1 genotypes and the baseline corrected QTc interval following FK228 treatment for ABCB1 2677G>A/T genotype in group 2 (P=0.015). Each symbol represents an individual patient, and horizontal lines represent median values.

FIG. 7A shows the clearance of FK228 as a function of ABCB1 2677G>T/A and 3435C>T allele combination in group 1 (P=0.51). Each symbol represents an individual patient, and horizontal lines represent median values.

FIG. 7B shows the clearance of FK228 as a function of ABCB1 2677G>T/A and 3435C>T allele combination in group 2 (P=0.46). Each symbol represents an individual patient, and horizontal lines represent median values.

### DETAILED DESCRIPTION OF THE INVENTION

A method of screening for an altered susceptibility for a drug-induced heart rhythm irregularity is provided. The method comprises screening a sample from a subject to detect the presence or absence of at least one polymorphic variant of at least one polymorphism of the ABCB1 gene, wherein the polymorphic variant is associated with an altered susceptibility for a heart rhythm irregularity induced by a drug that binds a protein encoded by the ABCB1 gene, and wherein the polymorphism comprises a polymorphism at position 49,910, 68,894, or 90,871 of SEQ ID NO: 1; or 1236, 2677, or 3435 of SEQ ID NO: 2; or a combination thereof. These polymorphisms are also identified as rs1128503, rs2032582, and rs1045642, respectively. The method further comprises diagnosing the altered susceptibility of the subject for the heart rhythm irregularity as induced by the drug based on the presence or absence of the polymorphic variant of the ABCB1 gene. Detecting such a variant does not require detecting the chromosomal DNA or the actual gene. Detection can be of any indicator of such a variant such as any one of, or a combination of, the genome, a genomic fragment, mRNA, a mRNA fragment, cDNA, a cDNA fragment, an encoded polypeptide, and a polypeptide fragment thereof. In an embodiment, the polymorphic variant is associated with an increase or decrease in the expression of ABCB1. In an embodiment, the polymorphic variant is associated with an increase or decrease in an activity of a protein encoded by the ABCB1 gene. That change in activity can be in form of an increased or decreased ability to transport a drug such as FK228. That change can be the result of an alteration of one or more amino acid residues. Such amino acid changes can alter the active site and/or the conformation of the ABCB1 gene product resulting in a more or less efficient drug effluxer. In some embodiments, the polymorphic variant is associated with both a change in expression and a change in an activity of ABCB1.

As used herein, a “gene” is a sequence of DNA present in a cell that directs the expression of a “gene product,” most commonly by transcription to produce RNA and translation to produce protein. An “allele” is a particular form of a gene. The term allele is relevant when there are two or more forms of a particular gene. Genes and alleles are not limited to the open reading frame of the genomic sequence or the cDNA sequence corresponding to processed RNA. A gene and allele can also include sequences upstream and downstream of the genomic sequence such as promoters and enhancers. The term “gene product” or “polymorphic variant allele product” refer to a product resulting from transcription of a gene. Gene and polymorphic variant allele products include partial, precursor, mature transcription products such as pre-mRNA and mRNA, and translation products with or without further processing including, without limitation, lipidation, phosphorylation, glycosylation, other modifications known in the art, and combinations of such processing. RNA may be modified without limitation by complexing with proteins, polyadenylation, splicing, capping or export from the nucleus.

A “polymorphism” is a site in the genome that varies between two or more individuals or within an individual in the case of a heterozygote. The frequency of the variation can be defined above a specific value for inclusion of variations generally observed in a population as opposed to random mutations. Polymorphisms that can be screened according to the invention include variation both inside and outside the open reading frame. When outside the reading frame the polymorphism can occur within 200, 500, 1000, 2000, 3000, 5000, or more of either the 5′ or 3′ end of the open reading frame. When inside the reading frame, the polymorphism may occur within an exon or intron, or overlapping an exon/intron boundary. A polymorphism could also overlap the open reading frame and a sequence outside of that frame. Many polymorphisms have been given a “rs” designation in the SNP database of NCBI's Entrez, some of these designations have been provided herein.

A “polymorphic variant” is a particular form or embodiment of a polymorphism. For example, if the polymorphism is a single nucleotide polymorphism, a particular variant could potentially be an “A” (adenosine), “G” (guanine), “T” (thymine), and “C” (cytosine). When the variant is a “T”, it is understood that a “U” can occur in those instances wherein the relevant nucleic acid molecule is RNA, and vice versa in respect to DNA. The convention “PositionNUC1>NUC2” is used to indicate a polymorphism contrasting one variant from another. For example, 242A>C would refer to a cytosine instead of an adenosine occurring at position 242 of a particular nucleic acid sequence. In some cases, the variation can be to two or more different bases, e.g., 242A>C/T. When 242A>C is used in respect to a mRNA/cDNA, it can also be used to represent the polymorphism as it occurs in the genomic DNA with the understanding that the position number will likely be different in the genome. Sequence and polymorphic location information for both coding domain sequence and genomic sequence is described herein for the genes relevant to the invention. “Polymorphic variant allele” refers to an allele comprising a particular polymeric variant or a particular set of polymorphic variants corresponding to a particular set of polymorphisms. Two alleles can both be considered the same polymorphic variant allele if they share the same variant or set of variants defined by the polymorphic variant allele even though they may differ in respect to other polymorphisms or variation outside the definition. For a mutation at the amino acid level, the convention “AA1PositionAA2” is used. For example, in the context of amino acid sequence, M726L, would indicate that the underlying, nucleotide level polymorphism(s) has resulted in a change from a methionine to a leucine at position 726 in the amino acid sequence.

A “genotype” can refer to a characterization of an individual's genome in respect to one or both alleles and/or one or more polymorphic variants within that allele. A subject can be characterized at the level that the subject contains a particular allele, or at the level of identifying both members of an allelic pair, the corresponding alleles on the set of two chromosomes. One can also be characterized at the level of having one or more polymorphic variants. The term “haplotype” refers to a cis arrangement of two or more polymorphic variants, on a particular chromosome such as in a particular gene. The haplotype preserves the information of the phase of the polymorphic nucleotides—that is, which set of polymorphic variants were inherited from one parent, and which from the other. Wherein methods, materials, and experiments are described for the invention in respect to polymorphic variants, one will understand that can also be adapted for use with an analogous haplotype. A “diplotype” is a haplotype that includes two polymorphisms.

A single nucleotide polymorphism (SNPs) refers to a variation at a single nucleotide location. In some cases the variations at the position could be any one of the four nucleotide bases, in others the variation is some subset of the four bases. For example, the variation could be between either purine base or either pyrimidine base. Simple-sequence length polymophisms (SSLPs) or short tandem repeat polymorphisms (STRPs) involve the repeat of a particular sequence of one or more nucleotides. A restriction fragment length polymorphism (RFLP) is a variation in the genetic sequence that results in the appearance or disappearance of an enzymatic cleavage site depending on which base(s) are present in a particular allele.

A diagnosis for a given susceptibility in accordance with this invention includes detection of homozygosity and/or heterozygosity for a given polymorphism(s). Heterozygosity and homozygosity are relevant wherein the cell, or extract thereof, tested has two chromosomal copies. In other contexts, such as in a sperm or egg, only a single chromosome is present so that the issue of homozygosity or heterozygosity does not directly present itself. In the some embodiments, such as those involving cancer, homozygosity or heterozygosity can be lost or at least obscured because of deletion or inactivation of one of the two gene copies.

In those embodiments where a sample is screened to detect the presence or absence of more than one polymorphic variant associated with a given condition, the combination of the polymorphic variants can be additive, synergistic, or even antagonists in regards to correlative strength—although not overly antagonistic if the susceptibility or drug effect probability is lost. When screening for multiple polymorphisms all can be heterozygous, all can be homozygous, or a combination with one or more polymorphism homozygous, and one or more polymorphism heterozygous, depending on the particular susceptibility relationship for a given set of polymorphic variants and a condition or drug response.

The polymorphic variants described herein can be associated with an altered susceptibility to one or more complications and/or therapeutic treatments. How a polymorphism is mechanistically associated with this susceptibility need not be known for the usefulness and operability of the invention. The polymorphism need not actually cause or contribute to etiology or severity of the condition. In some embodiments, the polymorphism can cause or contribute to the condition. In some embodiments, the polymorphism can serve as a marker for another polymorphism(s) responsible for causing or contributing to the condition. In such a situation, the polymorphism(s) screened for can be in linkage disequilibrium with the responsible polymorphism(s).

In those embodiments where the screened for polymorphic variant(s) is responsible in part or whole for the condition(s), the polymorphic variant(s) can result in a change in the steady state level of mRNA, for example, through a decrease in transcription and/or mRNA stability. Some polymorphic variants can alter the exon/intron boundary and/or effect how splicing occurs. When the polymorphic variant occurs within or overlaps with the protein-encoding sequence of the gene, the polymorphic variant may be silent resulting in no change at the amino acid level, result in a change of one or more amino acid residues, a deletion of one or more amino acids, addition of one or more amino acids, or some combination of such changes. For some polymorphic variants, the result is premature termination of translation. The effect may be neutral, beneficial, or detrimental, or both beneficial and detrimental, depending on the circumstances. Polymorphic variants occurring in noncoding regions can exert phenotypic effects indirectly via influence on replication, transcription, and/or translation. Polymorphic variants in DNA can affect the basal transcription or regulated transcription of a gene locus. Such polymorphic variants may be located in any part of the gene but are most likely to be located in the promoter region, the first intron, or in 5′ or 3′ flanking DNA, where enhancer or silencer elements may be located. A single polymorphism can affect more than one phenotypic trait. A single phenotypic trait may be affected by polymorphisms in different genes. Some polymorphisms predispose an individual to a distinct mutation that is causally related to a certain phenotype.

RNA polymorphic variants can affect a wide range of processes including RNA splicing, polyadenylation, capping, export from the nucleus, interaction with translation initiation, elongation or termination factors, or the ribosome, or interaction with cellular factors including regulatory proteins, or factors that may affect mRNA half life. An effect of polymorphic variants on RNA function can ultimately be measurable as an effect on RNA levels—either basal levels or regulated levels or levels in some abnormal cell state. One method for assessing the effect of RNA polymorphic variants on RNA function is to measure the levels of RNA produced by different alleles in one or more conditions of cell or tissue growth. Such measuring can be done by conventional methods such as Northern blots or RNAase protection assays, which can employ kits available from Ambion, Inc., or by methods such as the Taqman assay, or by using arrays of oligonucleotides or arrays of cDNAs or other nucleic acids attached to solid surfaces, such as a multiplex chip. Systems for arraying cDNAs are available commercially from companies such as Nanogen and General Scanning. Complete systems for gene expression analysis are available from companies such as Molecular Dynamics. See also supplement to volume 21 of Nature Genetics entitled “The Chipping Forecast.” Additional methods for analyzing the effect of polymorphic variants on RNA include secondary structure probing, and direct measurement of half life or turnover. Secondary structure can be determined by techniques such as enzymatic probing with use of enzymes such as T1, T2, and S1 nuclease, chemical probing or RNAase H probing using oligonucleotides. Some RNA structural assays can be performed in vitro or on cell extracts.

To determine if one or more polymorphic variants have an effect on protein levels and/or activity, a variety of techniques may be employed. The in vitro protein activity can be determined by transcription or translation in bacteria, yeast, baculovirus, COS cells (transient), CHO, or study directly in human cells. Further, one can perform pulse chase experiments for the determination of changes in protein stability such as half life measurements. One can manipulate the cell assay to address grouping the cells by genotypes or phenotypes. For example, identification of cells with different genotypes and phenotype can be performed using standardized laboratory molecular biological protocols. After identification and grouping, one skilled in the art could determine whether there exists a correlation between cellular genotype and cellular phenotype.

Correlation between one or more polymorphic variants can be performed for a population of individuals who have been screened for particular polymorphic variants. Correlation can be performed by standard statistical methods including, but not limited to, a chi-squared test. Analyses of polymorphic variants, parametric linkage analysis, non-parametric linkage analysis, etc. and statistically significant correlations between polymorphic form(s) and phenotypic characteristics also can be used.

ATP-binding cassette, sub-family B (MDR/TAP), member 1 (ABCB1) is a member of the ATP-binding cassette (ABC) family of transporters that couple ATP hydrolysis to active transport of substrates out of the cell. ABCB1 has been shown to serve a protective function in several tissues including heart, hematopoietic stem cells, and other tissues, where it effluxes endogenous and exogenous toxins. ABCB1 has the further aliases HGNC:40, ABC20, CD243, CLCS, GP170, MDR1, P-gp, PGY1. ABCB1 has the further designations: P-glycoprotein 1; multidrug resistance 1; colchicin sensitivity; doxorubicin resistance; MDR-1 and multidrug resistance 1. ABCB1 has been assigned Gene ID 5243, and is positioned on chromosome 7 at locus 7q21.1. Further information for ABCB1 is found on the NCBI website in the Entrez Gene database and Online Mendelian Inheritance in Man (OMIM) website under entry “*171050.”

ABCB1 nucleic acid and amino acid sequences relevant to the invention include genomic, cDNA, and fragments thereof. The particular sequences identified herein by sequence identification number and/or accession number are representative of ABCB1 sequences. One of skill in the art can appreciate that there can be variability in the gene or gene fragment distinct from the polymorphism(s) of interest and that such allelic variants still fall within the scope of the invention. As the polymorphism will be reflected in both strands of the DNA, the screening in the context of the invention can involve one or both of the strand sequences. Accordingly, where the sequence for a given strand is provided, the invention also includes the use of its complement.

ABCB1 polymorphisms of particular interest include those known in the art as the 1236, 2677, and 3435 polymorphisms as well as the particular polymorphic variants 1236C>T, 2677G>A/T, and 3435C>T. Other variants of these polymorphisms are also provided as are other polymorphisms in the ABCB1 gene. Polymorphic variants of adenosine (A), guanine (G), cytosine (C), thymine (T), uracil (I) and other applicable nucleotides of each polymorphism are provided. Such is provided not just for ABCB1 polymorphisms, but also for polymorphisms of other genes described herein as well. Other polymorphic variants of these polymorphisms as well as other polymorphisms can also be screened for. The 1236, 2677, and 3435 polymorphisms are given the designations rs1128503, rs2032582, and rs1045642 respectively in the SNP database of NCBI's Entrez. These polymorphisms and particular variants are exemplary and other ABCB1 polymorphisms and variants may also be screened for in accordance with the present invention. The following are representative genomic and cDNA sequences for ABCB1.

The ABCB1 genomic sequence is provided in SEQ ID NO: 1, derived from AY910577 from position 114998 to position 210947 inclusive. The 1236, 2677, and 3435 polymorphisms occur at positions 49,910; 68,894; and 90,871 of SEQ ID NO: 1 (corresponding to positions 164,900; 183884, and 205,861 respectively in AY910577). Screening with a genomic ABCB1 fragment of at least 5, 10, 20, 25, 30, 35, 40, and 50 nucleic acids is within the scope of the invention, as well as, smaller, larger, and intermediate fragments. Fragments can comprise the relevant polymorphism(s) and provide a sequence unique in the human genome. Examples of fragments include the following. SEQ ID NO: 3 comprises the “1236 polymorphism” at position 7. SEQ ID NO: 4 comprises the “2677 polymorphism” at position 7. SEQ ID NO: 5 comprises the “3435 polymorphism” at position 1. SEQ ID NO: 6 comprises the 1236 and 2677 polymorphisms at positions 1 and 18,895 respectively. SEQ ID NO: 7 comprises the 2677 and 3435 polymorphisms at positions 1 and 21,978 respectively. SEQ ID NO: 8 comprises the 1236, 2677, and 3435 polymorphisms at positions 1; 18,895; and 40,962 respectively. Other relevant genomic sequence information includes AF016534, AY910577, CH236949, M29422, M29423, M29424, M29425, M29426, M29427, M29428, M29429, M29430, M29431, M29432, M29433, M29434, M29435, M29436, M29437, M29438, M29439, M29440, M29441, M29442, M29443, M29444, M29445, M29446, M29447, M37724, M37725, X58723, fragments thereof, and sequences comprising the same.

The ABCB1 cDNA sequence is provided in SEQ ID NO: 2, derived from NM_{—}000927. The 1236, 2677, and 3435 polymorphisms occur at positions 1236, 2677, and 3435 of SEQ ID NO: 2. Screening with a cDNA ABCB1 fragment of at least 5, 10, 20, 25, 30, 35, 40, and 50 nucleic acids is within the scope of the invention, as well as, smaller, larger, and intermediate fragments. Fragments can comprise the relevant polymorphism(s) and provide a sequence unique in the human genome. Examples of fragments include the following. SEQ ID NO: 9 comprises the 1236 polymorphism at position 7. SEQ ID NO: 10 comprises the 2677 polymorphism at position 7. SEQ ID NO: 11 comprises the 3435 polymorphism at position 507. SEQ ID NO: 12 comprises the 1236 and 2677 polymorphisms at positions 1 and 1,442 respectively. SEQ ID NO: 13 comprises the 2677 and 3435 polymorphisms at positions 1 and 759 respectively. SEQ ID NO: 14 comprises the 1236, 2677, and 3435 polymorphisms at positions 1, 1,442, and 2,200 respectively. Other relevant sequence information include mRNA sequences AB208970, AF016535, AY425005, AY425006, BQ720763, BQ882401, BX509020, CB164676, M14758, fragments thereof, and sequences comprising the same.

The translation of the ABCB1 cDNA coding region is provided in SEQ ID NO: 15. Position 893 of SEQ ID NO: 15 can be amino acids such as alanine, serine, or threonine corresponding to the polymorphic variants of the 2677 polymorphism. Position 893 can also be any other amino acid. Fragments of the ABCB1 polypeptide sequence are also within the scope of the invention such as fragment recognized by ABCB1 specific antibodies and fragments recognized by antibodies specific to particular variants as manifested in the polypeptide sequence. Other relevant ABCB1 polypeptide sequence information includes AAB70218, AAW82430, EAL24173, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA59576, AAA88047, AAA88048, CAA41558, BAD92207, AAB69423, AAR91621, AAR91622, AAA59575, P08183, Q59GY9, Q6TBL4, fragments thereof, and sequences comprising the same.

In one aspect the polymorphic variant screened for is present in a single chromosomal copy of the gene, and wherein heterozygosity is associated with an altered susceptibility for the heart rhythm irregularity. In some embodiments, the heterozygosity for polymorphic variants of two or more polymorphisms is associated with an altered susceptibility for the heart rhythm irregularity. In another aspect, the polymorphic variant is present in both chromosomal copies of the gene, wherein homozygosity of the polymorphic variant is associated with an altered susceptibility for the heart rhythm irregularity if homozygosity of the polymorphic variant is detected. In some embodiments, homozygosity for polymorphic variants of two or more polymorphisms is associated with an altered susceptibility for the heart rhythm irregularity.

In one aspect, the method of screening is performed on a sample comprising a nucleic acid selected from the group consisting of (a) a nucleic acid encoding ABCB1, (b) a fragment of (a) comprising at least 5, 10, 15, 20, 25, 30, 35, 40, 45, 50, 75, 100, 150, 200, 250, 500, 1000, or 10,000 contiguous nucleotides of (a) wherein the contiguous nucleotides comprise the polymorphism, (c) a complement of (a) or (b), and (d) a combination of two or more of (a), (b), and (c). In some embodiments, the nucleic acid encoding ABCB1 comprises SEQ ID NOS: 1, 2, or a combination thereof. The polymorphism can be a polymorphism at position 49,910, 68,894, or 90,871 of SEQ ID NO: 1; or 1236, 2677, or 3435 of SEQ ID NO: 2; or a combination thereof.

The method can be performed by screening for one or more polymorphic variants of a single polymorphism of ABCB1. In some embodiments, the polymorphism is a polymorphism at position 49,910 of SEQ ID NO: 1; or 1236 of SEQ ID NO: 2, or a combination thereof. In such cases, the nucleic acid can comprise the sequence of SEQ ID NOS: 3, 9, or a combination thereof. In some embodiments, the polymorphism is a polymorphism at position 68,894 of SEQ ID NO: 1, or 2677 of SEQ ID NO: 2, or a combination thereof. In such cases, the nucleic acid can comprise the sequence of SEQ ID NOS: 4, 10, or a combination thereof. In some embodiments, the polymorphism is a polymorphism at position 90,871 of SEQ ID NO: 1, 3435 of SEQ ID NO: 2, or a combination thereof. In such cases, the nucleic acid can comprise the sequence of SEQ ID NOS: 5, 11, or a combination thereof.

The method can be performed by screening for one or more polymorphic variants of two or more polymorphisms of ABCB1. In some embodiments, the nucleic acid comprises first and second polymorphisms wherein the first polymorphism is a polymorphism at position 49,910 of SEQ ID NO: 1; or 1236 of SEQ ID NO: 2, or a combination thereof and the second polymorphism is a polymorphism at position 68,894 of SEQ ID NO: 1, or 2677 of SEQ ID NO: 2, or a combination thereof. In some such cases, the nucleic acid comprises the sequence of SEQ ID NO: 6, 12, or a combination thereof. In some embodiments, the nucleic acid comprises first and second polymorphisms wherein the first polymorphism is a polymorphism at position 68,894 of SEQ ID NO: 1, 2677, of SEQ ID NO: 2, or a combination thereof the second polymorphism is a polymorphism at position 90,871 of SEQ ID NO: 1, 3435 of SEQ ID NO: 2, or a combination thereof. In such cases, the nucleic acid can comprise the sequence of SEQ ID NOS: 7, 13, or a combination thereof.

In some embodiments, the nucleic acid comprises first, second and third polymorphisms wherein the first polymorphism is a polymorphism at position 49,910 of SEQ ID NO: 1; or 1236 of SEQ ID NO: 2, or a combination thereof, the second polymorphism is a polymorphism at position 68,894 of SEQ ID NO: 1, or 2677 of SEQ ID NO: 2, or a combination thereof, and the third polymorphism is a polymorphism at position 90,871 of SEQ ID NO: 1, 3435 of SEQ ID NO: 2, or a combination thereof. In such cases, the nucleic acid can comprise the sequence of SEQ ID NOS: 8, 14, or a combination thereof.

The method can be performed by screening wherein the polymorphic variant screened for is a thymine at least one polymorphism. In some embodiments, the polymorphism comprises a polymorphism at position 49,910 of SEQ ID NO: 1; or 1236 of SEQ ID NO: 2, or a combination thereof, and the subject is homozygous for thymine at that position. In some embodiments, the polymorphism comprises a polymorphism at position 68,894 of SEQ ID NO: 1, or 2677 of SEQ ID NO: 2, or a combination thereof and the subject is homozygous for thymine at that position. In some embodiments, the polymorphism comprises first, second, and third polymorphisms wherein the first polymorphism is a polymorphism at position 68,894 of SEQ ID NO: 1, 2677, of SEQ ID NO: 2, or a combination thereof the second polymorphism is 2677, and the third polymorphism is a polymorphism at position 90,871 of SEQ ID NO: 1, 3435 of SEQ ID NO: 2, or a combination thereof, and wherein the subject is homozygous for thymine at both positions.

Polymorphic variants to be screened for are principally located in or in close proximity to the ABCB1 gene. Representative, polymorphic variants that can be tested for in addition to ABCB1 variant(s), include those associated with the following described genes without limitation to polymorphic variant, polymorphism, allelic variant, or gene. In some embodiments, the screened for polymorphic variants are correlated with the same disease. In some embodiments, the screened for polymorphic variants are correlated with different diseases.

The invention provides screening for polymorphic variants in genes and sequence other than ABCB1 sequences. In some embodiments, the additional variant is in a sequence associated with another drug resistance related gene. In some embodiments, one or more variant in one or more organic anion transporting protein (OATP) family members and/or multidrug resistance associated protein ABCC1 (MRP1) are screened for. In some embodiments, the additional polymorphic variant is in a cytochrome P450 gene. The polymorphic variant can be associated with altered metabolism of the drug.

Cytochrome P450, Family 3, Subfamily A, Polypeptide 4 (CYP3A4) is a P450 enzyme for which FK228 is a substrate. CYP3A4 has the further alias HGNC:2637, CP33, CP34, CYP3A, CYP3A3, HLP, NF-25, P450C3, and P450PCN1. CYP3A4 has the further designations P450-III, steroid inducible; cytochrome P450, subfamily IIIA (niphedipine oxidase), polypeptide 3; cytochrome P450, subfamily IIIA (niphedipine oxidase), polypeptide 4; cytochrome P450, subfamily IIIA, polypeptide 4; glucocorticoid-inducible P450; and nifedipine oxidase. CYP3A4 has been assigned Gene ID 1576, and is positioned on chromosome 7 at locus 7q21.1. Further information for CYP3A4 is found on the NCBI website in the Entrez Gene database and Online Mendelian Inheritance in Man (OMIM) website under entry *124010. Polymorphic variants that can be screened for in addition to one or more of the ABCB1 polymorphic variants relevant to the invention include the polymorphic variant CYP3A4*1B.

CYP3A4 nucleic acid and amino acid sequences relevant to the invention include genomic, cDNA, and fragments thereof. The particular sequences identified herein by sequence identification number and/or accession number are representative of CYP3A4 sequences. One of skill in the art can appreciate that there can be variability in the gene or gene fragment distinct from the polymorphism(s) of interest and that such allelic variants still fall within the scope of the invention. As the polymorphism will be reflected in both strands of the DNA, the screening in the context of the invention can involve one or both of the strand sequences. Accordingly, where the sequence for a given strand is provided, the invention also includes the use of its complement. Screening with a CYP3A4 nucleic acid fragment of at least 5, 10, 20, 25, 30, 35, 40, and 50 nucleic acids is within the scope of the invention, as well as, smaller, larger, and intermediate fragments. Fragments can comprise the relevant polymorphism(s) and provide a sequence unique in the human genome. Examples of relevant cytochromes include CYP3A4 and CYP3A5. In some embodiments, the allelic variant CYP3A4*1B is screened for. In some embodiments, the alleleic variant CYP3A5*3C is screened for. Examples of CYP3A4 genomic sequences include AF209389, AF280107, AF307089, CH236956, D11131, fragments thereof, and sequences comprising the same. Examples of CYP3A4 mRNA sequences include AF182273, AJ563375, AJ563376, AJ563377, BC069418, D00003, J04449, M13785, M14096, M18907, X12387, fragments thereof, and sequences comprising the same. Examples of CYP3A4 amino acid sequences include AAF21034, AAG32290, AAG53948, EAL23866, AAF13598, CAD91343, CAD91645, CAD91345, AAH69418, BAA00001, AAA35747, AAA35742, AAA35744, AAA35745, CAA30944, P05184, P08684, Q6GRK0, Q7Z448, Q86SK2, Q86SK3, Q9BZM0, fragments thereof, and sequences comprising the same.

The following are representative sequences for CYP3A4. CYP3A4 has a 5′ genomic flanking sequence (SEQ ID NO: 16 as derived from D11131) and a genomic sequence beginning with exon 1 (SEQ ID NO: 17 as derived from positions 148,895 to 176,090 of NG_{—}000004). CYP3A4*1B is the allelic variant of CYP3A4 of particular relevance to the present invention. This allelic variant is found in the 5′ genomic flanking sequence at position 810 of SEQ ID NO: 16, and is the result of an A>G variance from the consensus sequence to the variant. Other nucleotides can also be at this position. The polymorphism at this position has been designated rs2740574. SEQ ID NO: 18 provides the cDNA sequence for CYP3A4. This sequence is derived from the complete CYP3A4 cDNA sequence, coding strand which has the Accession #M18907. The CYP3A4*1B polymorphism is not found in this sequence as it is prior to the transcription start site and is not found expressed in the mRNA. SEQ ID NO: 19 provides the polypeptide sequence for CYP3A4. This sequence is derived from the complete CYP3A4 protein sequence, which has the Accession #NP_{—}059488.

Cytochrome P450, Family 3, Subfamily A, Polypeptide 5 (CYP3A5) is a P450 enzyme for which FK228 is a substrate. CYP3A5 has the further aliases HGNC:2638, CP35, P450PCN3, and PCN3. CYP3A5 has the further designations aryl hydrocarbon hydroxylase; cytochrome P-450; cytochrome P450, subfamily IIIA (niphedipine oxidase), polypeptide 5; flavoprotein-linked monooxygenase; microsomal monooxygenase; niphedipine oxidase; and xenobiotic monooxygenase. CYP3A5 has been assigned Gene ID 1577, and is positioned on chromosome 7 at locus 7q21.1. Further information for CYP3A5 is found on the NCBI website in the Entrez Gene database and Online Mendelian Inheritance in Man (OMIM) website under entry *605325. Polymorphic variants that can be screened for in addition to one or more of the ABCB1 polymorphic variants relevant to the invention include the polymorphic variant CYP3A5*3C.

CYP3A5 nucleic acid and amino acid sequences relevant to the invention include genomic, cDNA, and fragments thereof. The particular sequences identified herein by sequence identification number and/or accession number are representative of CYP3A5 sequences. One of skill in the art can appreciate that there can be variability in the gene or gene fragment distinct from the polymorphism(s) of interest and that such allelic variants still fall within the scope of the invention. As the polymorphism will be reflected in both strands of the DNA, the screening in the context of the invention can involve one or both of the strand sequences. Accordingly, where the sequence for a given strand is provided, the invention also includes the use of its complement. Screening with a CYP3A5 nucleic acid fragment of at least 5, 10, 20, 25, 30, 35, 40, and 50 nucleic acids is within the scope of the invention, as well as, smaller, larger, and intermediate fragments. Fragments can comprise the relevant polymorphism(s) and provide a sequence unique in the human genome. Examples of CYP3A5 genomic sequences include AC005020, AF280107, AF355803, CH236956, L35912, fragments thereof, and sequences comprising the same. Examples of CYP3A5 mRNA sequences include AF355801, AJ563378, AJ563379, AK223008, BC022298, BC025176, BC026255, BC033862, BX537676, J04813, L26985, fragments thereof, and sequences comprising the same. Examples of CYP3A5 amino acid sequences include AAS02016, AAG32288, AAK73691, EAL23868, AAB00083, AAK73689, CAD91347, CAD91647, CAD91649, BAD96728, AAH33862, CAD97807, AAA02993, P20815, Q53GC3, Q75MV0, Q7Z3N0, Q7Z446, Q7Z447, Q86SK1, Q96RK6, fragments thereof, and sequences comprising the same.

The following are representative sequences for CYP3A5. The genomic DNA for CYP3A5 is shown in SEQ ID NO: 20 (corresponding to positions 253,080-288,849. The cDNA for CYP3A5 is provided in SEQ ID NO: 21 as derived from BC033862. CYP3A5*1B is the allelic variant of CYP3A5 of particular relevance to the present invention. The cDNA sequence for CYP3A5*1B is provided in SEQ ID NO: 22. The CYP3A5*3C allelic variant is a result of an A>G variance at position 7087 of SEQ ID NO: 20 (260167 of NG_{—}000004). Other nucleotides can also be at this position. The polymorphism at this position has been designated rs776746. The CYP3A5*3C polymorphism is contained in an intron and is not found expressed in the consensus mRNA sequence. However, the CYP3A5*3C polymorphic variant results in the inclusion of intron 3 in the spliced mRNA as it is contained within a cryptic splice site. The mRNA and cDNA corresponding to the CYP3A5*3C polymorphism therefore includes intron 3 (bases 258551-260403 in the CYP3A5 genomic DNA sequence; Accession #NG_{—}000004) between bases 307 and 308 in SEQ ID NO: 21. The CYP3A5*3C polymorphism in the cDNA sequence, SEQ ID NO: 22, occurs at position 1923.

Amino acid sequences for CYP3A5 and CYP3A5*1B are provided in SEQ ID NOS: 23 and 24 respectively. The following sequence contains a total of 502 amino acids. This sequence is derived from the complete CYP3A5 protein sequence, which has the Accession # NP_{—}000768. The protein is not expressed in individuals homozygous for the CYP3A5*3C polymorphism as the incorporation of intronic DNA results in premature truncation of the protein after amino acid 102 due to the presence of a stop codon within intron 3.

The invention also includes use of other polymorphic variants of the genes and proteins described herein. Use of both the nucleic acids described herein and their complements are within the scope of the invention. In connection with the provision and description of nucleic acid sequences, the references herein to gene names and to GenBank and OMIM reference numbers provide the relevant sequences, recognizing that the described sequences will, in most cases, also have other corresponding allelic variants. Although the referenced sequences may contain sequencing error, such error does not interfere with identification of a relevant gene or portion of a gene, and can be readily corrected by redundant sequencing of the relevant sequence (preferably using both strands of DNA). Nucleic acid molecules or sequences can be readily obtained or determined utilizing the reference sequences. Molecules such as nucleic acid hybridization probes and amplification primers can be provided and are described by the selected portion of the reference sequence with correction if appropriate. In some embodiments, probes comprise 5, 6, 10, 12, 13, 14, 15, 16, 17, 18, 19, 20, 23, 25, 27, 30, 35, 40, 45, 50, or more nucleotides.

The terms “disease” or “condition” are commonly recognized in the art and designate the presence of signs and/or symptoms in an individual or patient that are generally recognized as abnormal. Unless indicated as otherwise, the terms “disease,” “disease state,” condition,” “disorder,” and “complication” can be used interchangeably. Diseases or conditions can be diagnosed and categorized based on pathological changes. Signs can include any objective evidence of a disease such as changes that are evident by physical examination of a patient or the results of diagnostic tests which may include, among others, laboratory tests to determine the presence of polymorphic variants or variant forms of certain genes in a patient. Symptoms can include a patient's perception of an abnormal condition that differs from normal function, sensation, or appearance, which may include, for example, physical disabilities, morbidity, pain, and other changes from the normal condition experienced by an individual. Various diseases or conditions include, but are not limited to, those categorized in medical texts.

Unless otherwise indicated, the term “suffering from a disease or condition” can refer to a person that currently has signs and symptoms, or is more likely to develop such signs and symptoms than a normal person in the population. For example, a person suffering from a condition can include a developing fetus, a person subject to a treatment or environmental condition that enhances the likelihood of developing the signs or symptoms of a condition, or a person who is being given or will be given a treatment that increases the likelihood of the person developing a particular condition. Methods of the invention relating to treatments of patients can include primary treatments directed to a presently active disease or condition, secondary treatments that are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

Combined detection of several polymorphic variants typically increases the probability of an accurate diagnosis. Analysis of the polymorphisms of the invention can be combined with that of other polymorphisms or other risk factors such as family history. Polymorphisms can be used to diagnose a disease at the pre-symptomatic stage, as a method of post-symptomatic diagnosis, as a method of confirmation of diagnosis or as a post-mortem diagnosis. Ethical issues to be considered in screening and diagnosis are discussed generally in Reich, et al., Genet. Med., 5:133-143 (2003).

In some embodiments, the sample screened is from a subject who has previously experienced a heart rhythm irregularity. In some embodiment, the heart rhythm irregularity is a cardiac arrhythmia. The heart rhythm irregularity comprises at least one member selected from the group consisting of asymptomatic dysrhythmias and ventricular arrthymias. The heart rhythm irregularity can be characterized by at least one of ST/T wave flattening, torsade de pointes, and QT interval prolongation.

“Prolonged QT interval,” “QT interval prolongation” or “QT interval elongation” refers to the QT interval measured from QRS onset to T wave offset (QTo) and from QRS onset to T wave peak (QTm) adjusted to a heart rate of 60 beats per minute, which is QTc. “QTc” is also referred to as the Bazett corrected QT interval. See, e.g., Kligfield et al., J. Am. Coll. Cardiol, 28: 1547-55 (1996). Prolonged QT intervals can be induced directly or indirectly by one or more polymorphic variant of one or more polymorphism.

“Torsades de Pointes” or “TdP” is an uncommon variant of ventricular tachycardia (VT). The underlying etiology and management of TdP can be different from the more common ventricular tachycardia. TdP is a polymorphous ventricular tachycardia in which the morphology of the QRS complexes vary from beat to beat. The ventricular rate can range from about 150/min to about 250/min. In some cases, there is a constantly changing wave form, but there may not be regularity to the axis changes. Q-T interval can be markedly increased (usually to 600 msec or greater). Cases of polymorphic VT, which are not associated with a prolonged Q-T interval, can be treated as generic VT. TdP can occur in bursts that are not sustained. Accordingly, one can employ a rhythm strip showing the patient's base-line Q-T prolongation

Any applicable method or combination of methods can be used to screen for polymorphic variants in a sample. Screening methods can utilize one or more of a nucleic acid array, allele-specific-oligonucleotide (ASO) hybridization, PCR-RFLP analysis, PCR., a single-strand conformation polymorphic variant (SSCP) technique, an amplification refractory mutation system (ARMS) technique, nucleotide sequencing, an antibody specific to a polypeptide encoded by the polymorphic variant containing gene, mass spectrometry, and combinations thereof. The sample screened can comprise at least one of genomic DNA, cDNA, mRNA, other DNA, other RNA, a fragment thereof, and a combination thereof. The sample screened can be derived from any number of single or combined sample and/or cell or tissue sources. In some embodiments, the screened sample comprises blood. The sample need not be directly from a subject. One or more steps can be performed on the sample prior to, subsequent to, and/or as part of the screening. For example, one or more of the following: mRNA from a subject can be converted to cDNA, cDNA can be amplified using PCR, amplified DNA can be sequenced and/or assayed with one or more restriction enzymes, etc.

The molecules and probes relevant to the invention can be used in screening techniques. A variety of screening techniques are known in the art for detecting the presence of one or more copies of one or more polymorphic variants in a sample or from a subject. Many of these assays have been reviewed by Landegren et al., Genome Res., 8:769-776, 1998. Determination of polymorphic variants within a particular nucleotide sequence among a population can be determined by any method known in the art, for example and without limitation, direct sequencing, restriction length fragment polymorphism (RFLP), single-strand conformational analysis (SSCA), denaturing gradient gel electrophoresis (DGGE) [see, e.g., Van Orsouw et al., Genet Anal., 14(5-6):205-13 (1999)], heteroduplex analysis (HET) [see, e.g., Ganguly A, et al., Proc Natl Acad Sci USA. 90 (21):10325-9 (1993)], chemical cleavage analysis (CCM) [see, e.g., Ellis T P, et al., Human Mutation 11(5):345-53 (1998)] (either enzymatic as with T4 Endonuclease 7, or chemical as with osmium tetroxide and hydroxylamine) and ribonuclease cleavage. Screening for polymorphic variants can be performed when a polymorphic variant is already known to be associated with a particular disease or condition. In some embodiments, the screening is performed in pursuit of identifying one or more polymorphic variants and determining whether they are associated with a particular disease or condition.

In respect to DNA, polymorphic variant screening can include genomic DNA screening and/or cDNA screening. Genomic polymorphic variant detection can include screening the entire genomic segment spanning the gene from the transcription start site to the polyadenylation site. In some embodiments, genomic polymorphic variant detection can include the exons and some region around them containing the splicing signals, for example, but not all of the intronic sequences. In addition to screening introns and exons for polymorphic variants, regulatory DNA sequences can be screened for polymorphic variants. Promoter, enhancer, silencer and other regulatory elements have been described in human genes. The promoter is generally proximal to the transcription start site, although there may be several promoters and several transcription start sites. Enhancer, silencer and other regulatory elements can be intragenic or can lie outside the introns and exons, possibly at a considerable distance, such as 100 kb away. Polymorphic variants in such sequences can affect basal gene expression or regulation of gene expression.

The presence or absence of the at least one polymorphic variant can be determined by nucleotide sequencing. Sequencing can be carried out by any suitable method, for example, dideoxy sequencing [Sanger et al., Proc. Natl. Acad. Sci. USA, 74:5463-5467 (1977)], chemical sequencing [Maxam and Gilbert, Proc. Natl. Acad. Sci. USA, 74:560-564, (1977)] or variations thereof. Methods for sequencing can also be found in Ausubel et al., eds., Short Protocols in Molecular Biology, 0.3rd ed., Wiley, 1995 and Sambrook et al., Molecular Cloning, 2nd ed., Chap. 13, Cold Spring Harbor Laboratory Press, 1989. The sequencing can involve sequencing of a portion or portions of a gene and/or portions of a plurality of genes that includes at least one polymorphic variant site, and can include a plurality of such sites. The portion can be of sufficient length to discern whether the polymorphic variant(s) of interest is present. In some embodiments the portion is 500, 250, 100, 75, 65, 50, 45, 35, 25 nucleotides or less in length. Sequencing can also include the use of dye-labeled dideoxy nucleotides, and the use of mass spectrometric methods. Mass spectrometric methods can also be used to determine the nucleotide present at a polymorphic variant site.

RFLP analysis is useful for detecting the presence of genetic variants at a locus in a population when the variants differ in the size of a probed restriction fragment within the locus, such that the difference between the variants can be visualized by electrophoresis [see, e.g. U.S. Pat. Nos. 5,324,631 and 5,645,995]. Such differences will occur when a variant creates or eliminates a restriction site within the probed fragment. RFLP analysis is also useful for detecting a large insertion or deletion within the probed fragment. RFLP analysis is useful for detecting, for example, an Alu or other sequence insertion or deletion.

Single-strand conformational polymorphisms (SSCPs) can be detected in <220 bp PCR amplicons with high sensitivity. SSCP is usually paired with a DNA sequencing method, because the SSCP method does not provide the nucleotide identity of polymorphic variants. The SSCP technique can be used on genomic DNA as well as PCR amplified DNA as well. [Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6.]

Another method for detecting polymorphic variants is the T4 endonuclease VII (T4E7) mismatch cleavage method: T4E7 specifically cleaves heteroduplex DNA containing single base mismatches, deletions or insertions. Denaturing gradient gel electrophoresis (DGGE) can detect single base mutations based on differences in migration between homoduplexes and heteroduplexes [Myers et al., Nature, 313:495-498 (1985)]. In heteroduplex analysis (HET) [Keen et al., Trends Genet. 7:5 (1991)], genomic DNA is amplified by the polymerase chain reaction followed by an additional denaturing step that increases the chance of heteroduplex formation in heterozygous individuals. The PCR products are then separated on Hydrolink gels where the presence of the heteroduplex is observed as an additional band. Chemical cleavage analysis (CCM) is based on the chemical reactivity of thymine (T) when mismatched with cytosine, guanine or thymine and the chemical reactivity of cytosine(C) when mismatched with thymine, adenine or cytosine [Cotton et al., Proc. Natl. Acad. Sci. USA, 85:4397-4401 (1988)]. Ribonuclease cleavage involves enzymatic cleavage of RNA at a single base mismatch in an RNA:DNA hybrid (Myers et al., Science 230:1242-1246, 1985).

In addition to the physical methods described herein and others known to those skilled in the art, see, for example, Housman, U.S. Pat. No. 5,702,890; Housman et al., U.S. patent application Ser. No. 09/045,053, polymorphisms can be detected using computational methods, involving computer comparison of sequences from two or more different biological sources, which can be obtained in various ways, for example from public sequence databases. The term “polymorphic variant scanning” refers to a process of identifying sequence polymorphic variants using computer-based comparison and analysis of multiple representations of at least a portion of one or more genes. Computational polymorphic variant detection involves a process to distinguish true polymorphic variants from sequencing errors or other artifacts, and thus does not require perfectly accurate sequences. Such scanning can be performed in a variety of ways as known to those skilled in the art, preferably, for example, as described in U.S. patent application Ser. No. 09/300,747. The “gene” and “SNP” databases of Pubmed Entrez can also be utilized for identifying polymorphisms.

Genomic and cDNA sequences can both or in the alternative be used in identifying polymorphisms. Genomic sequences are useful where the detection of polymorphism in or near splice sites is sought, such polymorphism can be in introns, exons, or overlapping intron/exon boundaries. Nucleic acid sequences analyzed may represent full or partial genomic DNA sequences for a gene or genes. Partial cDNA sequences can also be utilized although this is less preferred. As described herein, the polymorphic variant scanning analysis can utilize sequence overlap regions, even from partial sequences. While the present description is provided by reference to DNA, for example, cDNA, some sequences can be provided as RNA sequences, for example, mRNA sequences.

Interpreting the location of the polymorphic variant in the gene can depend on the correct assignment of the initial ATG of the encoded protein (the translation start site). The correct ATG can be incorrect in GenBank, but that one skilled in the art will know how to carry out experiments to definitively identify the correct translation initiation codon (which is not always an ATG). In the event of any potential question concerning the proper identification of a gene or part of a gene, due for example, to an error in recording an identifier or the absence of one or more of the identifiers, the priority for use to resolve the ambiguity is GenBank accession number, OMIM identification number, HUGO identifier, common name identifier.

Allele and genotype frequencies can be compared between cases and controls using statistical software (for example, SAS PROC NLMIXED). The odds ratios can be calculated using a log linear model by the delta method [Agresti, New York: John Wiley & Sons (1990)] and statistical significance is assessed via the chi-square test. Likelihood ratios (G2) were used to assess goodness of fit of different models i.e., G2 provides a measure of the reliability of the odds ratio; small G2 P-values indicate a poor fit to the model being tested. Combined genotypes can be analyzed by estimating, maximum likelihood estimation, the gamete frequencies in cases and controls using a model of the four combinations of alleles as described by Weir, Sunderland, Mass.: Sinauer (1996). Gene-gene interactive effects can be tested using a series of non-hierarchical logistic models [Piegorsch et al., Stat. Med. 13:153-162 (1994)] to estimate interactive dominant and recessive effects. A sample size as large as possible from a relatively homogenous population to minimize variables outside the focus of the study.

Genomic DNA can be extracted from cases and controls using the QIAamp DNA Blood Mini Kit from Qiagen, UK. Genotyping of polymorphisms can be performed using PCR-RFLP (Restriction Fragment Length Polymorphism) using appropriate restriction sites for the gene(s) being studied [Frosst et al., Nature Genet., 10:111-113 (1995); Hol et al., Clin. Genet., 53:119-125 (1998); Brody et al., Am. J. Hum. Genet., 71:1207-1215 (2002)]. A polymorphism may be genotyped using an allele-specific primer extension assay and scored by matrix-assisted laser desorption/ionization-time of flight (MALDI-TOF) mass spectrometry (Sequenom, San Diego). Appropriate controls should be included in all assays. genotyping consistency can be tested by analyzing between 10-15% of samples in duplicate.

One type of assay has been termed an array hybridization assay, an example of which is the multiplexed allele-specific diagnostic assay (MASDA) (U.S. Pat. No. 5,834,181; Shuber et al., Hum. Molec. Genet., 6:337-347 (1997). In MASDA, samples from multiplex PCR are immobilized on a solid support. A single hybridization is conducted with a pool of labeled allele specific oligonucleotides (ASO). The support is then washed to remove unhybridized ASOs remaining in the pool. Labeled ASO remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end such that the dye pair interact via fluorescence resonance energy transfer (FRET).

An alternative to the TaqMan assay is the molecular beacons assay [U.S. Pat. No. 5,925,517; Tyagi et al., Nature Biotech., 16:49-53 (1998)]. High throughput screening for SNPs that affect restriction sites can be achieved by Microtiter Array Diagonal Gel Electrophoresis (MADGE) (Day and Humphries, Anal. Biochem., 222:389-395, 1994).

Additional assays depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA) [Sommer et al., Mayo Clin. Proc., 64:1361-1372 (1989); Sarker et al., Anal. Biochem. (1990), allele-specific amplification (ASA), allele-specific PCR, and amplification refractory mutation system (ARMS) [Newton et al., Nuc. Acids Res. (1989); Nichols et al., Genomics (1989); Wu et al., Proc. Natl. Acad. Sci. USA, (1989)]. In these methods, an oligonucleotide primer is designed that perfectly matches one allele but mismatches the other allele at or near the 3′ end. This results in the preferential amplification of one allele over the other. By using three primers that produce two differently sized products, it can be determine whether an individual is homozygous or heterozygous for the mutation [Dutton and Sommer, Bio Techniques, 11:700-702 (1991)]. In another method, termed bi-PASA, four primers are used; two outer primers that bind at different distances from the site of the SNP and two allele specific inner primers [Liu et al., Genome Res., 7:389-398 (1997)].

Another technique is the oligonucleotide ligation assay [Landegren et al., Science, 241:1077-1080 (1988)] and the ligase chain reaction [LCR; Barany, Proc. Natl. Acad. Sci. USA, 88:189-193 (1991)]. In OLA, the sequence surrounding the SNP is first amplified by PCR, whereas in LCR, genomic DNA can by used as a template. In one method for mass screening based on the OLA, amplified DNA templates are analyzed for their ability to serve as templates for ligation reactions between labeled oligonucleotide probes [Samotiaki et al., Genomics, 20:238-242, (1994)]. In alternative gel-based OLA assays, polymorphic variants can be detected simultaneously using multiplex PCR and multiplex ligation [U.S. Pat. No. 5,830,711; Day et al., Genomics, 29:152-162 (1995); Grossman et al., Nuc. Acids Res., 22:4527-4534, (1994)]. A further modification of the ligation assay has been termed the dye-labeled oligonucleotide ligation (DOL) assay [U.S. Pat. No. 5,945,283; Chen et al., Genome Res., 8:549-556 (1998)].

In another method for the detection of polymorphic variants termed minisequencing, the target-dependent addition by a polymerase of a specific nucleotide immediately downstream (3′) to a single primer is used to determine which allele is present (U.S. Pat. No. 5,846,710). Using this method, several variants can be analyzed in parallel by separating locus specific primers on the basis of size via electrophoresis and determining allele specific incorporation using labeled nucleotides. Determination of individual variants using solid phase minisequencing has been described by Syvanen et al., Am. J. Hum. Genet., 52:46-59 (1993). Minisequencing has also been adapted for use with microarrays [Shumaker et al., Human Mut., 7:346-354 (1996)]. In a variation of this method suitable for use with multiplex PCR, extension is accomplished with the use of the appropriate labeled ddNTP and unlabeled ddNTPs [Pastinen et al., Genome Res., 7:606-614 (1997)]. Solid phase minisequencing has also been used to detect multiple polymorphic nucleotides from different templates in an undivided sample [Pastinen et al., Clin. Chem., 42:1391-1397 (1996)]. Fluorescence resonance energy transfer (FRET) has been used in combination with minisequencing to detect polymorphic variants [U.S. Pat. No. 5,945,283; Chen et al., Proc. Natl. Acad. Sci. USA, 94:10756-10761 (1997)].

Many of the methods described involve amplification of DNA from target samples. This can be accomplished by e.g., PCR. Other suitable amplification methods include the ligase chain reaction (LCR) [see Wu and Wallace, Genomics 4, 560 (1989), Landegren et al., Science 241, 1077 (1988)], transcription amplification [Kwoh et al., Proc. Natl. Acad. Sci. USA 86, 1173 (1989)], self-sustained sequence replication [Guatelli et al., Proc. Nat. Acad. Sci. USA, 87, 1874 (1990)] and nucleic acid based sequence amplification (NASBA).

Single base extension methods are described by e.g., U.S. Pat. No. 5,846,710, U.S. Pat. No. 6,004,744, U.S. Pat. No. 5,888,819 and U.S. Pat. No. 5,856,092. Amplification products generated using the polymerase chain reaction can be analyzed by the use of denaturing gradient gel electrophoresis. Different alleles can be identified based on the different sequence-dependent melting properties and electrophoretic migration of DNA in solution. [Erlich, ed., PCR Technology, Principles and Applications for DNA Amplification, (W. H. Freeman and Co, New York, (1992)), Chapter 7.]

Arrays provide a high throughput technique that can assay a large number of polynucleotides in a sample. Techniques for constructing arrays and methods of using these arrays are described in, for example, Schena et al., (1996) Proc Natl Acad Sci USA. 93(20):10614-9; Schena et al., (1995) Science 270(5235):467-70; Shalon et al., (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

Screening may also be based on the functional or antigenic characteristics of the protein. Immunoassays designed to detect predisposing polymorphisms in proteins relevant to the invention can be used in screening. Antibodies specific for a polymorphism variant or gene products may be used in screening immunoassays. A sample is taken from a subject. Samples, as used herein, include biological fluids such as tracheal lavage, blood, cerebrospinal fluid, tears, saliva, lymph, dialysis fluid and the like; organ or tissue culture derived fluids; and fluids extracted from physiological tissues. Samples can also include derivatives and fractions of such fluids. In some embodiments, the sample is derived from a biopsy. The number of cells in a sample will generally be at least about 10³, usually at least 10⁴ more usually at least about 10⁵. The cells can be dissociated, in the case of solid tissues, or tissue sections may be analyzed. Alternatively a lysate of the cells can be prepared.

In some embodiments, detection utilizes staining of cells or histological sections, performed in accordance with conventional methods. An alternative method for diagnosis depends on the in vitro detection of binding between antibodies and protein encoded by the polymorphic variant in a lysate. Other immunoassays are known in the art and may find use as diagnostics. Ouchterlony plates provide a simple determination of antibody binding. Western blots can be performed on protein gels or protein spots on filters, using a detection system specific for polymorphic variant protein as desired, conveniently using a labeling method as described for the sandwich assay.

The invention provides a method for determining a genotype of an individual in relation to one or more polymorphic variants in one or more of the genes identified in above aspects by using mass spectrometric determination of a nucleic acid sequence that is a portion of a gene identified for other aspects of this invention or a complementary sequence. Such mass spectrometric methods are known to those skilled in the art.

The detection of the presence or absence of a polymorphic variant can involve contacting a nucleic acid sequence corresponding to one of the genes identified above or a product of such a gene with a probe. The probe is able to distinguish a particular form of the gene, gene product, polymorphic variant allele product, or allele product, or the presence or a particular polymorphic variant or polymorphic variants, for example, by differential binding or hybridization. The term “probe” refers to a molecule that can detectably distinguish between target molecules differing in structure. Detection can be accomplished in a variety of different ways depending on the type of probe used and the type of target molecule. Thus, for example, detection may be based on discrimination of activity levels of the target molecule, but preferably is based on detection of specific binding. Examples of such specific binding include antibody binding and nucleic acid probe hybridization. Probes can comprise one or more of the following, a protein, carbohydrate, polymer, or small molecule, that is capable of binding to one polymorphic variant or variant form of the gene or gene product to a greater extent than to a form of the gene having a different base at one or more polymorphic variant sites, such that the presence of the polymorphic variant or variant form of the gene can be determined. A probe can incorporate one or more markers including, but not limited to, radioactive labels, such as radionuclides, fluorophores or fluorochromes, peptides, enzymes, antigens, antibodies, vitamins or steroids. A probe can distinguished at least one of the polymeric variant described herein. The probe can also have specificity for the particular gene or gene product, at least to an extent such that binding to other genes or gene products does not prevent use of the assay to identify the presence or absence of the particular polymorphic variant or polymorphic variants of interest.

The nucleic acid molecules relevant to the invention can readily be obtained in a variety of ways, including, without limitation, chemical synthesis, cDNA or genomic library screening, expression library screening, and/or PCR amplification of cDNA. These methods and others useful for isolating such DNA are set forth, for example, by Sambrook, et al., “Molecular Cloning: A Laboratory Manual,” Cold Spring Harbor Laboratory Press, Cold Spring Harbor, N.Y. (1989), by Ausubel, et al., eds., “Current Protocols In Molecular Biology,” Current Protocols Press (1994), and by Berger and Kimmel, “Methods In Enzymology: Guide To Molecular Cloning Techniques,” vol. 152, Academic Press, Inc., San Diego, Calif. (1987). Nucleic acid sequences are mammalian sequences. In some embodiments, the nucleic acid sequences are human, rat, and mouse.

Chemical synthesis of a nucleic acid molecule can be accomplished using methods well known in the art, such as those set forth by Engels et al., Angew. Chem. Intl. Ed., 28:716-734 (1989). These methods include, inter alia, the phosphotriester, phosphoramidite and H-phosphonate methods of nucleic acid synthesis. Nucleic acids larger than about 100 nucleotides in length can be synthesized as several fragments, each fragment being up to about 100 nucleotides in length. The fragments can then be ligated together to form a full length nucleic acid encoding the polypeptide. A preferred method is polymer-supported synthesis using standard phosphoramidite chemistry.

Alternatively, the nucleic acid may be obtained by screening an appropriate cDNA library prepared from one or more tissue source(s) that express the polypeptide, or a genomic library from any subspecies. The source of the genomic library may be any tissue or tissues from any mammalian or other species believed to harbor a gene encoding a protein relevant to the invention. The library can be screened for the presence of a cDNA/gene using one or more nucleic acid probes (oligonucleotides, cDNA or genomic DNA fragments that possess an acceptable level of homology to the gene or gene homologue cDNA or gene to be cloned) that will hybridize selectively with the gene or gene homologue cDNA(s) or gene(s) that is(are) present in the library. The probes preferably are complementary to or encode a small region of the DNA sequence from the same or a similar species as the species from which the library can be prepared. Alternatively, the probes may be degenerate, as discussed below. After hybridization, the blot containing the library is washed at a suitable stringency, depending on several factors such as probe size, expected homology of probe to clone, type of library being screened, number of clones being screened, and the like. Stringent washing solutions are usually low in ionic strength and are used at relatively high temperatures.

Another suitable method for obtaining a nucleic acid in accordance with the invention is the polymerase chain reaction (PCR). In this method, poly(A)+ RNA or total RNA is extracted from a tissue that expresses the gene product. cDNA is then prepared from the RNA using the enzyme reverse transcriptase. Two primers typically complementary to two separate regions of the cDNA (oligonucleotides) are then added to the cDNA along with a polymerase such as Taq polymerase, and the polymerase amplifies the cDNA region between the two primers.

The invention provides for the use of isolated, purified or enriched nucleic acid sequences of 15 to 500 nucleotides in length, 15 to 100 nucleotides in length, 15 to 50 nucleotides in length, and 15 to 30 nucleotides in length, which have sequence that corresponds to a portion of one of the genes identified for aspects above. In some embodiments the nucleic acid is at least 17, 20, 22, or 25 nucleotides in length. In some embodiments, the nucleic acid sequence is 30 to 300 nucleotides in length, or 45 to 200 nucleotides in length, or 45 to 100 nucleotides in length. In some embodiments, the probe is a nucleic acid probe at least 15, 17 20, 22 25, 30, 35, 40, or more nucleotides in length, or 500, 250, 200, 100, 50, 40, 30 or fewer nucleotides in length. In preferred embodiments, the probe has a length in a range from any one of the above lengths to any other of the above lengths including endpoints. The nucleic acid sequence includes at least one polymorphic variant site. Such sequences can, for example, be amplification products of a sequence that spans or includes a polymorphic variant site in a gene identified herein. A nucleic acid with such a sequence can be utilized as a primer or amplification oligonucleotide that is able to bind to or extend through a polymorphic variant site in such a gene. Another example is a nucleic acid hybridization probe comprised of such a sequence. In such probes, primers, and amplification products, the nucleotide sequence can contain a sequence or site corresponding to a polymorphic variant site or sites, for example, a polymorphic variant site identified herein. The design and use of allele-specific probes for analyzing polymorphisms is known generally in the art, see, for example, Saiki et al., Nature 324:163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. A nucleic acid hybridization probe may span two or more polymorphic variant sites. Unless otherwise specified, a nucleic acid probe can include one or more nucleic acid analogs, labels or other substituents or moieties so long as the base-pairing function is retained. The nucleic acid sequence includes at least one polymorphic variant site. The probe may also comprise a detectable label, such as a radioactive or fluorescent label. A variety of other detectable labels are known to those skilled in the art. Nucleic acid probe can also include one or more nucleic acid analogs.

In connection with nucleic acid probe hybridization, the term “specifically hybridizes” indicates that the probe hybridizes to a sufficiently greater degree to the target sequence than to a sequence having a mismatched base at least one polymorphic variant site to allow distinguishing of such hybridization. The term “specifically hybridizes” means that the probe hybridizes to the target sequence, and not to non-target sequences, at a level which allows ready identification of probe/target sequence hybridization under selective hybridization conditions. “Selective hybridization conditions” refer to conditions that allow such differential binding. Similarly, the terms “specifically binds” and “selective binding conditions” refer to such differential binding of any type of probe, and to the conditions that allow such differential binding. Hybridization reactions to determine the status of variant sites in patient samples can be carried out with two different probes, one specific for each of the possible variant nucleotides. The complementary information derived from the two separate hybridization reactions is useful in corroborating the results.

A variety of variables can be adjusted to optimize the discrimination between two variant forms of a gene, including changes in salt concentration, temperature, pH and addition of various compounds that affect the differential affinity of GC vs. AT base pairs, such as tetramethyl ammonium chloride. [See Current Protocols in Molecular Biology, Ausubel et al. (Editors), John Wiley & Sons.] Hybridization conditions should be sufficiently stringent such that there is a significant difference in hybridization intensity between alleles, and preferably an essentially binary response, whereby a probe hybridizes to only one of the alleles. Hybridizations are usually performed under stringent conditions that allow for specific binding between an oligonucleotide and a target nucleic acid containing one of the polymorphic sites described herein or identified using the techniques described herein. Stringent conditions are defined as any suitable buffer concentrations and temperatures that allow specific hybridization of the oligonucleotide to highly homologous sequences spanning at least one polymorphic site and any washing conditions that remove non-specific binding of the oligonucleotide. For example, conditions of 5×SSPE (750 mM NaCl, 50 mM Na Phosphate, 5 mM EDTA, pH 7.4) and a temperature of 25-30° C. are suitable for allele-specific probe hybridizations. The washing conditions usually range from room temperature to 60° C. Some probes are designed to hybridize to a segment of target DNA such that the polymorphic site aligns with a central position of the probe. This probe design achieves good discrimination in hybridization between different allelic forms.

Allele-specific probes are can be used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence. The polymorphisms can also be identified by hybridization to nucleic acid arrays, some examples of which are described by WO 95/11995. Arrays may be provided in the form of a multiplex chip.

One use of probe(s) is as a primer(s) that hybridizes to a nucleic acid sequence containing at least one sequence polymorphic variant. Preferably such primers hybridize to a sequence not more than 300 nucleotides, more preferably not more than 200 nucleotides, still more preferably not more than 100 nucleotides, and most preferably not more than 50 nucleotides away from a polymorphic variant site which is to be analyzed. Preferably, a primer is 100 nucleotides or fewer in length, more preferably 50 nucleotides or fewer, still more preferable 30 nucleotides or fewer, and most preferably 20 or fewer nucleotides in length. In some embodiments, the set includes primers or amplification oligonucleotides adapted to bind to or extend through a plurality of sequence polymorphic variants in a gene(s) identified herein. In some embodiments, the plurality of polymorphic variants comprises a haplotype. In certain embodiments, the oligonucleotides are designed and selected to provide polymorphic variant-specific amplification.

Another type of probe is a peptide or protein, for example, an antibody or antibody fragment that specifically or preferentially binds to a polypeptide expressed by a particular form of a gene as characterized by the presence or absence of at least one polymorphic variant. Such antibodies may be polyclonal or monoclonal antibodies, and can be prepared by methods well-known in the art.

Antibodies can be used to probe for presence of a given polymorphism variant for those polymorphism variants that have an effect on the polypeptide encoded by the gene. For example, an antibody can recognize a change in one or more amino acid residues in the resulting protein. In some embodiments, the antibody is used to recognize polypeptides encoded by differential splice variants. If the polymorphism introduces or eliminates a surface feature of the protein such as a glycosylation site, lipid modification, etc., an antibody can also be used to identify a particular variant.

Polyclonal and/or monoclonal antibodies and antibody fragments capable of binding to a portion of the gene product relevant for identifying a given polymorphism variant are provided. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

The invention provides methods for choosing a relevant therapeutic strategy based on the detection of the presence or absence of one or more polymorphic variants. General methods of testing effects of a polymorphic variant for an effect on drug efficacy are known to those of skill in the art and are provided in various sources such as U.S. Pat. Nos. 6,537,759; 6,664,062; and 6,759,200.

A therapeutic agent, which can be a compound and/or a composition, relevant to the invention can comprise a small molecule, a nucleic acid, a protein, an antibody, or any other agent with one or more therapeutic property. The therapeutic agent can be formulated in any pharmaceutically acceptable manner. In some embodiments, the therapeutic agent is prepared in a depot form to allow for release into the body to which it is administered is controlled with respect to time and location within the body (see, for example, U.S. Pat. No. 4,450,150). Depot forms of therapeutic agents can be, for example, an implantable composition comprising the therapeutic agent and a porous or non-porous material, such as a polymer, wherein the therapeutic agent is encapsulated by or diffused throughout the material and/or degradation of the non-porous material. The depot is then implanted into the desired location within the body and the therapeutic agent is released from the implant at a predetermined rate.

The therapeutic agent that is used in the invention can be formed as a composition, such as a pharmaceutical composition comprising a carrier and a therapeutic compound. Pharmaceutical compositions containing the therapeutic agent can comprise more than one therapeutic agent. The pharmaceutical composition can alternatively comprise a therapeutic agent in combination with other pharmaceutically active agents or drugs, such as chemotherapeutic agents, for example, a cancer drug.

The carrier can be any suitable carrier. Preferably, the carrier is a pharmaceutically acceptable carrier. With respect to pharmaceutical compositions, the carrier can be any of those conventionally used and is limited only by chemico physical considerations, such as solubility and lack of reactivity with the active compound(s), and by the route of administration. In addition to the following described pharmaceutical composition, the therapeutic compounds of the present inventive methods can be formulated as inclusion complexes, such as cyclodextrin inclusion complexes, or liposomes.

The pharmaceutically acceptable carriers described herein, for example, vehicles, adjuvants, excipients, and diluents, are well-known to those skilled in the art and are readily available to the public. The pharmaceutically acceptable carrier can be chemically inert to the active agent(s) and one which has no detrimental side effects or toxicity under the conditions of use. The choice of carrier can be determined in part by the particular therapeutic agent, as well as by the particular method used to administer the therapeutic compound. There are a variety of suitable formulations of the pharmaceutical composition of the invention. The following formulations for oral, aerosol, parenteral, subcutaneous, transdermal, transmucosal, intestinal, parenteral, intramedullary injections, direct intraventricular, intravenous, intranasal, intraocular, intramuscular, intraarterial, intrathecal, interperitoneal, rectal, and vaginal administration are exemplary and are in no way limiting. More than one route can be used to administer the therapeutic agent, and in some instances, a particular route can provide a more immediate and more effective response than another route. Depending on the specific conditions being treated, such agents can be formulated and administered systemically or locally. Techniques for formulation and administration may be found in Remington's Pharmaceutical Sciences, 18th ed., Mack Publishing Co., Easton, Pa. (1990).

Formulations suitable for oral administration can include (a) liquid solutions, such as an effective amount of the inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the inhibitor in a flavor, usually sucrose and acacia or tragacanth, as well as pastilles comprising the inhibitor in an inert base, such as gelatin and glycerin, or sucrose and acacia, emulsions, gels, and the like containing, in addition to, such excipients as are known in the art.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The therapeutic agent, alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non pressured preparations, such as in a nebulizer or an atomizer. Such spray formulations also may be used to spray mucosa. Topical formulations are well known to those of skill in the art. Such formulations are particularly suitable in the context of the invention for application to the skin.

Injectable formulations are in accordance with the invention. The parameters for effective pharmaceutical carriers for injectable compositions are well-known to those of ordinary skill in the art [see, e.g., Pharmaceutics and Pharmacy Practice, J.B. Lippincott Company, Philadelphia, Pa., Banker and Chalmers, eds., pages 238 250 (1982), and ASHP Handbook on Injectable Drugs, Toissel, 4th ed., pages 622 630 (1986)]. For injection, the agents of the invention can be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Formulations suitable for parenteral administration include aqueous and non aqueous, isotonic sterile injection solutions, which can contain anti oxidants, buffers, bacteriostats, and solutes that render the formulation isotonic with the blood of the intended recipient, and aqueous and non aqueous sterile suspensions that can include suspending agents, solubilizers, thickening agents, stabilizers, and preservatives. The therapeutic agent can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the drug in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The therapeutic agent can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

The exact formulation, route of administration and dosage can be chosen by the individual physician in view of the patient's condition. [See, e.g., Fingl et. al., in The Pharmacological Basis of Therapeutics, 1975, Ch. 1 p. 1]. The attending physician can determine when to terminate, interrupt, or adjust administration due to toxicity, or to organ dysfunctions. Conversely, the attending physician can also adjust treatment to higher levels if the clinical response were not adequate, precluding toxicity. The magnitude of an administrated dose in the management of disorder of interest will vary with the severity of the condition to be treated and the route of administration. The severity of the condition may, for example, be evaluated, in part, by standard prognostic evaluation methods. The dose and perhaps dose frequency, can vary according to the age, body weight, and response of the individual patient. A program comparable to that discussed above can be used in veterinary medicine.

Use of pharmaceutically acceptable carriers to formulate the compounds herein disclosed for the practice of the invention into dosages suitable for systemic administration is within the scope of the invention. With proper choice of carrier and suitable manufacturing practice, the compositions relevant to the invention, in particular, those formulated as solutions, can be administered parenterally, such as by intravenous injection. The compounds can be formulated readily using pharmaceutically acceptable carriers well known in the art into dosages suitable for oral administration. Such carriers enable the compounds relevant to the invention to be formulated as tablets, pills, capsules, liquids, gels, syrups, slurries, tablets, dragees, solutions, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

The altered susceptibility can be either an increased or decreased susceptibility for a drug-induced heart rhythm irregularity. The relative susceptibility can be measured according to any acceptable medical parameters. Generally, the susceptibility is gauged relative to a subject that lacks the polymorphic variant or is heterozygous for the polymorphic variant. In some embodiments, the measure would be homozygous for the polymorphic variant or heterozygous for the polymorphic variant relative to a subject that is homozygous lacking the polymorphic variant. In some embodiments, two or more polymorphic variants for a give polymorphism are taken to be equivalent to each other relative to two or more polymorphic variants for the polymorphism.

According to one aspect, the method comprises not only screening and diagnosing steps, but also prescribing a treatment regimen based on the diagnosis. In some embodiments, the treatment regimen comprises increasing dosage of the drug in the presence of a polymorphic variant associated with a decreased susceptibility for the heart rhythm irregularity. In some embodiments, the treatment regimen comprises increasing dosage of the drug in the absence of a polymorphic variant associated with an increased susceptibility for the heart rhythm irregularity. In some embodiments, the treatment regimen comprises decreasing dosage of the drug in the presence of a polymorphic variant associated with an increased susceptibility for the heart rhythm irregularity. In some embodiments, the treatment regimen comprises decreasing dosage of the drug in the absence of a polymorphic variant associated with a decreased susceptibility for the heart rhythm irregularity. For example, one could decide based on the screening and diagnosis to not administer the heart rhythm irregularity inducing drug. In some such cases, a different drug is administered. In some embodiments, the drug does not bind ABCB1. In some embodiments, the treatment regimen comprises increased heart monitoring.

In another aspect, the screening and diagnosis result in the administration of one or more additional drug is administered. In some embodiments, the second drug ameliorates the heart rhythm irregularity.

The invention provides selecting a method of administration of an agent to a patient suffering from a disease or condition, by determining the presence or absence of at least one polymorphic variant in cells of the patient, where such presence or absence is indicative of an appropriate method of administration of the agent. The selection of a treatment regimen can involve selecting a dosage level or frequency of administration or route of administration of the agent(s) or combinations of those parameters. In some embodiments, two or more agents are administered, and the selecting involves selecting a method of administration for one, two, or more than two of the agents, jointly, concurrently, or separately. As understood by those skilled in the art, such plurality of agents is often used in combination therapy, and thus may be formulated in a single drug, or may be separate drugs administered concurrently, serially, or separately. Other embodiments are as indicated above for selection of second treatment methods, methods of identifying polymorphic variants, and methods of treatment as described for aspects above. The frequency of administration is generally selected to achieve a pharmacologically effective average or peak serum level without excessive deleterious effects. In some embodiments, the serum level of the drug is maintained within a therapeutic window of concentrations for the greatest percentage of time possible without such deleterious effects as would cause a prudent physician to reduce the frequency of administration for a particular dosage level. Administration of a particular treatment, for example, administration of a therapeutic compound or combination of compounds, is chosen depending on the disease or condition which is to be treated. In some embodiments, the disease or condition is one for which administration of a treatment is expected to provide a therapeutic benefit. In embodiments involving selection of a patient for a treatment, selection of a method or mode of administration of a treatment, and selection of a patient for a treatment or a method of treatment, the selection can be positive selection or negative selection. The methods can include modifying or eliminating a treatment for a patient, modifying or eliminating a method or mode of administration of a treatment to a patient, or modification or elimination of a patient for a treatment or method of treatment. A patient can be selected for a method of administration of a treatment, by detecting the presence or absence of at least one polymorphic variant in a gene as identified herein, where the presence or absence of the at least one polymorphic variant is indicative that the treatment or method of administration will be effective in the patient. If the at least one polymorphic variant is present in the patient's cells, then the patient is selected for administration of the treatment.

The term “drug” or “therapeutic agent” as used herein refers to a chemical entity or biological product, or combination of chemical entities or biological products, administered to a person to treat or prevent or control a disease or condition. In some embodiments, the chemical entity or biological product is a low molecular weight compound. A “low molecular weight compound” has a molecular weight<5,000 Da, <2500 Da, <1000 Da, or <700 Da. In some embodiments, the chemical entity is a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. In some embodiments, the biological product is a monoclonal or polyclonal antibody or fragment thereof such as a variable chain fragment cells; or an agent or product arising from recombinant technology, such as, without limitation, a recombinant protein, recombinant vaccine, or DNA construct developed for therapeutic use. The term “drug” or “therapeutic agent” can include, without limitation, compounds that are approved for sale as pharmaceutical products by government regulatory agencies such as the U.S. Food and Drug Administration (USFDA or FDA), the European Medicines Evaluation Agency (EMEA), and a world regulatory body governing the Intemation Conference of Harmonization (ICH) rules and guidelines, compounds that do not require approval by government regulatory agencies, food additives or supplements including compounds commonly characterized as vitamins, natural products, and completely or incompletely characterized mixtures of chemical entities including natural compounds or purified or partially purified natural products. In some embodiments, the drug is approved by a government agency for treatment of a specific disease or condition.

In treating a patient exhibiting a disorder of interest, a therapeutically effective amount of a agent or agents is administered. A therapeutically effective dose refers to that amount of the compound that results in amelioration of one or more symptoms or a prolongation of survival in a patient. The amount or dose of the therapeutic compound administered should be sufficient to affect a therapeutic response in the subject or animal over a reasonable time frame. For example, in the case of cancer, the dose of the therapeutic compound should be sufficient to inhibit metastasis, prevent metastasis, treat or prevent cancer in a period of from about 2 hours or longer, e.g., 12 to 24 or more hours, from the time of administration. In certain embodiments, the time period could be even longer. The dose can be determined by the efficacy of the particular therapeutic agent and the condition of the subject, as well as the body weight of the subject to be treated. Many assays for determining an administered dose are known in the art.

The dose of the therapeutic compound can also be determined by the existence, nature and extent of any adverse side effects that might accompany the administration of a particular therapeutic compound. The attending physician can decide the dosage of the inhibitor relevant to the invention with which to treat each individual patient using the correlation between polymorphic variant and disease and/or drug efficacies provided by the invention and taking into consideration a variety of factors, such as age, body weight, general health, diet, sex, inhibitor to be administered, route of administration, and the severity of the condition being treated. In some embodiments, the dose of the therapeutic compound is about 0.001 to about 1000 mg/kg body weight of the subject being treated/day, from about 0.01 to about 10 mg/kg body weight/day, about 0.01 mg to about 1 mg/kg body weight/day.

Toxicity and therapeutic efficacy of therapeutic agents can be determined by standard pharmaceutical procedures in cell cultures or experimental animals, for example, for determining the LD₅₀ and the ED₅₀. The dose ratio between toxic and therapeutic effects is the therapeutic index and it can be expressed as the ratio LD₅₀/ED₅₀. In some embodiments, compounds that exhibit large therapeutic indices are used. The data obtained from these cell culture assays and animal studies can be used in formulating a range of dosage for use in humans. The dosage of such compounds can lie within a range of circulating concentrations that can include the ED₅₀ with little or no toxicity. The dosage can vary within this range depending upon the dosage form and route of administration utilized. The therapeutically effective dose can be estimated initially from cell culture assays. For example, a dose can be formulated in animal models to achieve a circulating plasma concentration range that includes the IC₅₀ as determined in cell culture. Such information can be used to more accurately determine useful doses in humans. Levels in plasma may be measured, for example, by HPLC.

In connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by those of skill in the art.

In some embodiments, the drug is an anti-cancer agent. Examples of anti-cancer agents include actinomycin D, daunorubicin, docetaxel, doxorubicin, erlotinib, etoposide, gefitinib, imatinib, irinotecan, mitomycin c, mitoxantrone, paclitaxel, SN-38, teniposide, topotecan, vinblastine, vincristine, a pro drug thereof, a salt thereof, or a combination thereof. Another applicable cancer drug is a depsipeptide, e.g., FK228, as well as prodrugs, salts and combination thereof. FK228 is also known as romidepsin. In some embodiments, the FK228 is the isomer FR901228, which is (E)-(1S,4S,10S,21R)-7-[(Z)-ethylidene]-4,21-diisopropyl-2-oxa-12,13-dithia-5,8,20,23-tetraazabicyclo [8,7,6]-tricos-16-ene-3,6,19,22-pentanone (NSC 630176). FK228 compounds, salts, prodrugs, formulation, method of preparation, dosage, administration, and other FK228 parameters can be used in accordance with the materials and method of this invention. The salt of FK228, e.g., FR901228, is a biologically acceptable salt, which is generally non-toxic, and is exemplified by salts with base or acid addition salts, inclusive of salts with inorganic base such as alkali metal salt (e.g., a sodium salt, a potassium salt, etc.), alkaline earth metal salt (e.g., calcium salt, magnesium salt, etc.), ammonium salt, salts with organic base such as organic amine salt (e.g., triethylamine salt, diisopropylethylamine salt, pyridine salt, picoline salt, ethanolamine salt, diethanolamine salt, triethanolamine salt, dicyclohexylamine salt, N,N′-dibenzylethylenediamine salt, etc.), inorganic acid salt (e.g., hydrochloride, hydrobromide, sulfate, phosphate, etc.), organic carboxylic or sulfonic acid salt (e.g., formate, acetate, trifluoroacetate, maleate, tartrate, fumarate, methanesulfonate, benzenesulfonate, toulenesulfonate, etc.), salt with basic or acid amino acid (e.g., arginine, aspartic acid, glutamic acid, etc.), and the like. Examples of relevant FK228 parameters, as well as parameters for other depsipeptides and histone deacetylase inhibitors (HDIs), applicable to the invention are provided in U.S. Provisional Application Nos. 60/226,234 and 60/709,553; WO 02/15921; WO 03/084611; and WO 02/055688.

Drugs applicable to the method are not limited to anti-cancer drugs. The heart rhythm irregularity inducing drug can be an antacid. Examples of antacids include cimetidine, ranitidine, a prodrug thereof, a salt thereof, or a combination thereof. In some embodiments, the heart rhythm inducing drug is an antiarrhythmic. Examples of such antiarrthymics include amiodarone, digoxin, propafenone, quinidine, verapamil, a prodrug thereof, a salt thereof, or a combination thereof. The heart rhythm irregularity inducing drug can be an antibiotic. Examples of such antibiotics include clarithromycin, erythromycin, levofloxacin, rifampin, sparfloxacin, tetracycline, a prodrug thereof, a salt thereof, or a combination thereof. In some embodiments, the drug is an antidepressant, such as amitriptyline, fluoxetine, paroxetine, sertraline, St John's wort, a prodrug thereof, a salt thereof, or a combination thereof. The drug can be an antiemetic. Examples of such antiemetics include domperidon, ondansetron, a prodrug thereof, a salt thereof, or a combination thereof. In some embodiments, the drug is an antiepileptic such as phenobarbital, phenyloin, a prodrug thereof, a salt thereof, or a combination thereof. The drug can also be an antihypertensive. Examples of antihypertensives include carvedilol, celiprolol, diltiazem, losartan, nicardipine, reserpine, talinolol, a prodrug thereof, a salt thereof, or a combination thereof.

In some embodiments, the heart rhythm irregularity inducing drug is an antimycotic. Examples of such antimycotics include itraconazole, ketoconazole, a prodrug thereof, a salt thereof, or a combination thereof. The drug can be an antiviral agent. Examples of antiviral agents include amprenavir, indinavir, nelfinavir, ritonavir, saquinavir, a prodrug thereof, a salt thereof, or a combination thereof. The drug can be a glucocorticoid such as aldosterone, cortisol, dexamethasone, methylprednisolone, a prodrug thereof, a salt thereof, or a combination thereof. In some embodiments, the drug is an immunosuppressant. Examples of such immunosuppressants include cyclosporine, sirolimus, tacrolimus, valspodar, a pro drug thereof, a salt thereof, or a combination thereof. The drug can also be a neuroleptic such as chloropromazine, flupenthixol, phenothiazine, a prodrug thereof, a salt thereof, or a combination thereof. In some embodiments, the drug is an opioid. Examples of such opioid include methadone, morphine, pentazocine, a prodrug thereof, a salt thereof, or a combination thereof.

In some embodiments, the heart rhythm irregularity inducing drug is selected from the group consisting of torvastatin, bromocriptine, colchicine, dipyridamole, emetine, fexofenadine, ivermectin, loperamide, mefloquine, progesterone, retinoic acid, rhodamine 123, spironolactone, terfenadine, vecuronium, a prodrug thereof, a salt thereof, or a combination thereof.

Kits compatible with the methods are also provided. In one aspect, a kit is provided that includes a nucleic acid and a drug that binds a protein encoded ABCB1. The nucleic acid is for use in screening a sample from a subject to detect the presence or absence of at least one polymorphic variant of at least one polymorphism of the ABCB1 gene, wherein the polymorphic variant is associated with an altered susceptibility for a heart rhythm irregularity induced by a drug that binds a protein encoded by the ABCB1 gene, and wherein the nucleic acid specifically binds to ABCB1 sequence comprising the at least one polymorphism or a sequence adjacent to ABCB1 sequence comprising the at least one polymorphism. In one aspect, the polymorphism comprises polymorphism identified as rs1128503, rs2032582, rs1045642, or a combination thereof. In one aspect, the polymorphism comprises a polymorphism at position 49,910, 68,894, or 90,871 of SEQ ID NO: 1; or 1236, 2677, or 3435 of SEQ ID NO: 2; or a combination thereof. In another aspect, the drug is FK228 and/or another drug described herein. In some embodiments, the kit's nucleic acid comprises the nucleotide sequence of any one of SEQ ID NOS: 25-36 or a compliment thereof or a combination thereof.

The invention includes kits for the detection of polymorphic variants associated with disease states, conditions or complications. The kits can comprise a polynucleotide of at least 30 contiguous nucleotides of one of the variants described herein. In one embodiment, the polynucleotide contains at least one polymorphism of the invention. Alternatively, the 3′ end of the polynucleotide is immediately 5′ to a polymorphic site, preferably a polymorphic site of the invention. In one embodiment, the polymorphic site contains a genetic variant. In still another embodiment, the genetic variant is located at the 3′ end of the polynucleotide. In yet another embodiment, the polynucleotide of the kit contains a detectable label. Suitable labels include, but are not limited to, radioactive labels, such as radionucleotides, fluorophores or fluorochromes, peptides, enzymes, antigens, antibodies, vitamins or steroids. The kit may also contain additional materials for detection of the polymorphisms. A kit can contain one or more of the following: buffer solutions, enzymes, nucleotide triphosphates, and other reagents and materials useful for the detection of genetic polymorphisms. Kits can contain instructions for conducting analyses of samples for the presence of polymorphisms and for interpreting the results obtained.

In some embodiments, the kit contains one or more pairs of allele-specific oligonucleotides hybridizing to different forms of a polymorphism. In some embodiments, the kit contains at least one probe or at least one primer or both corresponding to a gene or genes relevant to the invention. The kit can be adapted and configured to be suitable for identification of the presence or absence of one or more polymorphic variants. The kit can contain a plurality of either or both of such probes and/or primers, for example, 2, 3, 4, 5, 6, or more of such probes and/or primers. The plurality of probes and/or primers are adapted to provide detection of a plurality of different sequence polymorphic variants in a gene or plurality of genes, for example, in 2, 3, 4, 5, or more genes or to sequence a nucleic acid sequence including at least one polymorphic variant site in a gene or genes. In some embodiments, the kit contains components for detection of a plurality of polymorphic variants indicative of the effectiveness of a treatment or treatment against a plurality of diseases. Additional kit components can include one or more of the following: a buffer or buffers, such as amplification buffers and hybridization buffers, which may be in liquid or dry form, a DNA polymerase, such as a polymerase suitable for carrying out PCR, and deoxy nucleotide triphosphases (dNTPs). Preferably a probe includes a detectable label, for example, a fluorescent label, enzyme label, light scattering label, or other label. Additional components of the kit can also include restriction enzymes, reverse-transcriptase or polymerase, the substrate nucleoside triphosphates, means used to label, for example, an avidin-enzyme conjugate and enzyme substrate and chromogen if the label is biotin, and the appropriate buffers for reverse transcription, PCR, or hybridization reactions.

In some kits, the allele-specific oligonucleotides are provided immobilized to a substrate. For example, the same substrate can comprise allele-specific oligonucleotide probes for detecting any or all of the polymorphism variants described herein. Accordingly, the kit may comprise an array including a nucleic acid array and/or a polypeptide array. The array can include a plurality of different antibodies, a plurality of different nucleic acid sequences. Sites in the array can allow capture and/or detection of nucleic acid sequences or gene products corresponding to different polymorphic variants in one or more different genes. The array can be arranged to provide polymorphic variant detection for a plurality of polymorphic variants in one or more genes which correlate with the effectiveness of one or more treatments of one or more diseases.

The kit also can contain instructions for carrying out the methods. In some embodiments, the instructions include a listing of the polymorphic variants correlating with a particular treatment or treatments for a disease of diseases. The kit components can be selected to allow detection of a polymorphic variant described herein, and/or detection of a polymorphic variant indicative of a treatment, for example, administration of a drug.

Uses of a drugs such as FK228 to manufacture a medicament are also provided. In one aspect, there is a use of a drug that binds a protein encoded by the ABCB1 gene to manufacture a medicament to treat a subject that that has been screened for the presence or absence of at least one polymorphic variant of at least one polymorphism of the ABCB1 gene, wherein the polymorphic variant is associated with an altered susceptibility for a heart rhythm irregularity induced by the drug. In one aspect, the polymorphism comprises polymorphism identified as rs1128503, rs2032582, rs1045642, or a combination thereof. In another aspect, the polymorphism comprises a polymorphism at position 49,910, 68,894, or 90,871 of SEQ ID NO: 1; or 1236, 2677, or 3435 of SEQ ID NO: 2, or a combination thereof. Other uses such as uses analogous to the methods described herein are also provided.

The following examples further illustrate the invention but, of course, should not be construed as in any way limiting its scope.

### Example 1

This example demonstrates that individuals with certain polymorphic variants in the ABCB1 gene encounter fewer heart rhythm irregularities typically induced by FK228 treatment.

Subject eligibility criteria used are in accordance with those described in Piekarz et al, Blood 98:2865-8 (2001). Eligible patients have a confirmed diagnosis of cutaneous T-cell lymphoma or relapsed peripheral T-cell lymphoma. Additional common eligibility criteria include: (i) a life expectancy of ≧12 weeks; (ii) an Eastern Cooperative Group performance status≦2; (iii) no chemotherapy, hormonal therapy or radiotherapy, within four weeks prior to treatment; (iv) age above 18 years; (v) adequate contraception for women of child-bearing potential; and (vi) adequate bone marrow function (absolute neutrophil count, >1.0×10⁹/L; platelets, platelet count, >100×10⁹/L), renal function [serum creatinine, ≦1.5× the upper limit of normal (ULN)], and hepatic function (serum bilirubin, ≦1.5×ULN; and aspartate aminotransferase, <3.0×ULN, unless impairment is due to organ involvement by lymphoma). The study protocol is approved by the local ethical review board, and all patients are provided written informed consent before study entry.

FK228 is supplied as a lyophilized powder by the Pharmaceutical Management Branch, Cancer Therapy Evaluation Program, Division of Cancer Treatment and Diagnosis, National Cancer Institute (Bethesda, Md.) in sterile vials containing 10 mg of drug and 20 mg of povidine as a bulking agent. Immediately prior to drug administration, FK228 is reconstituted in 2 mL of a diluent containing a mixture of propylene glycol and ethanol (4:1, vol/vol). This 5-mg/mL solution is diluted in 500 mL or 1000 mL of sodium chloride for injection, USP. FK228 is administered as a 4-hour continuous infusion on days 1, 8, and 15 via a portable infusion pump, with cycles repeated every 21 days. Provided toxic effects are not prohibitive, patients are eligible to continue treatment until there is evidence of progressive disease.

Complete blood cell counts with differential are obtained immediately prior to FK228 administration and on days 2, 9, and 16 to evaluate FK228-related myelosuppression. Multiple surface electrocardiograms (ECGs) are obtained immediately before FK228 administration, and at 4 hours after the start of FK228 administration, to evaluate the ability of FK228 to delay cardiac repolarization. This effect is manifested on the ECG as prolongation of the QT interval. The QT interval is transformed into the heart-rate independent corrected value known as the QTc interval. Prolongation of the QTc interval is the electrocardiographic finding associated with increased susceptibility to the development of cardiac arrythmias, including ventricular arrhythmias such as Torsade de Pointes. Because measurement of the baseline value is a factor that critically influences the observed variability in the mean QTc interval, values are computed as the mean of multiple ECGs to enhance the precision of the measurement. This computation is performed by collecting drug-free ECGs on three or more different days. The on-study time point for obtaining an ECG are selected to coincide with the maximum plasma concentration of FK228, as recommended in the preliminary FDA concept paper: The Clinical Evaluation Of Qt/Qtc Interval Prolongation And Proarrhythmic Potential For Non-Antiarrhythmic Drugs (Nov. 15, 2002) available at: http://www.fda.gov/ohrms/dockets/ac/03/briefing/pubs %5Cprelim.pdf.

To examine the pharmacokinetic profile of FK228 following its intravenous administration, blood samples are collected following the first administration from a peripheral site contra lateral to the venous access used for drug infusion, and immediately placed in an ice water bath. Samples are obtained before drug administration and at serial time points after the start of drug administration, including at the end of infusion (4 hours), and at 2, 7, 9, 11, 14, and 21 hours after the end of infusion. All samples are centrifuged in a refrigerated centrifuge, and then stored at or below −20° C. until the time of analytical analysis. FK228 concentrations in samples from patients treated with FK228 are quantitated by liquid chromatography with single-quadrupole mass spectrometric detection over the concentration range of 0.5 ng/mL to 100 ng/mL, according to a validated, previously published procedure. Hwang, et al, J. Chromatogr. B. Analyt. Technol. Biomed. Life Sci. 809:81-6 (2004). The values for precision and percent deviation from nominal (accuracy) are ≦7.88% and <3.33%, respectively.

Estimates of pharmacokinetic parameters for FK228 are derived from individual concentration-time data sets using model independent methods as implemented in the computer software program WinNonlin v5.0 (Pharsight Corporation, Mountain View, Calif.). The maximum plasma concentration (Cmax) and the time of maximum plasma concentration (Tmax) are the observed values. The area under the concentration-time curve (AUC) from time zero to the time of the final quantifiable sample (AUC[tf]) is calculated using the log-linear trapezoidal method. In addition, the AUC from time zero to infinity (AUC[inf]) is extrapolated to infinity by dividing the last measured concentration by the terminal rate constant, λ_{z}, which is determined from the slope of the terminal phase of the concentration-time curve using weighted least-squares as the estimation procedure, and inverse variance of the output error (linear) as the weighting option. In view of the linear pharmacokinetic profile of FK228 within the tested dose range, see Sandor et al., Br. J. Cancer 83:817-25 (2000), individual values for Cmax and AUC[inf] are normalized to a dose of 14 mg/m². The terminal half-life (t_{1/2,z}) is calculated as 0.693 divided by λ_{z}. Additional pharmacokinetic parameters include the volume of distribution at steady-state (Vₛₛ) and the systemic clearance (CL), which is calculated as dose divided by AUC[inf], with dose expressed in mg. The clearance is also calculated in units of L/h/m², by dividing CL by each patient's body-surface area (BSA).

Relationships between various exposure measures, for example, plasma AUC, and hematological and cardiac toxicity are evaluated by sigmoid maximum-effect models. Cardiac functional assessment is evaluated using base-line corrected QTc interval values (ΔQTc), as described by Sandor et al., Br. J. Cancer 83:817-25 (2000). Hematological pharmacodynamics are evaluated by analysis of the absolute nadir values of platelet counts or the relative thrombocytopenia, that is, the percent decrease in platelet count. Data are fitted to a sigmoid maximum-effect model based on the modified Hill equation, as follows: E=E₀+Eₘₐₓ×[(KP^{γ})/(KP^{γ}+KP₅₀^{γ})]. In this equation, E₀ is the minimum reduction possible, Eₘₐₓ is the maximum response (fixed to a value of 100), KP is the pharmacokinetic parameter of interest, KP₅₀ the value of the pharmacokinetic parameter predicted to result in half of the maximum response, and γ is the Hill constant, which describes the sigmoidicity of the curve. Models are evaluated for goodness of fit by minimization of sums of the squared residuals and by reduction of the estimated coefficient of variation for fitted parameters. Significance of the relationships are assessed by construction of contingency tables with subsequent chi-squared analysis.

Genomic deoxyribonucleic acid (DNA) is extracted from 1 mL of plasma using the QIAamp DNA Blood midi kit (Qiagen Inc, Valencia, Calif.), following the manufacturers instructions, and is reconstituted in a buffer containing 10 mM Tris (pH 7.6) and 1 mM EDTA. For analysis of ABCB1 variants, a 50-μL reaction is prepared for polymerase chain reaction (PCR) amplification using the PCR primer combinations listed in Table I. The reaction consists of 1 PCR buffer, 2 mM of each of the four deoxynucleotide triphosphates (dNTPs), 1.5 mM magnesium chloride, and 1 unit of Platinum Taq DNA polymerase from Invitrogen (Carlsbad, Calif.). PCR conditions are as follows: 94° C. for 5 minutes, followed by 40 cycles of 94° C. for 30 seconds, 68° C. for 30 seconds, and 72° C. for 30 seconds, with a final 7 minute cycle at 72° C. Direct nucleotide sequencing PCR is conducted using the Big Dye Terminator Cycle Sequencing Ready Reaction kit V1.1 (Applied Biosystems) using the sequencing primers listed in Table I. Sequences are generated on an ABI Prism 310 Genetic Analyzer. Variations in CYP3A4 (CYP3A4*1B) and CYP3A5 (CYP3A5*3C) are also analyzed using direct nucleotide sequencing, as described by Lepper et al., Clin Cancer Res., 11(20):7398-404 (2005). The genotype is called variant if it differed from the Refseq consensus sequence (rs) for the SNP position. Refseqs are available at http://www.ncbi.nlm.nih.gov/LocusLink/refseq.html.

TABLE I
Primers used for ABCB1 amplification and sequencing.

| | | |
|---|---|---|
| Region | PCR Primer Sequence | Sequencing Primer Sequence |

| | | | | |
|---|---|---|---|---|
| 1236C > T | F GTTCACTTCAGTTACCCATCTCG | (SEQ ID NO: 25) | F GTCAGTTCCTATATCCTGTGTCTG | (SEQ ID NO: 31) |
| | R TATCCTGTCCATCAACACTGACC | (SEQ ID NO: 26) | R TCCTGTCCATCAACACTGACCTG | (SEQ ID NO: 32) |
| 2677G > A/T | F AGGCTATAGGTTCCAGGCTTGC | (SEQ ID NO: 27) | F CCCATCATTGCAATAGCAGGAG | (SEQ ID NO: 33) |
| | R AGAACAGTGTGAAGACAATGGCC | (SEQ ID NO: 28) | R GAACAGTGTGAAGACAATGGCCT | (SEQ ID NO: 34) |
| 3435C > T | F ATCTCACAGTAACTTGGCAGTTTC | (SEQ ID NO: 29) | F GCTGGTCCTGAAGTTGATCTGTG | (SEQ ID NO: 35) |
| | R AACCCAAACAGGAAGTGTGGCC | (SEQ ID NO: 30) | R AAACAGGAAGTGTGGCCAGATGC | (SEQ ID NO: 36) |

All data are reported as median values with range, unless specified otherwise. Interindividual pharmacokinetic variability is calculated as the coefficient of variation, and expressed as a percentage. Genotype-frequency analysis of Hardy-Weinberg equilibrium is carried out using Clump version 1.9. The linkage between each pair of SNPs is determined in terms of the classical statistic D′. The absolute value for D′ (|D′|) of 1 denotes complete linkage disequilibrium, while a value of 0 denotes complete linkage equilibrium. The effects of the variant genotypes on ΔQTc, relative thrombocytopenia, dose-normalized AUC, apparent oral clearance, half-life, volume of distribution at steady-state are evaluated statistically with the nonparametric Kruskal-Wallis test. A post-hoc distribution-free multiple comparison procedure is performed using the Dunn test with Bonferroni correction to test pairs of median observations. All statistical analyses are performed using the NCSS software program (version 2001; NCSS, Kaysville, Utah). The a priori level of significance is set at 0.05.

FK228 is administered to 42 patients with T-cell lymphoma (17 female, 25 male) as a 4-hour continuous infusion at a dose of 14 mg/m² (n=37) or 18 mg/m² (n=5). The median age of the patients is 56 years (range, 27-79 years) and the median BSA is 1.93 m² (range, 1.43-2.46 m²). Thirty-three patients (79%) are Caucasian, 8 are African-American (19%), and 1 is Hispanic (2%). Pharmacokinetic data are available from all 42, patients; complete baseline and on-study measurements on blood cell counts and ΔQTc from 34 and 29 patients, respectively.

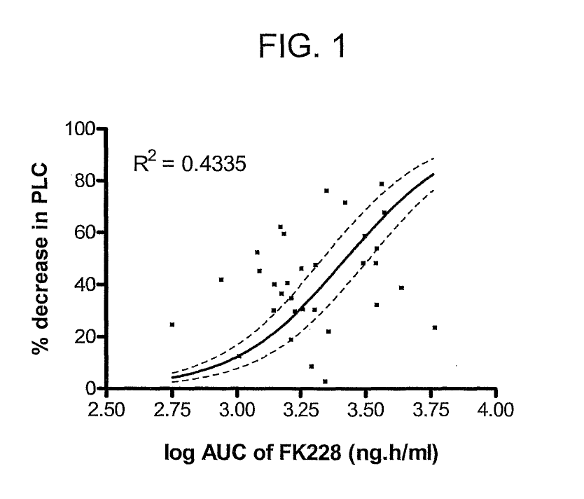

With the data from all patients combined, the mean (±standard deviation) values for FK228 clearance and terminal half-life are 17.5±12.7 L/h and 7.23±3.0 hours, respectively. This is within the range of values observed previously in patients treated with FK228 at doses of 12.7 mg/m2 and 17.8 mg/m2 as described in Sandor et al., Br. J. Cancer 83:817-25 (2000). The interindividual variability in drug clearance is relatively high, with a percent coefficient of variation of approximately 72%. Pharmacokinetic parameters of FK228 are not significantly different between men and women (P>0.12). The AUC of FK228 is weakly associated with the percentage decrease in platelet count (P<0.001; FIG. 1) using a sigmoid maximum effect model, but not with interindividual ΔQTc interval following FK28 treatment (P=0.62).

The observed ABCB1, CYP3A4, and CYP3A5 genotype frequencies are in Hardy-Weinberg equilibrium (P>0.13) (Table II). [Cascorbi et al, Clin. Pharmacol. Ther., 69:169-74 (2001); Lamba et al., Adv. Drug Deliv. Rev. 54:1271-94 (2002); Xie et al., Pharmacogenomics 5:243-72 (2004).] Strong linkage is observed between the 3 SNPs in ABCB1, with a D′ of 0.88 for the 1236C>T and 2677C>T/A loci (P<0.001); a D′ of 0.66 (P<0.001) for the 1236C>T and 3435C>T loci; and a D′ of 0.65 for the 2677G>T/A and 3435C>T loci (P<0.001). The overall linkage for the three loci is about 57%. The most frequently observed haplotypes in our population are C-G-C (44.3%; haplotype 1), T-T-T (31.4%; haplotype 2), and C-G-T (12.0%; haplotype 3), although in total 8 different haplotypes are observed.

TABLE II
Genotype and allele frequencies of the studied variants.

| | | | | | | Allele | |
|---|---|---|---|---|---|---|---|
| | | | Genotype frequencies^{a} | | | frequencies^{b} | |
| Polymorphism^{c} | Nomenclature | Effect^{d} | Wt^{e} | Het | Var | p | q |
| Caucasians | | | | | | | |
| ABCB1 1236C > T | N/a | G411G | 10 (33.6) | 14 (46.7) | 6 (20.0) | 0.567 | 0.433 |
| ABCB1 2677G > T | N/a | A893S | 9 (30.0) | 13 (43.3) | 6 (20.0) | 0.517 | 0.417 |
| ABCB1 2677G > A | N/a | A893T | 9 (30.0) | 2 (3.3) | 0 (0) | 0.517 | 0.033 |
| ABCB1 3435C > T | N/a | I1145I | 8 (26.7) | 14 (46.7) | 8 (26.7) | 0.500 | 0.500 |
| CYP3A4-392A > G | CYP3A4*1B | Promoter | 25 (78.2) | 3 (9.4) | 4 (12.5) | 0.828 | 0.172 |
| CYP3A5 6986A > G | CYP3A5*3C | Splice variant | 4 (12.5) | 6 (18.8) | 22 (68.8) | 0.219 | 0.781 |
| African Americans | | | | | | | |
| ABCB1 1236C > T | N/a | G411G | 5 (62.5) | 1 (12.5) | 2 (20.0) | 0.590 | 0.410 |
| ABCB1 2677G > T | N/a | A892S | 6 (75.0) | 1 (12.5) | 1 (12.5) | 0.813 | 0.187 |
| ABCB1 2677G > A | N/a | A893T | 0 (0) | 0 (0) | 0 (0) | 0.813 | 0.000 |
| ABCB1 3435C > T | N/a | I1145I | 1 (12.5) | 4 (50.0) | 3 (37.5) | 0.375 | 0.625 |
| CYP3A4-392A > G | CYP3A4*1B | M445T | 5 (62.5) | 0 (0) | 3 (37.5) | 0.625 | 0.375 |
| CYP3A5 6986A > G | CYP3A5*3C | Splice variant | 2 (25.0) | 1 (12.5) | 5 (62.5) | 0.312 | 0.688 |

^{a}Number represent number of patients with percentage in parenthesis; the difference in the total number of patients is due to the fact that not all samples yield sequencing data or showed PCR amplification;
^{b}Hardy-Weinberg notation for allele frequencies (p, frequency for wild type allele and q, frequency for variant allele);
^{c}Number represents position in nucleotide sequence;
^{d}Number represents amino acid codon;
^{e}Wt, Homozygous wild type patient;
Het, Heterozygous patient;
Var, Homozygous variant patient.

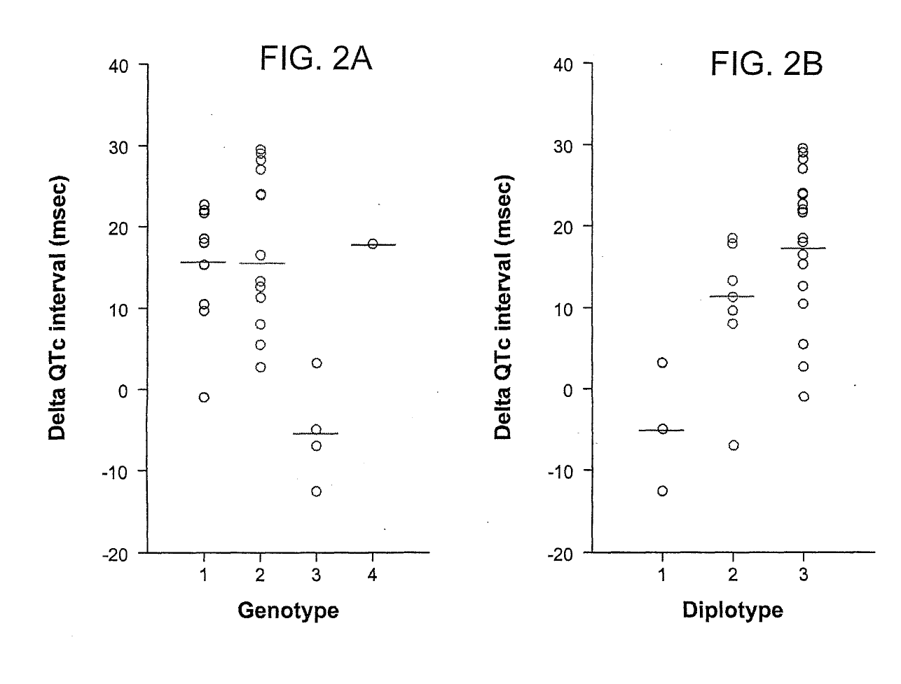

A significant association between ΔQTc at four hours and ABCB1 genotype at the 2677G>T/A locus is observed (P=0.024) (FIG. 2A). Patients carrying the 2677T/T genotype have a significantly lower ΔQTc (median ΔQTc, −5 msec; range, −12.5-3.25 msec; n=4) as compared to those with the 2677GG (ΔQTc, 18.3 msec; range, −1-22.7 msec; n=10), 2677GT (ΔQTc, 16.5 msec; range, 2.75-28.2 msec; n=14) or 2677GA genotypes (ΔQTc, 17.8 msec; n=1). A trend for similar observation is noted for the 1236C>T (P=0.10) and 3435C>T loci (P=0.079), although for these SNPs the associations are not statistically significant. Additional analyses indicate that consideration of haplotype 2 in this group of patients does not result in improved associations as compared to the single-phased SNPs (P=0.033). However, patients homozygous for the ABCB1 2677TT/3435TT diplotype (ΔQTc, −5.0 msec; range, −12.5-3.25; n=3) have a significantly lower ΔQTc (P=0.0084) compared with carriers of the heterozygote (ΔQTc, 11.3 msec; range, −7-17.8 msec; n=7) or homozygote diplotype (ΔQTc, 18.5 msec; range, −1=28.2 msec; n=19) (FIG. 2B).

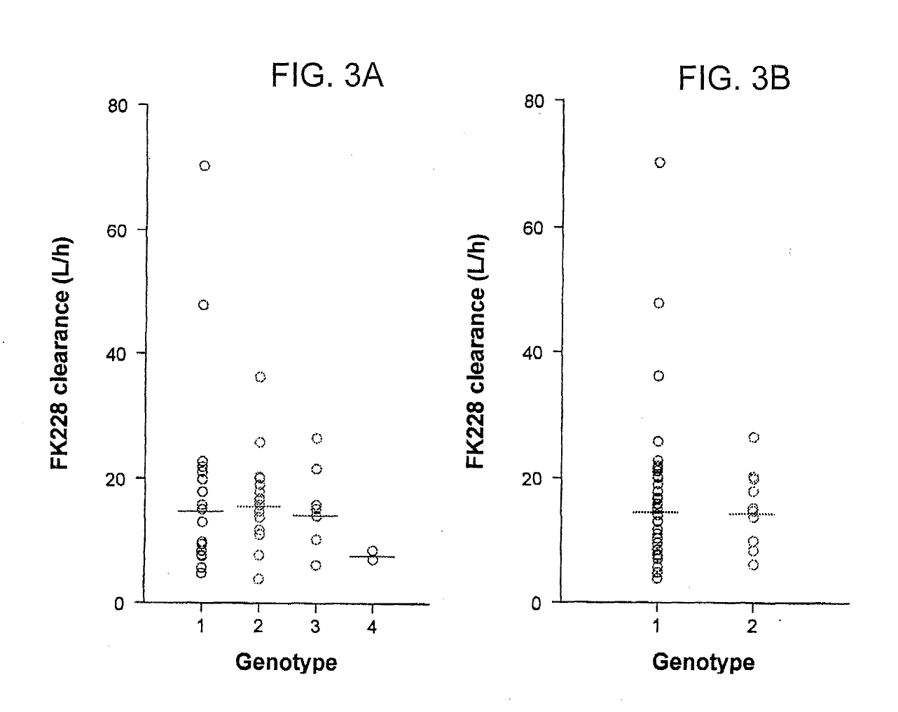

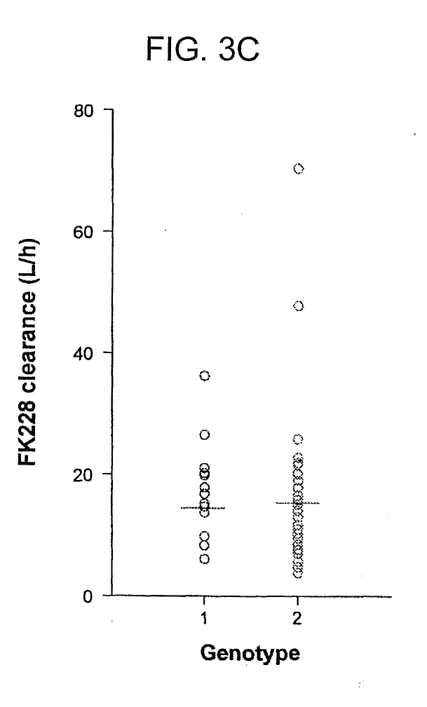

None of the variant ABCB1 or any of the ABCB1 haplotypes is significantly associated with the relative hematologic toxicity or FK228 clearance. The CYP3A4*1B and CYP3A5*3C alleles are also not statistically significantly associated with any measure of toxicity or FK228 clearance (FIG. 3). Differences in other pharmacokinetic parameters are also not statistically significantly different between the different genotype groups.

### Example 2

This example further demonstrates that individuals with certain polymorphic variants of the ABCB1 gene, e.g., ABCB1 2677G>T/A and 3435C>T, encounter fewer heart rhythm irregularities typically induced by FK228 (romidepsin, a cyclic depsipeptide) treatment and that QT and QTc interval prolongation associated with romidepsin treatment is linked to ABCB1 variants. This effect is unrelated to an altered plasma pharmacokinetic profile. Romidepsin is used as a model substrate for ABCB1.

Data from patients with T-cell lymphoma participating on a phase II clinical trial of romidepsin are initially evaluated (group 1). Eligibility criteria are consistent with those described in Example 1 and patients with evidence of heart disease are excluded from the trial. Toxicities are reported using the NCI Common Toxicity Criteria, version 2.0. The Inclusion Criteria required measurable disease; an age of 18 years or older; an Eastern Cooperative Oncology Group performance status of 0, 1, or 2; and a life expectancy of >12 weeks. Eligible laboratory values can include AGC≧1,000/AL, platelets≧100,000/AL, bilirubin<1.5× the institutional upper limit of normal, aspartate aminotransferase<3× upper limit of normal, and creatinine<1.5× upper limit of normal. Patients with a myocardial infarction within the previous 6 months, a left ventricular ejection fraction (LVEF) below normal (<45% if done by MUGA, or <50% if done by echocardiogram or cardiac magnetic resonance imaging), a corrected QT interval of >500 milliseconds, unstable angina, or third-degree heart block (unless with pacemaker) are excluded. Patients can be premedicated with ondansetron.

Confirmatory analysis (group 2) utilizes data from two sources: a) patients participating on the same multi-institutional trial as the initial analysis, but who are treated at institutions other than the NCI; and b) patients treated on the single-agent Phase I clinical trial of romidepsin previously conducted at the National Cancer Institute [Sandor et al., Clin Cancer Res 8:718-28 (2002)]. The common eligibility criteria are as described above for group 1, except that patients with malignancies other than T-cell lymphoma are also eligible.

Electrocardiograms (ECGs) are obtained immediately before romidepsin administration, and at 4 hours after the start of romidepsin administration (at the end of infusion and within 1 hour thereafter). Electrocardiograms can be obtained using an HP Pagewriter XLi or a GE Marquette MAC1200 and recorded at 25 mm/s, with an amplitude of 10 mm/mV and with 60-Hz filtering. They can be analyzed using Pagewriter A.04.01 electrocardiogram analysis software (Philips Medical Systems, Andover, Mass.). The QT interval measurement in this program can be made by averaging the five longest QT intervals with a T or T′ wave amplitude of >0.15 mV. The heart rate-corrected QT interval (QTc), indicating repolarization time, is calculated using Bazett's formula (QT divided by the square root of the preceding R—R interval) using the electrocardiogram machine software. QTc as calculated by Friderica's formula is the QT divided by the cubed root of the preceding R—R interval. QTc intervals of 480 ms or greater are independently reviewed by a cardiologist. Because measurement of the baseline value is a factor that critically influences the observed variability in the mean QTc interval, the initial analysis utilized baseline values that are computed as the mean of multiple ECGs to enhance the precision of the measurement. The on-study time point for obtaining an ECG is selected to coincide with the maximum plasma concentration of romidepsin, and multiple baseline ECGs are measured as recommended by the official guidelines of the FDA [Guidance for Industry E14 Clinical Evaluation of QT/QTc Interval Prolongation and Proarrhytmic Potential for Non-Antiarrhythmic Drugs; U.S. Department of Health and Human Services Food and Drug Administration: Center for Drug Evaluation and Research (CDER) and Center for Biologics Evaluation and Research (CBER) (October 2005), available at http://www.fda.gov/cber/gdlns/iche14qtc.pdf]. Confirmatory analysis utilizes the same design, but with only a single baseline ECG measurement obtained prior to administration of romidepsin as is conducted in most clinics. A clinical scoring system is also utilized wherein ECG abnormalities following romidepsin treatment are graded. A score of 0 indicates no change in the ECG wave, a score of 1 indicates T-wave flattening, and a score of 2 indicates ST segment depression of 2 mm or greater. Accordingly, grade 1 toxicity can be defined as nonspecific T-wave abnormalities (flattening or inversion without ST segment abnormalities), and grade 2 can be defined as ST segment depression of at least 1 mm in at least two leads. If both are observed, then the ECG is assigned a grade 2 toxicity.

Blood samples are obtained before drug administration, at the end of infusion (4 hours), and at 2, 7, 9, 11, 14, and 21 hours after the end of infusion. All samples are immediately centrifuged, and then stored at or below −20° C. until analysis. Romidepsin concentrations in plasma samples are determined by a validated method based on liquid chromatography with single-quadrupole mass spectrometric detection [Hwang et al., J. Chromatogr. B Analyt. Technol. Biomed. Life Sci., 809:81-6 (2004)]. Pharmacokinetic parameters for romidepsin are derived using non-compartmental analysis using WinNonlin v5.0 (Pharsight Corporation, Mountain View, Calif.). Since romidepsin delineates a linear pharmacokinetic profile within the tested dose range [Sandor et al., Clin. Cancer Res., 8:718-28 (2002)], individual values for peak concentration (Cmax) and AUC_{[inf]} are normalized to a dose of 14 mg/m² in order to eliminate drug dose as a variable affecting the parameter estimates.

Genomic deoxyribonucleic acid (DNA) is extracted from 1 mL of plasma using the QIAamp DNA Blood midi kit (Qiagen Inc, Valencia, Calif.), following the manufacturers instructions, and is reconstituted in a buffer containing 10 mM Tris (pH 7.6) and 1 mM EDTA. Variants in the ABCB1 and CYP3A5 genes are analyzed as described in Example 1. The reference genotype is defined as the Refseq consensus sequence for the SNP position, and allelic variants are those differing from the consensus sequence. Genotype-frequency analysis of Hardy-Weinberg equilibrium and inference of haplotypes is conducted using Helix Tree Software v4.4.1 (Golden Helix Inc., Montana). The linkage between each pair of SNPs is determined in terms of the classical statistic D′.

All data are reported as median values with range, unless specified otherwise. Changes in QTc interval from baseline (ΔQTc) as well as drug clearance are evaluated with respect to the presence of a trend in the association of these parameters according to the number of reference alleles in individual variant genotypes using the Jonckheere-Terpstra trend test. [Hollander et al., Nonparametric Statistical Methods, Second Edition. New York, John Wiley and Sons, Inc., (1999)]. Because of limited numbers of observations, subsequent analyses are based on grouping patients on the basis of the number of reference alleles in multiple loci, with these resulting two group statistical comparisons being evaluated using an exact Wilcoxon rank sum test, with a standard Bonferroni adjustment used for multiple comparisons in these evaluations. The simultaneous effects of genetic variants and clearance on ΔQTc are evaluated using a regression analysis using a backward selection algorithm, and should be interpreted as an exploratory finding because of limited power. Again, because of relatively limited amounts of data for analysis, comparisons between the distribution of clinical toxicity scores vs. categorized genotypes are performed using Mehta's modification to Fisher's exact test [Mehta et al., J. Am. Stat. Assoc., 78:427-34 (1983)].

The characteristics of all patients are reported in Table III. In the initial analysis (“group 1”), romidepsin is administered to 45 patients (42 patients as in Example 1 and 3 additional patients) with T-cell lymphoma. In the confirmatory analysis (“group 2”), romidepsin is administered to 29 patients. The 17 patients with T-cell lymphoma receive the same therapeutic regimen as the original 45 patients in group 1, while the remaining 12 patients receive FK288 at a dose of either 12.7 mg/m2 (N=3), 17.8 mg/m2 (N=7), or 24.3 mg/m2 (N=2; on a day 1 and 5 schedule). Pharmacokinetic data are available in all patients in both groups.

TABLE III
Patient Demographics and Dosages

| | Group 1 | Group 2 |
|---|---|---|
| Parameter^{a} | (N = 45) | (N = 29) |
| Age^{b} | 56 (27-79) | 63 (40-77) |
| Male/Female | 28/17 | 18/11 |
| Race: | | |
| Caucasian | 34 (76%) | 28 (97%) |
| African American | 9 (20%) | 1 (3%) |
| Hispanic | 1 (2%) | 0 |
| Unknown | 1 (2%) | 0 |
| Dose: | | |
| 12.7 mg/m² | 0 | 3 |
| 14.0 mg/m² | 41 | 17 |
| 17.8 mg/m² | 0 | 7 |
| 18.0 mg/m² | 4 | 0 |
| 24.3 mg/m² | 0 | 2 |

^{a}All patients are diagnosed with cutaneous T-cell lymphoma except for 12 patients in Group 2 who are diagnosed with various refractory cancers;
^{b}Data are presented as a median and range.

A summary of the pharmacokinetic parameter estimates is reported in Table IV. The observed values for romidepsin clearance are within the range observed previously in patients treated with romidepsin at doses of 12.7 mg/m² and 17.8 mg/m². [Sandor et al., Clin. Cancer Res., 8:718-28 (2002)] The interindividual variability in drug clearance is relatively high, with a percent coefficient of variation of approximately 72%. Pharmacokinetic parameters of romidepsin are not statistically significantly different between men and women (all P>0.10).

TABLE IV
Summary of plasma pharmacokinetic parameter estimates

| | | | |
|---|---|---|---|
| Parameter | Group 1 (N = 45) | Group 2 (N = 29) | All (N = 74) |

| | | | | | | |
|---|---|---|---|---|---|---|
| Clearance (L/h) | 15.1 | (3.8-70.3) | 13.9 | (2.7-35.8) | 14.3 | (2.7-70.3) |
| AUC (ng h/mL) | 1760 | (358-6072) | 1008 | (391-5237) | 1501 | (358-6072) |
| Cₘₐₓ (ng/mL) | 501 | (88.0-1599) | 322 | (113-1213) | 431 | (88.0-1599) |
| T_{1/2} (h) | 6.8 | (2.2-15.0) | 3.8 | (1.0-8.8) | 6.0 | (1.0-15.0) |
| Vss (L) | 129 | (30.8-621) | 64.9 | (15.0-329) | 93.6 | (15.0-621) |

Data are presented as median with range in parenthesis.
Abbreviations: AUC, area under the concentration-time curve extrapolated to infinity normalized to a dose of 14 mg/m²; Cₘₐₓ, peak plasma concentration normalized to a dose of 14 mg/m²; T_{1/2}, half-life of the terminal phase; Vss, volume of distribution at steady-state.

For the Caucasian population, the observed ABCB1 and CYP3A5 genotype frequencies are in Hardy-Weinberg equilibrium (P>0.15) (Table V). Strong linkage is observed between the 3 SNPs in ABCB1 in the Group 1 cohort, with a linkage statistic (D′) value of 0.90 for the 1236C>T and 2677G>T/A loci (P<0.001); a D′ of 0.56 (P<0.001) for the 1236C>T and 3435C>T loci; and a D′ of 0.68 for the 2677G>T/A and 3435C>T loci (P<0.001). The most frequently observed ABCB1 haplotypes in the Caucasian population are the 1236T-2677T-3435T (T-T-T; 37.0%; haplotype 1), C-G-C (33.6%; haplotype 2), and C-G-T (18.0%; haplotype 3), although in total 7 different haplotypes are observed. The variant genotypes observed in the African American patients are also in Hardy-Weinberg equilibrium (P>0.13) (Table V). Strong linkage is also observed between the 3 SNPs in ABCB1 in the Group 2 cohort, with a D′ of 1.0 for the 1236C>T and 2677C>T/A loci (P=0.002); a D′ of 0.89 (P=0.007) for the 1236C>T and 3435C>T loci; and a D′ of 1.0 for the 2677G>T/A and 3435C>T loci (P=0.012). The predominant haplotypes observed in the African American population are haplotype 2 (66.1%), haplotype 1 (33.3%), and haplotype 3 (5.6%).

TABLE V
Genotype and allele frequencies of the studied variants

| | | | | | | Allele | |
|---|---|---|---|---|---|---|---|
| | | | Genotype frequencies^{a} | | | frequencies^{b} | |
| Allelic variant^{c} | Effect^{d} | N^{e} | Ref^{f} | Het | Var | p | q |
| Caucasians (N = 62)^{g} | | | | | | | |
| ABCB1 1236C > T | G411G | 55 | 19 (34.5) | 22 (40.0) | 14 (25.5) | 0.545 | 0.455 |
| ABCB1 2677G > T | A893S | 54 | 15 (27.8) | 22 (40.7) | 15 (27.8) | 0.481 | 0.481 |
| ABCB1 2677G > A | A893T^{h} | 54 | 15 (27.8) | 2 (3.7) | 0 (0) | 0.481 | 0.019 |
| ABCB1 3435C > T | I1145I | 62 | 13 (21.0) | 28 (45.2) | 21 (33.9) | 0.435 | 0.565 |
| CYP3A5 6986A > Gⁱ | Splice variant | 55 | 1 (1.8) | 9 (16.4) | 45 (81.8) | 0.100 | 0.900 |
| African Americans (N = 10) | | | | | | | |
| ABCB1 1236C > T | G411G | 9 | 5 (55.6) | 1 (11.1) | 3 (3.33) | 0.611 | 0.389 |
| ABCB1 2677G > T | A893S | 9 | 6 (66.7) | 1 (11.1) | 2 (22.2) | 0.722 | 0.278 |
| ABCB1 2677G > A | A893T | 9 | 0 (0) | 0 (0) | 0 (0) | 0.722 | 0.000 |
| ABCB1 3435C > T | I1145I | 10 | 5 (50.0) | 1 (10.0) | 4 (40.0) | 0.550 | 0.450 |
| CYP3A5 6986A > Gⁱ | Splice variant | 8 | 5 (62.5) | 2 (25.0) | 1 (12.5) | 0.750 | 0.250 |

^{a}Number represent number of patients with percentage in parenthesis; the difference in the total number of patients is due to the fact that not all samples yielded sequencing data or showed PCR amplification;
^{b}Hardy-Weinberg notation for allele frequencies (p, frequency for wild type allele and q, frequency for variant allele);
^{c}Number represents position in nucleotide sequence;
^{d}Number represents amino acid codon;
^{e}genotype data are not available in all patients as not all samples yield sufficient DNA or PCR amplified;
^{f}Ref, Homozygous reference allele patient; Het, Heterozygous patient; Var, Homozygous variant patient;
^{g}A single Hispanic male is also included, and his genotype is 1236C > T, unknown; 2677G > T/A, wild-type; 3435C > T, wild-type;
^{h}The 2677G > T/A polymorphism is triallelic and two different SNPs are therefore presented;
ⁱThe CYP3A5 6986A > G transition is also known as the CYP3A5*3C polymorphism.

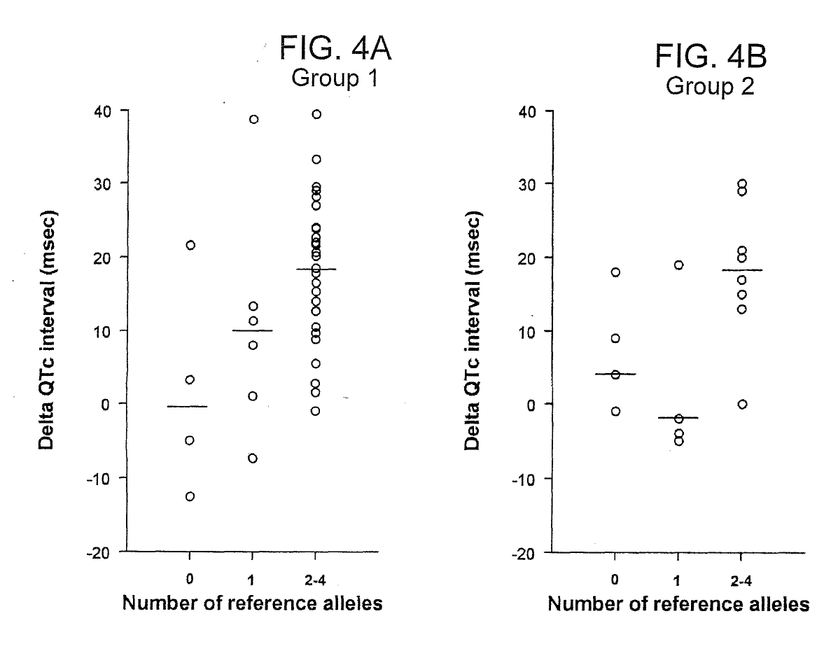

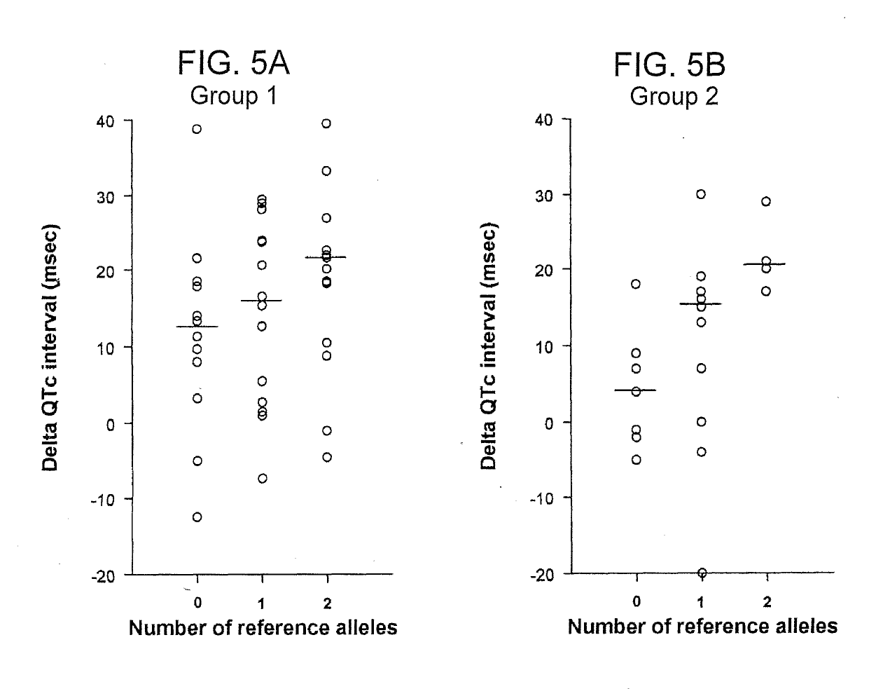

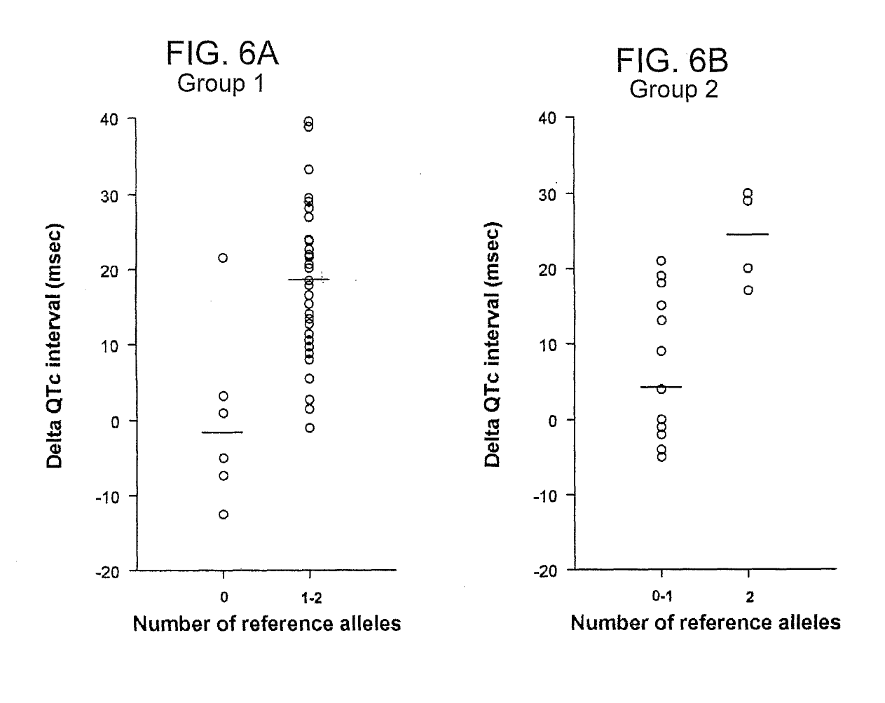

There is no association between the dosage of romidepsin and the ΔQTc in either group 1 (P=0.38 by Wilcoxon rank sum test comparing two dose levels), or in group 2 (P=0.30 by Wilcoxon rank sum test comparing doses up through 14 mg/m2, n=18, vs. doses of 17.8 mg/m2 and 24.9 mg/m2, n=7); thus, comparisons between genotype and ΔQTc are therefore made by grouping patients receiving different doses. In group 1, a significant trend toward increasing ΔQTc (i.e. the difference between pre- and post-treatment QT intervals at 4 hours) and increasing number of reference alleles of the ABCB1 genotype at the 2677G>T/A and 3435C>T loci is observed (P=0.011; FIG. 4A). Patients carrying a copy number of 0 reference alleles (i.e. “wild-type” alleles) at both loci have a significantly shorter ΔQTc (median ΔQTc, −1 msec; range, −12.5 to +21.6 msec; N=4) as compared to those patients with only a single reference allele at either locus (ΔQTc, 9.7 msec; range, −7.3 to +38.8 msec; N=6), or two or more reference allele copy numbers (ΔQTc, 18.5 msec; range, −1.0 to +39.5 msec; N=28). A similar, although weaker, trend is noted for the association of reference alleles of ABCB1 3435C>T locus and ΔQTc when it is considered separately (P=0.15; FIG. 5A). Additionally, patients carrying the 3435TT variant genotype have a higher median ΔQTc than patients carrying the 2677TT genotype suggesting that 2677 alleles have a greater impact on the association with ΔQTc. When the ABCB1 2677G>T/A allele is considered independently of the others with respect to its association with ΔQTc, a significant relationship is observed (P=0.0046, after adjustment for multiple comparisons). Those patients carrying no reference alleles at the ABCB1 2677G>T/A locus have a significantly shorter ΔQTc (median ΔQTc, −2.0 msec; range, −12.5 to +21.6 msec; N=6) compared to patients carrying one or more reference alleles (median ΔQTc, 18.2 msec; range, −1.0 to +39.5 msec; N=32) (FIG. 6A).

Similar trends are noted in group 2, wherein those patients carrying either 0 or 1 reference alleles at both the ABCB1 2677G>T/A and 3435C>T loci trend towards a smaller ΔQTc than those with 2-4 reference alleles (P=0.07; FIG. 4B). When the ABCB1 3435C>T allele is considered alone in association with ΔQTc in group 2, a statistically significant trend is noted whereby those patients carrying fewer copy numbers of the reference allele have a smaller ΔQTc after treatment with romidepsin (P=0.028; FIG. 5B). Similar results are also observed with patients carrying either 0 or 1 reference alleles at the ABCB1 2677G>T/A locus; these individuals have a statistically significant smaller ΔQTc (P=0.015, after adjustment for multiple comparisons; FIG. 6B). Those patients carrying 0 or 1 reference alleles at ABCB1 2677G>T/A have a significantly smaller ΔQTc (median ΔQTc, 4 msec; range −5 to +21 msec; N=14) as compared to patients carrying more than 1 reference allele (median ΔQTc, 24.5 msec; range 17 to +30 msec; N=4). Neither analysis includes the ABCB1 1236C>T transition as this SNP is in very strong linkage with the 2677G>T/A transition, and there is no evidence that the 1236C>T is involved in differential ABCB1 expression in heart tissue.

Neither the T-wave flattening nor the ST segment depression is associated with ABCB1 allelic variation based on the clinical scoring system utilized in this study. Based upon results from a generalized Fisher's exact test, the ABCB1 2677G>T/A allele is not associated with the scores obtained at baseline (P=0.46 for group 1; all scored 0 for group 2), or at 4-hours post treatment in either Groups 1 (=0.86) or 2 (p=0.18). Similar results at pre-treatment (P=0.086 for group 1; P=1.00 for group 2), or 4-hours (P=0.45 for group 1; P=0.47 for group 2) post treatment are observed with the ABCB1 3435C>T polymorphism. When the ABCB1 2677G>T/A and 3435C>T polymorphisms are considered in combination, the pre-treatment (P=0.067 for group 1; all score zero in group 2) toxicity score is marginally associated in group 1, while the post-treatment value at 4-hours (Group 1, P=0.10; Group 2, P=0.024) post treatment is found to be associated with the ECG abnormality score in Group 2.

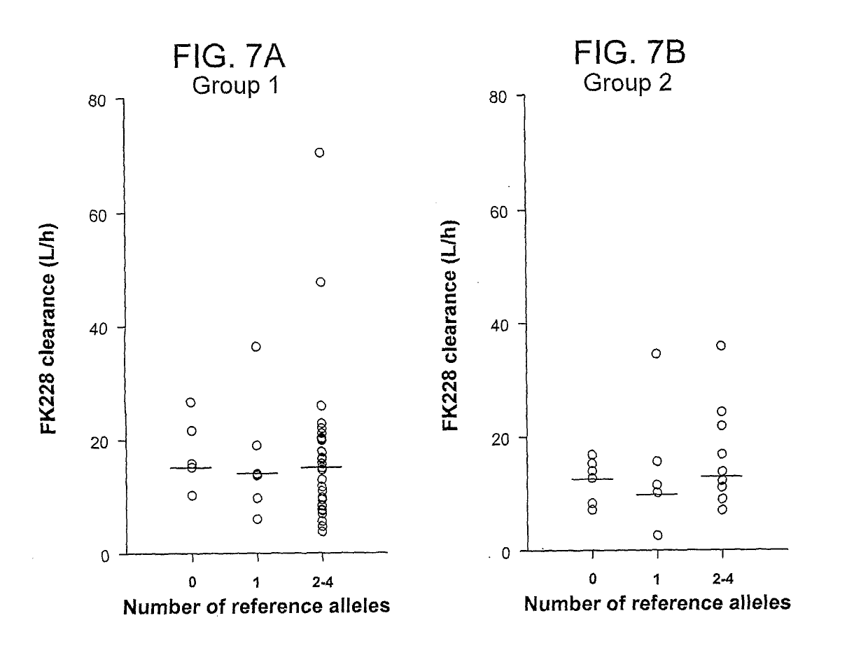

None of the variant ABCB1 SNPs, or combinations thereof is significantly associated with romidepsin clearance (P=0.51 for Group 1 and P=0.46 for Group 2; FIGS. 7A & 7B). Based on linear regression modelling using a backward selection algorithm, the ABCB1 2677G>T/A reference allele copy number is the sole parameter remaining in the model, and found to be a potentially important parameter in the determination of ΔQTc (P=0.0004 by t-test for whether parameter estimate is equal to zero). Systemic drug clearance is eliminated as a parameter for consideration in the model, with P>0.25 after adjusting for the ABCB1 2677G>T/A reference allele copy number. The CYP3A5*3C allele is also not statistically significantly associated with any measure of toxicity or romidepsin clearance (P>0.05). Differences in other pharmacokinetic parameters are also not statistically significantly different between the different genotype groups.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of screening for an altered susceptibility for a drug-induced heart rhythm irregularity, the method comprising:
(a) screening a sample from a subject to detect the presence or absence of at least one polymorphic variant of at least one polymorphism of the ABCB1 gene, wherein the polymorphic variant is associated with an altered susceptibility for a heart rhythm irregularity induced by a drug that binds a protein encoded by the ABCB1 gene, and wherein the polymorphism comprises a polymorphism at position 49,910, 68,894, or 90,871 of SEQ ID NO: 1, position 1236, 2677, or 3435 of SEQ ID NO: 2, or a combination thereof; and
(b) diagnosing the altered susceptibility of the subject for the heart rhythm irregularity as induced by the drug based on the presence or absence of the polymorphic variant of the ABCB1 gene.

2. The method of claim 1, wherein the drug is an anti-cancer agent.

3. The method of claim 1, wherein the drug is FK228, FR901228, a prodrug thereof, a salt thereof, or a combination thereof.

4. (canceled)

5. The method of claim 1, wherein the polymorphic variant is associated with:
(a) an increase or decrease in the expression of the ABCB1 gene,
(b) an increase or decrease in an activity of a protein encoded by the ABCB1 gene,
(c) an increased susceptibility for a drug-induced heart rhythm irregularity, or
(d) a decreased susceptibility for a drug-induced heart rhythm irregularity.

6.-8. (canceled)

9. The method of claim 1, wherein the method further comprises prescribing a treatment regimen based on the diagnosis.

10. The method of claim 9, wherein the treatment regimen comprises increasing dosage of the drug in the presence of a polymorphic variant associated with a decreased susceptibility for the heart rhythm irregularity.

11. The method of claim 9, wherein the treatment regimen comprises decreasing dosage of the drug in the absence of a polymorphic variant associated with a decreased susceptibility for the heart rhythm irregularity.

12. The method of claim 11, wherein the drug is not administered.

13. The method of claim 12, wherein a different drug is administered.

14. The method of claim 13, wherein the different drug does not bind a protein expressed by the ABCB1 gene.

15. The method of claim 9, wherein the treatment regimen comprises increased heart monitoring.

16. The method of claim 9, wherein a second, additional drug is administered.

17. The method of claim 16, wherein the second drug ameliorates the heart rhythm irregularity.

18. The method of claim 1, wherein the subject has previously experienced a heart rhythm irregularity.

19. The method of claim 1, wherein the heart rhythm irregularity is a cardiac arrhythmia.

20. The method of claim 1, wherein the heart rhythm irregularity comprises at least one member selected from the group consisting of asymptomatic dysrhythmias and ventricular arrthymias.

21. The method of claim 1, wherein the heart rhythm irregularity is characterized by at least one of ST/T wave flattening, torsade de pointes, and QT interval prolongation.

22.-23. (canceled)

24. The method of claim 1, wherein the polymorphic variant is present in a single chromosomal copy of the gene, and wherein heterozygosity is associated with an altered susceptibility for the heart rhythm irregularity.

25. The method of claim 24, wherein heterozygosity for polymorphic variants of two or more polymorphisms is associated with an altered susceptibility for the heart rhythm irregularity.

26. The method of claim 1, wherein the polymorphic variant is present in both chromosomal copies of the gene, wherein homozygosity of the polymorphic variant is associated with an altered susceptibility for the heart rhythm irregularity if homozygosity of the polymorphic variant is detected.

27. The method of claim 26, wherein homozygosity for polymorphic variants of two or more polymorphisms is associated with an altered susceptibility for the heart rhythm irregularity.

28. The method of claim 1, wherein the sample comprises a nucleic acid selected from the group consisting of (a) a nucleic acid encoding ABCB1, (b) a fragment of (a) comprising at least 20 contiguous nucleotides of (a) wherein the 20 contiguous nucleotides comprise the polymorphism, (c) a complement of (a) or (b), and (d) a combination of two or more of (a), (b), and (c).

29. The method of claim 28, wherein the nucleic acid encoding ABCB1 comprises SEQ ID NOS: 1, 2, or a combination thereof.

30. The method of claim 28, wherein the polymorphism is selected from the group consisting of:
(a) a polymorphism at position 49,910, 68,894, or 90,871 of SEQ ID NO: 1; or position 1236, 2677, or 3435 of SEQ ID NO: 2; or a combination thereof,
(b) a polymorphism at position 49,910 of SEQ ID NO: 1 or position 1236 of SEQ ID NO: 2, or a combination thereof;
(c) a polymorphism at position 68,894 of SEQ ID NO: 1 or position 2677 of SEQ ID NO: 2, or a combination thereof, and
(d) a polymorphism at position 90,871 of SEQ ID NO: 1 or position 3435 of SEQ ID NO: 2, or a combination thereof.

31. (canceled)

32. The method of claim 30, wherein the nucleic acid comprises the sequence of SEQ ID NOS: 3, 4, 5, 9, 10, or 11, or a combination thereof.

34.-36. (canceled)

37. The method of claim 28, wherein the nucleic acid comprises
(a) first and second polymorphisms wherein the first polymorphism is a polymorphism at position 49,910 of SEQ ID NO: 1 or position 1236 of SEQ ID NO: 2, and the second polymorphism is a polymorphism at position 68,894 of SEQ ID NO: 1 or position 2677 of SEQ ID NO: 2,
(b) first and second polymorphisms wherein the first polymorphism is a polymorphism at position 68,894 of SEQ ID NO: 1 or position 2677 of SEQ ID NO: 2, or a and wherein the second polymorphism is a polymorphism at position 90,871 of SEQ ID NO: 1, 3435 1 or position 3435 of SEQ ID NO: 2, or
(c) first and second polymorphisms wherein the first polymorphism is a polymorphism at position 68,894 of SEQ ID NO: or position 2677 of SEQ ID NO: 2, or a and wherein the second polymorphism is a polymorphism at position 90,871 of SEQ ID NO: 1, 3435 1 or position 3435 of SEQ ID NO: 2.

38. The method of claim 37, wherein the nucleic acid comprises the sequence of SEQ ID NO: 6, 7, 8, 12, 13, 14, or a combination thereof.

39.-42. (canceled)

43. The method of claim 28, wherein the polymorphic variant is a thymine at least one polymorphism.

44. The method of claim 28, wherein the polymorphism comprises a polymorphism at position 68,894 of SEQ ID NO: 1 or position 2677 of SEQ ID NO: 2, or a combination thereof and the subject is homozygous for thymine at that position.

45. The method of claim 28, wherein the polymorphism comprises first and second polymorphisms wherein the first polymorphism is a polymorphism at position 68,894 of SEQ ID NO: 1 or position 2677 of SEQ ID NO: 2, and the second polymorphism is a polymorphism at position 90,871 of SEQ ID NO: 1 or position 3435 of SEQ ID NO: 2, and wherein the subject is homozygous for thymine at both positions.

46.-55. (canceled)

56. A kit comprising:
(a) a nucleic acid for use in screening a sample from a subject to detect the presence or absence of at least one polymorphic variant of at least one polymorphism of the ABCB1 gene, wherein the polymorphic variant is associated with an altered susceptibility for a heart rhythm irregularity induced by a drug that binds a protein encoded by the ABCB1 gene, wherein the polymorphism comprises a polymorphism at position 49,910, 68,894, or 90,871 of SEQ ID NO: 1 or position 1236, 2677, or 3435 of SEQ ID NO: 2, or a combination thereof, and wherein the nucleic acid specifically binds to ABCB1 sequence comprising the at least one polymorphism or a sequence adjacent to ABCB1 sequence comprising the at least one polymorphism.
(b) a drug that binds a protein encoded by ABCB1.

57. The kit of claim 56, wherein the drug is FK228, FR901228, a prodrug thereof, a salt thereof, or a combination thereof.

58. (canceled)

59. The kit of claim 57, wherein the nucleic acid comprises the nucleotide sequence of any one of SEQ ID NOS: 25-36 or a complement thereof or a combination thereof.

60. (canceled)

61. A method of screening for a decreased susceptibility for FK228-induced QTc interval prolongation, the method comprising:
(a) screening a sample from a subject to detect the presence or absence of at least one polymorphic variant of at least one polymorphism of the ABCB1 gene, wherein the polymorphic variant is associated with a decreased susceptibility for QTc interval prolongation induced by FK228, and wherein the polymorphic variant comprises a thymine at position 2677 of SEQ ID NO: 2, or a thymine at position 3435 of SEQ ID NO: 2, or a combination thereof; and
(b) diagnosing decreased susceptibility of the subject for QTc interval prolongation as induced by FK228 based on the presence or absence of the polymorphic variant of the ABCB1 gene.

62. A method of screening for an altered susceptibility for a drug-induced heart rhythm irregularity, the method comprising:
(a) screening a sample from a subject to detect the presence or absence of at least one polymorphic variant of at least one polymorphism of the ABCB1 gene, wherein the polymorphic variant is associated with an altered susceptibility for a heart rhythm irregularity induced by a drug that binds a protein encoded by the ABCB1 gene, and wherein the polymorphism comprises a polymorphism identified as rs1128503, rs2032582, rs1045642, or a combination thereof; and
(b) diagnosing the altered susceptibility of the subject for the heart rhythm irregularity as induced by the drug based on the presence or absence of the polymorphic variant of the ABCB1 gene.


SEQUENCE LISTING (39 sequences)

SEQ ID NO: 1 - DNA, 95957 bp, Homo sapiens
  misc_feature (49910)..(49910): n may be any nucleotide
ggtcgggatggatcttgaaggggaccgcaatggaggagcaaagaagaagaacttttttaaactgaacaataaaaggtaactagcttgtttcattttcatagtttacatagttgcgagatttgagtaatttatttctagcctccagctctgaaataaatgacatgttgttgtttttaattatttttaagaaacgcaagctagcctttggaatcaatatccctgcttagagcagaagtttgttggctgagtggagcacagcatatgcattttccctgtcttttttgttctttcttttaatgatacataatattttacatatttatgaaatggggtacatggaagcgttttttacatgcccggaatgtgtaatgatcaagtccgggtatttgaaggatacatcaccttaggtatatttcatttctatgtgttgataacattttaagtcttctagctactttgaaatatacaatatattgctaactgtagtcaccctcgtctgctatcgaacattggaacttatttgtcctatccaaccgttcttagtcattcaccaacctcttttcatttcacctttttacccttcccggcctctttcccttagtcttggtgtgcctctttctcagctttcctgccccagacaggcggatgctcatatgtgtttctgtcttatgaacttctgcttttcaagtggtgttggtcgcccacacgtgagccatatgctgctggtgatctgctctgtggtccaggctcttgcttccggtaaatggctatgtaaacatcgcgtttgtggcctggctgatgagacagaaggtcaaaagtacatttaggttgttaactggcaataaatatctgtatataatattggtaatgtaatcatatagggaaaataattatttaaagtaaattttgatcatggtgctctgcctttatagaatatttaaaacttcactaaatagattcattgttagtagtaaattgtaaaatagactagtaagtttaataatattagaaactgtaatgtaaattataagataaattagtaaacacattaatattataagaaaccaagcttttcagtgtaagagaaaaaatacaaatgtggaaatcaaatacatttttaaaaataatgttaagtttgaattagaaatttcaatatgaattcataatcttttaatagttcattttcagtccactgaaagggacagtaacaatgagcactgttagtaccagatctgggtttctaaataccattcctccctaaaagaaatgaacccctcagtagctaatttcagccaggtctgggacaggaaaaatagaaagtgagcctggagtatcttgtggtacctgaaaataaggaagtggagtttaatggagtagtcgaaagcacactgaagctgtctggaagatgcttccaatggccaactctgggacaatttgagcatcaaagaacatcgtaactgtaactgattttgagatactgaatgctactgctgaatgcttaatattctgagagaagggaaactgaaagaaagtgtgagaggtagagaaaataaagcaatgatcttttagagaaaaattccagctatcattttggaacttccattgttataatagattcaggcaaggattgtcaatgaactctaaactatcagaaaaatgttgtatctaagaattttcttcagaatgtttactaattggaaggagaatatgcttctccaattaaagattttgcagttagcaccttaaccgagtgatcagacctggtatctctcacggtagaatggactgtagtaaatgactcatcacttggattttgagtaaacaacatcacctagcaaatattaatatatctgcaaaaacatttagtctgtatacaattaaacttgtagactggggctgtccaatggatatatcatgtgagccacatttgtaatttaaaattttctagtagcaacagtaaaaaatgaaaataaaaagataaagtaattttgtaaatatattttatagaactcaatatgtccaaaagattggattttctacatgtaatcaaaatgattaataagatagtttacattctcttttatgctaaatctcctgtttgatgtgtattttacatttattgcacctcccatttgtattcagtttgatacaggtatacccaagatgtgggacattctgtaagacagctgttcttgttcctttacaaaagtcaatgttaaaaaaataaaaggaggacttctagattaatagagactaaagagatataactaccaaacgtagtgtgtgaacattacaaaacaaaacaaaaacaatctaaaagttcaacaaggaaaaaatcctatggaaaacattcttgggagatttgaatgtgcattagatattagccaatatcagaattgtaatcacttttcttagatgtgataatggtaggagaacatgtaattgcaattgaaaatttttgtaacaaaaatgttcatataaaccatgaaaaagactccatagagtggagggtaggtattccactttttgtttttaactttgtatttttgaaaacttgatagcttaaagaattatgtacttatgatacaaacatttaggtagatttaactaagaaatattgagggtatgtgaagagaaacagtaaagaaaattgtatgatctttcacatatttctattattggttctatcatggcctatagactttttttttcaaattagagattatattttaaagtagttcttattaaattgtgttcattgccattgatttaaatctattttaacatgggattgtaaagaagcatatggctttggtaatagataatgctgggataatagtcctatttggaatatgactattagcaaagattctttattaaaatgatgtttgatgaatgacttgtctttctaagcactgttttttgtgctaatggggataagtaagttatctagtaggggtgagagtttgtgtgaaagtgcattttaatgtgatgcgatgcagtatctcagaaatctgaggttgcaccttatagtttggtggtcagggaacagacttggcatttaaatattggcttagttatagctgtgaaccttggacaagttaactcttctcactcttctacgtctctaaaagaacaaaaatgtgtcttcctcatagaattgtttatgaattgtgtgtgggaagtgcttacttagccagtgcctggaacattataacttctccagagtagcagctgttttagagaacaaaaaaataaataaaaggcttagagctaaaactcaactatttatggctatttttctcctttcatcctggttccagggatactgaaaccatgcctttactgggaattgggtgggaccaaacctgaagagttgtgtttgtgtattttatgtctgtctaacattactccaaagccaaatgggtaaactctggatttttttctttagaagtctcctcacctcttttgacctcacttagtgtaaagaacaaagacaaagatgaatttaccttatgaacttaaaaaccgtgtaaaaaataacacaaatcttttctaaaatagtttttcttttatacctacaaaaatacagatgaggcagatttgttacggttggtttgctttcacatcctaggtagcagttacacagagtagaaaatgggctacagagttagattcatttgtatctgggctcaaaataatagttgaccttatgcctgaggagaactttttgacaatttatagaacatcatttctgaccatagctttctagcgtgcatttatttcataatggtccatttaggactccagacatttttttcacaattagatttgcttataaggagtaatttaattttcttctgaggctagttatgcccagcatctaattgccacttctcttcactagaaagggaaggattaagaataatatctgggtagtggatggataaaatgtctactgccatcattaatcaaatgacaattaaggggattcataattaaggggaagcatgaatgattttctaattagaataaaaacagggaactcttttcagtatgtatatttttctaattgcaaagggtgtatgtatgtattcatcatagcaaatcaggaaatatcaaaggcacaaggaagaaaatatttttagagagattccagcactcagtgataaccacgtatgtagatttctaagatatatgtaaacatatttaaacattataaatggggtcatactattatcatcctgttttttaactgtataatatagacatatttcttcttcctattagtcattaaatatacattctatgttaatttgggtaatttaaaaaatatgtactttttaaagttttatgtaattccattgctgttttgcagtgaaaaagataagaaggaaaagaaaccaactgtcagtgtattttcaatggtgagttttgaatttattaactattcaaaatacttcggaaatttgacatctccttacatggaaaagagatatttcatctgatgtaagattttcgtatagggtatgttaatggagatgcaaaataaattggtttgatttagcttattttcaggagaatgctgatcataacttgctatctatattactaatctacagccaccattcctgacttagatttcaattcttctaatgaattatgttgccagtgatccattacctacacaagcacctctcccatctgcatagaggaaaccaatcacatagaatgctgtctttaatggctctgaaatttggcaggttttttaggtcaaagtcagttgctatcaataaaagttagcaatggattatttattactatgcaaattttaggtcatttaggttagtccattgcaaattttagttcaaaatccattactaacaataaaaggtagcaatggattatttattactatgatttcaaatatgtcatatgccattattcccctgtgttaaacagatgaaaatctagaattcgaaaaccttattgtaactctttatttcattttctggttgcacagtggtttttttagaagcttttctttaatcacacttggaatcagacatgtatcatatatgtgtggttggttgatgacctcagtgctgtgatcttggctcactgcaacctctgcctagttaatgacctcttatttacctggattcttacttccttttttctaagagtacaaatttaggtgaactgttaggtgaaagattccagagattagaacctggaagactactaaaccaattaagtccaacagcacatgctttataattcagtcataacacagcaatttttttaaggtaagggagtggatagattgtcttgcaaaatggattttaataaattgactataaaaatagttgtaggtagggaaaaaacagtcatgtgacttttacactgaggactctatggttcccttatgtgatcttcgtttctctgtgctaccattgctcttgccatgactcctgcttttattctatctctacgccctttggaattttggtgagatgagactatgtacttgagtcattcttaggctacttgagtaaccaagtgacagttttcattgttactttcttgtatggaaagaagcgtttaggttaaagttgtatttatccccaggatttcagtgctgtcactttgagactttcagtttttattatttgtagttaaatttttttaaaccttaggagtcttatattttagaaaatttaacagacctactatatagaggcaatgttatcaataattcattttaaggcacaaaagaataaaaatatggagctttaatatgaggtagtagtaatagcacacataaagcactgtgtgccaagcattgtcctaagtatttcatatttaatctcatttaatccttacaactccttttgagaaagatactattattattccagttttacacatgaagaaactggagccagacatgttcacttgtctgaggatgtacaatggggccaggatagagagaatatattctaagtctcttgatgaagtactctactacttagatagattttcctcacatctttttcctgaaaagtcaacatagaaaacattgaatgtaacttcaatataaaacttataaacacaagaacataataaaagtttaacaatatattggtgatgtcactggcattgcttattaataatagttttcagtgaagacaaaaaacattgtgtgttcagggtttccaagaagcccagcttcagtgattcagactaggaggacccagaggatttatggctaatgtttattacaacaaaaggatacaaggcaaaatcagccatggcaaaaggcgagtggggagaagtccagaagaaaccaggtgcaagctcctagagttctctcctaatggagtcacagggcacactgaatttccccaagcaatgagttaagacagcacttgtagaatgctgtttatcagagaagctcattagagactcagtgcccaaggattttattaggaactgatcatgtaggcacctacccagcatgcaccaaaatttcagactcatagaaggaaggcagaactgcagaatgaagaatcatattgtttgtacagatagtttggccatacttatcagttagaatggtggtaatcctccccaaatccaagttccagccaagggccaacctttgtatacaggcctttctaaacatggcagcctcagagctgctgtgttagctcttgtctgcacacaatgtctgttaatttatttccactatgagattttttagcaatcttaaaaaagtgatgaaagaagtaaggaatagattcaaaattctatactagccttttaaatttatatcagaccttgtttcaacatttacagaaacacaaaagtagaataaaatctaaagcaagtgaggaaatgtgacacatagagctgggctgtaatttctacacaaaatgaacttcttagagattatacactcttggtcaggtgctgtggctcacgcctgtaatcccagaactttggaaggctaaggtgggcagatcactagagccctgaagttcaagaccagcctgggcaacatgacaaaaccctgtctatgtgaaaagtacaaaaattagctgggcatggtggcacatgcctgtagtctcagctactcgggaggctgaggtgggaggatcacttgaacccaggaggtggagatgcagtgagccaagattgtgctactgcactctagcctgggtgacagagtgagacactgtctcaaaaaaaaaaaaaaaaagattatatattcttgcaagaattgtgcactgtagattgaactctgagctttctattatgtgtgtcttagggaaacattaatttttttctaaatagataatacctaacaatgttaagtgtttcccttgtaccaggcatttgttaaataccctatgtttattgtgtgacataatcttcatatagggacataagtacctgcaatttaggaacttgtgttattcttgtgttccagatgaagaacttgacttagagaagttaagtaatgcattcaaaatcacagggtcacatagctactaagcagggtagactcagatatgcccagttgcttttaacaactgtaatcatttatgactggttaaactgttatctctctggaattgaagagttctttattgctgctatcttttgatcttatgaccatctcatcatcaaagactacgccacctggctcagtatcctccctttccactttggtgctccaaccaacttgggtgacttttatggttatatggataatcactccttgagcttcacagctgcaataatcataaaaacctacctgtgtctgaatctgtttcctcctccttctttccagttatgacagagttttagtcttaagactaatcattcatactttggctcctatctcctctgccttctcagggaccttacatcattattaccactcattctcttctgtatcttccatctttccctctcaactggatctgtcccattggcattgacacatcaaggcactgatgcccttcatggagccacccctgccagctcccttcacacacaaaagtggcctttgcgttctgtcttgatttcgccattgacttagttctcaacctattgaagtttggctcctgttcctgtctctctctacattcagtggacattttaaaattctatcttgatctctcagcagcacttagactttctcctatattttttgtatttgcttctgttatgtcacggtttgctgattttcctccagctctttggctttttcttgtgctgccctcgcctgtaaagttaagttcctcaaccccttatcacaggagtcagagatgatgtggacattcctatagttccacaaagccattttcaaattttttttattctcatgcaaactcctttgtcatttgagtttcattctgcataattatactatcttctatctattttcggtggtatcctcgaagtatacccccacccccattccttgaggactttggcatttgattgcttctacttcattcaccttatgttcctgttattcttctgagtattttggtgaccttacaaataagtctcaacattctctgactgcttcagttccaacaacgacgctccataaattacatgagtaccttagtaaattgcacgtgtggacaggatcttgggcttattgtctgggtgagctttcctctatgaaggtataaacacacacacacgtgtatcagtctttgaccacaacctgtgattccttccagctctgccctgcctctttatcatccctcttttgacattgtgtattcatttcctattgctgctgtaggaaaatagcactcacttagtggcttaaaacaacacaagtttattaccttactgttctggaatcgaagttttactgggctaaagtcaaggtatcagcaggattgcatttcttctggaggctctgtttccttccctttttcagcttctagaggacacttgcatttctagactcacaggcccttccttgcatcactccaacctcttacttcaatcatcacatctcctactgactccaggccccctcctccctcttataagcacccttgtaattacatttgctctcctagataatccagggtaatcccctatctggagatcttttaaatctgcaaagtcccttttatcatgtaaagaaatatattcacaggttcctgggattaggatttggacaggctgtggggggagaattattctgtctaccacacctggtcaagactttactctctcaactgctctttcatctcctagtatccctccttcctttttgctcttctaaaaatttacccatagcagccacatcatatcaccttggtccctaccttaaaaccattaaatgctttcctttctgcttacacaggaagttcagtgtaaaacccagactccttcctgtcatcacaaaatgccctacatatctgacctctgttatttctgactttactgcagacaaatattccctctcactctgctcttgccacagtgttcattctctttggtccttgggatacagcaagctggtttttacctaagtacctttgcattggctcttcctgcctggaatgcactgtccccagatcatcacaggatgaactgtatccttcctgtcactgaagtctcatcatggatgttacctcctgagaaagacattctctgaccagccaatctaaaccaggtgaccctgcctgtctctgtgtcataccatgtatggcttttctcctgaatatttatcactgtctgatttcttttttatgtgtttgtcttctctctgaaggtaggctccgtggaggcaggagattttttgtatattttgttctctactataacccaaatgccctagaatggtgcctggaactttcatagcaacttaaatattgaatgaatgttgaatacatctattcccccttttagtgtatgcactagagtgtatgctttgtgaggataggtagcttttcttactcactgttgttaccagtacctagaaccaagcctgaccttattaggttctttcaaatatttgaaagatattttaaaatattcacatatcccttgaatgttaaatgaataaatgaatgaattaattctctctcaaatccaagtcatgtttgccatctaatctggtgaatgttacctaaacctctctcctgtcagtaccttctacttctttgccctgggaagcccagtctttggcattaggcacggatcaacatgccttttatgtggttctggttcaccttctctctctttcctttcagcatctaatcaaactttctttctttctctctctctctctctctctccctccctctctccctctctctctctctctctctctctctctctcctctctctctctctctttctttttttaggtcttaactctgtcacccaggctggagtacagtggcatgatcacagctcactgcaggttcaaccacccaggcttaagtgatcctaccaccccagcctctggagtagctaggaccccaggcacatgccatcacacccaaacaatttttaaatttttttgtagagagagggtctcctatgttgcccaggctgtaccaaactcctgggctcaagtgatcctcctgccctcagcctcccaaagtgttgggattacaggtgtgagccatcatgcccagcgcatttccttttttaaaagctcctattacaattagtttatacttctattttacatttcacctgtgttacaacgttttttggtctgcctgctttttgactttatgagagtgaaaagtcttgttaattattatattttcaaaaccagtaacattatctgcttatatcatatatttgcatattgtatgctcaataagtttcttgtattctcatttttaccctcataacagctatgtaaggttttctgtgtgtatgaatattttcctcattttgtaaattcagaaactgagactcaaggttattaagatttttttcccctaaagtctccttcctaataaactacagagtgagggcatttgactgaggcttttgtcctactcagcattatgttaccactttcttcaaatcttctcacccccttctctctagaaatcaatcttgcttcatgtcattaagaaatatgaggttctagcatttataaggaacctaaacaaatttacaaagaaaaaacaaacaacccaataaaaagatgggcaaaggacatgaatagagacttcttaaaagacatgtggccaacaattatataaaaaaagctcaacatcactgatcattagagaaatgcaaatcaaaaccactgtgagaaaccatttaacaccagtcagaatggctattattacaaagtaaaaaaataacagatgctggcaagattgtggagaaaaaggaacatttatacactgttcagccattgtggaagacagtgtggtgattcctcaaagacctaaagacagaaataccatttgacctattactgggtgtatacccaaaggaatataaatcattgtattataaagacacatgcattgtatgttcattgcagcactattcacaatagcaaagacatggaatcaacctaaatggccatcaattgtagactggataaagaaaatgtggtacacatagagcattgaacaccatgcagccataaaaagaaacgagatcacgtcctttgcagggcatggctggagctggaggccattatccttagcaaactaatacaggaacagaaaaccaaacaccacatgttctcacttaaaagtgggagctaaatgatgagaacacatggacacctagaggggaacaacacacattggggcctttcggagggtggagtgtgggaggagggagaggatcaggaaaaataactaacgagtactaggcttaatagctgggtgatgaaataatctgtacaacaaccccccatgaaacaagtttacgtatgtaacaaacctgcatttgtacccatgaacttaaaagttaaaataaacaaataaaaaataataaaaaaaaaaacaaaaggaaatatgaggttctgaactctttcaattttctgtctttattcctggaaaattagtactcactttaatctccttccttctagtctaagattaagagatgtaattccaattttcccagctcagtgaggggtgtcttttgatcagtcagctatttctgtttccacactttacaccccaaacttgagggtccccagtcttttttaagcacattgtgctcttttctgtctcagtttacttttctttttggcttaaatgccttttttttttttttttttttgagacagagtctcactctgtcgcccaggctggagtgcagtggtgcgatctcggctcactgcaagctccgcctcccaggttcacaccattctcctgcctcagcctcctgagtagctgggattacaggcgcctgccaccacatccggctaatttctttttttgtattttaatagagacggggtttcactgtgttagccaggatggtattgatctcctgacctcgtgatctgcccgccttggcctcccaaagtgctgggattacaggtgtgagccactgcacccagctacacttggtaaactttcaaagctggaaggaagcatcacctcttttatcatatgaagccttttcacaaaggagggaatgatgattgactcagttttgtgtctattctatacactgtactgtcaaagcatgcagagcatgtattgcatatacatttttctttaacctctctgaaggcagggttatatctttggcatttatctctggattttatctgtcattgaataggcattcagtaaatgtctactttcatgatccacttatttttattatggccagtgacagttctggaggtaaaatgtcctgtgttggtggtgatgggagaaattttttcatatcaactatcttctcttcctgtactaaggagtttgatattttatctactttaatataatctctaaaagaagacctcttccctttcatttttgtttaaggaagcagatgaattcttatctaagacaaagtacatagtgcaaaatttagccagtgtttcaatgaaatatgaaaaaatatgccaaaacgtgttgtcccattaatgttttaatctgtaaatatgatgatgtttttagatcatcaacccataaataacatggaaagttatgccatgaacccaaacttaagctcatttgataacacatttaagagctagaaaaagggtaaatgcaaatacttaagtatctattagatactcttttttacacaccatatcttaattttcattaattatttcataatttggctttactttttatctcctgttttatggactgccattcattactggtaataaccattctttatttctccactcggcagtccagtaccaaatcccccagttgctactgtaagattcatgtaagctaactctaggattggtttctatccccttgtgaagtatatgtaagttctaaaatcttgctgtctgtggctatgtcttttttgctgcctaatctttgttttggtttatatttcctgcctaggcctatgatttttttacccagtttcctccctgcattacaaacctgttgcctacagctacaccttgcatccacatgttaaccttctgccctgggatttatccatagacacccacagctggctttaccccatttctgttttttttccctttggttttctttcaatccttagctagttccttgagttatagggaagcagtattaatcacttcccaagggggccctgagcccattcctcttaactgtgtccttttcctgtaactttttgggttcagagaagcttttctttccttcctgttacctgcctacatttaaagagattatgcagatctccaacgtgggtcattcattattgccatattctgtcttctcagaaaaaatgttccttgacaagcatgcatcagactcattcctttcttttttctgctcattcttgaggcctaattatcttcaaatcattttacctttataccagttccattgtttggcaggtctttaacccaaaccactttcttaaagcaacacaatgtttcctccatggtatcctcagaggggcctttggcattcaagtttttgcccatgagatgataagtggcttctattcttagaaactagagcaacaaatactatattatataccattaaatacttttacagtttcccagaatgtcccatccttctcattagacttcatataagaagacagaccaagtccaatatgaagaattaggccagaactcaatggaagaatcaaatgtccatttctaattagactattgcaataatttagcaaaggtagagggtgtcttggactagggtggtatcagtagtagtctgaaaaatgtttggattttggatatattttgaaggaaaagcaaatcttccatgaaactgtactaatttttcctctgctattcttttttctctctttttagtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtaagtattgtttgtgtaccaattttattggaaacttgcagcaaaaacaagcacttagagtgatgtttatggattctttactaaatacacattagtgtgtttagtcctgtaggaggcagacagtctgagttataattcatatttgtagtgagttagaaaagataatatttacaagcattacaacacacaactgaagtgccagcagagatatgtagaaaaagagagagccctgtgaagtcagaaatattctggaaagcttcaagaagaaatataggaacttaattagaaccttgaatgataagtaggaatctgttgtgagatttcctaacagattgaacatgggtgtaagacgaaaagtcggggatgattctaaagtttttagccctagaaactgggaggatgcagttactatcaacagaaatgagagatggagaaggagagtggatttgagcaataaatcccagaaatttagttttggacattgttatatgttgggctttctggaagtagatgcaggaatggaaaagtggggcaaaattgtgtgtgtgtgtgtgggggggtattttaggataggataaataatagcttgtagcttgttttgtgtgttaatggaaatgatccaaaagaaaggagaaagtgatgtttgaatgaaagatggaagagatttgctgagtgatatccttgagaaaaaatggaatccagtgctgaggaggaaaacagtaacaggtgggaaaagtgaatttgagtgtcagtacccagtggtgggtagatgagaaggtgagaatctgtggaatttttctcctgataactttagttttctcagtgaagtaggaagcaggagaattggctgatagagacagagaaaacagatgtaacacagctctcaaggaaagtgggaaagtgaatagactacagaaataatctgggtgtaacatacatattctttcctctctctccctctctctctctctttctcgattggggaggaatgggttgatatctctgagtttcctaggttacctctggtcaagcctagattgatttaaactgagctactcaaaaacttacaatccagtttgcactcaggtggtcaaatgttgaaaatgcctgtttgataacgtacttgaaaatgtagtgataccacttagcttacaaggacctttcaacttttgaactttcaagtttctgatgaatgcaatgaattgcttccccagaaacatgcacatgaatattcataccaatctcgggtatatacggatcttttgatacttgtcagtgggtcctcagaaccacttcatgaccctaaaaagtacattgtttaccctggggtagaactacttcaattctgtggatgcaggtaatgagctgggccagaaaaatttttgtatgtttgagctatgtaaaattcttatgctttattacatcctttcacggcatccaaagagacctgcagaaagatgctgcaccacctctgaactggtacctttcttttacaccattcaccagaggaagtcctcaatgctttctgaattcattagttcctatacttgctatattggccaatattctatggcgttagctctcttactgcttcatagtggaagaatcaaatacttcatcactctctggtggctcagcaggcctaatgggtacatctctgtccatccagatcttgctcactttttaatgacttcccagccttcagaactgtgggaaataaatttttattgtttgtatgttaatacatacagtttatgtattttgttatatcatcccaaatggactaagacacacagtaaactcacttacttaatagcatgcatgagctcccaaacagatgatgatctagttgtgaagactagctcctttctggataggcccatgtgtctgtatgcctatcaaaatctcctaggttaaacaggggccacatcaccacagcttgccagctggtaatttagatttccatcttcatagatatctagtttctaggtgaggttgtaattcacattgcagtccttgccagtcattctcattcagctcactcttctagtcttgagaacgggaaagagaaagcttagagaaatatttgaaagtttacttttgttattctcttaagaggtggtatatttagtggttagcgaacatactctggagttggactgcctggctcaaaaaccacacccttaccaatttttaattttgaggtttagggaatttattcatactttagttattaagttggataacaatagaacctacttctcaggatggttgtgatatttaagtggattcattcattgaagaatcacagaacaaagtctagtcataagaagtactcaacaaatattagttactgttattatcaacaggctttcatgttcctcacaaatgaaattacataactttcaatgatgatatggttccttatataatttcttctttacaaaattctgtgatactacctcaattttcatagttgttgaaagtttgggacatacccaaagttactcagtacctattatatgatgtgaataatcatccctttaagtagaatttccttggttaccagccatgcctttcagggaaggtaggtatcctccaactatgacccctgggctaatttggcctgctgcctctttttgtacagggtgtgaactaagaatggattttacattttgaaatcattgaaaaaaaatcaaaagaagaaattatattttgtgacccgtggaaattatgtgaaatttaaatttcagtacctgtaaataaagtttaatgaaatgtagctacacctattcatttacattttatgaatgtaagcagcctatggctgcttctatgccacaatgacacagtagagtagttgtacaaaccgtatggtctgcagagcctaagatatttactatctggccctttatagaaaaaggttgccaatctctgaagtagggtattgagtgaggggttgttatcttcagtcaatccatcaattaatttgtattagagcattctgtgtgccagtcacagtacatgccctcatcattcccaactcttgaggagcttagtgtagtgggagggatggacaagggtgaaccaataaatacagtcgaatttgattagtgatataatacagagataaataaaatgcattggaaaaagagagaaaggacacctgactccatatagaggaatcatgggaggcttccaagaggaggtgataaccaaatatggtcagcatctatctcctacacaaagtgtacctattctgtgtaacagatgaaaccatttcaaaataaagattaaataagtaagattaatatccttccctcactgccactccttatttccactcccctcaccattgtctatccttccatgcctttccaaagacagcatgctatctttactgtgattttcactttaggacgcttactacactatcactaatctgcttgtctgcagattcagactgccagataccctcaagtctgcctggaaagcagggaatagcatcaaaggttttgatgcttgtcccagagatgagggcaaacatgctctgtcatgcctgtggagggagaaccagcctactaaattcttggggcccgagatacagtcagatcattcttctttttgcccttccctaggcagaacctcattatctctccatgggggtggactagataccagagcctccttattggaaaagtagtccttgcttaaggggacttggacttgcggaggctcttgaaagcctgggggtttttacaataaacataggatctaattagacagctctaaaaccttcttcagtaagcagatccttctctgttgctgcaatagaacccttgtaagttttctcgtcccaatactttgccttaattaacagacatcagcttttgttctgctgttttactgtggaccagatttcctgacagccactcccatctatctctggtttaattatagaatatgtgtagcctttgccttaaaatctctgatgactggagcctagggaactctctgattctgtcatatcacttttttttttgtaaacaaagcttaatcactgatgaatttctgtaatattctgtgaattctcatcttccttatgagactggccactatctggaaatgatcctagataaaggcgattgtggttttaaatgtgttgtggtgaagacggcaggtggggtgcctgtaaaatgacatccaggcagttgaagaaatgacactaaatcttggcataaagaacaggactagaagctgggtgtggtggtgtgtgcctgcagtcccagctattggagaggctgaggtgggaggatcacttgaggtcagaagttcaaggctagcctgggcaagatagtgagagtttgtctctaataaaacttaaaaggttaaaaaatgataaaatgtaaggaggaggtggaacaagatagccaaacaaaagcctccagaaatcatccccccctcccccatccctacaggaacaccaaattgaacaactattcacaaaacaaagcaccttcataagaactaaaaaccaggtgagcgaccacactatttggttttaacattatattaaggaaagaggcactgaagaaggtaggaaagagagtcttgaattgcctgcaccactcttcccctcccccaccagcagcacatactgaagaatgaatcacagtctcttaatagcaaaattgatcaagcagaaggaagaattagtgagcttgaagacaggctatttgaaatacacacactcagaggagacaaaagaaaaaagaatgaagcatgcccacaagacctacaaaatagccttaaaagggtaaatccaagagttattggccttaaataggaggtagacagagagattagagtagaaagtttatttaaaagggtaataacacagaatttcccaagcctaaagaaagatatcagtattcaagtacgagaatgttgtgggacaccaagcatatttaactcaaataagactacctcaagatatttaacaatcaaattcccaaaagtcaaggataaagaaaggatcctaaaaggagcaagagaaaaggaacaaataacatacaaaggagctccaacatgtctggaagcagacttctcagtggaaactttacaggtctgaagagagtagcgtgacatatttaaagtgctgaaagaaaaacactttttatcctaaaatagaatatccaatgaaaatatccttcaaacatgaaggagaaataaatactttctcagatgaacaaaagctgagggatttattcaacaccagacctgtctagacaagaaatgctaaagggagttctttaatcagaaagaaaatgatgttaatgaacaataagaaatcatctgaaggtacaaaactcactggtaataataagtactcagaaaaacacagaatattacaacactgtaattgtggtgtataacttactcatatcttgagtagaaaggctaaaaaatgaacctataaaaaataataactacaataactgttcaagacacagtctaataagatataaacagatacagatataaaaagtagggagctgaagttaaagtgtagagtttgtattagttttccctttgcttgtttgtgtatgtaatcagcattaagttgccatcagtttaaaaatgagttatatgatatcatttgcacacctgatggtaacctcaaataaaataacatacagcagatacataaaaaattaaaagcaagacattaaaacataccaccagagaagatcaccttcactaaaaggaagacaagaaagaacgaaagaaggaagagaagatcacaaaaaacaacaaacaaaatggcaggagtaagtcttcacttatcaataataacattgaatctaaatggactaaattctctaatcaaaagacatagagtggctgaatggattaaaaaaaacacattaaaatattatttaagaccaaatggggtttatcccagggatgcaaggattgttcatcgtatgcaaactaatcaatgtgatatgtcatatcaacagaatgaaggatataaaatatatgataatttcagttgatgatgaaaaggcctttgataaaattcaccacccttcatgataaaaactgggtatagaaggaacattcataaacaaaataaaagccacataaacagacccacagctagtatcacaccaaatgaggaaaaactgaaagcctttccttcaagatctggaacaagacaaggatgctcactttcactagttattcagcataatactgaaagtcctagcgagggcaatcagacaagagaaagaaaaaaaaaggcatctgaattggaaaggaagaagtcaaattatccctgttggcagatgatacaatcttatatttggaaaaacctaaagactccaacaaataactattaggagtgataaatgaattcagttaagttgcaagatacaaaatcaacatacaacaatcagtagcatttctacatggtgacagcaaacaatctgaaaaggtaatcaagaaagtaatctcggccgggcgcggtggctcacgcctgtaatcccagcactttgggaggccgaggcaggtggatcacgaggtcaggagatcgagactatcccggctaaaacggtgaaaccctgtctctactaaaaatacaaaaaattagccgggcgtggtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaagaaagtagtctcatttactattgctacacatgaaataaatacctaggaataacttttactcaataaatgagagatctctacaatgaaaactgtagaacattgatgtaaaaaattgaagaggacacaaaaaatggaaagatactccatgttcattgattggaagaatggatatttttaaaatgtccatactaccaaagcaatctacagacagtgcaatcgctatcaaaataccagtgacattcttcacagaaatagaaaaaagaatcctaacatttacatggaaccacgaaaaaaacagaatagccaaagctatcctcagcaaaaagaacaaaactggagaaatcacattacctgacttcaaattatactacagagctagtaaccaaaacaccatggtacaaagaaaagagacacatagaccaatggaacagaatagagaatgcagacataaatccacaaatctacagtgaactcatttttgacaaagtttctaagaatgtatattggggaaaagaccgtctcttcaaaaaatggtggtgggagaactggatatccatatgcataaaaatgaaactaaatccctttctctcaccatatacaaaaataaaatcaatatggattaaagacttaaatctatgacctgaaactatgaaactactaaaagaagacactggaaaaactctctaggacattagattgggcaaagatttcttgagtaatacccctacaagcacagggaaccaaaacaaaaatggacaaatggatcacatcaagttaaaaaccttctggactgcagaggaaacaatcagcaaagtgaagagacaacctgcaaaatgggagaaagtatttgcaagctatccatctgacatcagattaataaccagaatatatgaggaactcaaacaactcaataggaaaaatctccaattaacaaatgggcaaaagatctgaatagacatttctcaaaagaagctatacaaatggcaaacaggtatatgaaaatatgctcaacatcattgatcatcagagaaatgcaagtcaaaactataatgcgatatcatttcaccccagttaaaatggcttttagccaaagataggcaataacaaatgctggcgaggatgtggagaaaagaaaacccttgtacactgttgtacaacttgtacatttttgagaatgtaaattagtacaaccatgatggagaacagtatgggggttcctcaaaaaatggaaaattaaactaccatatgatccagttatccctctgctggggaaatactcaaaagaaaggaaaccagtatatcgaacctgcactcccatgtttcttgcatcactattcacaatagccaggatttgggagcaacctaagtgtccatcagcaagtaaatggatgaagaaattatgatacatacacacaatggagtactattcatccttaaaaaaatgagatcctgtcatttgcaacaacaaaaatggaactggaggagattatattaagtgaaataagccaggcacagaaagacgaacttcacatttctcacttatttgtgggagctaaaaaattaaaacaattggacttatagagatagtagaatgacatttaccagaggccgggaagtgtagtggggtggcagaggtagaagtggagatggttaaaggatacaaaaatatatagttagctagaatgaatatcatctagtatttgatcacacaacagggtgactacagtcaacaacaatttattgcacatttaaaaataactaaaagggtataactggattgtttgtaacacaaagaaaggataaatttttgaggtgatagataccccattcaccctaatgtgattattatccattgtatgcctatgtcaaaatatctcatacaccctgtaaatatatacatctactatgtaccacccaaattaaaaataaattaaaattaaaattaaataaatttaaaaataaaactaaaaaaaccaggactatcaacttggctttatttaagttgaatgtgatgttaaatcctctgggatagaatatggagaaaagtagaaagaatgggagccccaagccttgaggaagaactcaactgttagcaataactgaagccaaccctcattgaacatttactatttgctgggtcttgttcttggagctttacatgcagtaactcttttaattcttaaatacacatgagattaatactattatatatgtgtgtaaaagtatctatatatacttacatataattatattttatatataatatatacttacacacacacttatatatagtgtgtgtgtatatgtatagctctgtgaaaccatttgcaaattttttgaataggttaagttatagcagatacaacaggttgggcactggatatataaagataaacaaaatatagtacagttttctaagaccataataatatatggtataataatatttttggttaagggttttcaagccaaaaagtagaattagttacaatgtaatctctgcaatatattagaaaaacagcaaaaaaaaaaaagatcaatatacagatagtacctaacccattctaggtgcttaagaaattggatgaataaataaaagactgaataatagaattttgtaggtgaaaaggggcttagatgttatctagcctgaacccataatttatagacatacatgcccaggaggttgaattaatgtggccataatggaattgcttgcgggttgacagagctgaattcctgggccaggaatcctactttttagtctggtatcttttccactctgctagtggttttcacactctgcctaaattggaagcaaatgccagcacttggtcttttaaatctcttggatgctgccctgtaagtttgatttttagtgtgtcaggaaggtcaggcaagaggcacagctcttcaggccagaggtatataaacagctcacataaaaagactgtagttcctggaatcatctctgtcacctgcagtaagcccaactacactgagtcacagccttcagtttgggccaatgccatagggatttatcatttaggcaaaagatccaaattccccttccctgcggttgatggagctttctgcacaagaagagtagtttattggtttattagagcttcttaaatgggtagaaaagctaaatgaaaaaaaaacggaagttcctgggaaaaactgtgctaaataggtcagcagtgttaccctgtcacctgaagtttgaggcctaaaagtgaaaggatttcttacttgaacagaaatccgatgatattagagaaagtggctctcattctgggtttatcttcagtgtctggaatgatgcctggcatgaagtctctgtggaagacagtttgaaaaccaccttttcaatataatttcagtttaccaattagggaattatcaatgcatcatcaaaggataagactatactaatgtatccatgttatatattttttattcatactgttcttaagtgttcagttattaaagttactaaatgtactgaagtatttaggtcaaataaaaaatccctaaatggtacagtcttatcctttaaagtcttgcttgggtttagaaaaggcttttcttgccaggtagaaaggtctaatgtatctctctttaatcttaaaatatgtgagacacttgtatacaaagtaggagaatggttggatggaatattttgataaatggtctggcttgtgtagtagctctgtgaaaccactcgtaaattttttgaatatgctaagttatagcagatacaacaggttgggcactggatatataaagataaactagatatagttcagttttctatgacctcacaccagtgggagtgggaggaagagataggtgtaaatagtcatacttctatttttgtatgacttagtaagttgaatcatctgtcaattactacggagttctattgcaagatgagtcactctgctaggggtaagatgatgtaatactttagtttattcatggaggaagagtatattccaggagaaaaattttgataaagggcattccagttagagaaagcagaatggataaagccttgaaggaagaaaataggatagttagggaacagtaaactcttcggtgtgcttacagccagggctacacagaagtgagcaaagctggtgaagatggggatgggggagtggagtgagttgacgctagaaagggatgtagcaaatgtaactattattccagaatccaagtgtgcaaaaatcataacttttattttgtataacataatttctattttgagtccctcttcagtaaatgtggcaaaaacccaatctcagttattttggggtagctctcacctttccctggggtgtgtcttggaggccaacatagtttcagatgagtgagaaggccaagtgttgttcagcatcatcatctacataggcattccgtcaagctctctcgtcctcctctcttagaagctttgatgtcattctgttccttccgtgtctctctaggtccaaggagattcttggaagctgttctaaagccgtcccttcaactcactacatatttccttttctgaggtcttgctctagggtctgcttaggtggaaggaggcaggcttcttttacctggaccctccagtgcctatgtagtcagcaattctcaactacttattttcttctcagcatgtggaaaatattttttcagtctaaagggtggagcttaatctcatgaaatttaaacaaaatattctgtcatgctcagtttcctctccaaccctcaaaggcataggctttgggaacattttatatatatatatcgtgtaggtttcttcttggcttttattacatatataaatttttttaatgcagcagcagctctctctgaagacagtgactgcacagtgctctagctactgaaatgaggagataggtgtgggggtaaaggaaatatctggtaaatcattgttggtactttaaacatattttctttcatgtgtatattgtggcaagattgcaaaagaactaatatgatgtatcatggatcttgatctttatcctttatgcagtggactgtcattgaaggttatcaatggaagcactatgattatacttttgctttcaaatataaaatctggggacaatatgaagacctaactggggtagaaagattagttacagtagtcttgttacaggagaggtaagaaatgatggggacttgaaccagagcattaactgtgaagagggagaggatttgaatagctctagatatattttggagtcactgaagctgtggaagtggctgaggtcagcctggagcctgcatagagttctgggaaacatcagtgtgtaggaaagagggagagcataaggggtcagtaactgagactaagggagaagcatcagagtggtggaagtaggagaaccaggataatgaggtcctacaaactggtgataaccttcaatgacagtccactgcataaaggatgatggttttattaatgggagttcccctgcatgtgccctcttgactgtcgtcatgtaagacatgcctttgcccctcctttgccttctgccatgattgtgaggcctccccagccatgtggaactatgagtgcattaaacctctttttctttataaattacccagtctcaggtatgtttttatcagcagcatacgaatagactaatatagttacataggtaaatgtgtgtcatggggtttttgtgtagagattatttcatcacccaggtattaagcctagtacccactagttatttttcctgattctctccctccccccaccctccacactccaaaagtctccagtgtgtgttgtttccctctgtgtgtccatgtgttctcatcatttagatcctacttataagtgaaaacgtggtatttggttttctcttcctgtgttagtttgctaaggataatggcctccagccccctccctgtccctgcaaaggacatgatctcattcttttttatggctgcatagtattccatggcatctatgtaccacattttctttatccagtctgtcattgattggcatttaggtttattgcttttgtgaatagtgctacaatgaacatatgcatgcatgtgtctttataatagaattatttatacttctttgttgaatgcccaatttttattttgactggtacactttgaaaggtgaagcacaaataaaaggaacttggtgctagacttgttttctatgtattttgatggctgaagctataggtatggtgtaattggacatacaaacataaaggaggcaattttgaccttgaacaaaattttttactttttgagctatttagaaatgactgtagacttagaagacatcattgctgaagaacatttatttaaattatcttggtttcttatcttaagaattgagaaaactggtagaaacaatgagtagaaaacagatatttagttagacttgaaaactggtcttcatttggtagtgacatactggcaacaattaatactatatagagacatttcctggtaaatacagaagatacataatgtaagctttcccaagattgatttggagaaaaatcctcacctgactattcaatgtcctgtttcatttttgaaaacgtggagcagaagaaatctcacaactgatttaaataaaaattcaatctgaaaaagtttattgttgtattgacataaggcaaagcagtcaagaatccctcttgcatgtttttctaagcttctgcatctctttgtttcttccttaccttctgtcttaccctttaatggagttttctccaggattctgtgcttggcctgctgttttctccctatctgctggcctgtccttatgggtaaggagagatggatttagctatggcctctagcacatgaatcctaaatctttagatctgaactgtctctaaaactggaaagttacggtctgaatacttattggacatcttcaattgaatgtgccaggcactgcaaaacaaaaatgttcaagaccaaactaataatctctcctctgaccagggtagctctttttgctgacttcttatttcccaccatgacactagcattctccaaatgcttaggcttgatgttttgatcatttttaaaatttttactctctctacttatattctatcagttgctaagtcctgtaacttcttcctctataagtctcataagtatttgctttcttttctattctcatttagactctctttccctacatagttatagtagctgaagtcccttagtctctccacattccagttaatcgtatacattgctggtggctaaactctcgtagaacatacttctaattatatcctattttgctcaaaactgtcgaacaactgctggtctgttttcttgcaccatacctgcaggacccagattatctttccagctacataaccacctccctccatccacacaacacacttcctacccctgtcaaaaacaggcttacccattcttcaaacttgccgttgacttttgtccatattgttcactttgctagaaatagtttttcccctcttttccatacctgttgaaatcagactcaactgaaacaggccctttcattttaacataactttttgcagtctcgattgtgataatttatcctagaaaatatttactctttgtacatttataacatatgtgtataagtatgtttaacacagcattgcttgtaacagtaaaatattggaaacaagtgaaatagttgcaacattgaaaaaatagatacacacatatatatactgacaggaaaaaatttggccactaaatgtaaactatgactccattgttaaaaaataagtgtgtgtgcttgtgtgtatatacatgcgtacataaaatctgaaaatgtacacacaaaaatgttgaattgtgctcgtgtgtgtatatgtgtctgagtgtgtgtgtgtgtgtctgtgtgttaggggtaaagttatgaaatacttttataatgtactttttatccaaaaatgttagcatttgctaatattgggtgatgagctcactggtgtttattacactgttctttgtatttgatatttttaaataagaataaaactggaaaaatgctaagattttaaatatataccttagtaccaagtactcaatggcatttatcaggacaaaagatttttctttagtgggatcttggagtgatattccaaaaaatattttaattcaaggacactgatatttctgctgacagactacctcttaccaaatgtgggtttcttcttgcaaataaacatcactgagttggtgtatatgtggttgcacacagtcagcagagaagtatttgaatgaatgccataatgcttacacacaattaaaactgagtcagttcgacctatttttatgtaaatcattaaatgaaatgagtttgattcatttttacatgtttatttttaatggagactaaagagacataaatggtatgtttgttttgtggtggtctaggtgatatcaatgatacagggttcttcatgaatctggaggaagacatgaccaggtaattagacattctccttactattgttaagtttttctatattcatcaagttgtagaaatgtttaaaactttgcattatcatcacagaaattttaaggagaacaatactcttgatagtttttagaagagtatatgtagactttttaagaaaagaattggctgcataaagtatacaaaagttagagttaagcttaaaatggacatatatgcattgatcaatgtagaatatcatattaatatataaggaaattagaggagtttaaggtagtcttttaaaatgcagttgaattaagaatcattattttctataatatatatgtgccaggtgtggtggcacatagctacagtcctagctacttgggaggctgatgtgagaggattgcttgagcccaggagttcaaagctgcagtgagctatgatcacatcactgcactccagcctgggggaaagagcaggaccctgtcccccacacccccccacaaaaaaagagagagacagaataattattttcaatggcttataattattaatttgtttcctggatgttttcatacgacatgacaatgaaagcagccaaaagaaatttttttttaaactcaacatctgattctgaacaataaaataaaacatcccagaaaaaatacagtctctctattcttaactcacactggggaagcttttggttaatgatttacatatcttgaagtttattctccagaacagcttataacagatgtccaaaggtacgtgtaggtgaatcagaagaaaatgattttacattactgtgtaaatgatctgggtacatttgaaaaaaatagatttttttttttttttttttttgagacagagtgtcactctgtcccccaggctggagtgcagtggtgcgatctcggctcactgcgaccttcgattcctggcttcaagcgattcttgtgtctcagcttcccaagtggctgggattacaggcgtgtgtcactacacccagctaatttttgtatttttagtagagatggggtttcaccatgttacccaggctggccttgaacttctgacctcaggtgatccacccaccttggccttccaaagtgctgggattacgggtgtgagccgccatgccctgccgaaaatggagacactttatcatattttgaaagctgaggaccaaatacctccaaatattcctgtggagaggcattgttttttacattggggaaaatgaaacagaatgtcttttaatcatcatgtacatgcttttgagtaaacatacactgacagtttaaaatacattaacttattctcaaagcatgagttttccttgtgatgtggggtttctattagctatctcttgctatataatgaacacttagatagaatactatctcttgttgtatcatgaacacttagatagtaattagttattgagttatctgttaactaaaacttagtgttctaattacatcttccttgataaatgcattgtaagttacaaggtagagtttggggaaaacttgtttcccaagattcctgatgggctgagtgctgaattgtgaacagcaatgtcagagactgtctgcttgttacctagccatctttatagcttctgcatgattcaaacacacagtttaatttgaagatggtaagtagtgaagtttaaaaagtgggaatattggaatactgtattttattttagaatgtttgcctttaacctaaaaattaattgtttagtgaagcccaagaattgtttgcattttttttttttgagatggagttttactctgtcacccaggttggagtgtagtggcacgatctctgtgcactgcaacttctgccttcaagcgattctcctgcctcagcctcctgagtagctgggattacaggtgtgcaccaccacacccggctaatttttattattattattattattattattgtcgtatttttagtagagacgggatttcaccactttggtcaggctggtctcgaactcctgacttcatgatccgcccaccttggcctcccgaagtgctgggattacaggtgtgaattgtttgcttttctttttttttttttgagatggagtccagctctgtcacccaggctggagtgcagtggcacgatctcggctcccggcaacctccgcttcctgggctcaagcgattcttctgcctcagcctcccaagtaaccaagtagctgggactacagtcgcacgccaccacgcttggctaatttttttgtattttttgtagagacagggtttcatcatattggccagggtggtctccaactcctgaccttgtgatctgtctgcctcggcctcccaaagtgctgggattataggcgtgagccaccacgcctggcctgtttgcttttttaaatatgaaaactcagagcaaggaaacattaattcaggatttctagattgattaagggtctgttttttaagacaatactcgtgttttttaatgctagaatttaaattggaagggccactatttgattcgttaagcattttaggataactcacgatggccagttgggcaaatgaaataaaactactttttaaaaatttcatttgtctcttggcatctaaactctactagattaatagcccaagggataatccctagcccaaaaggccaagggattatttccatttcttgatcccactttctgacaagttctgtcacttcttttcatccccaatgtacagagaccaacccaatgcacttatcaactgatgctgatgtaataaaaatattacagtggtgggtcttacctgatgctgtgattccccaatataccacaccatctttcacagtaactggcattctctgagtgcttactatgtgctgggcactggtccaagtatatttcatgcatgagtatttttatctctgttttactaattagaaaactgaggcttagaaaagttaagcagcttgcctaagattacataacagtgaagtgaagagctgggatttgagccaaagcagaaatcattcttaaccattccaccatattggtgcaaacatagttggatggtgactatcaactctgcttgttacaaccccttcttatttgttaaggatggcgagatacccagataagtgaaaaaataggaatgagtggtctctttggaaaaggtacattgattaggaaacagagagcatgtgcagtgttcttgttgtttttcaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagagtaagtatttagttttatgttgaacttgggtgtcgttcttatccttagtaaaatgaaatagatgtcatcacatctgttaggaggtgttaatgtatcattcaaaggtacttatgagacaaaattccttctaagcagcaacaatgtcgtgtgcatccttttgttcccagtgccttgacagggtatggggggacctgcatgactagcattaaatgaaggactgggctttgccagaatgaagaaatcctctgagaatgtgcagtagagcaaaacaagatactttctgaggaaatttctgagcaatttgaaattcctaggttgaatacttcttgtgtacacgatgtccatttcctggggccatgtgggctatggatttttgttgttaatgacaaatatcctagtagaaacttctaccctgctaaataaaacaaagcataggcacaaaatactctagccataaactaccctacactcaaaacaggcttcacgagaaaagttgatgtttacaattctgacaattatttctaacactatctgttctttcagtgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaaggtaggtgaagcctgtgaatccagattttgaactgcaccttctcccttcctgctctcaccctacggaaaggtctttttactacatgatgactctgattctgtacttgttacttttacgttttctgttcaaaatcagctggataagcatatccagcctcactgagtactacccctgccaagactccatgctgttcttggccatgtgattatttggggaagggatgagaaagaagaatgtagctctcatatctaccttctgcctttgtaagaaaacgagcaaataaacaaacaaaaacaaaaacaaaacaaaaaaaaaaaacttgaactccaaagaaccatgctctctagtggatgatgccccattcacacggcaagtagagactcaagtttagtgaactacagatgtcccattcaattaacattttttttttactttaatgtttttggctttctagatttttgtttaagttttaaattgattttacagttttgtcagcattcaaacacagttaaatttttggttataaaataatttaagaaaacagatactttctagtccctaaggatgcatataaatccttggccttcagctctctctcctgtcatgaaggcagagaggtagaagtggccctctctaccccagcacacgtttccagtgatcttttaaaacttattttccatttcaaggtgaacatggtgttaattttaaactgatttgatatgagtgtattttcagtagagaaattggcccagtatgtaatttttgtggagttcaacacaactcagataaggcaatataaattcatgtagcaaaagtaatgaatggacaaccaaaaccaagcatcacacaaaataagtcaggtagtaagtattaagaaactgtagaaattttaacctgtataatggatattattttgtttctgcaaataataattatcatttattgagcatttattgtcacgcaaatgcctgtgttatctttctgaagtataggtattatctcactttacagagaaagaaactgaagttctcaggtctctgttaattttttgctcagagtaacatagagttcaaataatgtttctggggattatcagttaactaaaatatttggcaaaagcttcttgtcaattgatttttctgtatactagaatgttaagcaagttaggtggcaaactcttcacatcagtatttgttgaaaaattacttaaaatgttttgatgtctgttttaagattaaaaacagaccatgagttaatcttttaatgattaattatgcattgcttcacatgccatttatttagtaaaaggagccccaggctggctgctagagacctaagctttaatcctcactcagcattacacttttggtatcatttcaaacaaatttcttaaattctttggatgtcaatcatatcaaaaaagaaatgtagttcactctgtgtctgggaagcctctttgtgtctgtgagcctgaggctctaccttcaaacctagagacacagaagcaaatagatatagcagaaagggacccaatcacagcaagtgacctggcccaggaagcctcttttgtactttgtgcccaagactgtcttcccagcgcccagagacactgggccactagagggcattggtacaggaaaccaccacactcttcccgacctggcagtctgatcttgaaaaaccactttcactccctcaagcagcagaagcaggggccagtgggagccccagaagcttcacataaatcaagccgaccaaaataatactacaaacactctgaatattaaaccgctcctggaaccacagctcacaaaagtagaccaatactatcatgctaaacttcaatggaacaactgccggctagagtaaggcaggtttattttattttaactttttaaaacttttattttaagttcaggggtgtaagtgcaggtctgttccataggaaacttgtgtcatgggcatttgttatacagattatttcattacccaagtattaagcctagtacccattagttatttttaatgactgtctccctcctcccactctccaccctctaataggccccagtgtgtgttgttcccctctatgtatccatgtgttctcatcatttagctcccacttataagtgagaacatgcagtatttagttttctgttcctgagttagtttgctaagcataatggcctccagctccatccatgtccctgcaaaggacatgatctcattttttttatggctgcatagtattccatggtgtctatgtaccacattttttttttatccagtctatcattgatgggcatttaggttgattccatccttgttttgtgccagttttcaaggggaatgcttccagcttttgcccattcagtataatgttagctgtaagtttgtcatagatggcttttattattttgaggtgtgttcctttaataccttgtttgttgagagttttgatcatgagtggatgttgcattttattgaaagactttttgcatctattgagataagcaagtgcttttgtttatgtgatgaatcacatttattgatttacatctgttgagccaaacttgcatcctgaggataaagcctacttgatcatggtagataagctttttgatgtgctgctgaattcagtttgccaggattttgctgaggatatttgtatcatatttgttcatcaaggatactggcccaaagtttttttgttgttgtatctctgccaggttttggtatcaggattattctggcctcatagaatgggtcagagaggagtccctccacctaaattttttggaacattttcagtaggaatggtatcagctcttctttttacatttggcagaattcagccatgaattcatctggtcctgggcttttttagggggtggtaagctattattactgcctcaatttcagaactctttattggtctgttcagggattttatttcttcctggttcagtcttgggaggatgtatgtgttcagtaatttatccatttattctagattttctagtttatgtgcacagaagcattcataatattctctgatgggatccttgtgactgaattgttctgcatcatgactgtgttgtgtactaccatgtccagctagtttttaatttttttgtagagatggggtttctatgtgttgcccaggctggtcgtgaactcttggcctcaagtgattctcccacttgtcaccccaaagtcctgggactataggtgtgagtcatcacacctggccttttcatatccttgataataggataggttagagggtctttttttttttttttttcttttcacagacatgatctcctgaaaaacacaacacttaatgaagattcaaccatacttttgtctgcttataatgatggaaggtcatagaaggaaaagggagctagatttgtagaattcattgtggtgtttgatatattaataataaattaaaataacatttttaaacactcctaactttaaagtatttctttaaagtatcactttgaagtgatcttcctatttttggttgtctccattgtggcaattgttgtggcaccatgaggtttcagaaggagcagaaaaacattcctaaagcatgttgtatgagcctcctctattctacagatgtttgttaagggatggcgctggccaccctatctcagacctcctaccgtgaatggttctgcaaatatggtgggacagccccaggaagattgctttgagaagtcaggaatgtctagtgaatcaatggaatattattttgggtactaaagaggctaggtacgtgcagcacaatgtctgcacttagaacataactaatcaatgttcatttgccttcatccacccattcctctagctgtcatagctccttttactttcacacacaggaaagggagaaatccactgtcaaaacccaaacaactttgagctttttatgccaagaagggatagatcaatgttacttacataaatgagtccacaagtgcctatgaattttttattgaaagaatataattctatttaatcagaatttttaaaataaattggttttgtgtaattttgggggaggggtggggagaacagggtctcactctgttgcccaggctggactgcagtggcacaaccactgctcacggcagcctcgtcctcctaggctcaaacgatcctcccacctcagcctcctgagtagctgagacaataggcatgtgccaccacacctagctaatttttaatttttttgtagaggcggggtcttgccatgctgcccaggctggtctagaattcctgggctcaagcaattattcatctttggcatcctaaaatgttaggattacaagtgtgagccaccacacctggcctgtcatgtgtaatctttatttaggtagttgaccacttcagcattctaggtacaataacgttagcccttttcccattgcaattgatccagctttcataataggaccctctgggacccaagttcatgcatcagtggctggtttcagggagaggtttacttcagtggctgttaatacagaccaaaggcaagtaaaagacagattttgctctacacatgcattaatgtataagcagcatttatgtatacatttatttttactttacaaaagataaaattaacgtgtttttttcatgatccaaaattgtatttttaaacagatactatcttcatttactgataaagaactcttagcgtatgcaaaagctggagcagtagctgaagaggtcttggcagcaattagaactgtgattgcatttggaggacaaaagaaagaacttgaaaggtttgagtttcttttttaaatggatagatatgttaaattctgtcttctcaaatgcccttcagattgacagtgttatttataagcattttctcccatatatatgttttcttaaatagcttaatggatgtattacagcataactaatattttggagaagttgcaattctaattgtacttttcttttatctgctaattcaaagagttttctagatgggcaataacataaaaatagtttccaaagactgaaatgtattttatacctctgatttttttcctctatcaagttaattagtgattctctaatattgactgagtattttgtgtaagatctatggggacattttaatccgtggttcattttcctttctgcccctattcagctgacatatgctacatgatttggaagataaaaatataaccaaaaagcacttatgtgtacaaaagttcaaaatcttttatattatgtgttgtttagtattatgcttacaactgttcccttttattagatttctgaaagcatagatggtaatttattgaaatttagaatataaatcagattactgatttctaagcagcaacagattttatgtaaaattgaatataaattataatattctgatgctataacacttcaatcttatgaatataaatatctttgaaggtatgaaaaagtaaactgtattggttaagggggttatacattgtttggtacagccatgaagaaaaccaagggaattttcagaaatcagaataatggttagctctagggaagatgagaagttgtgtaaggagagccgaacacacagcaggggattctgtggttctggcagtgttctgtttcttgatctcaggtatacttacatgagtattcattgcataatttttttaactgtgcatatgtgtttggagtacttttgttctatggtatattcacacatataaatatatgtcttctgagaataatttataatggttaagtgccatagttcaaaggaaagcaatatctatgttccattttgatttttgctaaagtctttgcttttgctttaaaagatgtctggctaataaaatgaggagtcctgcttaatgactgagcaaatattcaatgaaataaatccccggctctcatggagcttaaattctaatgggggtggtaaggttacagacaaccacaaataatcaaataggtaatacaccagttggggtaaatattatttggggaaataaaataagagaatcatgaggatactatttagataagttattcagaaaaatattttctgattagttgatatttgagcagagacataatgaagttttgagtttggaattcttttccagttttcctcagagaaaaggtggaccagaaaaataatatgcccaacagcccaacagcattggaagacagacaaaacttaacagcttcttcctggtttctcagctggaggaaaacatctccccatagaagttagattctcaggcctgactgctatctgcagatggggtctgaatttatactactctgtgaagtaggaaacaatctctctccaaagctgaggatttcacattaaaaatgagtcctaatccagtgataccagagtatctggtagaagaaatacaaaattattgaaaattaaagaactaaacatctaaatcaagaagttagggaaagaaaaataaaatattagaaggaaataataagggtaagagcaaaaattaatttaatagaaaataaaggcaaaagagtggaagattgacatatccataaactaattctttaaaaagacttataaataactattttatatgtttaatcaggaacaaaaaaagaatgaatagggaaaacttgtaacagattaaaaaatctatttttaaattgcagctttataccaataaatttaaaaaccctcatgatatagataatatgctagaaaaatgtaggttaaaactctcacttaagaaggagaagatctgagtaaacccataatcgttaaaataattgaattcaaagttaaaaatctccattttccactgcttctctcccacaagcagaaatcacaaaacaagtctggataattttgtaggcaagttctaccacaacttcaagaaagagataaccctatcttacataaattgttgtagaaaatggaaaaaaaaagttgtgagttcattttatggggctagtgtaactttgatactatattaccaggcagatataatatgagaaaggaaaactaaaggacagtttcacctaaaacatagatgcagaaattataaataaataaataattagctaaataaatccatgtgaaaaataccaaattgaaaacatcctgaacatgttggctttatcctatgaatggaaagatggttttaacattaggaaatctattaatataattaaccatattaatagactaaaagaaaaaatttgttgtgatgtaaggggaaaagcatgaattatatatgattcatgacaaaaactcgtaaaactaggaataagaaacaaccttcttaatatattagagacccgcaggaaacatttagtggtgaaacattaaaaatattttaacacttaaaataatgaatgaaataaatacatactattttgtctgtcaatttaaaaaataaaaaacaaataatgaatgaggcaaatgtgtttactatcatttctatgtactgttgtactagtactagataataagaccaataaaacaaataagagtaagaatgaaaaaaagaaaacgaaaaagaaaaacagtaagtattggaaaataggaagaaaactgtcgttattcacagacgataagattgtctacacagaaattccagtaggttttataagctaacaaaatacaataagatctctgggtacctatcattattcagaataaatggcattcctgtaaaccaggaatgaacaattagaaaatatatagtatttcatccattcagaatgaatccatcatctctgaaatggaaataaacctaacagtcagtgccatcttaaatttgctatggatctggattgaagtccaattctatcaggcaggatttatgtttgcttctgctattcaccaagggcactgctaacctgagaccaccttacattaaattatgtgtctagttgtttttggacaacactggtagtgtgaattcatgctcagacctgcatgagtcctactttgtggtccagattctcagggaggtttttttccttccttcacctggttggtgccaagactgagacatgcaagttctgctgtgtgttgcttaatatactgaggtatagccctttgagtcctagctgcatatggaatgttctattaaactcatttcttaatgctttataaaatgacagaataccagtacttgctacaattaaagacatcttaaatttgaggaactgacaatatttttaaggacattattagcaacaaaaactataatgatggatggaataaaacctgagaaggaggaaatggacattgtgggagtgaagcccaacatttgtaacttagataaatctgatattttagttcactgaaagtttaaagggattcagatttaatggagtgctttaattagttagatatgaaaatatttttcctagcaaaaatgatcaaattttaattagaagaataaacaaatggacttacacagctatatccagagattcctatatttgctctaagtagtattttacatgtggaatcatttcttcagaaaatataagttacttgaatgtatctttgaaagggcagtctccttcagagaactgaaactaattttgcttggagatcaagaattgacgtaagactacaaactgatggcctaatgtccttcccaagttggccatttctttaaagtttatttatttgacaaatttattcagcacctacaacatgccaggcactgtgctaggttctaggttttccttttcttttcttttttattttataaagttctaaacaaagctagggctctaggttttcagctgtgaatcagaaacagaggttattttgtgatcttcagtcaaaatagtaaaatggataaaaatggaaggaatggagaaagggaggaagaaatataaggaatcgtcaaagaaaaaaataaaaatattgatttaaaaatgtgatctgaaagaatcaaagagtgtgaggatgatgaatggtcaagttaactttaaatacggtggttggggcaggcctctttgaggaggtttcatctcagctgagacctgagggatgagaaaaagcaagccatggtgggaacagagatgggatgggtttgaagaagagtcttccaagtttcctggggtggccaagaatttagtatgttctaggaaacgtcataaggcttgcatgactgaagctcagttgaatgaggggagattggggtgggcaggggccagaatgtggggatgatgggccatgatgaagagttaggattgtattctaagggcacaggggagtggttgagggtgttaaaaagaggaatgactatccacgttatgttttcagattgtttgtctgtaatgggtctcatgaatggatgcgagaagtcaaagggtggaagcagggagaccagtggggaggccggcagaatggtccaggccagagcatctccctgatctggagcagaggcaggagaggggtgacaggtacctagatttgggatataacttgaaagtagaattgatgactattcttagaccagacataatcagttttctttcctgttcttctccttcccttctctgcttctacataaagtaattgaggtcttaatctggctttctcttcaaattagcagtgcaggagggaagctggctctctttgaatggaaatttaaccagaagttaaaataaattccattcaatcgtatagaatagttttgttccttttcacttaaaaatatttttctctcttttatgtgccttgaaaatatatttatctattaatatttatttctgttggcttaaccaaaattgttccttattcttaaagtagttacttgcattattgataaatattttcacaatatttaagaagagtcatatttcctacataccaatggttttgttatataactttagtctcttttagtcattcctactttcatgaccatttctgtactaaagcctttgatgaatggatgattttaatatctagttttatgtatggcatttgtatggaaaaacaacaaagagcttatggaaaaggagtattgtttatatctcatttccctttgcccccttctttcctttatttctcctttctgatgtctaaatgattttcttccttgtgcttctgtgcaaatgtttcctctggctgcataggacaacatcagactggtttctacgttagtcttggtaagcgagaactgaaataaaggagataacagagtatagtacatatgtattaaatgtccttaaatatattcttatgtaagttatgcccagtggtagcttttacctcagatcaactcataaagtgagagatatgtatgtgtctgtgtgttgtgtgtgtgtgcatatgtgtatataaataattgaaacgtttctcaaaaataatttttctgttcagatgataactggcaagttgaggatttacattttggggggtagaaactaatgatgtgttgctatgttgtcactatgaagttctgtttaaatttcatctttagtgaacacggtagtgatgtaaatttcacttggcatgcttttgtaactctgtaatgtacctagttaaagtagtcatgaaagcaagaaacaatgcatgttcatatctggacagtgcttgagctctgctgtacatgctaatggaacaagttctccaaaggaaattaatataatgctatataatatttatatagttgtgaatttatggatcaaagaccttaaagtatgacttttatatggaagtaagttcatgaatttcaacaattattagctcctcctctatccaaggcaatgctctaggttctgcagggagttgcgattaagaaaatacagacagatttaaggcaatctgtcctgtttctctaatccttttaccttggttctacatttgcaaaattccttatttttctttaactctgggctccattagcttatctccatagctatcctcttaaacagagtagtcttctctgttcctttcaaatgaaggtcgagttatagacatcatctcctttaactttaccgtcaaaaattgaccaccctctgagggtttaaaaaatatttgaattatcagttgggaataacctgtaagtcattagaactgcagaaataaaatctgtcagtgactccaacattgtttttcgattttttttcatttgctcatttttgccccttttgtacaatttcctatatttgctctaagaagtattaatgggtcctcaccaggtttggttaatgtagggtaggcctttgtgctatgaatgggttcagggctatagatatgctaataatttaatttttctcagcccttgagttagtcagaagccgtggatggcccaagccaccaggtcagatcctgttagttcagcagctggcagctatggcatgacaagtgttgggagcattaatagagggaaggacacatagtcaatatgtagaaaggagaaggaagagagggaatgctgatgaataatgatgtgcttttataaacctgtaagccaaataatttttgtaggtcagtgatttttaaaataaatgtctacggaagtaagataaattgtacagcttatttaaatgtaaaaagtattacatggcaattcagaactctcaaaatgctctcacacatgtaacctctcagttggtcattccgacagtcctagaggaaggcaggacagagagcaaactgtctccaaacttccttaatgttcagagatttaaatgactgtcccttttttttttttttagtactttctaacattatctctccattttattaattaaagaggcatgcagattttgaaggaaagacacaggttttttccatggcttttactttgtcttgtgtatgaaacaatgtaagttttagaagtttgctaaatattgcttcacttatttgctattgctaaagccagagaaggaaattttagaagactcccccacaaataagttaatctctataaatgtcagtttcttcttctgcaaaaagggagtaaaacagtactactatatagggtcaatgtatgagcagcaggacaaaatgcatgtatatcacaggactgaacacatcctagccactctgtaactatcatgctatatcgacatgttttcataaaatgtatatgtgatcatttttgttctttttctcaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtaagtggtttacattgagaaatgaaccattattataaaatgccaatgaaatccagtaatgttggcttgactttagaagaatatattttgaacaactgttgaatactacagacatatgcactactaggttaaaaagtggaaggccaggcacggtggctcacacctgtaatcccagcactttgggaggccgaggcgggcagatcacgaggtcaggagatcgagaccatcctggctaacatggtgaaaccccgtctctacaaaaaatacaaaaaattagccgggcgtggtggcgggcgcctgtagtcccagctactcaggaggctgaggcaagagaatggcgttaacccgggaggcggagcttgcagtgacctgagattgcgccactgcaccgcagcctgggtgacacagcaagactgtctcaaaaaaaaaaaaaaaaaaaaaaagtggaaaaaaattagatgcatccgacaaccagttttttcttttatttgcttgtttgtttttgcagatatcttgcacaatttatcctgtagcctaaataatattaacgttagcaatctcaatagcatgaacttttatttgttgagattatttgtgccaaacttgttgctaaacacttcacttttattttctgatttactattcacaaaaatggcaaaatacacattttgacttcttttcaaattatttcgaatggcagctaatccacctcacaaattatggaagtataaattgttcctcttcctaaaagattgtttggaagttttctgcattatagacctttgtgcaagcacagaagtcttgccatttaatgttatcctcttttattttaaagacaatgaaataatttgcaaaccaaactcaattcatatctggatctaataaaatacactgttagaagagtcacttagttcctttttcgtaggccaagcagaaatagattgaggcaatgagagatgatattttgtagaatcccttaaaacaattccctttttcaggagccagatttgtaatatggattcaataatcacttctttttttttttttttcaaatttgccttatccaacattgctcagtggggaatggcttttcccatgaagttttttttttttagtatagtgatgctgtgggtgatccttggttgtcataacagatgttttctgtttaactgtgattgagataagcagtttaaatccagtgttcactgggcatgaatatacttcacaaactggaggcatccaaaatagggagtgtggggtttgatatcggaccaacttgattcaaatggcagctttgtcacttattagctgtatgatttagggcagccagctaaacttttcaagcctctgctacctcttctgccaaatgatatgacaggaatgaaccctaactttcagcgtgttttgaggactaactgagataatgaatatagaatcacttagcaaagttccttctgtgtaaattattgagcaatataattccatgtgatttagaaaaaatgaataatgacattaattctcatcaaccccaaagagaaacacagttaagtctttaccaataagaaactgatttgcacaaaacaaaaaaacaaaaggcctaaagagaacacggccttgtggcaaaaaagacaacctgataaaggatcagtgtcttaggtgtgtgtgctttataattaacaaaaaatgttcatatacactgttttatcatgatacaacatctctatgattgctgggtagggagaacagatagaatcttgcctgtttttgccaatgaagaaattgaagccaaagattctaagagaatggcctaacaacatataactagttattactaatttaggattcagaactgggtcttctgactcctctgctagcatttatttccactttaccttactgatgatctaaataaaaaaaaaactagacattgaagaaatataaaaatatcctattataatattctagccctgtgaaatttaaatcattgtttattgagcaagtaagactaattctgcattctaaattgaaaagtatttatatttccaaaatatttttagtatgggcaacatagtgagatctcatctctataatttttttttttaattagccagatatggtggcacttgtctgtagtcccagctacttcggagactgaggtgagagggttgtctgaacccagaggttgaagttgcagtgagccaagattgcaccactgcagtccaggctgggcaacaaagcaagaccttgtcttaaaatttttttaaaaaaattttatgctatttttaattgacaaatcataattgcattacacttacggaatacaatgtgatgtttatgtatacacacacatacacacaaaatgtggagtgatcagagcaagctaattaacatatctatcacctcacttgacaccttttgaggtgacacatttgaaatttactcttggctgggtgtggtggcccatgcctgtgatcccagcacttttgcaggctgaggcaggaggattgcttgaggccaggagaccaggctgggacacgtagcgagaccctgtctctagaaaaaataaaaaaaattagtcaggtgtgatggtgcatgcttgtagtcctagctacttgggagactgaggcaggaggattgcttgagcccaggaggtagaggttatgacgagctatgattgcaccactgcactccatcctgggtgacagaatgagaacctgtctcttaaaaaggaaaaaaaatgaaaaaaggaattaaatgtactgttagttacaaacttttaaaatatttttaacacctagtatttatatgttgcctcgccattttaaattttaattacctatgttagaaaaagacagaagaagtatgaaaaaaaattgctatcactatctcagtagcctgatggtttttcttcacattcctcaggtattcttttctgtattaattggggcttttagtgttggacaggcatctccaagcattgaagcatttgcaaatgcaagaggagcagcttatgaaatcttcaagataattgataatgtaagtctgagttggccatctatccacctatctaaaagattgtccagttaagtcaatttctttgtcactttatccagctctccacaaaatatcactaaaagtagttattgtaacctagtaatctcttaaaatttgattctgtttagaagccaagtattgacagctattcgaagagtgggcacaaaccagataatattaagggaaatttggaattcagaaatgttcacttcagttacccatctcgaaaagaagttaaggtacagtgataaatgattaatcaacaattaatctattgaatgaagagtttctgatgttttcttgtagagattataaaaaagtgcatgtatatttaaacctagtgaacagtcagttcctatatcctgtgtctgtgaattgccttgaagtttttttctcactcgtcctggtagatcttgaagggnctgaacctgaaggtgcagagtgggcagacggtggccctggttggaaacagtggctgtgggaagagcacaacagtccagctgatgcagaggctctatgaccccacagaggggatggtgagatgacccatgcgagctagaccctgcggtgatcagcagtcacattgcacatctttctgatgttgccctttcaattacaaatgtatgaaagtcacacttactttttattccaggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcatgtaagttgtccttgccctttgcctttctagaggtgcaaaaaataaaatgcaggcctactatgcaggaagttaggaaactactataaatcggaagaagggaaatcctaagaagggaaagtaagattacttcagatttgaaagctctagcagtatcaactggtcgtagatacatttttaaaaactgaggttggttattgtgttaaataagatttaaagaactggacctgtattacttgtgagacttgggctgtgtataggattccttaccaatttaaaatatgagctgagatagcttgtccttatgctaaatcattctgggttttctgtggtagaaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggtcagtgaggcttagttcaaaccaacctgatttataagcataagaacattctactactaattcttgttaatattggtcttagaaaaggaaatttctgatagcttctaggtgattccttcagctattaaaataaaagcattgggcctctttgaaatctttttctatttgtttgttttattgttcaatttctatttatttctctgatcttattttaaatgttgatgaatacattttcatttgaagacacttgctaatcttttaaattaaaaaatagaaatatagacacatgtgaaagttcatcttcattgtgatcttcaaaacttgactatgtggataaccctgttatttaggttttgagagtttgtaatattgccaagaagagaaaaatacaacctgaaggtccatatataattttccaggtgttgaatgccacttgaagactctatgcgaaataagaaacctcttatttccaggaaaggggcagatagcctgtgatactgaaaacctacctaagccatgacaggttattgactatcaacagagtttgactgtcctgcaattctggagtccatatgactcattcaccaaatagcatttgagtgtttgccgtgtgccgggcactgtgcctttgatctccagcacgtgatagtaacggggataattctgtgaggaccgagaatgtggagatggagacattataaccaaaggtgttccaagttgagatgtcacagtagaattcaaagatgaactcatatttgtttcaactctccctgtctctaataaaacaacacttgaatgttccttaacatcctgtcaatgtgcttaataaatttttgagagagaaaaaaagcagcttactaaacattctgtgaaccaaaatagaggccgatgggattctggttactatttttcccctcattttgcttaatctgtgatttcatctctgtgtttttctttttctttctttatttccttccttccttccttccctccctccctcaatccctccctctcttgctcttcctcttcctttcctttctttcctttcctttcctgaccttcccttcctttcatttcctttcccttcccttccctttctttcccttcccttcccttcctttcccttcccttcccttcctttcccttcccttcccttcctttcccttcccttcccttcctttccctccccttcccttccctccccttcccttccctccccttccctcccctcccatcccctcccctcccttttcttttcttttttctcttctcttctcttcctctcctctcctgtcttttcttttcttatcttatcttatcttttcttttcttgtttcttttctcactctgtcaccaggctgaagtgcagttgtgccatcatggccactacaacctctgctgcccagtctcaagtgatcctcccacctcagcctcacaagtagctgggaccacaggtgtgtgccaccatgccaaggttttttttttttttttttttttgagatggagtctcgctctgtcgcccaggctggagtgcagtgggacaatcttggctcactgcaacctctgcctcctgcctcagcctcctgagtagctaggattacaggcatgcaccgccacacctggctaatttttgtatttttagtaaagacagggtttcgtcatgtggcccacgctggtcttgaactcctgacctcaggtgatccacctgcctcggcctcccaaagtgctgggattacacgcgtgagctaccgtgcccagcccccagctattttttgatatatttgtagagatgaggtctcactatcttgccccgactggtctcagactcctgggctcaagcaatcctcccgcctcagcctcccaaagtgctggaattacaggagtgagccactgcttactggtttgcttatctgtgtttccttattaatctatagtgaaactatgtattaaattataaataaaaacaattttaaaaggttatattttaaaatactttagggtgtaaattttgaggggaaattccacataccccttttttcttaaaaagatacaaaaattgatctattttcttctgtattttctagtttctaccacctaatttttccttgtgtattttttctttttgaagttttccacttctacttatcctatggatcctgaaaatgttgtgtgttggttttgagaattgtattgctagttattagagagacatatagagtaacaaaaattatgagcattgggaaagttacaaaggttagagaagtctcagacaaggcctggatatctggctctgttcctttatgaaaataaaagagacttacgtgactcttcaatttttccataattcttcaacctaggaataagtatcactaactatggataaggcacagtgttgagtactttatgtgctttattttatttagtcatcacaactactctaagaagtaaatactattattatccccattttacatatgaataaattgagtctcacacagtttccttggataaaatattttattggataaaataaattcataaatttatttcaggtcagtgtgacattgaggtctggactttgctgcctcacatttattgtctgtcttgttcatccagggggtcatgcgggataggatattataattcctagggctgattactagccggtgtgtatcagtacagcacaatggcctgtgtttgttttgattggccaacgcctggtctgtaggaatttgttggtttgtacaagcccctgattattattatttttttattttttattttttttttttgagatggagtctcactctgtcacccaggctggagtgcagtggtgcgatcttggctcactgcaagctccacctcccaagtagcggggactacaggcacccgccaccatgcccagctaattttttgtatttttagtagagatggggtttcactgtgttagccaggatggtcttgatctcctgaccttgtgatccacccaccttggcctcccaaagtgctgggattacaggcgtgagccaccacgcccggcccaagcccctgattattactgcaaatttaggttaaataaaatatttgggggcttacataatattaatatgtgactgttatatttgtgtttgtatttattacaaggaaacatcatttttaactattatcaattgtctatacatttattgaagtcagaggctatcttatatagatttgatggttttacaatgcccacagcattggttcagtaaatatatgttgaatggttaagtttcttcaggtaattgttaatgtattcaaaaaccaaatttctctctctttaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcaggtatagtttaacttcagaattttcctaagtcatctcagtgataaactgattttgcatttaatgctaaaaataaatattatttgatttgattaccttacaaagtaggaaacaacacctgggggattcaggatgagaccagtgtttaagattttttttctctcttgaaagaggggaaaataaagaaggataaacagataaaaaaattaaaaggtttcaaggtgagttatccgttatagtagtcagtagccacatgtgtctgtacagcatttaaatggtagtgaatctgaattggaatgttttaagtagagtgtgcacatcttgttctgaagacttagtacaaaaaatgcaaaatatctcaatttttatattgattacatgttgaaacgatatcatgggatatcaataatatattggatatattaggtgaaataaaatatataactaaaattaatttcacttctttctttttacatttgttaacatggcttctagaaaatttaaaataacatggctcatagcatggcttgtgccatatttctgttgaaagcactgatccagagtaaacttgattgagtcaaaacacccacaaaaaatggcgtgagaagataagatacatggaatgaatgccagattatttcagtatatgcagtgggaattcttctttggtggttttcgttttcaaatatcacacacacacacagacacacacagacacacataaaacaacacagcagattagctttatccttttctgcagttactaaacaaattgctgttttccttgatagctgacattcagtgattagctttcattggttaacacacagcctaatgagcttttgcatatttcttatttattttagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggtatgaagggagatgcggagtttgttttaatctcactaactgtggttccctagtttggtgggctagggctacggtaggagtgggaacaagagaggttatccagaatcctcctgtcctatcccccagaatgtcaacattttagaatcaggttagaatttaaaagtattaatttacacagcagaatttttagaattaaaatttatagtgtaaagagactatagcgggtcttcaaatatcaaaatttccattctgtttactcctgcttataaatactcttgtcaggttctgagtaccaaataaaagtaagtgtttgtggaaacatcatttattttttaaaaaataaaggagtattgtagcaaaatttgtcaacatttttttgaagctaaataataatcactatgacattttttaaagcaaaattgtcatcacttattcattgataaggaataaggataggatatattcctttactaatttttgtgcgtatgtaagaattgtacatataaaagtttttaactagcctgttgtaataatttgtgttttctaggatgaaagtatacctccagtttccttttggaggattatgaagctaaatttaactgaatggccttattttgttgttggtgtattttgtgccattataaatggaggcctgcaaccagcatttgcaataatattttcaaagattataggggtaagtgtgatgcccatttgtgtgatttacttgtgaatcctgatggaagaatgaacgaataaagtgcttgtgtctgaactgggatacaaaaacaacaaaatccacgtatctccatatgaaactaattggcttgaagatgtaagaatagcagtctggtttgtgataggagagctaactttgtgaaccttcctttgtcatcacagttggtttctaacctgggcaactaaagaccagcttcctgaaatagtcaatgaaagtgatgagatgactttctcatacctgccaacatttggctctgaacaaatcttgtgaactttcatttcaccaatgtgcaaagagagttcctcaaatgtttgtcaggctggcttgagaatgcactgttctctaacatacatactttccaggatgtgagtaagcctagcatgcttcttttgtaagatgtaattttcatcccacttgctaagattaaaacaaataggaacggcttcctaaagattgaggttgacatgtgttagttccacctcatcttaatggtggcattttcaacagtaaagttacaatctgaaaggaatgctctctgtttagtgacatatgtccaaacatcaagctgaggacacccttgccataactgctgaaaacattgtggagaaagagattgatcaccacaagaagcataagctaaacactgacttttgattctacaaaatatatatgactattcaggtctactcatgcctggttagcttatttcagtttaatatattcattcatgaattcatgtttaatgacattcatgcttaatattaaatatatttattcaagaattgttattattttttgagtctcgctcactctcgcccaggctagagtgcagtggtgcgaactcggctcactgcagcctctgcctcctgggttcaagcgattctcctgcctcagcctcctgtgtagctgggattataggcctgcaccaccatgcctggctaatttttgtatttttagtagagataggacttcaccatgttggccagggtggtcttgaactcctaacctcaagtaatccacctactggcatgagctgccatgcctggcctctagaattattttttgtgtctgctctgtgccaggtatttttctagttgttgaaaaaattccgagaatgtgatcaaatgtctgattctgtagagtatatggtccagggcaggagtcagcaaactttttctattaacagtaagatggtaaatattttaggctttgcatgccacatgatttctgttgcagatactcaactctgacctagtagtacacaacagcaatgtggaaatgaatgaacacgactgtgttccattaaaactttatataaacatttggccagtggcccatagtttgctgatccttgcactaacatgtgtcttgcaggttttgggggttggcgggggtagactgtggtgtcatcagagaaaaaaaaaaaaaggaaatggttaagttaggaatgggtttggttgtaacaaaacctcaacttatgattttatacttttctcattcttcatatgacaataagtctggaggtaagtgacccagggctgggatattagctcttagatattgagtaggcaaccagacatctgcctcattgaagatagtgggctctgcatcggattgcctggatttatatccttgagtcatcccttgctggctgtgttaacttgagcaaattaattaccttctctgtacttcagtttctatctgtgtaaaaccgtgatgataataaatcatacttctggagatggttgccaggattagaggaatagtatatatgaatttgcttagaataatgcctggcatagagtaagaatttggtaattgttatgatttttgttggactgactagtcaaaaaaaattatttaatctcttcaaaaatccttgttgttgttgttatttgtttgtttttttatgaaggataaagttgtctgtcctgggctaggaatgttactatcttacaagactgattttggcccactgtgaagggagatacgtgttttctttccctgaggaatggttatcctctgtgttccttgagtccaaaacatcctattagcccttataactatctctggaaaaaatttcaggtatggtgctaggtcaggtagctgtagtttccaactggctctcttaactggccatcctaccaagaaggcaggctcaaggaaattcctttctttttgaaaggcctgggctgagtgaatgaccaccactctgtggttcaccaaaaaaccacatcaggttttccccagacaccttgggacagtttgaaatgtccaaatagtaaagcaatgaactgccataaatgtagttccccaccaagggaagaggagagtatttgttctgatttcaactttacacagttgaggtggcactcatgactccaagagggaatccgaacagaaaaaatactggatcaatttatgaatgatagacctatccttggcctccacagaagggagaatgcagacacctttaaggctggcaataggaaagccaaccccaatttccagcgtactaaggctctcctgacccctgaatactgggccagatgggaaatgggtcaggtccaggatgggttcttcactgattgaactaaaaatcctttaaatgttttctcacaggtttttacaagaattgatgatcctgaaacaaaacgacagaatagtaacttgttttcactattgtttctagcccttggaattatttcttttattacatttttccttcaggtaaatgtttccattttcactatattcattttgagaaatgcatcattattcaactggggggatttataaacatcagtgtaattggccttttagtagaacttctctattaacatggctactaacagccaagtttttctgacatagtgaaaaaagatgtttgcctcctctggctcccttgcttaccttctcctctccacccttacctcccgcaatgaaaccagggggaacagagtatttggcctgatatgatgattggaggtgaaaggcagggacttcaaaatggggtgtgggggagccctggatgtaagtttggatataagtattgccagtaaatgtaaatactcaaagaaatgtcttcccgtgttctaaaagcaacaacaaacaaacaaaaccccataagcgggtcacatgggtttaaaaagggtttgaagagtttatcgttcaacttttgttcaaggagaaagaatcatttcagtgatggaagaatgtcaatcctccaacaaaaaatgtaggaaacatttgtaaagagtcagatttttacagcttgcaaatcatttggctgaaatgaaatggcaaggaattaagtactctaaaagtttagtatgagggccaggtgtggtggcttatgcctgtaatcccagcactttgggaggccgaggtgagtggatcacctgaggtcaggagtttgagaccagactggccaacatggcggaactctgtctccgctaaaaatacaaaaattaacagggcgtggtgatccacacctgtagtctcagctactcagtaggctgaagcaggaaatgtctcaggaacccaggaggccaggaggaggaggttgctgtgagccatgatcgtgccactgcactccagcctgggcaacagagtgagtccctgtctctaaataaataaataaataaataaatactgtttggtatggcaagacagtattggttttggttcaagtgctccttgtacctgtttctgattttgtgtccttgggcacataagtaaactgtctaagcctctgtttccttagcggtaaactagggatgcaggtacctgcctcttgatgattaaaaggatcatgtgaccaagtgctcaggcctgcatgaggcagtagtaggtaatcacttattaattgaatgattagccacctgtaatttttaaagataaaactgtgactagatctctttatttaacatgtaagcatgtattacatttttaaaaaatagaatatttttctggacattataagaggtgcaaaagaaaaaacagactgaattctttctttactgagtacttacagcctcactggggaatttgaccttactagaataaatgtgacactctaactacttataggattgccataccaactattaataccacaggtgtttggaggaggtaaagattgctcaatgtaaatttggttaagaaagttggagtgaggtgggctttgagctggtcccttaaatgttgacgatttgaattggtgaagcaaaagaagttagaggactaccttcataacatggtgcttggggggactggaacctagcttgcctagaaaacaggtgagaacaaagtctatgagttataggaccttagaagtctagagatagcaaaatgcctaaagatgttagaccacccaactcctcatcttagtaacatggggagggagtccagtgggtaattaaaagctcaggctctggattcccattctggttaggattctcatgcatctggattaagattccaactctgtgaccttataactgtgggctatggatcttatcaggctctctaagcctcagtttcttctgtaaagtgggcttttctgtctacaccacccgtacagccttgtgccatggcacacagtcacagaaacatagcaagcccttgaaatcaggctttctgactttgtctaatctcctgctttagcaaagacatcaattctccctccttttatttaaatggtggctgggtccctgacaaggtatgttcctgcccacagggtttcacatttggcaaagctggagagatcctcaccaagcggctccgatacatggttttccgatccatgctcagacaggtatgtctatcgagggctgtgccctgggatgtgtagaactccccatgtgtgccccttggactcagacagtgggagctctgtcatgtttcccaggcctagctcctatattggttgtccctcagtctcacgtcagagatgcggttgaaccgcccactaggcacagatgaggctctactgtctgtcaggtctgggtgggcacaaggaactggaccagtttgagacagagcctccagcgtggattcacctccagcctgtgtctcagcagtggtgggcagtgcaggcagaaggcatctttgatacccctcatccctttcctctctttccttctttctcctggcaaacccagggcacgagtgtttcttcccaccaagagatgccctctgtactctttctttccgcaaatcaaaactcatccctgtgtcctgttcttcactgcccattttcttttctgaggctagtctgaaatactagttcaagctgcagtgttactggctgataatgggtttaatggaatgcttctcacattttgcagcttcaaaaccctaaccattgacacgtgtgaatgttttcctggggaaatggggaaggaaattagaggaatgtaactcagagcagcctggttcaaaggggaaagttcctttaacaatcatgaaaattttgtatgtgacctaataactttccgttttaaaaatcactaatcacttgccattgagtaaaatgatgctttagaagtctgccccagatgtgccaggggtactcgaaccctggctaagaggcatcagtttggtgtgttaggctttctagagggcattcacattttaccagctgtctctggttcctcagttcttccccattcctcccatcaccatttagaaagaaaatgtatttatgggaattgctagctagtgactgacagagccaggactgaatctaagtgagagagcacagtttgatgggaaaccctgtccttggactgtcaggtcgaactgtatttataagtcagattccacttaggtctacactgaccttgctccagggccaaatttcccattacccaaccagcctccaggccaactgctgtgcccattatactttggcagctgagctgatggtttgtggaatgtctcctccataaattgttaagtagggcaagcatttattaagtgccttctgcttacaaggtctagtacttagtattgtaaggcattcaaacctgaatggtccctgtgttcaaggaagttacattcctgtgtggagacacttttaaccacttaagaatattgaaaagcaagttgatacatactataataaattatagcactattttttcttaaatatttttgggaaaaatgatagatacttctttaatgtaaataaccagtttagataacttcttagggaccacagttatttgtaaaacatataaattcatacttcaaattcacaatcatgttagtatctgtgtatttaaaaatataaatggttttacaaagaaaatcttattttatgttgaatgttatattttaatctggattacttttgctgatttgtttttgactcaaatcacttatactgccctgagctacatttatctaactgcttattcaacctctctattggatgtctaataagagtttcactgtttaacatttccaaaatggagcttttgactcttcccctccccaccagccttattccttacccactcttcccctatatcagtaagcgacagctccgttctactagttgtttgggctaaaaaccttgaagccatctttgactcttctctttctgttaacattgcaaaagcttcctaactgatctccctacctccatttttatcccaatcctgtagattcacctaaaaacagccacagtcatttttctaaaatagaaatcaaatcatatcccactgcccaaacctgctgaaggctgcctgtagctcacagggtaaagtctggaatcttgacagtcgcctgtaagaccatacactatgtggcccccactctctctcaaatctcatctcctataaccgcctttttccaatacaccgtctactccataaacagtggctcactgcccaagagtaggggaggggaggatcagaggagccaaatcagaacacaaatctgtccagaggaagggggtggcttttacaaattcatacaaaggttctttttaggctgctggcagcctttaaggctttcttgctggtgtatgaacagattaggtatgcctttgcctcagggtctttgcacatgctgtttctgctttccctgaggtattctcatcctttccttcattcaggcctctgttcagatgtccccttagaaaggccttctgtttcccctccccagtctctaaaatagcacctcccctcacttttctgttcttcttaccctgctcaatttttcttttatttgtaccttccatctctagaatctaaacttcatgaaagcagctactttgtcttttttgttcgtacaggtccaacacttagaaatcatgcgtaggagaaagtaggcactcagaaatttttctgacaaatgaaatgatctatttatgtgtttttatattaagtttctttcttgtgtattgaatgtcacatcctgagtactaaatgcagggggtataagtataaacaaaactgaccccatcgctgccctcttggagctgagagtctcataaacagctttaaggtaataaaatcattttctgtgccacaggatgtgagttggtttgatgaccctaaaaacaccactggagcattgactaccaggctcgccaatgatgctgctcaagttaaaggggtacgtgcctcctttctactggtgtttgtcttaattggccattttggaccccagcatgaaactaattttctccttacgggtgttagttatcatcattaagaaaatgttgaataaatatctaacctacgaatatatcacatgctttttgtagcaacatgttaactatttaaacattatatactgtagagcatatagataacttataaaccatttgctattgctgttattcatgctattaacaagatgcatgtagaatagttatttagaaaagagagtataaagtgctcaatcaacataaaacagtaattgctactgaagaaaggatgtatttaattgctgtaagaaagtttagagtcactatggttacagaagggagggaagacaatcctctaaaatataggttgaaggaaatgaaaagcacattaaaaaattaaggcaagaatagaataacttcagtctttatctttaataactttaaactttaataattttaataacttaaattttgctactgtatgaatctcttgatataactagatactattgaaccagcaggttttgatttttggctgaagtgacaatttcttctacaactgtttatggcaaaagtccacaaaatgatgtagaatttgaaaaaattcatgtaatctctggtgtgtcttttcccctcttgaaccttatccatctttatctttaaatcttttctgtaagttagtactatactaacatttcttctatctaatatatggggcttctttaagaataaattaagctataaatgaggaaatacatagagttataacgttgaaatataaaccttaggagtccctctttttctattgtttggaatagtttcagaaggcatagtaccagctcctctttgtaattgttgttttaatacaaacttctttgcctaaagcaaacaaaacaataaaaatcaaggtttagatcaagttgtatagaatgtaattacaggtgcacgcctgtaatcccagctactcgggaggctgaggtacgagaattacttgaactcaggaggtggaggttgcagagctgagattgcaccactgcactccagcctgggtgacaaagcaagactatgtctcaaaaaaaaaaaaaatgcaaagaagacagagtggctggaataaagtgagtgaaaagaagagtcataagtgtgttaaggtcagcattatatccagaagtagatggaaaaccactgtagggttttgaacacagaagtgacatgatctgaaattttgaaaggatcactatagaaactgtgtgaataggccgaagggggcaagcatagaaggagtctgttgcagtaatccaggaggagatgatagtgttttagactaatttggttatacaaaaggctataagatataattaattctggatatattttagatgtacagccaacaatgtgttggttggataagatgatggatatgagagaaagggtatttaaagatgactccaaattcttttacctgcacagtggaaaaaaaaatggagtttttttttttttttttttttttaaagagacagggtctctctttgtagcccaggccagactgcagtggcatgattacagctcactataacctagaactcatgggctcaagcaatcctcccttctcagccttcctggtagctgggaccacaggtgtgcaccaccatgtcgagctaatttttaaattttttgtagagacagggtctccctatgttgcccagtctggtcttgaactcctgggctcaagtgatcctccctgcctcagcctcccaaagtgttgagattacaggtgtgagctgccatgcccagctggagttgatctttatgaaattcagaagcctgttggagaagcaggcttgggggagaatggagagttctgtatgagacatggtaagtttgtgacgtctgtattagacatccatatgtagatgtcaagaaggctgaaatttgcactgctaactttagtaaaccttttttataattggtttactaaataagttttactatgcttctccattcattttggtcctcacaactctatagactcctactctgtaggaggaatatacatgagacagtgagcattagtctttggaataaaggaacagtaaccagtgcaatgtgacattgcacaatatgacacagaccctgtggtatgggctcatgtgctttgatggacaaatatcccgcatcacatttgacgtggaagacacaatcctggaggcaactcagtcttttgcagcagaacccagagcagatgtattcagggcattctgtattgcagtttttcttcctgcctctcaaattcctctggtgttatgacctgccttaggccacagtgagttcctatatttgtaaaattggtggtcactttttccctccatagtgctgtttgtgaggccctttgccatattcactgtctctaattgcctgctggggtcaaccttctgcttttcacttgtttcctcaaagacacctcaaacttggccctgccaagatggctgcactcaccttatatgagccactcaagcaaaactgttttccagaacttaaaacagtggctaaaaaggaaagaccagggaagaggaaatgagcaggttggcaaacattgtcaacctaggaaagcccaatgtagctgttatcacagattgactgagagaggactgttggatctactttacaccactagctgacctgtttttggctgacaggttttagttcctcccctcaaccctagtctttacaatgaaaattttctggctactaatctgtgctcttccattcttcttagtccccatatttctatagactcctactctgtagaaatagatgtaggacagtgagtgttaatcttcagaataaaggaatagtaatcagtgtaatgtctgaagtgctaaatcaaatgaagccgaggggttaaatctccttgcagtttaaggtcttagacttcttagttactctttttggtacattaaagaatttgccatctttgatccatcttatttatttctgtggcccttcactactatccaaagctgcacatttatatgtctgaaaagttaaatagccaggtaattcctctgccatgaactcacacctagaagtgattctaggtgatattctaagagcttctctcacaagcccataagtcagtagctgcagtgggaaatgagctctgagtagaacttgtagatactgagaacaggagcaacttatatcatccctgtgccacagccgttctccagcttgcatcctttgaccttcaaactaaagatgtgaaatgcagagagctggctttgaaaaccattccagtctgatttatcccaaatttggcaaatcatccctttaatataagctcaatgattgcatcaacaaaatatacaggtgttctattatttatgatacctctttcagccctggtgcagctccatggtatggaatttaatgtatttaaaggctactgatagttatagcactcttttcctaaatactagtctggtgtttatatcagacaggtgttaaatgattttgtagggattaatgtagggaattataaaggattctattgttactagaaaggggtcccgatgcagaccttgaaagagggttcttggatcttgtacaagaaagaattcaaggcgaatccacagagtaaagtgaaagcaagtttattaagaaagtgaaaaaattaagaatagctacttcataggcagaacagcagcatgggatgctcagctgcttatacttattgttacttcttgattacatgctaaacaaggggtggattattcatgagtttcctaggaaaaggggtgggctcttctcagaactgagggttcctcccatttttagaccatatagggtaacgttgccatggcatttgtaaattgtcatggcgctggtgtgtctttgagcatgctaatacattataattagcgtataatgagcagtgagaacaaccagaggtcacttttgtcaccatcttgattttggtaggatttggctggcttctttaccacagcttgttttatcagcaaggtcttagcgacctgtatcttgtgctgacctcctgtcgcatcctgtgacttagaatgcctaacctcctgggaatgtagcccagtagatctcagccttattttacccagccccttttcagaaaaggcaggtctggggccagggcaggcatttggaatggcttgagggcacagaatatttccagggtagaggagatgtgctggactaaattatagagtgatggactaaactgactttgtagcttgacttttggtttcagagttggaaatctggtttacagttggacattatacagtggtggattaaattcactttgcagtttgacttctggctttagagctggaactcagtgcacactagtgaaagtcatgcttcagactccctctggaattcaaggggaagataataatgcgcgcttactatcttaaattaaattccataattttcagctctgtattttttccaaattaaacattatatctcaaacagacccagatatatttgaatattattaatgacaaacgttaggcttaaattacaaataataatatacctaacattggaaatttccatcattcctagtttgtcagactcctttatcttgctaatttgcagatattgctttagtaatgttgccgtgattaatgaaggttttcttggtattaaaagatccaaagagataggaatatgtaattgaactctagattgttgatattctactttcagcattctgaagtcatggaaattcttactgtagaaactcaataaacttacaagtagacctttactttttagttcattactgataaaataatgaatatagtctcatgaaggtgagttttcagaaaatagaagcatgagttgtgaagataatatttttaaaatttctctaatttgttttgttttgcaggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtnctgggaaggtgagtcaaactaaatatgattgattaattaagtagagtaaagtattctaatcagtgttattttgttactccctactgcttactatgctctaagaatgtgtttataaccattcctcaaagcaatctttttcatgcttattcagtaaattagaaacttacagaaagtagcaaagccagttcttggactcaaaaactgataattaactttaacagactttttcagttttcaggccattgtcttcacactgttcttccttcctccccactttcctccttcccttagttattttcttctttcttttctctcactttcactctctctccactccttccttccttctttccttccttccttctttccttcttttctttccttttttccttccttcccttcttccttcttttctttttctttcttttctttctttctttctttctttctttctttctttcttgctttcttgctttcttgctttcttgctttcttgctttcttgctttctttcttttctttcaagcttaaatccattcctttattgaggaaagtaagcccattttatttgtacatgtgaggggggagattaaatatggaaaaatgctaggggtatttattatatctgttttaaattactaccactctttctttttttttatcatgctcctcccttcatcctatttctgtttatctttaccctttttttacttcttttttttcccctgcatacttgtcttttttttcccattatttaacaaatgcttatgtggcatttactgtgtttccaggcaaatgtcttattccttatagcaaccatatgggggtctattatctcatttttctagtggggctgaggcacaggcaggtcatggctctggactttagagctgataggtcttggagctgggattcaagctcagacagttgtgctccaaagttgtttctctttctgttataaaacaaagagttcctctgatggcagaatgcagtctgatatcacatgatctgtatcatagtggaaatgagaggtcagagcagggctgactgccataactaacatttaggacagggatatatgtgtgatgaacattctgagattcccaggagttagggcagggactcacacagatcagagtggctctggttgtcagtaggcccagctacctcaccaagtgaatgatgaagaaggccccagatgttggtcattgccactaatgctttgtcctttacctctctgctatctcttcagactctttatcccatttttggggggttccctctggaaaatcttttgggccagctgtagtacctccccagccagtgtctgtaggggacaaacctcatgcgaggcatcctatcaggctcccggaggcctatttctggaggtacatcaggatggtgctgacccacagggttctattgtggtctggctcaagtaacgttggcccctgccagggaatacattctgtgccaatgacttcctttaatattgtactttgggggtctcccccatggtgttgactcttcccctgagaagatggaaaaatctaggacaaaatataaaaaaaggagaaagcattctagttaacagtgtcattatttaatgaggtatttgaaatgtcccagaagggagtcttagattgcttttaggaagaagatgatacaatctgatcctaagctaattcatgcaaaccacaggttagcacctttgaagtgacacatgagatttaagactcttggagttctgtcagaggggccaaaggaaggggagcagatggaaatgaattatgtgtggatatggcagattttctgagcaaaggtcagggatgtgatacattttctaattcaggggtggctcatgagagggacagaagtaggtagggaaagtagtggatctgcaccaaccagtccacataatattgaaaatcaacttccacctgaacacacttctcaagctgctatttagtacttcttatataacaagtatcattctaacagctctaaatacttgatctaatttaatgctgtcaataaccctgttgaattatccctgttggatagataatgaaacccaagattcagagagatcaagtaacttgctcacagtcacacagcaacagcctcctaaccaatctccctgcttcaacttcttccattcttatccaactatagtctacattcttacagaagtcattctgacctctcataaataaaaatctaataatgccaacccttacttaaaactcccaaatggcttctgatcatacttggaacaaaatctaatcttttatcatggattaccaaactctgtatgatctaactcctgatgacctttatggccttatctggcatcacagtctcttatgcctgtgtgccctggcccagtgggcattcttgcaattcctggaatgttttaagctgattgcctctgatcttctcttggctgactttacgttattcatatcccaaaattattacctcttcagagaggccattcctgacacctgaattaattcatgatacttcgcagcccttgatgtttctttcctatttctttgtttattggttaatggtgtaccatttgtttattgattaatggtgtatgtgacattagagcccacacttattctgactgcatgatgtcaaaacctgctcataacctctaccctgcctgtgatctgtgctctgcagtttttcagcacattattgtggactagtgagcatttttataaaatacaggtgacccttgaacagcacgggtttgaactgagtgggtccacttatatgtaggtttttttcaataaatgtattggaaaattttttgaagacttgcaacaatttgaaaaaactcacaagccatgtagcctagaaatatcaaaaaattaagaaaaagttaagtatgttataaatgcataaaatatatgtagttactcgtcttattatttactaccataagatatacacaaacctataattaaaaaaagttaaaagtgatcaaattttatgcacacaaacacttacagaccatataagtcaccattcacagtcaagacacatgtcaacaactgtaaagatacagtgttacatgttttggctctgtgtcctcatccaaatctcaccttgaatggtaataatcttcacatgttatgggagggtcccagtgggaggtaaatgaatcctgggagtgggtttttcccatgctattctcatgatagtgaataagtctcatgagatctgatgattttataaaggggagttcccctgcatatgctgtcttgcctgctgccatgtgagatgtccctctgctcttccttcatcttccgccatgattgtgaggcctccccagctatgtggaactgtgagtccattaaacctctttcctttataaattacccaatcttgggcatgtctttattagcagcatgagaacagactaatacatagtgttaaatcataaaataaaaccatagtatgtactgtactaccccagtaattttgtagccacttcctgttgctactgcagtgagttcaagtgttgtacctgcttaaaatgcagtgacactaacaatctcagcaggagcagttcatctctccagtaaattgcatattgcaataaaaagtgatctctcatgattcttgcgtattttaaaaatcatgtttagtgcaataccataaacattaaataacaccagggaacccatatcaagtgacactagtgttgctggaagtgctcccaacaagcagagaaaagtcatgacattacaggaaaaagttgaactgcttgatacatactatagatgtgaggtctgcagctgcagttagttgcctgccatttcaagataaatgaatctagcataagaaccattgtaaaagaaaacaaaacaaaacaaaaacaaaattcgtaaaagccatcactgcagctacaacagcaggtgcaaaaaccttgcactttttgtacaatacatttatctcatattgaaaatgcagctttcacatgggtgcaggattgctgtgagaaaggcatacctatagactctaatatgattagaggaaaagcaaagtcaattcaaattaaaagtaagattaaggatcagagctgggtcatttaatgccagcaaaggatggtttgataattttagaaagatatttggctttaaaaaaatgtcaagatagcaggagaaggagcttctgccaatcaagaggcagcagaaatgttcctagatgctattaaaaaaaatcattgagaagaaaggatatctgcctgaaaaggtttttaatgcagtcgaaagtgccctattttagaaaaaaaaaatgccacaaaggacttttattaataaggaagagaagcaagtccaaggatttatgtcagaaaggagtaggctaactctactgttttgtataaatgcaatccggtttaggatgagtccatatctatagatatgctcttatgtgtaaagctgttcatccctgatccttgaagggaaaagataagcctcagctgccagtattttggttgtacaacaagaaggcatggacaatgagagcactttttccggattggttacattgatgctttctttgttcttgaagtcagaaagtacttagacagtaacggactgccttttaaagttcttttgatattagacaatgctcctggccacccagaacccatgagtttaacatcaaaggtattgaagtggtctgcttgcctcctaaacatgtctctaattcagcctttagatgagggggttataagaatctttttttttttaataaattttttgtagacatgaggtctcactgtgttgcctgggctggtctcgaactcctgagctcaagcgatcctcctgccttagtctcccaaagtgctaggattacaggtgggagccactgtgcctcgccactcataaggatttttaaggctcatcacaaagcacatggtactctacagaaaggattgttaatgctatggtagagagccaccagagagaagaacatcatgcaagtctgtaatcctaaaacgataaatttctgctggagaaaactgtgtccagatgtacatgatttcacaggatttatgacagagccaatcaaggaaaccacaaaaagaaataggagatatgaaaaaaaaaaaaaggtggtgggtgaaggctttcaaggtatagattttggagaacctcaagagcaaataaacatcacaccagaggaattaacagaagatgacttgataaagatgagtgtttccaaaccactgccaaacaatgcggaagaagatataggagaagcagtgccagaaaactaactttacgcaatctggcaaaagagtttcggttattcaggactgcttttaacttcttttatgacatggacccttctgtgatacaggcactgaaactgaagcaaacagtggaggaaggattggtaccatattgaaacatttttagagaaataaaaaaggaaaaagtcagacagagaccacaatgcatgtcagtcacaccaggtgtgcctgcctttcccgcctccccttccacttcctctgcctcttcccctctgccacccctgagacagcaagaccaatccctcttcttcctccttttccttagcctattcaatataaagatgatgaagataaagacctttaaatgatccacttccgcttaatgaatagtaaatatattttctcttccttgtgattttcttaataacattttcttccctctagcttactttattataaaaatacaaatatgtattacatataacatacaaaatacatgttaattgactgtttatgttattggtaagacttctggttgacagcaggctattagtagttaagtttctggggagtcagaagttatatgtgaattttcaactgcacgaggggtcagtctccctaacccccatgttgtccaaggctccattgtagatgcttttttacttttcagaaataaaagccataaacttgtttatttgagggtaggaagagtaatgtcaggaggctattttttctttctagaaacatacatttttatttcttcaaaattatttagtacacaacagtttccgagggaaactcgatgctatttgttcttggattagaaattctctctcctgatggtgattactgagtctccatttaaaactcttggaataaacaaagctgtgaggatgcggggtgctaattaagccttttcctaattgcttcattgtgcgtcaagatgaagacagtcctgaagttattatactgttttactcaccacttttaggtctgtgactcaaactcccacttttattcggctatatacacactaaaaagcaatgacatttacaaaccaatctcagaccagacactcctgccttagaacatggtgcacagaaaatatttcttaaaaccattacactgaaatatacagtaaaatctgtttttcagcagacattgttatagtctgttgaattttcttactaatctagaaaacctgtttgttagaattctgataattagaaatatttctttttttttttgcttgtgaaacttcagcttttattttatttttttatttttattatactttaagttttaggatacatgtacacaacgtgcaggttagttacatatgtatacatgtgccatgttggtgtgctgtacccaccaactcgtcatttaacattaggtatatctcctaatgctatccctccccccttccctctaccccacaacaggccccagtgtgtgatgttccgcttcctgtgtccatgtgttctcattgttcagttcccacctatgagtgagaacatacggtgtttggttttttgtccttgcgatagtttgctgagaatgatggttctaatgtgtctgacaaaaggattgtctttgggaatttgaaggtgaattttttctcctcaccttttgctttctgcacttttacgattttctaaagtgactatatatcatttttataatgtgtaaaagaagtttatacaatattttaaaataaacctgccattttcctaattttctaagtatcttgtggtaaacataattcaatcttcttggcctgtcagtgtaagaataatattttaaattttatttttaaataagtttttgtttctaagaatgttactaatttttttttttacttctgatagattttgatattagtcttcaaaactgacttaatgtcttatgaaatgcttgctgttatgtttgaagttaggtaatttatgtaagattcagtgaagaataagtggaattccatgtttatgattttaagctataaaacactctaaattaaatgtgtctttattagaatctgttctgaccagtgcagaggccaaagagaggaagaacatcttaaacaataaagagtcatttctcttggtgacttataattctggaagttatttctcttaaaatcatagcattaaaagggactttagagaccctctagtccatcgtcctcattttgcaaatgaggaaaatgagacagcatgttggttcaaggtggtgcggctgatgtaggctgaaatctcatcttgtacactggtgttctttgctttttccatatccctttactcagactccagaggtgatgaaggatgtatgtttcctaatcagattgccttgttggaagtaacatttgattacaacataattgaatgatggaaactttctttttaagatggagtctcactctgttgcccaggctagagtgcagtgacgccatcttgactcactgcaatctctgtctcgccagttcgagcgcttcctctgcctcccagtagcatgggattaaggcatgtgccaccatgcccagctaatttttgtatttttagtagagatggggtttcaccatgttgatggtggctggtctcaaactccttacttcaaatgatccacctgcctccatctcccaaactgctgggattacaggcatgagccaccatacccagcccaaaactttctggaaaacagattgatagtatgtgccacattccttaaaaaattaaaaaaattaattcaagccaggtgtagtgccatgtgcttgtagtcttagctacttggcagactgaggcaggaggattgcttacccaggtgtgtgaggctgcagtgagctatgatgatcacacctgtgaattgccactgcactgcagcctgggcaacagagcaagaccccgtttctaaaaaaaaaagttagttttctttgacttattaatttcaccttaagaaattttcctaataaaccaattcaatatggacaaatgtttaggtacaaagatgtttatctcaccactatttttaataaaaaaggaattgaaaacctggctcaacaataaaggaatacttaattggttatgatattaaaggactcattacacatctcattattaatgtgtatttaatgaacttggaaaatgcttttgatatgaaggtaaaaataatgatatagagctaaatatagagtttcattccaatctttttaaatatatttatgcacttaggaaaaaaacaatatggaaatgtgtaaaatatactttttttttaaaaaaaaggacacatttattcagcattatgatcagactattacatttaacaatcaacagtatgggtgccaaaaaaaatctacattaaaaccctttgttgtaatgctttacactttccacagaacagaaactaaaagaatctgttacacaattagtcacaaatatagtcctcgagttttttacccatacacatgagtatttgtctaaaacatgtcttcttgtagcacttaggccctgccaccactgtgcttgtctgagttcacaaatctgttgtaaactgtagcttccctgtcacttctctggctcttatctcctgctaagatttgtttcctggcagtaatttaaaatcttctgccactgctgtagctactgctgctactggaactgccatagccaccttggtttcatggtttggcaaagtactggcctgtaccagcataggggccagagcttctgcctccaaagtttcctcccttcatgggtccaaaatgtaaaactaattgttgtaattgccaaaatcattacaccacctccaaaattgcttccatgattaccaaatccattatagccatccccactgccactatatccaccaccaccacagctgccaccaaagccacaatgaccactgaagtttcctccacgaccaaagttgtcattcccaccgaaactacctccacgaccaccaccaaagttcccagagctgctttgcctctttggctggatgaagcactcaccatctcttgctttgacagggctttcctaacttcacaagtgtggccattcacagtatgggtatttctcagtgacagtcttacccatggagtcatggtcgtcaaaagttactaaagcaaagccccttttcttatcactgccttggtcagtcatgatttcaatcacttcaatttttccatactattcaaaataatctcctaggtgatgttcttcagtgtctttaatgccaccaacaaatatctttttcacagttgggtgggcatctggtctttgagaatcttctcttgagacagctctctttggttccacaactcttccatccaccttgtatggccttgcattcatggctgcatccacctcctccacagtggcatatgtgacaaacccaaagtcccttgagcgcttggtatttggatctctcattaccacgcaatccatgagcattccccattgctcaaaatggctcctcaggctctcattggtcaaccaatgtagagctcaacctccaatgaagaatttcctcagctgtttgggctcattaggagactctgacttagacatgacggcaggggaaagagagactttaaggatgcttccttggtggcgtccacgggcagaaaggggtaagcgtccacaggcagagaggagtaagcctttgaatgtatctaaaatttactttttattgcctgcattctttcactattttccaaacattcttcaattgtcacaaatggcaatgataaagagaaaaatataaacatcacattttaaaaataagtgtaaaataactgtgaacttaaaatgtgatcatcatagaagaaagagcactgcaatagaagtactgtgagttctctggttaattttgcgaagccacgtaaagctgtgtggctttaagcaatgccataatccatttaagtcttagcttctatttctgcaaaggagaagtttgaattagtcaatcttccttccagatccataactcagtttgataaattacttagtatctttttctacagagaaaatgctcatacataataataaatatgtaatcataaaattattttcattagtctgttttatagaattcaaattaatcaccactatttactcttgtgcctcttggtgatcggtgctgtctgttacagatcgctactgaagcaatagaaaacttccgaaccgttgtttctttgactcaggagcagaagtttgaacatatgtatgctcagagtttgcaggtaccatacaggtaataaccgctgaagagtgggaggagagtgtgaataatttttcaatcatcatatttgttttcagagggattactttggctagaaggtagggagcaagtggagaaagtgctcgaaggtaaaccattgagaaacagttgtaattatgcaggagagaaagtacaagaccctgaactaaggcagggacatctctgaggtagaacctgtaagaatgggtcactgatgagaagggagggagacatgatgctgagaatgactatctgatgtccaggtaggatatgaccctataatttgctctagttgaaaatgagttatttatggaacctgaaatttgaggtacctgtgggacacagagaggatggtgcttcctgggtttacctgtctttctgctttttaccccttctccagtgcagaccttcttcctttaatacagtcatcccagtgagggctctaataagtggtgtgaacataagcaagcccaacctttctgagtctgtttcttataataaaaggaaagctggactttattgatggatttttaagcttttatttaaaaaaaaatgatggtagagcctttttgtctaaaaaaaaattatttgaaatcttcatatgtgatgaaggtaaaagcagagttgatctggtagcaggaggggttggaagcccagggctgcccacttgctaacctgcccccacccacacctccatatcactgagctggatccatatcatgattctagaatgtcaacccctttttaaagccttctctgagacccccgaagaatttagtgcttctcctttcttcctataacagattattcataaggcacctctaatgcataaatagtaattcaactcaagtcaggtctcctaagtcaagaacatgcctgtttcctctatgtcaacccatcatggcccctgcaccttgtaggtgttgggtaactgtgtgcagaatgaatatttacgtagagtacttccatactgtgtgtccaaaagtggggagaaagggagataacttttacttattgatccctaaccggctctttacagtcattggattattttctctttacatcaacactttgaggtgtagttaattctacattaaagataaagacactgagtctcagaggttaggcagtttcccaaatttgcacaactgttaagtgtaaagtgaggaccaaacttagttctttggaaacgaaacccatttttgttggtcatgcctctgtgccctactgccaacctatcaaaagttacattttaaggactgagaaatgaaagtggaatgagttttcaaatgtcttcttttcagaaactctttgaggaaagcacacatctttggaattacattttccttcacccaggcaatgatgtatttttcctatgctggatgtttccggtttggagcctacttggtggcacataaactcatgagctttgaggatgttctgttgtaagtattgggctattatttagttaagctctaaaaataaagctgggatgaacatgcttcatgtctagataggaaaccctactgtgaagcctcatgaagagattctggtgattcctaaatcggcatttctgcctctgagtcttcatgtgccaccattgaagcaccccctttcatttggaggagcagtaacttctttcctcattgctggctcacacatagttgaccttttcaaatctgtgactgagtggagtgattccattcggagattttgagaaggccttggcatttgggaagaagcctagccctgagcaaggagtctgactggctccttttaaaggactttcttacagagcaagtaaaatacagatgtgttgtactaagttctgcaagcctttggcaattccaggatatgtttactttcccttgataagagaggaattggaaggtaagagccaaatgaattcagaaatgacaaaggaaaagttatattgggatttttctgctacattgttctgaatgtagataattgtacctcggtcagaggaagataagcctgaagcaattatactacaaaagtacccaaatatgcaatttggtggtcaaaagtatgtgtaatctttctgagcttctagatttggaggtgggtaagattctgcctcttgatagcataacataattagtagcgatataattttatatttaaaccagaatcatataagcctggcagtataatgtgttagtatagtatttctgtcctttttaaacattgagttgttcatgcattacagtttgctcaggatgacccccaaaaagacatctaaatttccattaaagatgtacattggacaaatgatatgcaggtgctatttgtgattatggcctagagatcaaaaccaagtatcactggcattggggctttgattagataattatttgatatattgctttactccaaaaaattgaattgatgaaagttgctgacattggggcatttaggtttgcaaaatcaaccttccaagttaaggaaaaggaagacctgtctgaagaacagtgcattagaaaaaggtcccataggtttaaatgatcacaagtccaagattaactatcggtattttatttagtgctagttaaacaaacaaacaaacaaaaactaatgacacaatatgctacacaaacataggcatagagtccagaggaaagaaagcgatgaattccctgtattctgggcgggttcccaagtattatgtctgttccaggcttgtgtttaaaaaaagcatattaataaatgtgtctagagaagggcagtgaaaggagttatcaaagaagcaaaggagtttttagcaatatttcaagacttgtaggcctgtcttatgtaaaaggaagtaaattttttcttcagtttgaaaaagaactgtttaacaatagacaatcaaacatgctacttcctaaaacagaggatggaagccaatttaggggaggtgtccaggcacgaacatggagagctggacttgatacctgtaaggtccttcccaactttaagttgctgtgattcccatgtcatagataagaacgtcaatgcatcttaagagcaacatgatatctggctctgtaagaaactttcttttggttgcacaattcataggtttttaagaatctgatgtaattccaacatcactgactgtatccgttgttgattaccacaataatgctgtgtaacaaccagccactaaacctcagagacatacatatttttgcccacaaacctatgggttagctgtgaagttctactgatctggattaggcatagtggatctcggctggacttactcatgtgtctgtagtggattgtgtctgtagctggttggttgggattcagacagctctcctccatgtgtgcctcatcctccagcaggctatcctgggtttgagacagtggcagaattctgagagagagagagagagaaagagagagagaacacatgcatgcatgctcacaaagcatataaggccccctgagtcataggcttggaactggcaaaagtgatttccaccatatcctgttggccaaagcaagtcacaaggccagcccagattcaaagggtgctgcaaaggtcacagtgcaagaagatgaggatacagagagaggtatacagagggaaaaaaatgggccattttgcaatcaatctatcacatagacatgaacttataaggaaatgtgtttgttttatttttaacatctgttttataactattagaggcaaagctctctggttatagaagtgtcaacttttggccaggcatggtggctcacacctataatcccggcacttttggaggctgaagcaggaggttcacttcagcccggtagttcgagaccagcctgggcaatatagggagacccccatctctacaaaaaataaaacaattagctggtgtggtcatgcacagttgtagtcccagctactacagaggctgaggtgggaagatcgcttaagccctggagatcatatctttagtgagctctgattgcactactgcactccagcctgggcaacacaggaagaccccacctcaaaaaaaaaaaaaaaaaaaaaaaggagtgtcagctttctagcattgtgatggtaatgctgtgcacatgttttgtgtttgtgctttccagagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccggtgagtttgatgtttcaactgtttgatctactcctgactcctgaatgaaagtattttaagtggaaacttaataaaatttgtactttcaaatatgctgatgataaaataaaacttcctagatcatagattcctttcaattactgctaataatatacatcaacattcagtacttttacgtagcaaaggttatagggaaataggaatactgctcactttataagcaaaacctattaatcagattttttaaaaacaatttttttttagagacagagtcttactctgtcatccaggctggagtgcagcagtatgatcatagctcactgcagccttgatcttctgggctcaagcgatcctcctgcctcagcctcccaagtaactgggactacaagcgtgtgccatcatgcccagctaattttttaattatttgtagagacagggtctcgttatgttgcccaggctggttatcagattttattgtatgtaagttactgtattcctgaggaacagatttgagttattgtagctgtattgcatattcatattgtcttaacaatacatgctatgaaagcttttactcttttagatctcatttattaaattctagcagtctgaggtcaagcacagtggctcatgcctgtaatcccagcactttgggaggccaaggtgggtggatcacgtgaggtcaggagttcgagaccagcttggccaatatggtgaaactccatctcaaataaaaataaaaaaaaaaagattagctgggtgtggtggcacacgcctgtaatcccagctacttgggaggctgaggtacgagaattgcttgaacctggggggtggaggttgcagtgagctgagatcatgccactgcactccagcctgggtgacagagtgagactctgtctcaaaaaaataaaaaataaataaataataaaattaaaataaaataaattctagcagtttgaagtgaagccaattgtaacacaaattaattatcttctgacacctggtaatcgagagagttagctatacactttattttcagtattgcagcattcaaatttactgttattcttctcattgcagaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtggtgagcacactttcacatttagctcagttcaggttttcatcatccaaatgtctgaatgtatttaattctcaactataagccatgttttttcaaacctttaaacaacagtcccacttggataaagtctgagagcctaaatatggtctccaagtggtgtcatctgtcccagccaacttctccaggctcccctcaacactacccctctaccctcctttgcaagcaccctttgtaccacctgtctcccttgctgtacttaggtttcagctgacttgaaaagaaccaacaaaaatggaagtagccagaaagatacaggacaggtgctaggagtgagatgaaagccaaggatggagactgtttcaaagatggtggtcagcaatgacagatggtatagagagattgggtaaggaggagactgagaagacatgatagaatctagcaatgaatgggctatttctgacattttaaaatattcttagtgaagtagtgatggtaggaaacagttaagggtggctgaagtatgattaggggaagaaatggagatgtaagtgagcatagattatcaagaagatggaaagtaaaagaaaggcaaataaggacagcacttgagtggggaggcagagtccagggaaaatgtttaggggttgaactcttgagcatttttatgactggggatgagacaaaatcattgatggagagagggcattgaaacatcatctgaggaaaaaaaagtagaatcaacaaatttgggaaatgtaaaaagaacatggaagtatgcccttttgtcttagttgctggggatgagggaagtgtctggcagggagtagtgagtttaaacgactatgatgggggataggaaagaagacaaactaggaaggaatagaattaagagcaagacattttcattatgtaggcatgtaattagttgtactgagaagtgatatttaaaattcttaacaatgggaacagcatggacactgaccaatcagaatggatgcctgctagtacaagtcagatggatgcccacccgctgtgtatggacgctggtagcatttaatgactcaaatgaaaagagagttttcaatctttagtctaggactctttttacttcctcacagcggtcactcttactcataaatgtttgttattgtattcaaagacagttatgcctttattcagtttactcttaaatcattacaagcttgttccctacttttactctcttgctttatattttctgtgaactctgattgtgtccttcaattttgtggtgaacttaaaaacaggactctaaataaagacgtcgctgacagccaaagcaattagaggaggaaatgttagctctcagacactgattatggccctcaacagacagttattaagtggtgtgttaaagattgtgctataatgaattgtagggcatgatatctgccctcaaatactttaactgaggagatggggcatatagttttgtgagttaaaggagataggcctagagctagttcttaagagataaggatttgcatatgggtgaacagtagggatgacattcaatgtaagagaaaatacaagctgcgatcagtgatttgttcccaatgcagcaaataggacactttggctgaaatgggagtttagtttgagtagtggttctcaaacttctctgatgagaactcatgtaggaaagatttaaagatgtcaccatccactaatgtactcatagtcactttccagtgggaaaaacacttggtgataacaaaatgttggagttgaaaacaacaatacaatacagcacaataatgatacaataaggaataattaatgattattctgcaagtatataaacacaagcagaacataataaaacatggacagcatcagttacagttcagagtaggaaacagaaactgccctaggtatttcaagtagataagtatttaataaagggaattacatgcttacaaaatctttggaagggctggaagagtgaaaggaggctatttgctcccagacttccaaatcacagcactgcagctgtgattctgtaaccaagaagctgcagaaaattatgccgatatcacagctgttccaaatttaaagacacgagactagaatctggctgttgcagaaattcttgtctgccaaagatgagttaagccgttagcttaccacagctgctgaaggagagctggtgtctacctcccagattttacactgtgcatctccctcgtagaccctgacttacttccagaaccaagggaaactgggaaatagtttttagccttctagtcccttgatgtataaaaggtcagacacagaagtatatgaaaatgaatgctgagtgcataggacagtaagcatcccaagacattattgataatgcactgaaaacaaagtgtcctcatttattgcaggaatatccattatcttctcctgtgcatttgcactagggcttggaaatactgccagatggtgcactgcagccagacttctcagcagaaagtgtccacaataaactctagtaaatgtacaaagttttgtttatggacatcaatgagtagccctgaataactcagtggattactactaattagtttttattgccatgtaaaaatgagtctgtggatttgagcataagaattaaagaaaggattggaccctaaggaccccctgaagtcaggaaattctcgtgaccatcagtggaccttgaatcccatgttgaaaaacattgacctgaaatggtggttctaaagcttcggtgaatattagaatggcctcaagagctagtaaaaaacacagccggcctggattattcaagtaggctagggtttggccttttatttttataatattccgaggtgattctgatgccaaccatcattcgagagccactgaactggtaaatttgaagaaagatttaggttaaattgtggagagtcttgaataattaagctatgaaattgtgaagatgggaggatcctccaagttttgagggacctgaaggttatacaattgtagggattcaacttaaagtaaaaaaaaaaaattttttttttaatgcaaaattagatacaaaggtggatatttacttcaaatgaaaaaaaaatcataaccaaatactggaagcttaaagattctggtcccttttcatttttaggtaatttatcatcaatacttacagggaagagcttcctgactacagccttgcctccctcccacccagaaaactgttcgacttcccagaatttacatgtaattgagggctcttgaagcttaaccttcatcagtgtcactgttaaatcacccctgctttgagtcttccccttacctttcctttctcccctttccagtctctcccatatacaacagtcagaaccccctttgggatacaattagaagctgcttgtcattgtctggttataacccaagaagtgcctgagccaagatgggggcattcccctttcaaggggggacacaaaatatctttcagttgtgaggtattagtgttaccaaaacccaacctagagtgtttagatccagtcttggttttcccccaattactaattgtgtgaccttgtataagttacttaactttgctcatttccttatcagtaaaatgggaattaacatcacagtgaccttataagtgttgctatgaaggttaaacaagagactgtatgagacatgcttatcacagaacctacaacgttgtcagtgcttgatttttaaaaatcaattattatttcacagtaaatatgcatagaagaatacacatttaacatttacatttttgcgctaatgaaagcaagcaatagacttaacagtctacaatagagatagccaatcattttaatctggcctttcattatttcttaccaataggtttactttcctagttatatttttatttaggccaggattttttgagcacttctcatagattccccagagttccttggtctttcctcagaccaacaccattcctcagccaaagaagctctgcttctttgatctgttttacattctaggcatctaaacttgttttacttaaagaaagaattcttcagtcaacacaggttttaaatagtttgcaattctagggtattatgcggggacgaggaggcccaagagtgtattcagtgtaatgaagaaactcattataacttgctgagatcattcagatttgcctgtgattatattagttaggcagtgtctgttttcctcccaggggtataaacctggaaaattacaacaaaaacaaaagcaaaaaccaaactcctcctttccttaaatttccacttctcaagtacagtgttttatctaacaagatctgctgcttagccacatccttgtttggctttcactgtctctctccaccctctactttccaccttcatttattaacattgtaaggaagcctggagcatacatgtgtggaatagtctccatggcaactcagcttggaactaataaggttatgggagagcttccatccctgcacctgccagtgtcacaagcagaagccatgttcctgtagcaaagattgtactctactgctaggcagctgtccccttgagccacccagccaggcacatgggatacagagagtatatggctcagcacgcactagtcactactattagaaaagctcaaagctgagtcactggtgccttcttcagaagggatgaacagctctctcacttgaatgccagaaaattatcttgcaaagcagacctatctgatagacatatttgcatcagagtagggcttgttatcagcaaggctgtaaggtgccctccccagtcttctgcaggataatccagggagccagattatagagaaagggcagccctctgttcctactcatctggctcagtattgaggatctccattcaccctttcctacccctgttcacctatccatcccctgtagttcctgacctgcaaagctattatgtggtcatagttgttatttatttccatgctaatcctgggcactgtttctctgaaaaatggagatttaagaataaggctgtcaggatacatctcagaagtattaggcgttgtattagtgtggctgctataaccacttgccacaaattcagtggcttaaaacaacaccaatttatgactttacacttctggaggtcagaagtctaaaataggtctcagtgggctcatatcaagtgtcaggagggctgtgttccttctagaggctccaaaggatgatctgttttcttgcttgtggccccttcctccattttcaaaaccagcagtggctggttgagtctttctcacactgcatttgctgatactcttctccctccttcttcttcagttaagagcccttatgattacattagtttcaccaagataattcaggataatcttattttacagtcagctgattagcaaccttacatctactactttagtttctttttgccatgtaacataacacattcacaggatccagggattaggacacagatgtcttgtgggagagggaacattattctgcctaccacatgcatacatcagaaaccatggttgaaacacaggaaacatgacagttcctcaaggcatacaattatgaccttgttgggttaaccttcactatccaaattttaatcacacaaacttttccttaatctcacagtaacttggcagtttcagtgtaagaaataatgatgttaattgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatngtgagggcagcaaaggaggccaacatacatgccttcatcgagtcactgcctaatgtaagtctctcttcaaataaacagcctgggagcatgtggcagcctctctggcctatagtttgatttataaggggctggtttcccagaagtgaagagaaattagcaaccaaatcacacccttacctgtatacaagcatctggccacacttcctgtttgggttagttgttacctttacctgatcacctgaccctccttgtgaggaagggatgaaagtgttcgaccacttcaggtttaggagagaggaacatttctgggataggagaactggaacaattgtcttgatccaaagctataggcttgaggctccacctttgtcagccttaggggtaagtacaatatctggaaagcctttcactttaagtccaagtacagagtctgggtccccacctgcacatgctgcttccggcctgctgaggaagtaggcatgactgtctctccccatgtctctcccctatttctcttccttcttcctccttgcagctctctcccaagcctcaaaactcactgtggagtcagtccagtccagagggctgaacccatgccagaaagagtgctctctctgaataagggttgaaccattaaagtgaccatggtatcatgtaatctctcatcgtcatacatttcatgggccccaaactttgtttacagtgaatggttgaaatcttttctagggctccctgggctgtggtcctatgggtgtcctctgaattcctcctaggaagttggataaatggaacctctcatcctctctcaagccctcttaggtcctgtgtattagtttttgtgttttgttttgttttggaatacaagttgactattccttgtccaaaatacttgggaccagaagtgttttggattttggaatatttgcatatacataattcacttatatttcatgtataccttatacacatagccagaaggcaattttatacaaatttttaataactttgagcatgaaacaaagttttgactgtgttttgactgtgacctgtcacatgagatcatgtgtgaaattttccacttgtggcatcatgttggtgctcaaaaagtttcagaatttcagattttgaattttttggattatggatgctcaacctgtattaaaagtccaaaattagattttttccaacctttattttagacttagggggtacatgtacaggtttattatacctgggtatattgcatgatgctgaggtttcagggtacaaatgatcctgtcacctaggtactgagcacagaacccaataggtagtttttcaacacttaccccccttccctccctacactaatagtcctcagtttctattgttgccacttttcatctatgagtacctgatgtttagctgccacttataagtgagaacatgcagtatttggctttctgttcctgcattactttgcttgggataatggcctccagctgcatccatgttgctgcaaaagttaggtcctatattttgaagtgcttctcaacctaggtaatctacctcacccattatcaccagttatgctctttattctaaggaagtgccccctaaaacaaagctcaggagcctcaacccggcggggaagacagtttcctcacgaggcaggcaagcaacaccaggtggctctctttcccaagattcccttcttccataggctcttctttggataactgctgacaccaatctcattaagtcccagggaggtggccttgtctcccactctcctttcacagtctagcccaactagataattgacccttctccaggctcaaacatttaaaacagaaagctccaatcccctttactgatgagaggatagtggttctcaagtatagacacggatcagcatcacctgggaacctgttaggactgcagatccagcaatccacgttttaagagcccaggtaatactgttgcaggctcaaattggggaaccactgggctaggggttaataaaacacaaacacagagagtaactattatctatctttttaaatatactttgcctctctggcatttgtctaccacaggacaaatcctggtctctaatctgtcacacagaacacacctgtagacaccataattctcttcctcagtgtctagaccaggagagaaaaggtaaaacccctaagtcctattacctaaggatccacttctcctaagtgattaatactaggatttacctcaccctgtaggtggctggtttagctacctccttgtaagactgccgcactgcctttatctgcctcaacaggatcagtactatatcaagagtgcaactagtaaaccctccactcacatgagatacacttcagtgctcctgccttctgagacagccacagctctggggtagagccagtcccaagcatctgccaaaacctcatctggcgtcttctccctctatttacagggggaaacccagggctgagtgccacaaaactagggttgccagatatagcaaataaaaataggcaaccctgtactaaaatttcagcctggaaataataagatagtattgttttctaggttacaagtgcagtcacatcctgtttctgctctctcctgagtaacctttaactgctgtccctatcatcatagtgaatagttggtgacaattatccattgtgggaaacaacacgggaagaattttgggagcactctaacaacaaaaatgtttaaaaaggagatgttcattttcaaagttagatatcaaaatggaatccattttgatagcatccattaaaattgtttaagtgttggagatactttcataaaaacagacactaaaaggctgatatcccaagaatgtctttcctcaatttaaaatcatgcagagagtgtcacctgctggttacaagtaagatgagcttcaattattttattatgaatactttgttcaaatcaaatttcctttctcctgccaatctttgtcccaaatataatgaaatcagaagatggaagatactaacattgcaacaatattcttcttcattaaaatgctgacctgtttacactaaaacatatcatcttaaggcccaaacatttattttcaaatagatagtaaacctgccattttgggcttttttactgttctttccttaattattcatgaaccattcttagcttctgaacctaaatttttggagtatatagaatcgtctatcctactttctataagcaagctgttagaactttactttcagttctactttcataacaacaaaaccttatttacagaaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggtaagaatttaaattgggttcatccatacttgaatcagtcatccttaaaaatgctgtattctccatagtaaagaacaaaatgcagccagattattgacagttacttacatcacctttcagtaaaatgtaaacactttctgtagctgttattcaggagagtaaagatgacacaaggcagattagtcttagagcacatatgcttcacaaaccatcactagatcagtttgcgttgacctaagaagctgctttcaggaaccatatttcctacttctactggcagcatctcttcagcaaccataaggaaaagtggtagtgtaagacagtgttaaaatctggctgtaccttttaagataaatattagcaatcatttctagtcagagaaaacgttaaaaattcagattatctgaaagacgaactcagtatgaggtattctctacaataaaaaaattcgggagtctgtatttggcctttgctcaggcttcttgaatttactgtgagctggatttgaggtctgttttcaaggatgttgtaacatccttgaaactaaggtaaacacattagtaaatgtttttaaatgagtcagtgaactgtatttactgccttaactttaagagtagatgactgctggccggcacctactgccgtggctcacacctgtaaccccaacactttggaaggctgaggtgggaggatcacttgagcccaggatttttgagaccagcctgggtaacacagagagacattgtctctacaaaaaataaaataattagctaggcatggtggtgggaacttgtagtcccagctactcagaaggctgaggtggaagaatcgcttgagcccaggaggttgaggctgcagtaagccatgattgtaccactacactccaccctggtgacagagtgaggccctgtttcaaataaaaaaaaaaagttagggatagataactgctgcctcggaactaacccaaattagggttcagtcatctgatatgacaaatcctttgttttcaaaaacctgttctatttcactgacaccttgttaagaaggatctattgttgtattgttgtggagcttttttttttttttttctttgagatggagtctcgctctgtcgcccaggctggagtgcagtggcgcaatcttggctccctgcaagctctgcctcccgggttcacaccattctcctgcctcagcctcccgactagctgggactacaggcacccaccaccaagcccagctaattttttgtatttttgatagagatggggtttcaccgtgttagccaggatggtctcaatctgaccttgtgatctgcccgccttggcctcccaaagtgctgggattacaggcgtgagccaccatgcccgtcctactgtggagctttttatggaagaggaattagggaaaagaactattatgagaattaatctatgtgattatggaataggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtga

SEQ ID NO: 2 - DNA, 3843 bp, Homo sapiens
  CDS (1)..(3843)
  misc_feature (1236)..(1236): n may be any nucleotide
atggatcttgaaggggaccgcaatggaggagcaaagaagaagaacttttttaaactgaacaataaaagtgaaaaagataagaaggaaaagaaaccaactgtcagtgtattttcaatgtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtgatatcaatgatacagggttcttcatgaatctggaggaagacatgaccaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagatgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaagatactatcttcatttactgataaagaactcttagcgtatgcaaaagctggagcagtagctgaagaggtcttggcagcaattagaactgtgattgcatttggaggacaaaagaaagaacttgaaaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtattcttttctgtattaattggggcttttagtgttggacaggcatctccaagcattgaagcatttgcaaatgcaagaggagcagcttatgaaatcttcaagataattgataataagccaagtattgacagctattcgaagagtgggcacaaaccagataatattaagggaaatttggaattcagaaatgttcacttcagttacccatctcgaaaagaagttaagatcttgaagggnctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtnctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatngtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtga

SEQ ID NO: 3 - protein, 1280 aa, Homo sapiens
  misc_feature (893)..(893): The ′Xaa′ at location 893 stands for Thr, Ala, Pro, or Ser.
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGXGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEXVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 4 - DNA, 20 bp, Homo sapiens
  misc_feature (7)..(7): n may be any nucleotide
gaagggnctgaacctgaagg

SEQ ID NO: 5 - DNA, 20 bp, Homo sapiens
  misc_feature (7)..(7): n may be any nucleotide
gaaggtnctgggaaggtgag

SEQ ID NO: 6 - DNA, 57 bp, Homo sapiens
  misc_feature (1)..(1): n may be any nucleotide
ngtgagggcagcaaaggaggccaacatacatgccttcatcgagtcactgcctaatgt

SEQ ID NO: 7 - DNA, 18985 bp, Homo sapiens
  misc_feature (1)..(1): n may be any nucleotide
nctgaacctgaaggtgcagagtgggcagacggtggccctggttggaaacagtggctgtgggaagagcacaacagtccagctgatgcagaggctctatgaccccacagaggggatggtgagatgacccatgcgagctagaccctgcggtgatcagcagtcacattgcacatctttctgatgttgccctttcaattacaaatgtatgaaagtcacacttactttttattccaggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcatgtaagttgtccttgccctttgcctttctagaggtgcaaaaaataaaatgcaggcctactatgcaggaagttaggaaactactataaatcggaagaagggaaatcctaagaagggaaagtaagattacttcagatttgaaagctctagcagtatcaactggtcgtagatacatttttaaaaactgaggttggttattgtgttaaataagatttaaagaactggacctgtattacttgtgagacttgggctgtgtataggattccttaccaatttaaaatatgagctgagatagcttgtccttatgctaaatcattctgggttttctgtggtagaaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggtcagtgaggcttagttcaaaccaacctgatttataagcataagaacattctactactaattcttgttaatattggtcttagaaaaggaaatttctgatagcttctaggtgattccttcagctattaaaataaaagcattgggcctctttgaaatctttttctatttgtttgttttattgttcaatttctatttatttctctgatcttattttaaatgttgatgaatacattttcatttgaagacacttgctaatcttttaaattaaaaaatagaaatatagacacatgtgaaagttcatcttcattgtgatcttcaaaacttgactatgtggataaccctgttatttaggttttgagagtttgtaatattgccaagaagagaaaaatacaacctgaaggtccatatataattttccaggtgttgaatgccacttgaagactctatgcgaaataagaaacctcttatttccaggaaaggggcagatagcctgtgatactgaaaacctacctaagccatgacaggttattgactatcaacagagtttgactgtcctgcaattctggagtccatatgactcattcaccaaatagcatttgagtgtttgccgtgtgccgggcactgtgcctttgatctccagcacgtgatagtaacggggataattctgtgaggaccgagaatgtggagatggagacattataaccaaaggtgttccaagttgagatgtcacagtagaattcaaagatgaactcatatttgtttcaactctccctgtctctaataaaacaacacttgaatgttccttaacatcctgtcaatgtgcttaataaatttttgagagagaaaaaaagcagcttactaaacattctgtgaaccaaaatagaggccgatgggattctggttactatttttcccctcattttgcttaatctgtgatttcatctctgtgtttttctttttctttctttatttccttccttccttccttccctccctccctcaatccctccctctcttgctcttcctcttcctttcctttctttcctttcctttcctgaccttcccttcctttcatttcctttcccttcccttccctttctttcccttcccttcccttcctttcccttcccttcccttcctttcccttcccttcccttcctttcccttcccttcccttcctttccctccccttcccttccctccccttcccttccctccccttccctcccctcccatcccctcccctcccttttcttttcttttttctcttctcttctcttcctctcctctcctgtcttttcttttcttatcttatcttatcttttcttttcttgtttcttttctcactctgtcaccaggctgaagtgcagttgtgccatcatggccactacaacctctgctgcccagtctcaagtgatcctcccacctcagcctcacaagtagctgggaccacaggtgtgtgccaccatgccaaggttttttttttttttttttttttgagatggagtctcgctctgtcgcccaggctggagtgcagtgggacaatcttggctcactgcaacctctgcctcctgcctcagcctcctgagtagctaggattacaggcatgcaccgccacacctggctaatttttgtatttttagtaaagacagggtttcgtcatgtggcccacgctggtcttgaactcctgacctcaggtgatccacctgcctcggcctcccaaagtgctgggattacacgcgtgagctaccgtgcccagcccccagctattttttgatatatttgtagagatgaggtctcactatcttgccccgactggtctcagactcctgggctcaagcaatcctcccgcctcagcctcccaaagtgctggaattacaggagtgagccactgcttactggtttgcttatctgtgtttccttattaatctatagtgaaactatgtattaaattataaataaaaacaattttaaaaggttatattttaaaatactttagggtgtaaattttgaggggaaattccacataccccttttttcttaaaaagatacaaaaattgatctattttcttctgtattttctagtttctaccacctaatttttccttgtgtattttttctttttgaagttttccacttctacttatcctatggatcctgaaaatgttgtgtgttggttttgagaattgtattgctagttattagagagacatatagagtaacaaaaattatgagcattgggaaagttacaaaggttagagaagtctcagacaaggcctggatatctggctctgttcctttatgaaaataaaagagacttacgtgactcttcaatttttccataattcttcaacctaggaataagtatcactaactatggataaggcacagtgttgagtactttatgtgctttattttatttagtcatcacaactactctaagaagtaaatactattattatccccattttacatatgaataaattgagtctcacacagtttccttggataaaatattttattggataaaataaattcataaatttatttcaggtcagtgtgacattgaggtctggactttgctgcctcacatttattgtctgtcttgttcatccagggggtcatgcgggataggatattataattcctagggctgattactagccggtgtgtatcagtacagcacaatggcctgtgtttgttttgattggccaacgcctggtctgtaggaatttgttggtttgtacaagcccctgattattattatttttttattttttattttttttttttgagatggagtctcactctgtcacccaggctggagtgcagtggtgcgatcttggctcactgcaagctccacctcccaagtagcggggactacaggcacccgccaccatgcccagctaattttttgtatttttagtagagatggggtttcactgtgttagccaggatggtcttgatctcctgaccttgtgatccacccaccttggcctcccaaagtgctgggattacaggcgtgagccaccacgcccggcccaagcccctgattattactgcaaatttaggttaaataaaatatttgggggcttacataatattaatatgtgactgttatatttgtgtttgtatttattacaaggaaacatcatttttaactattatcaattgtctatacatttattgaagtcagaggctatcttatatagatttgatggttttacaatgcccacagcattggttcagtaaatatatgttgaatggttaagtttcttcaggtaattgttaatgtattcaaaaaccaaatttctctctctttaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcaggtatagtttaacttcagaattttcctaagtcatctcagtgataaactgattttgcatttaatgctaaaaataaatattatttgatttgattaccttacaaagtaggaaacaacacctgggggattcaggatgagaccagtgtttaagattttttttctctcttgaaagaggggaaaataaagaaggataaacagataaaaaaattaaaaggtttcaaggtgagttatccgttatagtagtcagtagccacatgtgtctgtacagcatttaaatggtagtgaatctgaattggaatgttttaagtagagtgtgcacatcttgttctgaagacttagtacaaaaaatgcaaaatatctcaatttttatattgattacatgttgaaacgatatcatgggatatcaataatatattggatatattaggtgaaataaaatatataactaaaattaatttcacttctttctttttacatttgttaacatggcttctagaaaatttaaaataacatggctcatagcatggcttgtgccatatttctgttgaaagcactgatccagagtaaacttgattgagtcaaaacacccacaaaaaatggcgtgagaagataagatacatggaatgaatgccagattatttcagtatatgcagtgggaattcttctttggtggttttcgttttcaaatatcacacacacacacagacacacacagacacacataaaacaacacagcagattagctttatccttttctgcagttactaaacaaattgctgttttccttgatagctgacattcagtgattagctttcattggttaacacacagcctaatgagcttttgcatatttcttatttattttagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggtatgaagggagatgcggagtttgttttaatctcactaactgtggttccctagtttggtgggctagggctacggtaggagtgggaacaagagaggttatccagaatcctcctgtcctatcccccagaatgtcaacattttagaatcaggttagaatttaaaagtattaatttacacagcagaatttttagaattaaaatttatagtgtaaagagactatagcgggtcttcaaatatcaaaatttccattctgtttactcctgcttataaatactcttgtcaggttctgagtaccaaataaaagtaagtgtttgtggaaacatcatttattttttaaaaaataaaggagtattgtagcaaaatttgtcaacatttttttgaagctaaataataatcactatgacattttttaaagcaaaattgtcatcacttattcattgataaggaataaggataggatatattcctttactaatttttgtgcgtatgtaagaattgtacatataaaagtttttaactagcctgttgtaataatttgtgttttctaggatgaaagtatacctccagtttccttttggaggattatgaagctaaatttaactgaatggccttattttgttgttggtgtattttgtgccattataaatggaggcctgcaaccagcatttgcaataatattttcaaagattataggggtaagtgtgatgcccatttgtgtgatttacttgtgaatcctgatggaagaatgaacgaataaagtgcttgtgtctgaactgggatacaaaaacaacaaaatccacgtatctccatatgaaactaattggcttgaagatgtaagaatagcagtctggtttgtgataggagagctaactttgtgaaccttcctttgtcatcacagttggtttctaacctgggcaactaaagaccagcttcctgaaatagtcaatgaaagtgatgagatgactttctcatacctgccaacatttggctctgaacaaatcttgtgaactttcatttcaccaatgtgcaaagagagttcctcaaatgtttgtcaggctggcttgagaatgcactgttctctaacatacatactttccaggatgtgagtaagcctagcatgcttcttttgtaagatgtaattttcatcccacttgctaagattaaaacaaataggaacggcttcctaaagattgaggttgacatgtgttagttccacctcatcttaatggtggcattttcaacagtaaagttacaatctgaaaggaatgctctctgtttagtgacatatgtccaaacatcaagctgaggacacccttgccataactgctgaaaacattgtggagaaagagattgatcaccacaagaagcataagctaaacactgacttttgattctacaaaatatatatgactattcaggtctactcatgcctggttagcttatttcagtttaatatattcattcatgaattcatgtttaatgacattcatgcttaatattaaatatatttattcaagaattgttattattttttgagtctcgctcactctcgcccaggctagagtgcagtggtgcgaactcggctcactgcagcctctgcctcctgggttcaagcgattctcctgcctcagcctcctgtgtagctgggattataggcctgcaccaccatgcctggctaatttttgtatttttagtagagataggacttcaccatgttggccagggtggtcttgaactcctaacctcaagtaatccacctactggcatgagctgccatgcctggcctctagaattattttttgtgtctgctctgtgccaggtatttttctagttgttgaaaaaattccgagaatgtgatcaaatgtctgattctgtagagtatatggtccagggcaggagtcagcaaactttttctattaacagtaagatggtaaatattttaggctttgcatgccacatgatttctgttgcagatactcaactctgacctagtagtacacaacagcaatgtggaaatgaatgaacacgactgtgttccattaaaactttatataaacatttggccagtggcccatagtttgctgatccttgcactaacatgtgtcttgcaggttttgggggttggcgggggtagactgtggtgtcatcagagaaaaaaaaaaaaaggaaatggttaagttaggaatgggtttggttgtaacaaaacctcaacttatgattttatacttttctcattcttcatatgacaataagtctggaggtaagtgacccagggctgggatattagctcttagatattgagtaggcaaccagacatctgcctcattgaagatagtgggctctgcatcggattgcctggatttatatccttgagtcatcccttgctggctgtgttaacttgagcaaattaattaccttctctgtacttcagtttctatctgtgtaaaaccgtgatgataataaatcatacttctggagatggttgccaggattagaggaatagtatatatgaatttgcttagaataatgcctggcatagagtaagaatttggtaattgttatgatttttgttggactgactagtcaaaaaaaattatttaatctcttcaaaaatccttgttgttgttgttatttgtttgtttttttatgaaggataaagttgtctgtcctgggctaggaatgttactatcttacaagactgattttggcccactgtgaagggagatacgtgttttctttccctgaggaatggttatcctctgtgttccttgagtccaaaacatcctattagcccttataactatctctggaaaaaatttcaggtatggtgctaggtcaggtagctgtagtttccaactggctctcttaactggccatcctaccaagaaggcaggctcaaggaaattcctttctttttgaaaggcctgggctgagtgaatgaccaccactctgtggttcaccaaaaaaccacatcaggttttccccagacaccttgggacagtttgaaatgtccaaatagtaaagcaatgaactgccataaatgtagttccccaccaagggaagaggagagtatttgttctgatttcaactttacacagttgaggtggcactcatgactccaagagggaatccgaacagaaaaaatactggatcaatttatgaatgatagacctatccttggcctccacagaagggagaatgcagacacctttaaggctggcaataggaaagccaaccccaatttccagcgtactaaggctctcctgacccctgaatactgggccagatgggaaatgggtcaggtccaggatgggttcttcactgattgaactaaaaatcctttaaatgttttctcacaggtttttacaagaattgatgatcctgaaacaaaacgacagaatagtaacttgttttcactattgtttctagcccttggaattatttcttttattacatttttccttcaggtaaatgtttccattttcactatattcattttgagaaatgcatcattattcaactggggggatttataaacatcagtgtaattggccttttagtagaacttctctattaacatggctactaacagccaagtttttctgacatagtgaaaaaagatgtttgcctcctctggctcccttgcttaccttctcctctccacccttacctcccgcaatgaaaccagggggaacagagtatttggcctgatatgatgattggaggtgaaaggcagggacttcaaaatggggtgtgggggagccctggatgtaagtttggatataagtattgccagtaaatgtaaatactcaaagaaatgtcttcccgtgttctaaaagcaacaacaaacaaacaaaaccccataagcgggtcacatgggtttaaaaagggtttgaagagtttatcgttcaacttttgttcaaggagaaagaatcatttcagtgatggaagaatgtcaatcctccaacaaaaaatgtaggaaacatttgtaaagagtcagatttttacagcttgcaaatcatttggctgaaatgaaatggcaaggaattaagtactctaaaagtttagtatgagggccaggtgtggtggcttatgcctgtaatcccagcactttgggaggccgaggtgagtggatcacctgaggtcaggagtttgagaccagactggccaacatggcggaactctgtctccgctaaaaatacaaaaattaacagggcgtggtgatccacacctgtagtctcagctactcagtaggctgaagcaggaaatgtctcaggaacccaggaggccaggaggaggaggttgctgtgagccatgatcgtgccactgcactccagcctgggcaacagagtgagtccctgtctctaaataaataaataaataaataaatactgtttggtatggcaagacagtattggttttggttcaagtgctccttgtacctgtttctgattttgtgtccttgggcacataagtaaactgtctaagcctctgtttccttagcggtaaactagggatgcaggtacctgcctcttgatgattaaaaggatcatgtgaccaagtgctcaggcctgcatgaggcagtagtaggtaatcacttattaattgaatgattagccacctgtaatttttaaagataaaactgtgactagatctctttatttaacatgtaagcatgtattacatttttaaaaaatagaatatttttctggacattataagaggtgcaaaagaaaaaacagactgaattctttctttactgagtacttacagcctcactggggaatttgaccttactagaataaatgtgacactctaactacttataggattgccataccaactattaataccacaggtgtttggaggaggtaaagattgctcaatgtaaatttggttaagaaagttggagtgaggtgggctttgagctggtcccttaaatgttgacgatttgaattggtgaagcaaaagaagttagaggactaccttcataacatggtgcttggggggactggaacctagcttgcctagaaaacaggtgagaacaaagtctatgagttataggaccttagaagtctagagatagcaaaatgcctaaagatgttagaccacccaactcctcatcttagtaacatggggagggagtccagtgggtaattaaaagctcaggctctggattcccattctggttaggattctcatgcatctggattaagattccaactctgtgaccttataactgtgggctatggatcttatcaggctctctaagcctcagtttcttctgtaaagtgggcttttctgtctacaccacccgtacagccttgtgccatggcacacagtcacagaaacatagcaagcccttgaaatcaggctttctgactttgtctaatctcctgctttagcaaagacatcaattctccctccttttatttaaatggtggctgggtccctgacaaggtatgttcctgcccacagggtttcacatttggcaaagctggagagatcctcaccaagcggctccgatacatggttttccgatccatgctcagacaggtatgtctatcgagggctgtgccctgggatgtgtagaactccccatgtgtgccccttggactcagacagtgggagctctgtcatgtttcccaggcctagctcctatattggttgtccctcagtctcacgtcagagatgcggttgaaccgcccactaggcacagatgaggctctactgtctgtcaggtctgggtgggcacaaggaactggaccagtttgagacagagcctccagcgtggattcacctccagcctgtgtctcagcagtggtgggcagtgcaggcagaaggcatctttgatacccctcatccctttcctctctttccttctttctcctggcaaacccagggcacgagtgtttcttcccaccaagagatgccctctgtactctttctttccgcaaatcaaaactcatccctgtgtcctgttcttcactgcccattttcttttctgaggctagtctgaaatactagttcaagctgcagtgttactggctgataatgggtttaatggaatgcttctcacattttgcagcttcaaaaccctaaccattgacacgtgtgaatgttttcctggggaaatggggaaggaaattagaggaatgtaactcagagcagcctggttcaaaggggaaagttcctttaacaatcatgaaaattttgtatgtgacctaataactttccgttttaaaaatcactaatcacttgccattgagtaaaatgatgctttagaagtctgccccagatgtgccaggggtactcgaaccctggctaagaggcatcagtttggtgtgttaggctttctagagggcattcacattttaccagctgtctctggttcctcagttcttccccattcctcccatcaccatttagaaagaaaatgtatttatgggaattgctagctagtgactgacagagccaggactgaatctaagtgagagagcacagtttgatgggaaaccctgtccttggactgtcaggtcgaactgtatttataagtcagattccacttaggtctacactgaccttgctccagggccaaatttcccattacccaaccagcctccaggccaactgctgtgcccattatactttggcagctgagctgatggtttgtggaatgtctcctccataaattgttaagtagggcaagcatttattaagtgccttctgcttacaaggtctagtacttagtattgtaaggcattcaaacctgaatggtccctgtgttcaaggaagttacattcctgtgtggagacacttttaaccacttaagaatattgaaaagcaagttgatacatactataataaattatagcactattttttcttaaatatttttgggaaaaatgatagatacttctttaatgtaaataaccagtttagataacttcttagggaccacagttatttgtaaaacatataaattcatacttcaaattcacaatcatgttagtatctgtgtatttaaaaatataaatggttttacaaagaaaatcttattttatgttgaatgttatattttaatctggattacttttgctgatttgtttttgactcaaatcacttatactgccctgagctacatttatctaactgcttattcaacctctctattggatgtctaataagagtttcactgtttaacatttccaaaatggagcttttgactcttcccctccccaccagccttattccttacccactcttcccctatatcagtaagcgacagctccgttctactagttgtttgggctaaaaaccttgaagccatctttgactcttctctttctgttaacattgcaaaagcttcctaactgatctccctacctccatttttatcccaatcctgtagattcacctaaaaacagccacagtcatttttctaaaatagaaatcaaatcatatcccactgcccaaacctgctgaaggctgcctgtagctcacagggtaaagtctggaatcttgacagtcgcctgtaagaccatacactatgtggcccccactctctctcaaatctcatctcctataaccgcctttttccaatacaccgtctactccataaacagtggctcactgcccaagagtaggggaggggaggatcagaggagccaaatcagaacacaaatctgtccagaggaagggggtggcttttacaaattcatacaaaggttctttttaggctgctggcagcctttaaggctttcttgctggtgtatgaacagattaggtatgcctttgcctcagggtctttgcacatgctgtttctgctttccctgaggtattctcatcctttccttcattcaggcctctgttcagatgtccccttagaaaggccttctgtttcccctccccagtctctaaaatagcacctcccctcacttttctgttcttcttaccctgctcaatttttcttttatttgtaccttccatctctagaatctaaacttcatgaaagcagctactttgtcttttttgttcgtacaggtccaacacttagaaatcatgcgtaggagaaagtaggcactcagaaatttttctgacaaatgaaatgatctatttatgtgtttttatattaagtttctttcttgtgtattgaatgtcacatcctgagtactaaatgcagggggtataagtataaacaaaactgaccccatcgctgccctcttggagctgagagtctcataaacagctttaaggtaataaaatcattttctgtgccacaggatgtgagttggtttgatgaccctaaaaacaccactggagcattgactaccaggctcgccaatgatgctgctcaagttaaaggggtacgtgcctcctttctactggtgtttgtcttaattggccattttggaccccagcatgaaactaattttctccttacgggtgttagttatcatcattaagaaaatgttgaataaatatctaacctacgaatatatcacatgctttttgtagcaacatgttaactatttaaacattatatactgtagagcatatagataacttataaaccatttgctattgctgttattcatgctattaacaagatgcatgtagaatagttatttagaaaagagagtataaagtgctcaatcaacataaaacagtaattgctactgaagaaaggatgtatttaattgctgtaagaaagtttagagtcactatggttacagaagggagggaagacaatcctctaaaatataggttgaaggaaatgaaaagcacattaaaaaattaaggcaagaatagaataacttcagtctttatctttaataactttaaactttaataattttaataacttaaattttgctactgtatgaatctcttgatataactagatactattgaaccagcaggttttgatttttggctgaagtgacaatttcttctacaactgtttatggcaaaagtccacaaaatgatgtagaatttgaaaaaattcatgtaatctctggtgtgtcttttcccctcttgaaccttatccatctttatctttaaatcttttctgtaagttagtactatactaacatttcttctatctaatatatggggcttctttaagaataaattaagctataaatgaggaaatacatagagttataacgttgaaatataaaccttaggagtccctctttttctattgtttggaatagtttcagaaggcatagtaccagctcctctttgtaattgttgttttaatacaaacttctttgcctaaagcaaacaaaacaataaaaatcaaggtttagatcaagttgtatagaatgtaattacaggtgcacgcctgtaatcccagctactcgggaggctgaggtacgagaattacttgaactcaggaggtggaggttgcagagctgagattgcaccactgcactccagcctgggtgacaaagcaagactatgtctcaaaaaaaaaaaaaatgcaaagaagacagagtggctggaataaagtgagtgaaaagaagagtcataagtgtgttaaggtcagcattatatccagaagtagatggaaaaccactgtagggttttgaacacagaagtgacatgatctgaaattttgaaaggatcactatagaaactgtgtgaataggccgaagggggcaagcatagaaggagtctgttgcagtaatccaggaggagatgatagtgttttagactaatttggttatacaaaaggctataagatataattaattctggatatattttagatgtacagccaacaatgtgttggttggataagatgatggatatgagagaaagggtatttaaagatgactccaaattcttttacctgcacagtggaaaaaaaaatggagtttttttttttttttttttttttaaagagacagggtctctctttgtagcccaggccagactgcagtggcatgattacagctcactataacctagaactcatgggctcaagcaatcctcccttctcagccttcctggtagctgggaccacaggtgtgcaccaccatgtcgagctaatttttaaattttttgtagagacagggtctccctatgttgcccagtctggtcttgaactcctgggctcaagtgatcctccctgcctcagcctcccaaagtgttgagattacaggtgtgagctgccatgcccagctggagttgatctttatgaaattcagaagcctgttggagaagcaggcttgggggagaatggagagttctgtatgagacatggtaagtttgtgacgtctgtattagacatccatatgtagatgtcaagaaggctgaaatttgcactgctaactttagtaaaccttttttataattggtttactaaataagttttactatgcttctccattcattttggtcctcacaactctatagactcctactctgtaggaggaatatacatgagacagtgagcattagtctttggaataaaggaacagtaaccagtgcaatgtgacattgcacaatatgacacagaccctgtggtatgggctcatgtgctttgatggacaaatatcccgcatcacatttgacgtggaagacacaatcctggaggcaactcagtcttttgcagcagaacccagagcagatgtattcagggcattctgtattgcagtttttcttcctgcctctcaaattcctctggtgttatgacctgccttaggccacagtgagttcctatatttgtaaaattggtggtcactttttccctccatagtgctgtttgtgaggccctttgccatattcactgtctctaattgcctgctggggtcaaccttctgcttttcacttgtttcctcaaagacacctcaaacttggccctgccaagatggctgcactcaccttatatgagccactcaagcaaaactgttttccagaacttaaaacagtggctaaaaaggaaagaccagggaagaggaaatgagcaggttggcaaacattgtcaacctaggaaagcccaatgtagctgttatcacagattgactgagagaggactgttggatctactttacaccactagctgacctgtttttggctgacaggttttagttcctcccctcaaccctagtctttacaatgaaaattttctggctactaatctgtgctcttccattcttcttagtccccatatttctatagactcctactctgtagaaatagatgtaggacagtgagtgttaatcttcagaataaaggaatagtaatcagtgtaatgtctgaagtgctaaatcaaatgaagccgaggggttaaatctccttgcagtttaaggtcttagacttcttagttactctttttggtacattaaagaatttgccatctttgatccatcttatttatttctgtggcccttcactactatccaaagctgcacatttatatgtctgaaaagttaaatagccaggtaattcctctgccatgaactcacacctagaagtgattctaggtgatattctaagagcttctctcacaagcccataagtcagtagctgcagtgggaaatgagctctgagtagaacttgtagatactgagaacaggagcaacttatatcatccctgtgccacagccgttctccagcttgcatcctttgaccttcaaactaaagatgtgaaatgcagagagctggctttgaaaaccattccagtctgatttatcccaaatttggcaaatcatccctttaatataagctcaatgattgcatcaacaaaatatacaggtgttctattatttatgatacctctttcagccctggtgcagctccatggtatggaatttaatgtatttaaaggctactgatagttatagcactcttttcctaaatactagtctggtgtttatatcagacaggtgttaaatgattttgtagggattaatgtagggaattataaaggattctattgttactagaaaggggtcccgatgcagaccttgaaagagggttcttggatcttgtacaagaaagaattcaaggcgaatccacagagtaaagtgaaagcaagtttattaagaaagtgaaaaaattaagaatagctacttcataggcagaacagcagcatgggatgctcagctgcttatacttattgttacttcttgattacatgctaaacaaggggtggattattcatgagtttcctaggaaaaggggtgggctcttctcagaactgagggttcctcccatttttagaccatatagggtaacgttgccatggcatttgtaaattgtcatggcgctggtgtgtctttgagcatgctaatacattataattagcgtataatgagcagtgagaacaaccagaggtcacttttgtcaccatcttgattttggtaggatttggctggcttctttaccacagcttgttttatcagcaaggtcttagcgacctgtatcttgtgctgacctcctgtcgcatcctgtgacttagaatgcctaacctcctgggaatgtagcccagtagatctcagccttattttacccagccccttttcagaaaaggcaggtctggggccagggcaggcatttggaatggcttgagggcacagaatatttccagggtagaggagatgtgctggactaaattatagagtgatggactaaactgactttgtagcttgacttttggtttcagagttggaaatctggtttacagttggacattatacagtggtggattaaattcactttgcagtttgacttctggctttagagctggaactcagtgcacactagtgaaagtcatgcttcagactccctctggaattcaaggggaagataataatgcgcgcttactatcttaaattaaattccataattttcagctctgtattttttccaaattaaacattatatctcaaacagacccagatatatttgaatattattaatgacaaacgttaggcttaaattacaaataataatatacctaacattggaaatttccatcattcctagtttgtcagactcctttatcttgctaatttgcagatattgctttagtaatgttgccgtgattaatgaaggttttcttggtattaaaagatccaaagagataggaatatgtaattgaactctagattgttgatattctactttcagcattctgaagtcatggaaattcttactgtagaaactcaataaacttacaagtagacctttactttttagttcattactgataaaataatgaatatagtctcatgaaggtgagttttcagaaaatagaagcatgagttgtgaagataatatttttaaaatttctctaatttgttttgttttgcaggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtn

SEQ ID NO: 8 - DNA, 21978 bp, Homo sapiens
  misc_feature (1)..(1): n may be any nucleotide
nctgggaaggtgagtcaaactaaatatgattgattaattaagtagagtaaagtattctaatcagtgttattttgttactccctactgcttactatgctctaagaatgtgtttataaccattcctcaaagcaatctttttcatgcttattcagtaaattagaaacttacagaaagtagcaaagccagttcttggactcaaaaactgataattaactttaacagactttttcagttttcaggccattgtcttcacactgttcttccttcctccccactttcctccttcccttagttattttcttctttcttttctctcactttcactctctctccactccttccttccttctttccttccttccttctttccttcttttctttccttttttccttccttcccttcttccttcttttctttttctttcttttctttctttctttctttctttctttctttctttcttgctttcttgctttcttgctttcttgctttcttgctttcttgctttctttcttttctttcaagcttaaatccattcctttattgaggaaagtaagcccattttatttgtacatgtgaggggggagattaaatatggaaaaatgctaggggtatttattatatctgttttaaattactaccactctttctttttttttatcatgctcctcccttcatcctatttctgtttatctttaccctttttttacttcttttttttcccctgcatacttgtcttttttttcccattatttaacaaatgcttatgtggcatttactgtgtttccaggcaaatgtcttattccttatagcaaccatatgggggtctattatctcatttttctagtggggctgaggcacaggcaggtcatggctctggactttagagctgataggtcttggagctgggattcaagctcagacagttgtgctccaaagttgtttctctttctgttataaaacaaagagttcctctgatggcagaatgcagtctgatatcacatgatctgtatcatagtggaaatgagaggtcagagcagggctgactgccataactaacatttaggacagggatatatgtgtgatgaacattctgagattcccaggagttagggcagggactcacacagatcagagtggctctggttgtcagtaggcccagctacctcaccaagtgaatgatgaagaaggccccagatgttggtcattgccactaatgctttgtcctttacctctctgctatctcttcagactctttatcccatttttggggggttccctctggaaaatcttttgggccagctgtagtacctccccagccagtgtctgtaggggacaaacctcatgcgaggcatcctatcaggctcccggaggcctatttctggaggtacatcaggatggtgctgacccacagggttctattgtggtctggctcaagtaacgttggcccctgccagggaatacattctgtgccaatgacttcctttaatattgtactttgggggtctcccccatggtgttgactcttcccctgagaagatggaaaaatctaggacaaaatataaaaaaaggagaaagcattctagttaacagtgtcattatttaatgaggtatttgaaatgtcccagaagggagtcttagattgcttttaggaagaagatgatacaatctgatcctaagctaattcatgcaaaccacaggttagcacctttgaagtgacacatgagatttaagactcttggagttctgtcagaggggccaaaggaaggggagcagatggaaatgaattatgtgtggatatggcagattttctgagcaaaggtcagggatgtgatacattttctaattcaggggtggctcatgagagggacagaagtaggtagggaaagtagtggatctgcaccaaccagtccacataatattgaaaatcaacttccacctgaacacacttctcaagctgctatttagtacttcttatataacaagtatcattctaacagctctaaatacttgatctaatttaatgctgtcaataaccctgttgaattatccctgttggatagataatgaaacccaagattcagagagatcaagtaacttgctcacagtcacacagcaacagcctcctaaccaatctccctgcttcaacttcttccattcttatccaactatagtctacattcttacagaagtcattctgacctctcataaataaaaatctaataatgccaacccttacttaaaactcccaaatggcttctgatcatacttggaacaaaatctaatcttttatcatggattaccaaactctgtatgatctaactcctgatgacctttatggccttatctggcatcacagtctcttatgcctgtgtgccctggcccagtgggcattcttgcaattcctggaatgttttaagctgattgcctctgatcttctcttggctgactttacgttattcatatcccaaaattattacctcttcagagaggccattcctgacacctgaattaattcatgatacttcgcagcccttgatgtttctttcctatttctttgtttattggttaatggtgtaccatttgtttattgattaatggtgtatgtgacattagagcccacacttattctgactgcatgatgtcaaaacctgctcataacctctaccctgcctgtgatctgtgctctgcagtttttcagcacattattgtggactagtgagcatttttataaaatacaggtgacccttgaacagcacgggtttgaactgagtgggtccacttatatgtaggtttttttcaataaatgtattggaaaattttttgaagacttgcaacaatttgaaaaaactcacaagccatgtagcctagaaatatcaaaaaattaagaaaaagttaagtatgttataaatgcataaaatatatgtagttactcgtcttattatttactaccataagatatacacaaacctataattaaaaaaagttaaaagtgatcaaattttatgcacacaaacacttacagaccatataagtcaccattcacagtcaagacacatgtcaacaactgtaaagatacagtgttacatgttttggctctgtgtcctcatccaaatctcaccttgaatggtaataatcttcacatgttatgggagggtcccagtgggaggtaaatgaatcctgggagtgggtttttcccatgctattctcatgatagtgaataagtctcatgagatctgatgattttataaaggggagttcccctgcatatgctgtcttgcctgctgccatgtgagatgtccctctgctcttccttcatcttccgccatgattgtgaggcctccccagctatgtggaactgtgagtccattaaacctctttcctttataaattacccaatcttgggcatgtctttattagcagcatgagaacagactaatacatagtgttaaatcataaaataaaaccatagtatgtactgtactaccccagtaattttgtagccacttcctgttgctactgcagtgagttcaagtgttgtacctgcttaaaatgcagtgacactaacaatctcagcaggagcagttcatctctccagtaaattgcatattgcaataaaaagtgatctctcatgattcttgcgtattttaaaaatcatgtttagtgcaataccataaacattaaataacaccagggaacccatatcaagtgacactagtgttgctggaagtgctcccaacaagcagagaaaagtcatgacattacaggaaaaagttgaactgcttgatacatactatagatgtgaggtctgcagctgcagttagttgcctgccatttcaagataaatgaatctagcataagaaccattgtaaaagaaaacaaaacaaaacaaaaacaaaattcgtaaaagccatcactgcagctacaacagcaggtgcaaaaaccttgcactttttgtacaatacatttatctcatattgaaaatgcagctttcacatgggtgcaggattgctgtgagaaaggcatacctatagactctaatatgattagaggaaaagcaaagtcaattcaaattaaaagtaagattaaggatcagagctgggtcatttaatgccagcaaaggatggtttgataattttagaaagatatttggctttaaaaaaatgtcaagatagcaggagaaggagcttctgccaatcaagaggcagcagaaatgttcctagatgctattaaaaaaaatcattgagaagaaaggatatctgcctgaaaaggtttttaatgcagtcgaaagtgccctattttagaaaaaaaaaatgccacaaaggacttttattaataaggaagagaagcaagtccaaggatttatgtcagaaaggagtaggctaactctactgttttgtataaatgcaatccggtttaggatgagtccatatctatagatatgctcttatgtgtaaagctgttcatccctgatccttgaagggaaaagataagcctcagctgccagtattttggttgtacaacaagaaggcatggacaatgagagcactttttccggattggttacattgatgctttctttgttcttgaagtcagaaagtacttagacagtaacggactgccttttaaagttcttttgatattagacaatgctcctggccacccagaacccatgagtttaacatcaaaggtattgaagtggtctgcttgcctcctaaacatgtctctaattcagcctttagatgagggggttataagaatctttttttttttaataaattttttgtagacatgaggtctcactgtgttgcctgggctggtctcgaactcctgagctcaagcgatcctcctgccttagtctcccaaagtgctaggattacaggtgggagccactgtgcctcgccactcataaggatttttaaggctcatcacaaagcacatggtactctacagaaaggattgttaatgctatggtagagagccaccagagagaagaacatcatgcaagtctgtaatcctaaaacgataaatttctgctggagaaaactgtgtccagatgtacatgatttcacaggatttatgacagagccaatcaaggaaaccacaaaaagaaataggagatatgaaaaaaaaaaaaaggtggtgggtgaaggctttcaaggtatagattttggagaacctcaagagcaaataaacatcacaccagaggaattaacagaagatgacttgataaagatgagtgtttccaaaccactgccaaacaatgcggaagaagatataggagaagcagtgccagaaaactaactttacgcaatctggcaaaagagtttcggttattcaggactgcttttaacttcttttatgacatggacccttctgtgatacaggcactgaaactgaagcaaacagtggaggaaggattggtaccatattgaaacatttttagagaaataaaaaaggaaaaagtcagacagagaccacaatgcatgtcagtcacaccaggtgtgcctgcctttcccgcctccccttccacttcctctgcctcttcccctctgccacccctgagacagcaagaccaatccctcttcttcctccttttccttagcctattcaatataaagatgatgaagataaagacctttaaatgatccacttccgcttaatgaatagtaaatatattttctcttccttgtgattttcttaataacattttcttccctctagcttactttattataaaaatacaaatatgtattacatataacatacaaaatacatgttaattgactgtttatgttattggtaagacttctggttgacagcaggctattagtagttaagtttctggggagtcagaagttatatgtgaattttcaactgcacgaggggtcagtctccctaacccccatgttgtccaaggctccattgtagatgcttttttacttttcagaaataaaagccataaacttgtttatttgagggtaggaagagtaatgtcaggaggctattttttctttctagaaacatacatttttatttcttcaaaattatttagtacacaacagtttccgagggaaactcgatgctatttgttcttggattagaaattctctctcctgatggtgattactgagtctccatttaaaactcttggaataaacaaagctgtgaggatgcggggtgctaattaagccttttcctaattgcttcattgtgcgtcaagatgaagacagtcctgaagttattatactgttttactcaccacttttaggtctgtgactcaaactcccacttttattcggctatatacacactaaaaagcaatgacatttacaaaccaatctcagaccagacactcctgccttagaacatggtgcacagaaaatatttcttaaaaccattacactgaaatatacagtaaaatctgtttttcagcagacattgttatagtctgttgaattttcttactaatctagaaaacctgtttgttagaattctgataattagaaatatttctttttttttttgcttgtgaaacttcagcttttattttatttttttatttttattatactttaagttttaggatacatgtacacaacgtgcaggttagttacatatgtatacatgtgccatgttggtgtgctgtacccaccaactcgtcatttaacattaggtatatctcctaatgctatccctccccccttccctctaccccacaacaggccccagtgtgtgatgttccgcttcctgtgtccatgtgttctcattgttcagttcccacctatgagtgagaacatacggtgtttggttttttgtccttgcgatagtttgctgagaatgatggttctaatgtgtctgacaaaaggattgtctttgggaatttgaaggtgaattttttctcctcaccttttgctttctgcacttttacgattttctaaagtgactatatatcatttttataatgtgtaaaagaagtttatacaatattttaaaataaacctgccattttcctaattttctaagtatcttgtggtaaacataattcaatcttcttggcctgtcagtgtaagaataatattttaaattttatttttaaataagtttttgtttctaagaatgttactaatttttttttttacttctgatagattttgatattagtcttcaaaactgacttaatgtcttatgaaatgcttgctgttatgtttgaagttaggtaatttatgtaagattcagtgaagaataagtggaattccatgtttatgattttaagctataaaacactctaaattaaatgtgtctttattagaatctgttctgaccagtgcagaggccaaagagaggaagaacatcttaaacaataaagagtcatttctcttggtgacttataattctggaagttatttctcttaaaatcatagcattaaaagggactttagagaccctctagtccatcgtcctcattttgcaaatgaggaaaatgagacagcatgttggttcaaggtggtgcggctgatgtaggctgaaatctcatcttgtacactggtgttctttgctttttccatatccctttactcagactccagaggtgatgaaggatgtatgtttcctaatcagattgccttgttggaagtaacatttgattacaacataattgaatgatggaaactttctttttaagatggagtctcactctgttgcccaggctagagtgcagtgacgccatcttgactcactgcaatctctgtctcgccagttcgagcgcttcctctgcctcccagtagcatgggattaaggcatgtgccaccatgcccagctaatttttgtatttttagtagagatggggtttcaccatgttgatggtggctggtctcaaactccttacttcaaatgatccacctgcctccatctcccaaactgctgggattacaggcatgagccaccatacccagcccaaaactttctggaaaacagattgatagtatgtgccacattccttaaaaaattaaaaaaattaattcaagccaggtgtagtgccatgtgcttgtagtcttagctacttggcagactgaggcaggaggattgcttacccaggtgtgtgaggctgcagtgagctatgatgatcacacctgtgaattgccactgcactgcagcctgggcaacagagcaagaccccgtttctaaaaaaaaaagttagttttctttgacttattaatttcaccttaagaaattttcctaataaaccaattcaatatggacaaatgtttaggtacaaagatgtttatctcaccactatttttaataaaaaaggaattgaaaacctggctcaacaataaaggaatacttaattggttatgatattaaaggactcattacacatctcattattaatgtgtatttaatgaacttggaaaatgcttttgatatgaaggtaaaaataatgatatagagctaaatatagagtttcattccaatctttttaaatatatttatgcacttaggaaaaaaacaatatggaaatgtgtaaaatatactttttttttaaaaaaaaggacacatttattcagcattatgatcagactattacatttaacaatcaacagtatgggtgccaaaaaaaatctacattaaaaccctttgttgtaatgctttacactttccacagaacagaaactaaaagaatctgttacacaattagtcacaaatatagtcctcgagttttttacccatacacatgagtatttgtctaaaacatgtcttcttgtagcacttaggccctgccaccactgtgcttgtctgagttcacaaatctgttgtaaactgtagcttccctgtcacttctctggctcttatctcctgctaagatttgtttcctggcagtaatttaaaatcttctgccactgctgtagctactgctgctactggaactgccatagccaccttggtttcatggtttggcaaagtactggcctgtaccagcataggggccagagcttctgcctccaaagtttcctcccttcatgggtccaaaatgtaaaactaattgttgtaattgccaaaatcattacaccacctccaaaattgcttccatgattaccaaatccattatagccatccccactgccactatatccaccaccaccacagctgccaccaaagccacaatgaccactgaagtttcctccacgaccaaagttgtcattcccaccgaaactacctccacgaccaccaccaaagttcccagagctgctttgcctctttggctggatgaagcactcaccatctcttgctttgacagggctttcctaacttcacaagtgtggccattcacagtatgggtatttctcagtgacagtcttacccatggagtcatggtcgtcaaaagttactaaagcaaagccccttttcttatcactgccttggtcagtcatgatttcaatcacttcaatttttccatactattcaaaataatctcctaggtgatgttcttcagtgtctttaatgccaccaacaaatatctttttcacagttgggtgggcatctggtctttgagaatcttctcttgagacagctctctttggttccacaactcttccatccaccttgtatggccttgcattcatggctgcatccacctcctccacagtggcatatgtgacaaacccaaagtcccttgagcgcttggtatttggatctctcattaccacgcaatccatgagcattccccattgctcaaaatggctcctcaggctctcattggtcaaccaatgtagagctcaacctccaatgaagaatttcctcagctgtttgggctcattaggagactctgacttagacatgacggcaggggaaagagagactttaaggatgcttccttggtggcgtccacgggcagaaaggggtaagcgtccacaggcagagaggagtaagcctttgaatgtatctaaaatttactttttattgcctgcattctttcactattttccaaacattcttcaattgtcacaaatggcaatgataaagagaaaaatataaacatcacattttaaaaataagtgtaaaataactgtgaacttaaaatgtgatcatcatagaagaaagagcactgcaatagaagtactgtgagttctctggttaattttgcgaagccacgtaaagctgtgtggctttaagcaatgccataatccatttaagtcttagcttctatttctgcaaaggagaagtttgaattagtcaatcttccttccagatccataactcagtttgataaattacttagtatctttttctacagagaaaatgctcatacataataataaatatgtaatcataaaattattttcattagtctgttttatagaattcaaattaatcaccactatttactcttgtgcctcttggtgatcggtgctgtctgttacagatcgctactgaagcaatagaaaacttccgaaccgttgtttctttgactcaggagcagaagtttgaacatatgtatgctcagagtttgcaggtaccatacaggtaataaccgctgaagagtgggaggagagtgtgaataatttttcaatcatcatatttgttttcagagggattactttggctagaaggtagggagcaagtggagaaagtgctcgaaggtaaaccattgagaaacagttgtaattatgcaggagagaaagtacaagaccctgaactaaggcagggacatctctgaggtagaacctgtaagaatgggtcactgatgagaagggagggagacatgatgctgagaatgactatctgatgtccaggtaggatatgaccctataatttgctctagttgaaaatgagttatttatggaacctgaaatttgaggtacctgtgggacacagagaggatggtgcttcctgggtttacctgtctttctgctttttaccccttctccagtgcagaccttcttcctttaatacagtcatcccagtgagggctctaataagtggtgtgaacataagcaagcccaacctttctgagtctgtttcttataataaaaggaaagctggactttattgatggatttttaagcttttatttaaaaaaaaatgatggtagagcctttttgtctaaaaaaaaattatttgaaatcttcatatgtgatgaaggtaaaagcagagttgatctggtagcaggaggggttggaagcccagggctgcccacttgctaacctgcccccacccacacctccatatcactgagctggatccatatcatgattctagaatgtcaacccctttttaaagccttctctgagacccccgaagaatttagtgcttctcctttcttcctataacagattattcataaggcacctctaatgcataaatagtaattcaactcaagtcaggtctcctaagtcaagaacatgcctgtttcctctatgtcaacccatcatggcccctgcaccttgtaggtgttgggtaactgtgtgcagaatgaatatttacgtagagtacttccatactgtgtgtccaaaagtggggagaaagggagataacttttacttattgatccctaaccggctctttacagtcattggattattttctctttacatcaacactttgaggtgtagttaattctacattaaagataaagacactgagtctcagaggttaggcagtttcccaaatttgcacaactgttaagtgtaaagtgaggaccaaacttagttctttggaaacgaaacccatttttgttggtcatgcctctgtgccctactgccaacctatcaaaagttacattttaaggactgagaaatgaaagtggaatgagttttcaaatgtcttcttttcagaaactctttgaggaaagcacacatctttggaattacattttccttcacccaggcaatgatgtatttttcctatgctggatgtttccggtttggagcctacttggtggcacataaactcatgagctttgaggatgttctgttgtaagtattgggctattatttagttaagctctaaaaataaagctgggatgaacatgcttcatgtctagataggaaaccctactgtgaagcctcatgaagagattctggtgattcctaaatcggcatttctgcctctgagtcttcatgtgccaccattgaagcaccccctttcatttggaggagcagtaacttctttcctcattgctggctcacacatagttgaccttttcaaatctgtgactgagtggagtgattccattcggagattttgagaaggccttggcatttgggaagaagcctagccctgagcaaggagtctgactggctccttttaaaggactttcttacagagcaagtaaaatacagatgtgttgtactaagttctgcaagcctttggcaattccaggatatgtttactttcccttgataagagaggaattggaaggtaagagccaaatgaattcagaaatgacaaaggaaaagttatattgggatttttctgctacattgttctgaatgtagataattgtacctcggtcagaggaagataagcctgaagcaattatactacaaaagtacccaaatatgcaatttggtggtcaaaagtatgtgtaatctttctgagcttctagatttggaggtgggtaagattctgcctcttgatagcataacataattagtagcgatataattttatatttaaaccagaatcatataagcctggcagtataatgtgttagtatagtatttctgtcctttttaaacattgagttgttcatgcattacagtttgctcaggatgacccccaaaaagacatctaaatttccattaaagatgtacattggacaaatgatatgcaggtgctatttgtgattatggcctagagatcaaaaccaagtatcactggcattggggctttgattagataattatttgatatattgctttactccaaaaaattgaattgatgaaagttgctgacattggggcatttaggtttgcaaaatcaaccttccaagttaaggaaaaggaagacctgtctgaagaacagtgcattagaaaaaggtcccataggtttaaatgatcacaagtccaagattaactatcggtattttatttagtgctagttaaacaaacaaacaaacaaaaactaatgacacaatatgctacacaaacataggcatagagtccagaggaaagaaagcgatgaattccctgtattctgggcgggttcccaagtattatgtctgttccaggcttgtgtttaaaaaaagcatattaataaatgtgtctagagaagggcagtgaaaggagttatcaaagaagcaaaggagtttttagcaatatttcaagacttgtaggcctgtcttatgtaaaaggaagtaaattttttcttcagtttgaaaaagaactgtttaacaatagacaatcaaacatgctacttcctaaaacagaggatggaagccaatttaggggaggtgtccaggcacgaacatggagagctggacttgatacctgtaaggtccttcccaactttaagttgctgtgattcccatgtcatagataagaacgtcaatgcatcttaagagcaacatgatatctggctctgtaagaaactttcttttggttgcacaattcataggtttttaagaatctgatgtaattccaacatcactgactgtatccgttgttgattaccacaataatgctgtgtaacaaccagccactaaacctcagagacatacatatttttgcccacaaacctatgggttagctgtgaagttctactgatctggattaggcatagtggatctcggctggacttactcatgtgtctgtagtggattgtgtctgtagctggttggttgggattcagacagctctcctccatgtgtgcctcatcctccagcaggctatcctgggtttgagacagtggcagaattctgagagagagagagagagaaagagagagagaacacatgcatgcatgctcacaaagcatataaggccccctgagtcataggcttggaactggcaaaagtgatttccaccatatcctgttggccaaagcaagtcacaaggccagcccagattcaaagggtgctgcaaaggtcacagtgcaagaagatgaggatacagagagaggtatacagagggaaaaaaatgggccattttgcaatcaatctatcacatagacatgaacttataaggaaatgtgtttgttttatttttaacatctgttttataactattagaggcaaagctctctggttatagaagtgtcaacttttggccaggcatggtggctcacacctataatcccggcacttttggaggctgaagcaggaggttcacttcagcccggtagttcgagaccagcctgggcaatatagggagacccccatctctacaaaaaataaaacaattagctggtgtggtcatgcacagttgtagtcccagctactacagaggctgaggtgggaagatcgcttaagccctggagatcatatctttagtgagctctgattgcactactgcactccagcctgggcaacacaggaagaccccacctcaaaaaaaaaaaaaaaaaaaaaaaggagtgtcagctttctagcattgtgatggtaatgctgtgcacatgttttgtgtttgtgctttccagagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccggtgagtttgatgtttcaactgtttgatctactcctgactcctgaatgaaagtattttaagtggaaacttaataaaatttgtactttcaaatatgctgatgataaaataaaacttcctagatcatagattcctttcaattactgctaataatatacatcaacattcagtacttttacgtagcaaaggttatagggaaataggaatactgctcactttataagcaaaacctattaatcagattttttaaaaacaatttttttttagagacagagtcttactctgtcatccaggctggagtgcagcagtatgatcatagctcactgcagccttgatcttctgggctcaagcgatcctcctgcctcagcctcccaagtaactgggactacaagcgtgtgccatcatgcccagctaattttttaattatttgtagagacagggtctcgttatgttgcccaggctggttatcagattttattgtatgtaagttactgtattcctgaggaacagatttgagttattgtagctgtattgcatattcatattgtcttaacaatacatgctatgaaagcttttactcttttagatctcatttattaaattctagcagtctgaggtcaagcacagtggctcatgcctgtaatcccagcactttgggaggccaaggtgggtggatcacgtgaggtcaggagttcgagaccagcttggccaatatggtgaaactccatctcaaataaaaataaaaaaaaaaagattagctgggtgtggtggcacacgcctgtaatcccagctacttgggaggctgaggtacgagaattgcttgaacctggggggtggaggttgcagtgagctgagatcatgccactgcactccagcctgggtgacagagtgagactctgtctcaaaaaaataaaaaataaataaataataaaattaaaataaaataaattctagcagtttgaagtgaagccaattgtaacacaaattaattatcttctgacacctggtaatcgagagagttagctatacactttattttcagtattgcagcattcaaatttactgttattcttctcattgcagaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtggtgagcacactttcacatttagctcagttcaggttttcatcatccaaatgtctgaatgtatttaattctcaactataagccatgttttttcaaacctttaaacaacagtcccacttggataaagtctgagagcctaaatatggtctccaagtggtgtcatctgtcccagccaacttctccaggctcccctcaacactacccctctaccctcctttgcaagcaccctttgtaccacctgtctcccttgctgtacttaggtttcagctgacttgaaaagaaccaacaaaaatggaagtagccagaaagatacaggacaggtgctaggagtgagatgaaagccaaggatggagactgtttcaaagatggtggtcagcaatgacagatggtatagagagattgggtaaggaggagactgagaagacatgatagaatctagcaatgaatgggctatttctgacattttaaaatattcttagtgaagtagtgatggtaggaaacagttaagggtggctgaagtatgattaggggaagaaatggagatgtaagtgagcatagattatcaagaagatggaaagtaaaagaaaggcaaataaggacagcacttgagtggggaggcagagtccagggaaaatgtttaggggttgaactcttgagcatttttatgactggggatgagacaaaatcattgatggagagagggcattgaaacatcatctgaggaaaaaaaagtagaatcaacaaatttgggaaatgtaaaaagaacatggaagtatgcccttttgtcttagttgctggggatgagggaagtgtctggcagggagtagtgagtttaaacgactatgatgggggataggaaagaagacaaactaggaaggaatagaattaagagcaagacattttcattatgtaggcatgtaattagttgtactgagaagtgatatttaaaattcttaacaatgggaacagcatggacactgaccaatcagaatggatgcctgctagtacaagtcagatggatgcccacccgctgtgtatggacgctggtagcatttaatgactcaaatgaaaagagagttttcaatctttagtctaggactctttttacttcctcacagcggtcactcttactcataaatgtttgttattgtattcaaagacagttatgcctttattcagtttactcttaaatcattacaagcttgttccctacttttactctcttgctttatattttctgtgaactctgattgtgtccttcaattttgtggtgaacttaaaaacaggactctaaataaagacgtcgctgacagccaaagcaattagaggaggaaatgttagctctcagacactgattatggccctcaacagacagttattaagtggtgtgttaaagattgtgctataatgaattgtagggcatgatatctgccctcaaatactttaactgaggagatggggcatatagttttgtgagttaaaggagataggcctagagctagttcttaagagataaggatttgcatatgggtgaacagtagggatgacattcaatgtaagagaaaatacaagctgcgatcagtgatttgttcccaatgcagcaaataggacactttggctgaaatgggagtttagtttgagtagtggttctcaaacttctctgatgagaactcatgtaggaaagatttaaagatgtcaccatccactaatgtactcatagtcactttccagtgggaaaaacacttggtgataacaaaatgttggagttgaaaacaacaatacaatacagcacaataatgatacaataaggaataattaatgattattctgcaagtatataaacacaagcagaacataataaaacatggacagcatcagttacagttcagagtaggaaacagaaactgccctaggtatttcaagtagataagtatttaataaagggaattacatgcttacaaaatctttggaagggctggaagagtgaaaggaggctatttgctcccagacttccaaatcacagcactgcagctgtgattctgtaaccaagaagctgcagaaaattatgccgatatcacagctgttccaaatttaaagacacgagactagaatctggctgttgcagaaattcttgtctgccaaagatgagttaagccgttagcttaccacagctgctgaaggagagctggtgtctacctcccagattttacactgtgcatctccctcgtagaccctgacttacttccagaaccaagggaaactgggaaatagtttttagccttctagtcccttgatgtataaaaggtcagacacagaagtatatgaaaatgaatgctgagtgcataggacagtaagcatcccaagacattattgataatgcactgaaaacaaagtgtcctcatttattgcaggaatatccattatcttctcctgtgcatttgcactagggcttggaaatactgccagatggtgcactgcagccagacttctcagcagaaagtgtccacaataaactctagtaaatgtacaaagttttgtttatggacatcaatgagtagccctgaataactcagtggattactactaattagtttttattgccatgtaaaaatgagtctgtggatttgagcataagaattaaagaaaggattggaccctaaggaccccctgaagtcaggaaattctcgtgaccatcagtggaccttgaatcccatgttgaaaaacattgacctgaaatggtggttctaaagcttcggtgaatattagaatggcctcaagagctagtaaaaaacacagccggcctggattattcaagtaggctagggtttggccttttatttttataatattccgaggtgattctgatgccaaccatcattcgagagccactgaactggtaaatttgaagaaagatttaggttaaattgtggagagtcttgaataattaagctatgaaattgtgaagatgggaggatcctccaagttttgagggacctgaaggttatacaattgtagggattcaacttaaagtaaaaaaaaaaaattttttttttaatgcaaaattagatacaaaggtggatatttacttcaaatgaaaaaaaaatcataaccaaatactggaagcttaaagattctggtcccttttcatttttaggtaatttatcatcaatacttacagggaagagcttcctgactacagccttgcctccctcccacccagaaaactgttcgacttcccagaatttacatgtaattgagggctcttgaagcttaaccttcatcagtgtcactgttaaatcacccctgctttgagtcttccccttacctttcctttctcccctttccagtctctcccatatacaacagtcagaaccccctttgggatacaattagaagctgcttgtcattgtctggttataacccaagaagtgcctgagccaagatgggggcattcccctttcaaggggggacacaaaatatctttcagttgtgaggtattagtgttaccaaaacccaacctagagtgtttagatccagtcttggttttcccccaattactaattgtgtgaccttgtataagttacttaactttgctcatttccttatcagtaaaatgggaattaacatcacagtgaccttataagtgttgctatgaaggttaaacaagagactgtatgagacatgcttatcacagaacctacaacgttgtcagtgcttgatttttaaaaatcaattattatttcacagtaaatatgcatagaagaatacacatttaacatttacatttttgcgctaatgaaagcaagcaatagacttaacagtctacaatagagatagccaatcattttaatctggcctttcattatttcttaccaataggtttactttcctagttatatttttatttaggccaggattttttgagcacttctcatagattccccagagttccttggtctttcctcagaccaacaccattcctcagccaaagaagctctgcttctttgatctgttttacattctaggcatctaaacttgttttacttaaagaaagaattcttcagtcaacacaggttttaaatagtttgcaattctagggtattatgcggggacgaggaggcccaagagtgtattcagtgtaatgaagaaactcattataacttgctgagatcattcagatttgcctgtgattatattagttaggcagtgtctgttttcctcccaggggtataaacctggaaaattacaacaaaaacaaaagcaaaaaccaaactcctcctttccttaaatttccacttctcaagtacagtgttttatctaacaagatctgctgcttagccacatccttgtttggctttcactgtctctctccaccctctactttccaccttcatttattaacattgtaaggaagcctggagcatacatgtgtggaatagtctccatggcaactcagcttggaactaataaggttatgggagagcttccatccctgcacctgccagtgtcacaagcagaagccatgttcctgtagcaaagattgtactctactgctaggcagctgtccccttgagccacccagccaggcacatgggatacagagagtatatggctcagcacgcactagtcactactattagaaaagctcaaagctgagtcactggtgccttcttcagaagggatgaacagctctctcacttgaatgccagaaaattatcttgcaaagcagacctatctgatagacatatttgcatcagagtagggcttgttatcagcaaggctgtaaggtgccctccccagtcttctgcaggataatccagggagccagattatagagaaagggcagccctctgttcctactcatctggctcagtattgaggatctccattcaccctttcctacccctgttcacctatccatcccctgtagttcctgacctgcaaagctattatgtggtcatagttgttatttatttccatgctaatcctgggcactgtttctctgaaaaatggagatttaagaataaggctgtcaggatacatctcagaagtattaggcgttgtattagtgtggctgctataaccacttgccacaaattcagtggcttaaaacaacaccaatttatgactttacacttctggaggtcagaagtctaaaataggtctcagtgggctcatatcaagtgtcaggagggctgtgttccttctagaggctccaaaggatgatctgttttcttgcttgtggccccttcctccattttcaaaaccagcagtggctggttgagtctttctcacactgcatttgctgatactcttctccctccttcttcttcagttaagagcccttatgattacattagtttcaccaagataattcaggataatcttattttacagtcagctgattagcaaccttacatctactactttagtttctttttgccatgtaacataacacattcacaggatccagggattaggacacagatgtcttgtgggagagggaacattattctgcctaccacatgcatacatcagaaaccatggttgaaacacaggaaacatgacagttcctcaaggcatacaattatgaccttgttgggttaaccttcactatccaaattttaatcacacaaacttttccttaatctcacagtaacttggcagtttcagtgtaagaaataatgatgttaattgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatn

SEQ ID NO: 9 - DNA, 40962 bp, Homo sapiens
  misc_feature (1)..(1): n may be any nucleotide
nctgaacctgaaggtgcagagtgggcagacggtggccctggttggaaacagtggctgtgggaagagcacaacagtccagctgatgcagaggctctatgaccccacagaggggatggtgagatgacccatgcgagctagaccctgcggtgatcagcagtcacattgcacatctttctgatgttgccctttcaattacaaatgtatgaaagtcacacttactttttattccaggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcatgtaagttgtccttgccctttgcctttctagaggtgcaaaaaataaaatgcaggcctactatgcaggaagttaggaaactactataaatcggaagaagggaaatcctaagaagggaaagtaagattacttcagatttgaaagctctagcagtatcaactggtcgtagatacatttttaaaaactgaggttggttattgtgttaaataagatttaaagaactggacctgtattacttgtgagacttgggctgtgtataggattccttaccaatttaaaatatgagctgagatagcttgtccttatgctaaatcattctgggttttctgtggtagaaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggtcagtgaggcttagttcaaaccaacctgatttataagcataagaacattctactactaattcttgttaatattggtcttagaaaaggaaatttctgatagcttctaggtgattccttcagctattaaaataaaagcattgggcctctttgaaatctttttctatttgtttgttttattgttcaatttctatttatttctctgatcttattttaaatgttgatgaatacattttcatttgaagacacttgctaatcttttaaattaaaaaatagaaatatagacacatgtgaaagttcatcttcattgtgatcttcaaaacttgactatgtggataaccctgttatttaggttttgagagtttgtaatattgccaagaagagaaaaatacaacctgaaggtccatatataattttccaggtgttgaatgccacttgaagactctatgcgaaataagaaacctcttatttccaggaaaggggcagatagcctgtgatactgaaaacctacctaagccatgacaggttattgactatcaacagagtttgactgtcctgcaattctggagtccatatgactcattcaccaaatagcatttgagtgtttgccgtgtgccgggcactgtgcctttgatctccagcacgtgatagtaacggggataattctgtgaggaccgagaatgtggagatggagacattataaccaaaggtgttccaagttgagatgtcacagtagaattcaaagatgaactcatatttgtttcaactctccctgtctctaataaaacaacacttgaatgttccttaacatcctgtcaatgtgcttaataaatttttgagagagaaaaaaagcagcttactaaacattctgtgaaccaaaatagaggccgatgggattctggttactatttttcccctcattttgcttaatctgtgatttcatctctgtgtttttctttttctttctttatttccttccttccttccttccctccctccctcaatccctccctctcttgctcttcctcttcctttcctttctttcctttcctttcctgaccttcccttcctttcatttcctttcccttcccttccctttctttcccttcccttcccttcctttcccttcccttcccttcctttcccttcccttcccttcctttcccttcccttcccttcctttccctccccttcccttccctccccttcccttccctccccttccctcccctcccatcccctcccctcccttttcttttcttttttctcttctcttctcttcctctcctctcctgtcttttcttttcttatcttatcttatcttttcttttcttgtttcttttctcactctgtcaccaggctgaagtgcagttgtgccatcatggccactacaacctctgctgcccagtctcaagtgatcctcccacctcagcctcacaagtagctgggaccacaggtgtgtgccaccatgccaaggttttttttttttttttttttttgagatggagtctcgctctgtcgcccaggctggagtgcagtgggacaatcttggctcactgcaacctctgcctcctgcctcagcctcctgagtagctaggattacaggcatgcaccgccacacctggctaatttttgtatttttagtaaagacagggtttcgtcatgtggcccacgctggtcttgaactcctgacctcaggtgatccacctgcctcggcctcccaaagtgctgggattacacgcgtgagctaccgtgcccagcccccagctattttttgatatatttgtagagatgaggtctcactatcttgccccgactggtctcagactcctgggctcaagcaatcctcccgcctcagcctcccaaagtgctggaattacaggagtgagccactgcttactggtttgcttatctgtgtttccttattaatctatagtgaaactatgtattaaattataaataaaaacaattttaaaaggttatattttaaaatactttagggtgtaaattttgaggggaaattccacataccccttttttcttaaaaagatacaaaaattgatctattttcttctgtattttctagtttctaccacctaatttttccttgtgtattttttctttttgaagttttccacttctacttatcctatggatcctgaaaatgttgtgtgttggttttgagaattgtattgctagttattagagagacatatagagtaacaaaaattatgagcattgggaaagttacaaaggttagagaagtctcagacaaggcctggatatctggctctgttcctttatgaaaataaaagagacttacgtgactcttcaatttttccataattcttcaacctaggaataagtatcactaactatggataaggcacagtgttgagtactttatgtgctttattttatttagtcatcacaactactctaagaagtaaatactattattatccccattttacatatgaataaattgagtctcacacagtttccttggataaaatattttattggataaaataaattcataaatttatttcaggtcagtgtgacattgaggtctggactttgctgcctcacatttattgtctgtcttgttcatccagggggtcatgcgggataggatattataattcctagggctgattactagccggtgtgtatcagtacagcacaatggcctgtgtttgttttgattggccaacgcctggtctgtaggaatttgttggtttgtacaagcccctgattattattatttttttattttttattttttttttttgagatggagtctcactctgtcacccaggctggagtgcagtggtgcgatcttggctcactgcaagctccacctcccaagtagcggggactacaggcacccgccaccatgcccagctaattttttgtatttttagtagagatggggtttcactgtgttagccaggatggtcttgatctcctgaccttgtgatccacccaccttggcctcccaaagtgctgggattacaggcgtgagccaccacgcccggcccaagcccctgattattactgcaaatttaggttaaataaaatatttgggggcttacataatattaatatgtgactgttatatttgtgtttgtatttattacaaggaaacatcatttttaactattatcaattgtctatacatttattgaagtcagaggctatcttatatagatttgatggttttacaatgcccacagcattggttcagtaaatatatgttgaatggttaagtttcttcaggtaattgttaatgtattcaaaaaccaaatttctctctctttaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcaggtatagtttaacttcagaattttcctaagtcatctcagtgataaactgattttgcatttaatgctaaaaataaatattatttgatttgattaccttacaaagtaggaaacaacacctgggggattcaggatgagaccagtgtttaagattttttttctctcttgaaagaggggaaaataaagaaggataaacagataaaaaaattaaaaggtttcaaggtgagttatccgttatagtagtcagtagccacatgtgtctgtacagcatttaaatggtagtgaatctgaattggaatgttttaagtagagtgtgcacatcttgttctgaagacttagtacaaaaaatgcaaaatatctcaatttttatattgattacatgttgaaacgatatcatgggatatcaataatatattggatatattaggtgaaataaaatatataactaaaattaatttcacttctttctttttacatttgttaacatggcttctagaaaatttaaaataacatggctcatagcatggcttgtgccatatttctgttgaaagcactgatccagagtaaacttgattgagtcaaaacacccacaaaaaatggcgtgagaagataagatacatggaatgaatgccagattatttcagtatatgcagtgggaattcttctttggtggttttcgttttcaaatatcacacacacacacagacacacacagacacacataaaacaacacagcagattagctttatccttttctgcagttactaaacaaattgctgttttccttgatagctgacattcagtgattagctttcattggttaacacacagcctaatgagcttttgcatatttcttatttattttagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggtatgaagggagatgcggagtttgttttaatctcactaactgtggttccctagtttggtgggctagggctacggtaggagtgggaacaagagaggttatccagaatcctcctgtcctatcccccagaatgtcaacattttagaatcaggttagaatttaaaagtattaatttacacagcagaatttttagaattaaaatttatagtgtaaagagactatagcgggtcttcaaatatcaaaatttccattctgtttactcctgcttataaatactcttgtcaggttctgagtaccaaataaaagtaagtgtttgtggaaacatcatttattttttaaaaaataaaggagtattgtagcaaaatttgtcaacatttttttgaagctaaataataatcactatgacattttttaaagcaaaattgtcatcacttattcattgataaggaataaggataggatatattcctttactaatttttgtgcgtatgtaagaattgtacatataaaagtttttaactagcctgttgtaataatttgtgttttctaggatgaaagtatacctccagtttccttttggaggattatgaagctaaatttaactgaatggccttattttgttgttggtgtattttgtgccattataaatggaggcctgcaaccagcatttgcaataatattttcaaagattataggggtaagtgtgatgcccatttgtgtgatttacttgtgaatcctgatggaagaatgaacgaataaagtgcttgtgtctgaactgggatacaaaaacaacaaaatccacgtatctccatatgaaactaattggcttgaagatgtaagaatagcagtctggtttgtgataggagagctaactttgtgaaccttcctttgtcatcacagttggtttctaacctgggcaactaaagaccagcttcctgaaatagtcaatgaaagtgatgagatgactttctcatacctgccaacatttggctctgaacaaatcttgtgaactttcatttcaccaatgtgcaaagagagttcctcaaatgtttgtcaggctggcttgagaatgcactgttctctaacatacatactttccaggatgtgagtaagcctagcatgcttcttttgtaagatgtaattttcatcccacttgctaagattaaaacaaataggaacggcttcctaaagattgaggttgacatgtgttagttccacctcatcttaatggtggcattttcaacagtaaagttacaatctgaaaggaatgctctctgtttagtgacatatgtccaaacatcaagctgaggacacccttgccataactgctgaaaacattgtggagaaagagattgatcaccacaagaagcataagctaaacactgacttttgattctacaaaatatatatgactattcaggtctactcatgcctggttagcttatttcagtttaatatattcattcatgaattcatgtttaatgacattcatgcttaatattaaatatatttattcaagaattgttattattttttgagtctcgctcactctcgcccaggctagagtgcagtggtgcgaactcggctcactgcagcctctgcctcctgggttcaagcgattctcctgcctcagcctcctgtgtagctgggattataggcctgcaccaccatgcctggctaatttttgtatttttagtagagataggacttcaccatgttggccagggtggtcttgaactcctaacctcaagtaatccacctactggcatgagctgccatgcctggcctctagaattattttttgtgtctgctctgtgccaggtatttttctagttgttgaaaaaattccgagaatgtgatcaaatgtctgattctgtagagtatatggtccagggcaggagtcagcaaactttttctattaacagtaagatggtaaatattttaggctttgcatgccacatgatttctgttgcagatactcaactctgacctagtagtacacaacagcaatgtggaaatgaatgaacacgactgtgttccattaaaactttatataaacatttggccagtggcccatagtttgctgatccttgcactaacatgtgtcttgcaggttttgggggttggcgggggtagactgtggtgtcatcagagaaaaaaaaaaaaaggaaatggttaagttaggaatgggtttggttgtaacaaaacctcaacttatgattttatacttttctcattcttcatatgacaataagtctggaggtaagtgacccagggctgggatattagctcttagatattgagtaggcaaccagacatctgcctcattgaagatagtgggctctgcatcggattgcctggatttatatccttgagtcatcccttgctggctgtgttaacttgagcaaattaattaccttctctgtacttcagtttctatctgtgtaaaaccgtgatgataataaatcatacttctggagatggttgccaggattagaggaatagtatatatgaatttgcttagaataatgcctggcatagagtaagaatttggtaattgttatgatttttgttggactgactagtcaaaaaaaattatttaatctcttcaaaaatccttgttgttgttgttatttgtttgtttttttatgaaggataaagttgtctgtcctgggctaggaatgttactatcttacaagactgattttggcccactgtgaagggagatacgtgttttctttccctgaggaatggttatcctctgtgttccttgagtccaaaacatcctattagcccttataactatctctggaaaaaatttcaggtatggtgctaggtcaggtagctgtagtttccaactggctctcttaactggccatcctaccaagaaggcaggctcaaggaaattcctttctttttgaaaggcctgggctgagtgaatgaccaccactctgtggttcaccaaaaaaccacatcaggttttccccagacaccttgggacagtttgaaatgtccaaatagtaaagcaatgaactgccataaatgtagttccccaccaagggaagaggagagtatttgttctgatttcaactttacacagttgaggtggcactcatgactccaagagggaatccgaacagaaaaaatactggatcaatttatgaatgatagacctatccttggcctccacagaagggagaatgcagacacctttaaggctggcaataggaaagccaaccccaatttccagcgtactaaggctctcctgacccctgaatactgggccagatgggaaatgggtcaggtccaggatgggttcttcactgattgaactaaaaatcctttaaatgttttctcacaggtttttacaagaattgatgatcctgaaacaaaacgacagaatagtaacttgttttcactattgtttctagcccttggaattatttcttttattacatttttccttcaggtaaatgtttccattttcactatattcattttgagaaatgcatcattattcaactggggggatttataaacatcagtgtaattggccttttagtagaacttctctattaacatggctactaacagccaagtttttctgacatagtgaaaaaagatgtttgcctcctctggctcccttgcttaccttctcctctccacccttacctcccgcaatgaaaccagggggaacagagtatttggcctgatatgatgattggaggtgaaaggcagggacttcaaaatggggtgtgggggagccctggatgtaagtttggatataagtattgccagtaaatgtaaatactcaaagaaatgtcttcccgtgttctaaaagcaacaacaaacaaacaaaaccccataagcgggtcacatgggtttaaaaagggtttgaagagtttatcgttcaacttttgttcaaggagaaagaatcatttcagtgatggaagaatgtcaatcctccaacaaaaaatgtaggaaacatttgtaaagagtcagatttttacagcttgcaaatcatttggctgaaatgaaatggcaaggaattaagtactctaaaagtttagtatgagggccaggtgtggtggcttatgcctgtaatcccagcactttgggaggccgaggtgagtggatcacctgaggtcaggagtttgagaccagactggccaacatggcggaactctgtctccgctaaaaatacaaaaattaacagggcgtggtgatccacacctgtagtctcagctactcagtaggctgaagcaggaaatgtctcaggaacccaggaggccaggaggaggaggttgctgtgagccatgatcgtgccactgcactccagcctgggcaacagagtgagtccctgtctctaaataaataaataaataaataaatactgtttggtatggcaagacagtattggttttggttcaagtgctccttgtacctgtttctgattttgtgtccttgggcacataagtaaactgtctaagcctctgtttccttagcggtaaactagggatgcaggtacctgcctcttgatgattaaaaggatcatgtgaccaagtgctcaggcctgcatgaggcagtagtaggtaatcacttattaattgaatgattagccacctgtaatttttaaagataaaactgtgactagatctctttatttaacatgtaagcatgtattacatttttaaaaaatagaatatttttctggacattataagaggtgcaaaagaaaaaacagactgaattctttctttactgagtacttacagcctcactggggaatttgaccttactagaataaatgtgacactctaactacttataggattgccataccaactattaataccacaggtgtttggaggaggtaaagattgctcaatgtaaatttggttaagaaagttggagtgaggtgggctttgagctggtcccttaaatgttgacgatttgaattggtgaagcaaaagaagttagaggactaccttcataacatggtgcttggggggactggaacctagcttgcctagaaaacaggtgagaacaaagtctatgagttataggaccttagaagtctagagatagcaaaatgcctaaagatgttagaccacccaactcctcatcttagtaacatggggagggagtccagtgggtaattaaaagctcaggctctggattcccattctggttaggattctcatgcatctggattaagattccaactctgtgaccttataactgtgggctatggatcttatcaggctctctaagcctcagtttcttctgtaaagtgggcttttctgtctacaccacccgtacagccttgtgccatggcacacagtcacagaaacatagcaagcccttgaaatcaggctttctgactttgtctaatctcctgctttagcaaagacatcaattctccctccttttatttaaatggtggctgggtccctgacaaggtatgttcctgcccacagggtttcacatttggcaaagctggagagatcctcaccaagcggctccgatacatggttttccgatccatgctcagacaggtatgtctatcgagggctgtgccctgggatgtgtagaactccccatgtgtgccccttggactcagacagtgggagctctgtcatgtttcccaggcctagctcctatattggttgtccctcagtctcacgtcagagatgcggttgaaccgcccactaggcacagatgaggctctactgtctgtcaggtctgggtgggcacaaggaactggaccagtttgagacagagcctccagcgtggattcacctccagcctgtgtctcagcagtggtgggcagtgcaggcagaaggcatctttgatacccctcatccctttcctctctttccttctttctcctggcaaacccagggcacgagtgtttcttcccaccaagagatgccctctgtactctttctttccgcaaatcaaaactcatccctgtgtcctgttcttcactgcccattttcttttctgaggctagtctgaaatactagttcaagctgcagtgttactggctgataatgggtttaatggaatgcttctcacattttgcagcttcaaaaccctaaccattgacacgtgtgaatgttttcctggggaaatggggaaggaaattagaggaatgtaactcagagcagcctggttcaaaggggaaagttcctttaacaatcatgaaaattttgtatgtgacctaataactttccgttttaaaaatcactaatcacttgccattgagtaaaatgatgctttagaagtctgccccagatgtgccaggggtactcgaaccctggctaagaggcatcagtttggtgtgttaggctttctagagggcattcacattttaccagctgtctctggttcctcagttcttccccattcctcccatcaccatttagaaagaaaatgtatttatgggaattgctagctagtgactgacagagccaggactgaatctaagtgagagagcacagtttgatgggaaaccctgtccttggactgtcaggtcgaactgtatttataagtcagattccacttaggtctacactgaccttgctccagggccaaatttcccattacccaaccagcctccaggccaactgctgtgcccattatactttggcagctgagctgatggtttgtggaatgtctcctccataaattgttaagtagggcaagcatttattaagtgccttctgcttacaaggtctagtacttagtattgtaaggcattcaaacctgaatggtccctgtgttcaaggaagttacattcctgtgtggagacacttttaaccacttaagaatattgaaaagcaagttgatacatactataataaattatagcactattttttcttaaatatttttgggaaaaatgatagatacttctttaatgtaaataaccagtttagataacttcttagggaccacagttatttgtaaaacatataaattcatacttcaaattcacaatcatgttagtatctgtgtatttaaaaatataaatggttttacaaagaaaatcttattttatgttgaatgttatattttaatctggattacttttgctgatttgtttttgactcaaatcacttatactgccctgagctacatttatctaactgcttattcaacctctctattggatgtctaataagagtttcactgtttaacatttccaaaatggagcttttgactcttcccctccccaccagccttattccttacccactcttcccctatatcagtaagcgacagctccgttctactagttgtttgggctaaaaaccttgaagccatctttgactcttctctttctgttaacattgcaaaagcttcctaactgatctccctacctccatttttatcccaatcctgtagattcacctaaaaacagccacagtcatttttctaaaatagaaatcaaatcatatcccactgcccaaacctgctgaaggctgcctgtagctcacagggtaaagtctggaatcttgacagtcgcctgtaagaccatacactatgtggcccccactctctctcaaatctcatctcctataaccgcctttttccaatacaccgtctactccataaacagtggctcactgcccaagagtaggggaggggaggatcagaggagccaaatcagaacacaaatctgtccagaggaagggggtggcttttacaaattcatacaaaggttctttttaggctgctggcagcctttaaggctttcttgctggtgtatgaacagattaggtatgcctttgcctcagggtctttgcacatgctgtttctgctttccctgaggtattctcatcctttccttcattcaggcctctgttcagatgtccccttagaaaggccttctgtttcccctccccagtctctaaaatagcacctcccctcacttttctgttcttcttaccctgctcaatttttcttttatttgtaccttccatctctagaatctaaacttcatgaaagcagctactttgtcttttttgttcgtacaggtccaacacttagaaatcatgcgtaggagaaagtaggcactcagaaatttttctgacaaatgaaatgatctatttatgtgtttttatattaagtttctttcttgtgtattgaatgtcacatcctgagtactaaatgcagggggtataagtataaacaaaactgaccccatcgctgccctcttggagctgagagtctcataaacagctttaaggtaataaaatcattttctgtgccacaggatgtgagttggtttgatgaccctaaaaacaccactggagcattgactaccaggctcgccaatgatgctgctcaagttaaaggggtacgtgcctcctttctactggtgtttgtcttaattggccattttggaccccagcatgaaactaattttctccttacgggtgttagttatcatcattaagaaaatgttgaataaatatctaacctacgaatatatcacatgctttttgtagcaacatgttaactatttaaacattatatactgtagagcatatagataacttataaaccatttgctattgctgttattcatgctattaacaagatgcatgtagaatagttatttagaaaagagagtataaagtgctcaatcaacataaaacagtaattgctactgaagaaaggatgtatttaattgctgtaagaaagtttagagtcactatggttacagaagggagggaagacaatcctctaaaatataggttgaaggaaatgaaaagcacattaaaaaattaaggcaagaatagaataacttcagtctttatctttaataactttaaactttaataattttaataacttaaattttgctactgtatgaatctcttgatataactagatactattgaaccagcaggttttgatttttggctgaagtgacaatttcttctacaactgtttatggcaaaagtccacaaaatgatgtagaatttgaaaaaattcatgtaatctctggtgtgtcttttcccctcttgaaccttatccatctttatctttaaatcttttctgtaagttagtactatactaacatttcttctatctaatatatggggcttctttaagaataaattaagctataaatgaggaaatacatagagttataacgttgaaatataaaccttaggagtccctctttttctattgtttggaatagtttcagaaggcatagtaccagctcctctttgtaattgttgttttaatacaaacttctttgcctaaagcaaacaaaacaataaaaatcaaggtttagatcaagttgtatagaatgtaattacaggtgcacgcctgtaatcccagctactcgggaggctgaggtacgagaattacttgaactcaggaggtggaggttgcagagctgagattgcaccactgcactccagcctgggtgacaaagcaagactatgtctcaaaaaaaaaaaaaatgcaaagaagacagagtggctggaataaagtgagtgaaaagaagagtcataagtgtgttaaggtcagcattatatccagaagtagatggaaaaccactgtagggttttgaacacagaagtgacatgatctgaaattttgaaaggatcactatagaaactgtgtgaataggccgaagggggcaagcatagaaggagtctgttgcagtaatccaggaggagatgatagtgttttagactaatttggttatacaaaaggctataagatataattaattctggatatattttagatgtacagccaacaatgtgttggttggataagatgatggatatgagagaaagggtatttaaagatgactccaaattcttttacctgcacagtggaaaaaaaaatggagtttttttttttttttttttttttaaagagacagggtctctctttgtagcccaggccagactgcagtggcatgattacagctcactataacctagaactcatgggctcaagcaatcctcccttctcagccttcctggtagctgggaccacaggtgtgcaccaccatgtcgagctaatttttaaattttttgtagagacagggtctccctatgttgcccagtctggtcttgaactcctgggctcaagtgatcctccctgcctcagcctcccaaagtgttgagattacaggtgtgagctgccatgcccagctggagttgatctttatgaaattcagaagcctgttggagaagcaggcttgggggagaatggagagttctgtatgagacatggtaagtttgtgacgtctgtattagacatccatatgtagatgtcaagaaggctgaaatttgcactgctaactttagtaaaccttttttataattggtttactaaataagttttactatgcttctccattcattttggtcctcacaactctatagactcctactctgtaggaggaatatacatgagacagtgagcattagtctttggaataaaggaacagtaaccagtgcaatgtgacattgcacaatatgacacagaccctgtggtatgggctcatgtgctttgatggacaaatatcccgcatcacatttgacgtggaagacacaatcctggaggcaactcagtcttttgcagcagaacccagagcagatgtattcagggcattctgtattgcagtttttcttcctgcctctcaaattcctctggtgttatgacctgccttaggccacagtgagttcctatatttgtaaaattggtggtcactttttccctccatagtgctgtttgtgaggccctttgccatattcactgtctctaattgcctgctggggtcaaccttctgcttttcacttgtttcctcaaagacacctcaaacttggccctgccaagatggctgcactcaccttatatgagccactcaagcaaaactgttttccagaacttaaaacagtggctaaaaaggaaagaccagggaagaggaaatgagcaggttggcaaacattgtcaacctaggaaagcccaatgtagctgttatcacagattgactgagagaggactgttggatctactttacaccactagctgacctgtttttggctgacaggttttagttcctcccctcaaccctagtctttacaatgaaaattttctggctactaatctgtgctcttccattcttcttagtccccatatttctatagactcctactctgtagaaatagatgtaggacagtgagtgttaatcttcagaataaaggaatagtaatcagtgtaatgtctgaagtgctaaatcaaatgaagccgaggggttaaatctccttgcagtttaaggtcttagacttcttagttactctttttggtacattaaagaatttgccatctttgatccatcttatttatttctgtggcccttcactactatccaaagctgcacatttatatgtctgaaaagttaaatagccaggtaattcctctgccatgaactcacacctagaagtgattctaggtgatattctaagagcttctctcacaagcccataagtcagtagctgcagtgggaaatgagctctgagtagaacttgtagatactgagaacaggagcaacttatatcatccctgtgccacagccgttctccagcttgcatcctttgaccttcaaactaaagatgtgaaatgcagagagctggctttgaaaaccattccagtctgatttatcccaaatttggcaaatcatccctttaatataagctcaatgattgcatcaacaaaatatacaggtgttctattatttatgatacctctttcagccctggtgcagctccatggtatggaatttaatgtatttaaaggctactgatagttatagcactcttttcctaaatactagtctggtgtttatatcagacaggtgttaaatgattttgtagggattaatgtagggaattataaaggattctattgttactagaaaggggtcccgatgcagaccttgaaagagggttcttggatcttgtacaagaaagaattcaaggcgaatccacagagtaaagtgaaagcaagtttattaagaaagtgaaaaaattaagaatagctacttcataggcagaacagcagcatgggatgctcagctgcttatacttattgttacttcttgattacatgctaaacaaggggtggattattcatgagtttcctaggaaaaggggtgggctcttctcagaactgagggttcctcccatttttagaccatatagggtaacgttgccatggcatttgtaaattgtcatggcgctggtgtgtctttgagcatgctaatacattataattagcgtataatgagcagtgagaacaaccagaggtcacttttgtcaccatcttgattttggtaggatttggctggcttctttaccacagcttgttttatcagcaaggtcttagcgacctgtatcttgtgctgacctcctgtcgcatcctgtgacttagaatgcctaacctcctgggaatgtagcccagtagatctcagccttattttacccagccccttttcagaaaaggcaggtctggggccagggcaggcatttggaatggcttgagggcacagaatatttccagggtagaggagatgtgctggactaaattatagagtgatggactaaactgactttgtagcttgacttttggtttcagagttggaaatctggtttacagttggacattatacagtggtggattaaattcactttgcagtttgacttctggctttagagctggaactcagtgcacactagtgaaagtcatgcttcagactccctctggaattcaaggggaagataataatgcgcgcttactatcttaaattaaattccataattttcagctctgtattttttccaaattaaacattatatctcaaacagacccagatatatttgaatattattaatgacaaacgttaggcttaaattacaaataataatatacctaacattggaaatttccatcattcctagtttgtcagactcctttatcttgctaatttgcagatattgctttagtaatgttgccgtgattaatgaaggttttcttggtattaaaagatccaaagagataggaatatgtaattgaactctagattgttgatattctactttcagcattctgaagtcatggaaattcttactgtagaaactcaataaacttacaagtagacctttactttttagttcattactgataaaataatgaatatagtctcatgaaggtgagttttcagaaaatagaagcatgagttgtgaagataatatttttaaaatttctctaatttgttttgttttgcaggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtnctgggaaggtgagtcaaactaaatatgattgattaattaagtagagtaaagtattctaatcagtgttattttgttactccctactgcttactatgctctaagaatgtgtttataaccattcctcaaagcaatctttttcatgcttattcagtaaattagaaacttacagaaagtagcaaagccagttcttggactcaaaaactgataattaactttaacagactttttcagttttcaggccattgtcttcacactgttcttccttcctccccactttcctccttcccttagttattttcttctttcttttctctcactttcactctctctccactccttccttccttctttccttccttccttctttccttcttttctttccttttttccttccttcccttcttccttcttttctttttctttcttttctttctttctttctttctttctttctttctttcttgctttcttgctttcttgctttcttgctttcttgctttcttgctttctttcttttctttcaagcttaaatccattcctttattgaggaaagtaagcccattttatttgtacatgtgaggggggagattaaatatggaaaaatgctaggggtatttattatatctgttttaaattactaccactctttctttttttttatcatgctcctcccttcatcctatttctgtttatctttaccctttttttacttcttttttttcccctgcatacttgtcttttttttcccattatttaacaaatgcttatgtggcatttactgtgtttccaggcaaatgtcttattccttatagcaaccatatgggggtctattatctcatttttctagtggggctgaggcacaggcaggtcatggctctggactttagagctgataggtcttggagctgggattcaagctcagacagttgtgctccaaagttgtttctctttctgttataaaacaaagagttcctctgatggcagaatgcagtctgatatcacatgatctgtatcatagtggaaatgagaggtcagagcagggctgactgccataactaacatttaggacagggatatatgtgtgatgaacattctgagattcccaggagttagggcagggactcacacagatcagagtggctctggttgtcagtaggcccagctacctcaccaagtgaatgatgaagaaggccccagatgttggtcattgccactaatgctttgtcctttacctctctgctatctcttcagactctttatcccatttttggggggttccctctggaaaatcttttgggccagctgtagtacctccccagccagtgtctgtaggggacaaacctcatgcgaggcatcctatcaggctcccggaggcctatttctggaggtacatcaggatggtgctgacccacagggttctattgtggtctggctcaagtaacgttggcccctgccagggaatacattctgtgccaatgacttcctttaatattgtactttgggggtctcccccatggtgttgactcttcccctgagaagatggaaaaatctaggacaaaatataaaaaaaggagaaagcattctagttaacagtgtcattatttaatgaggtatttgaaatgtcccagaagggagtcttagattgcttttaggaagaagatgatacaatctgatcctaagctaattcatgcaaaccacaggttagcacctttgaagtgacacatgagatttaagactcttggagttctgtcagaggggccaaaggaaggggagcagatggaaatgaattatgtgtggatatggcagattttctgagcaaaggtcagggatgtgatacattttctaattcaggggtggctcatgagagggacagaagtaggtagggaaagtagtggatctgcaccaaccagtccacataatattgaaaatcaacttccacctgaacacacttctcaagctgctatttagtacttcttatataacaagtatcattctaacagctctaaatacttgatctaatttaatgctgtcaataaccctgttgaattatccctgttggatagataatgaaacccaagattcagagagatcaagtaacttgctcacagtcacacagcaacagcctcctaaccaatctccctgcttcaacttcttccattcttatccaactatagtctacattcttacagaagtcattctgacctctcataaataaaaatctaataatgccaacccttacttaaaactcccaaatggcttctgatcatacttggaacaaaatctaatcttttatcatggattaccaaactctgtatgatctaactcctgatgacctttatggccttatctggcatcacagtctcttatgcctgtgtgccctggcccagtgggcattcttgcaattcctggaatgttttaagctgattgcctctgatcttctcttggctgactttacgttattcatatcccaaaattattacctcttcagagaggccattcctgacacctgaattaattcatgatacttcgcagcccttgatgtttctttcctatttctttgtttattggttaatggtgtaccatttgtttattgattaatggtgtatgtgacattagagcccacacttattctgactgcatgatgtcaaaacctgctcataacctctaccctgcctgtgatctgtgctctgcagtttttcagcacattattgtggactagtgagcatttttataaaatacaggtgacccttgaacagcacgggtttgaactgagtgggtccacttatatgtaggtttttttcaataaatgtattggaaaattttttgaagacttgcaacaatttgaaaaaactcacaagccatgtagcctagaaatatcaaaaaattaagaaaaagttaagtatgttataaatgcataaaatatatgtagttactcgtcttattatttactaccataagatatacacaaacctataattaaaaaaagttaaaagtgatcaaattttatgcacacaaacacttacagaccatataagtcaccattcacagtcaagacacatgtcaacaactgtaaagatacagtgttacatgttttggctctgtgtcctcatccaaatctcaccttgaatggtaataatcttcacatgttatgggagggtcccagtgggaggtaaatgaatcctgggagtgggtttttcccatgctattctcatgatagtgaataagtctcatgagatctgatgattttataaaggggagttcccctgcatatgctgtcttgcctgctgccatgtgagatgtccctctgctcttccttcatcttccgccatgattgtgaggcctccccagctatgtggaactgtgagtccattaaacctctttcctttataaattacccaatcttgggcatgtctttattagcagcatgagaacagactaatacatagtgttaaatcataaaataaaaccatagtatgtactgtactaccccagtaattttgtagccacttcctgttgctactgcagtgagttcaagtgttgtacctgcttaaaatgcagtgacactaacaatctcagcaggagcagttcatctctccagtaaattgcatattgcaataaaaagtgatctctcatgattcttgcgtattttaaaaatcatgtttagtgcaataccataaacattaaataacaccagggaacccatatcaagtgacactagtgttgctggaagtgctcccaacaagcagagaaaagtcatgacattacaggaaaaagttgaactgcttgatacatactatagatgtgaggtctgcagctgcagttagttgcctgccatttcaagataaatgaatctagcataagaaccattgtaaaagaaaacaaaacaaaacaaaaacaaaattcgtaaaagccatcactgcagctacaacagcaggtgcaaaaaccttgcactttttgtacaatacatttatctcatattgaaaatgcagctttcacatgggtgcaggattgctgtgagaaaggcatacctatagactctaatatgattagaggaaaagcaaagtcaattcaaattaaaagtaagattaaggatcagagctgggtcatttaatgccagcaaaggatggtttgataattttagaaagatatttggctttaaaaaaatgtcaagatagcaggagaaggagcttctgccaatcaagaggcagcagaaatgttcctagatgctattaaaaaaaatcattgagaagaaaggatatctgcctgaaaaggtttttaatgcagtcgaaagtgccctattttagaaaaaaaaaatgccacaaaggacttttattaataaggaagagaagcaagtccaaggatttatgtcagaaaggagtaggctaactctactgttttgtataaatgcaatccggtttaggatgagtccatatctatagatatgctcttatgtgtaaagctgttcatccctgatccttgaagggaaaagataagcctcagctgccagtattttggttgtacaacaagaaggcatggacaatgagagcactttttccggattggttacattgatgctttctttgttcttgaagtcagaaagtacttagacagtaacggactgccttttaaagttcttttgatattagacaatgctcctggccacccagaacccatgagtttaacatcaaaggtattgaagtggtctgcttgcctcctaaacatgtctctaattcagcctttagatgagggggttataagaatctttttttttttaataaattttttgtagacatgaggtctcactgtgttgcctgggctggtctcgaactcctgagctcaagcgatcctcctgccttagtctcccaaagtgctaggattacaggtgggagccactgtgcctcgccactcataaggatttttaaggctcatcacaaagcacatggtactctacagaaaggattgttaatgctatggtagagagccaccagagagaagaacatcatgcaagtctgtaatcctaaaacgataaatttctgctggagaaaactgtgtccagatgtacatgatttcacaggatttatgacagagccaatcaaggaaaccacaaaaagaaataggagatatgaaaaaaaaaaaaaggtggtgggtgaaggctttcaaggtatagattttggagaacctcaagagcaaataaacatcacaccagaggaattaacagaagatgacttgataaagatgagtgtttccaaaccactgccaaacaatgcggaagaagatataggagaagcagtgccagaaaactaactttacgcaatctggcaaaagagtttcggttattcaggactgcttttaacttcttttatgacatggacccttctgtgatacaggcactgaaactgaagcaaacagtggaggaaggattggtaccatattgaaacatttttagagaaataaaaaaggaaaaagtcagacagagaccacaatgcatgtcagtcacaccaggtgtgcctgcctttcccgcctccccttccacttcctctgcctcttcccctctgccacccctgagacagcaagaccaatccctcttcttcctccttttccttagcctattcaatataaagatgatgaagataaagacctttaaatgatccacttccgcttaatgaatagtaaatatattttctcttccttgtgattttcttaataacattttcttccctctagcttactttattataaaaatacaaatatgtattacatataacatacaaaatacatgttaattgactgtttatgttattggtaagacttctggttgacagcaggctattagtagttaagtttctggggagtcagaagttatatgtgaattttcaactgcacgaggggtcagtctccctaacccccatgttgtccaaggctccattgtagatgcttttttacttttcagaaataaaagccataaacttgtttatttgagggtaggaagagtaatgtcaggaggctattttttctttctagaaacatacatttttatttcttcaaaattatttagtacacaacagtttccgagggaaactcgatgctatttgttcttggattagaaattctctctcctgatggtgattactgagtctccatttaaaactcttggaataaacaaagctgtgaggatgcggggtgctaattaagccttttcctaattgcttcattgtgcgtcaagatgaagacagtcctgaagttattatactgttttactcaccacttttaggtctgtgactcaaactcccacttttattcggctatatacacactaaaaagcaatgacatttacaaaccaatctcagaccagacactcctgccttagaacatggtgcacagaaaatatttcttaaaaccattacactgaaatatacagtaaaatctgtttttcagcagacattgttatagtctgttgaattttcttactaatctagaaaacctgtttgttagaattctgataattagaaatatttctttttttttttgcttgtgaaacttcagcttttattttatttttttatttttattatactttaagttttaggatacatgtacacaacgtgcaggttagttacatatgtatacatgtgccatgttggtgtgctgtacccaccaactcgtcatttaacattaggtatatctcctaatgctatccctccccccttccctctaccccacaacaggccccagtgtgtgatgttccgcttcctgtgtccatgtgttctcattgttcagttcccacctatgagtgagaacatacggtgtttggttttttgtccttgcgatagtttgctgagaatgatggttctaatgtgtctgacaaaaggattgtctttgggaatttgaaggtgaattttttctcctcaccttttgctttctgcacttttacgattttctaaagtgactatatatcatttttataatgtgtaaaagaagtttatacaatattttaaaataaacctgccattttcctaattttctaagtatcttgtggtaaacataattcaatcttcttggcctgtcagtgtaagaataatattttaaattttatttttaaataagtttttgtttctaagaatgttactaatttttttttttacttctgatagattttgatattagtcttcaaaactgacttaatgtcttatgaaatgcttgctgttatgtttgaagttaggtaatttatgtaagattcagtgaagaataagtggaattccatgtttatgattttaagctataaaacactctaaattaaatgtgtctttattagaatctgttctgaccagtgcagaggccaaagagaggaagaacatcttaaacaataaagagtcatttctcttggtgacttataattctggaagttatttctcttaaaatcatagcattaaaagggactttagagaccctctagtccatcgtcctcattttgcaaatgaggaaaatgagacagcatgttggttcaaggtggtgcggctgatgtaggctgaaatctcatcttgtacactggtgttctttgctttttccatatccctttactcagactccagaggtgatgaaggatgtatgtttcctaatcagattgccttgttggaagtaacatttgattacaacataattgaatgatggaaactttctttttaagatggagtctcactctgttgcccaggctagagtgcagtgacgccatcttgactcactgcaatctctgtctcgccagttcgagcgcttcctctgcctcccagtagcatgggattaaggcatgtgccaccatgcccagctaatttttgtatttttagtagagatggggtttcaccatgttgatggtggctggtctcaaactccttacttcaaatgatccacctgcctccatctcccaaactgctgggattacaggcatgagccaccatacccagcccaaaactttctggaaaacagattgatagtatgtgccacattccttaaaaaattaaaaaaattaattcaagccaggtgtagtgccatgtgcttgtagtcttagctacttggcagactgaggcaggaggattgcttacccaggtgtgtgaggctgcagtgagctatgatgatcacacctgtgaattgccactgcactgcagcctgggcaacagagcaagaccccgtttctaaaaaaaaaagttagttttctttgacttattaatttcaccttaagaaattttcctaataaaccaattcaatatggacaaatgtttaggtacaaagatgtttatctcaccactatttttaataaaaaaggaattgaaaacctggctcaacaataaaggaatacttaattggttatgatattaaaggactcattacacatctcattattaatgtgtatttaatgaacttggaaaatgcttttgatatgaaggtaaaaataatgatatagagctaaatatagagtttcattccaatctttttaaatatatttatgcacttaggaaaaaaacaatatggaaatgtgtaaaatatactttttttttaaaaaaaaggacacatttattcagcattatgatcagactattacatttaacaatcaacagtatgggtgccaaaaaaaatctacattaaaaccctttgttgtaatgctttacactttccacagaacagaaactaaaagaatctgttacacaattagtcacaaatatagtcctcgagttttttacccatacacatgagtatttgtctaaaacatgtcttcttgtagcacttaggccctgccaccactgtgcttgtctgagttcacaaatctgttgtaaactgtagcttccctgtcacttctctggctcttatctcctgctaagatttgtttcctggcagtaatttaaaatcttctgccactgctgtagctactgctgctactggaactgccatagccaccttggtttcatggtttggcaaagtactggcctgtaccagcataggggccagagcttctgcctccaaagtttcctcccttcatgggtccaaaatgtaaaactaattgttgtaattgccaaaatcattacaccacctccaaaattgcttccatgattaccaaatccattatagccatccccactgccactatatccaccaccaccacagctgccaccaaagccacaatgaccactgaagtttcctccacgaccaaagttgtcattcccaccgaaactacctccacgaccaccaccaaagttcccagagctgctttgcctctttggctggatgaagcactcaccatctcttgctttgacagggctttcctaacttcacaagtgtggccattcacagtatgggtatttctcagtgacagtcttacccatggagtcatggtcgtcaaaagttactaaagcaaagccccttttcttatcactgccttggtcagtcatgatttcaatcacttcaatttttccatactattcaaaataatctcctaggtgatgttcttcagtgtctttaatgccaccaacaaatatctttttcacagttgggtgggcatctggtctttgagaatcttctcttgagacagctctctttggttccacaactcttccatccaccttgtatggccttgcattcatggctgcatccacctcctccacagtggcatatgtgacaaacccaaagtcccttgagcgcttggtatttggatctctcattaccacgcaatccatgagcattccccattgctcaaaatggctcctcaggctctcattggtcaaccaatgtagagctcaacctccaatgaagaatttcctcagctgtttgggctcattaggagactctgacttagacatgacggcaggggaaagagagactttaaggatgcttccttggtggcgtccacgggcagaaaggggtaagcgtccacaggcagagaggagtaagcctttgaatgtatctaaaatttactttttattgcctgcattctttcactattttccaaacattcttcaattgtcacaaatggcaatgataaagagaaaaatataaacatcacattttaaaaataagtgtaaaataactgtgaacttaaaatgtgatcatcatagaagaaagagcactgcaatagaagtactgtgagttctctggttaattttgcgaagccacgtaaagctgtgtggctttaagcaatgccataatccatttaagtcttagcttctatttctgcaaaggagaagtttgaattagtcaatcttccttccagatccataactcagtttgataaattacttagtatctttttctacagagaaaatgctcatacataataataaatatgtaatcataaaattattttcattagtctgttttatagaattcaaattaatcaccactatttactcttgtgcctcttggtgatcggtgctgtctgttacagatcgctactgaagcaatagaaaacttccgaaccgttgtttctttgactcaggagcagaagtttgaacatatgtatgctcagagtttgcaggtaccatacaggtaataaccgctgaagagtgggaggagagtgtgaataatttttcaatcatcatatttgttttcagagggattactttggctagaaggtagggagcaagtggagaaagtgctcgaaggtaaaccattgagaaacagttgtaattatgcaggagagaaagtacaagaccctgaactaaggcagggacatctctgaggtagaacctgtaagaatgggtcactgatgagaagggagggagacatgatgctgagaatgactatctgatgtccaggtaggatatgaccctataatttgctctagttgaaaatgagttatttatggaacctgaaatttgaggtacctgtgggacacagagaggatggtgcttcctgggtttacctgtctttctgctttttaccccttctccagtgcagaccttcttcctttaatacagtcatcccagtgagggctctaataagtggtgtgaacataagcaagcccaacctttctgagtctgtttcttataataaaaggaaagctggactttattgatggatttttaagcttttatttaaaaaaaaatgatggtagagcctttttgtctaaaaaaaaattatttgaaatcttcatatgtgatgaaggtaaaagcagagttgatctggtagcaggaggggttggaagcccagggctgcccacttgctaacctgcccccacccacacctccatatcactgagctggatccatatcatgattctagaatgtcaacccctttttaaagccttctctgagacccccgaagaatttagtgcttctcctttcttcctataacagattattcataaggcacctctaatgcataaatagtaattcaactcaagtcaggtctcctaagtcaagaacatgcctgtttcctctatgtcaacccatcatggcccctgcaccttgtaggtgttgggtaactgtgtgcagaatgaatatttacgtagagtacttccatactgtgtgtccaaaagtggggagaaagggagataacttttacttattgatccctaaccggctctttacagtcattggattattttctctttacatcaacactttgaggtgtagttaattctacattaaagataaagacactgagtctcagaggttaggcagtttcccaaatttgcacaactgttaagtgtaaagtgaggaccaaacttagttctttggaaacgaaacccatttttgttggtcatgcctctgtgccctactgccaacctatcaaaagttacattttaaggactgagaaatgaaagtggaatgagttttcaaatgtcttcttttcagaaactctttgaggaaagcacacatctttggaattacattttccttcacccaggcaatgatgtatttttcctatgctggatgtttccggtttggagcctacttggtggcacataaactcatgagctttgaggatgttctgttgtaagtattgggctattatttagttaagctctaaaaataaagctgggatgaacatgcttcatgtctagataggaaaccctactgtgaagcctcatgaagagattctggtgattcctaaatcggcatttctgcctctgagtcttcatgtgccaccattgaagcaccccctttcatttggaggagcagtaacttctttcctcattgctggctcacacatagttgaccttttcaaatctgtgactgagtggagtgattccattcggagattttgagaaggccttggcatttgggaagaagcctagccctgagcaaggagtctgactggctccttttaaaggactttcttacagagcaagtaaaatacagatgtgttgtactaagttctgcaagcctttggcaattccaggatatgtttactttcccttgataagagaggaattggaaggtaagagccaaatgaattcagaaatgacaaaggaaaagttatattgggatttttctgctacattgttctgaatgtagataattgtacctcggtcagaggaagataagcctgaagcaattatactacaaaagtacccaaatatgcaatttggtggtcaaaagtatgtgtaatctttctgagcttctagatttggaggtgggtaagattctgcctcttgatagcataacataattagtagcgatataattttatatttaaaccagaatcatataagcctggcagtataatgtgttagtatagtatttctgtcctttttaaacattgagttgttcatgcattacagtttgctcaggatgacccccaaaaagacatctaaatttccattaaagatgtacattggacaaatgatatgcaggtgctatttgtgattatggcctagagatcaaaaccaagtatcactggcattggggctttgattagataattatttgatatattgctttactccaaaaaattgaattgatgaaagttgctgacattggggcatttaggtttgcaaaatcaaccttccaagttaaggaaaaggaagacctgtctgaagaacagtgcattagaaaaaggtcccataggtttaaatgatcacaagtccaagattaactatcggtattttatttagtgctagttaaacaaacaaacaaacaaaaactaatgacacaatatgctacacaaacataggcatagagtccagaggaaagaaagcgatgaattccctgtattctgggcgggttcccaagtattatgtctgttccaggcttgtgtttaaaaaaagcatattaataaatgtgtctagagaagggcagtgaaaggagttatcaaagaagcaaaggagtttttagcaatatttcaagacttgtaggcctgtcttatgtaaaaggaagtaaattttttcttcagtttgaaaaagaactgtttaacaatagacaatcaaacatgctacttcctaaaacagaggatggaagccaatttaggggaggtgtccaggcacgaacatggagagctggacttgatacctgtaaggtccttcccaactttaagttgctgtgattcccatgtcatagataagaacgtcaatgcatcttaagagcaacatgatatctggctctgtaagaaactttcttttggttgcacaattcataggtttttaagaatctgatgtaattccaacatcactgactgtatccgttgttgattaccacaataatgctgtgtaacaaccagccactaaacctcagagacatacatatttttgcccacaaacctatgggttagctgtgaagttctactgatctggattaggcatagtggatctcggctggacttactcatgtgtctgtagtggattgtgtctgtagctggttggttgggattcagacagctctcctccatgtgtgcctcatcctccagcaggctatcctgggtttgagacagtggcagaattctgagagagagagagagagaaagagagagagaacacatgcatgcatgctcacaaagcatataaggccccctgagtcataggcttggaactggcaaaagtgatttccaccatatcctgttggccaaagcaagtcacaaggccagcccagattcaaagggtgctgcaaaggtcacagtgcaagaagatgaggatacagagagaggtatacagagggaaaaaaatgggccattttgcaatcaatctatcacatagacatgaacttataaggaaatgtgtttgttttatttttaacatctgttttataactattagaggcaaagctctctggttatagaagtgtcaacttttggccaggcatggtggctcacacctataatcccggcacttttggaggctgaagcaggaggttcacttcagcccggtagttcgagaccagcctgggcaatatagggagacccccatctctacaaaaaataaaacaattagctggtgtggtcatgcacagttgtagtcccagctactacagaggctgaggtgggaagatcgcttaagccctggagatcatatctttagtgagctctgattgcactactgcactccagcctgggcaacacaggaagaccccacctcaaaaaaaaaaaaaaaaaaaaaaaggagtgtcagctttctagcattgtgatggtaatgctgtgcacatgttttgtgtttgtgctttccagagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccggtgagtttgatgtttcaactgtttgatctactcctgactcctgaatgaaagtattttaagtggaaacttaataaaatttgtactttcaaatatgctgatgataaaataaaacttcctagatcatagattcctttcaattactgctaataatatacatcaacattcagtacttttacgtagcaaaggttatagggaaataggaatactgctcactttataagcaaaacctattaatcagattttttaaaaacaatttttttttagagacagagtcttactctgtcatccaggctggagtgcagcagtatgatcatagctcactgcagccttgatcttctgggctcaagcgatcctcctgcctcagcctcccaagtaactgggactacaagcgtgtgccatcatgcccagctaattttttaattatttgtagagacagggtctcgttatgttgcccaggctggttatcagattttattgtatgtaagttactgtattcctgaggaacagatttgagttattgtagctgtattgcatattcatattgtcttaacaatacatgctatgaaagcttttactcttttagatctcatttattaaattctagcagtctgaggtcaagcacagtggctcatgcctgtaatcccagcactttgggaggccaaggtgggtggatcacgtgaggtcaggagttcgagaccagcttggccaatatggtgaaactccatctcaaataaaaataaaaaaaaaaagattagctgggtgtggtggcacacgcctgtaatcccagctacttgggaggctgaggtacgagaattgcttgaacctggggggtggaggttgcagtgagctgagatcatgccactgcactccagcctgggtgacagagtgagactctgtctcaaaaaaataaaaaataaataaataataaaattaaaataaaataaattctagcagtttgaagtgaagccaattgtaacacaaattaattatcttctgacacctggtaatcgagagagttagctatacactttattttcagtattgcagcattcaaatttactgttattcttctcattgcagaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtggtgagcacactttcacatttagctcagttcaggttttcatcatccaaatgtctgaatgtatttaattctcaactataagccatgttttttcaaacctttaaacaacagtcccacttggataaagtctgagagcctaaatatggtctccaagtggtgtcatctgtcccagccaacttctccaggctcccctcaacactacccctctaccctcctttgcaagcaccctttgtaccacctgtctcccttgctgtacttaggtttcagctgacttgaaaagaaccaacaaaaatggaagtagccagaaagatacaggacaggtgctaggagtgagatgaaagccaaggatggagactgtttcaaagatggtggtcagcaatgacagatggtatagagagattgggtaaggaggagactgagaagacatgatagaatctagcaatgaatgggctatttctgacattttaaaatattcttagtgaagtagtgatggtaggaaacagttaagggtggctgaagtatgattaggggaagaaatggagatgtaagtgagcatagattatcaagaagatggaaagtaaaagaaaggcaaataaggacagcacttgagtggggaggcagagtccagggaaaatgtttaggggttgaactcttgagcatttttatgactggggatgagacaaaatcattgatggagagagggcattgaaacatcatctgaggaaaaaaaagtagaatcaacaaatttgggaaatgtaaaaagaacatggaagtatgcccttttgtcttagttgctggggatgagggaagtgtctggcagggagtagtgagtttaaacgactatgatgggggataggaaagaagacaaactaggaaggaatagaattaagagcaagacattttcattatgtaggcatgtaattagttgtactgagaagtgatatttaaaattcttaacaatgggaacagcatggacactgaccaatcagaatggatgcctgctagtacaagtcagatggatgcccacccgctgtgtatggacgctggtagcatttaatgactcaaatgaaaagagagttttcaatctttagtctaggactctttttacttcctcacagcggtcactcttactcataaatgtttgttattgtattcaaagacagttatgcctttattcagtttactcttaaatcattacaagcttgttccctacttttactctcttgctttatattttctgtgaactctgattgtgtccttcaattttgtggtgaacttaaaaacaggactctaaataaagacgtcgctgacagccaaagcaattagaggaggaaatgttagctctcagacactgattatggccctcaacagacagttattaagtggtgtgttaaagattgtgctataatgaattgtagggcatgatatctgccctcaaatactttaactgaggagatggggcatatagttttgtgagttaaaggagataggcctagagctagttcttaagagataaggatttgcatatgggtgaacagtagggatgacattcaatgtaagagaaaatacaagctgcgatcagtgatttgttcccaatgcagcaaataggacactttggctgaaatgggagtttagtttgagtagtggttctcaaacttctctgatgagaactcatgtaggaaagatttaaagatgtcaccatccactaatgtactcatagtcactttccagtgggaaaaacacttggtgataacaaaatgttggagttgaaaacaacaatacaatacagcacaataatgatacaataaggaataattaatgattattctgcaagtatataaacacaagcagaacataataaaacatggacagcatcagttacagttcagagtaggaaacagaaactgccctaggtatttcaagtagataagtatttaataaagggaattacatgcttacaaaatctttggaagggctggaagagtgaaaggaggctatttgctcccagacttccaaatcacagcactgcagctgtgattctgtaaccaagaagctgcagaaaattatgccgatatcacagctgttccaaatttaaagacacgagactagaatctggctgttgcagaaattcttgtctgccaaagatgagttaagccgttagcttaccacagctgctgaaggagagctggtgtctacctcccagattttacactgtgcatctccctcgtagaccctgacttacttccagaaccaagggaaactgggaaatagtttttagccttctagtcccttgatgtataaaaggtcagacacagaagtatatgaaaatgaatgctgagtgcataggacagtaagcatcccaagacattattgataatgcactgaaaacaaagtgtcctcatttattgcaggaatatccattatcttctcctgtgcatttgcactagggcttggaaatactgccagatggtgcactgcagccagacttctcagcagaaagtgtccacaataaactctagtaaatgtacaaagttttgtttatggacatcaatgagtagccctgaataactcagtggattactactaattagtttttattgccatgtaaaaatgagtctgtggatttgagcataagaattaaagaaaggattggaccctaaggaccccctgaagtcaggaaattctcgtgaccatcagtggaccttgaatcccatgttgaaaaacattgacctgaaatggtggttctaaagcttcggtgaatattagaatggcctcaagagctagtaaaaaacacagccggcctggattattcaagtaggctagggtttggccttttatttttataatattccgaggtgattctgatgccaaccatcattcgagagccactgaactggtaaatttgaagaaagatttaggttaaattgtggagagtcttgaataattaagctatgaaattgtgaagatgggaggatcctccaagttttgagggacctgaaggttatacaattgtagggattcaacttaaagtaaaaaaaaaaaattttttttttaatgcaaaattagatacaaaggtggatatttacttcaaatgaaaaaaaaatcataaccaaatactggaagcttaaagattctggtcccttttcatttttaggtaatttatcatcaatacttacagggaagagcttcctgactacagccttgcctccctcccacccagaaaactgttcgacttcccagaatttacatgtaattgagggctcttgaagcttaaccttcatcagtgtcactgttaaatcacccctgctttgagtcttccccttacctttcctttctcccctttccagtctctcccatatacaacagtcagaaccccctttgggatacaattagaagctgcttgtcattgtctggttataacccaagaagtgcctgagccaagatgggggcattcccctttcaaggggggacacaaaatatctttcagttgtgaggtattagtgttaccaaaacccaacctagagtgtttagatccagtcttggttttcccccaattactaattgtgtgaccttgtataagttacttaactttgctcatttccttatcagtaaaatgggaattaacatcacagtgaccttataagtgttgctatgaaggttaaacaagagactgtatgagacatgcttatcacagaacctacaacgttgtcagtgcttgatttttaaaaatcaattattatttcacagtaaatatgcatagaagaatacacatttaacatttacatttttgcgctaatgaaagcaagcaatagacttaacagtctacaatagagatagccaatcattttaatctggcctttcattatttcttaccaataggtttactttcctagttatatttttatttaggccaggattttttgagcacttctcatagattccccagagttccttggtctttcctcagaccaacaccattcctcagccaaagaagctctgcttctttgatctgttttacattctaggcatctaaacttgttttacttaaagaaagaattcttcagtcaacacaggttttaaatagtttgcaattctagggtattatgcggggacgaggaggcccaagagtgtattcagtgtaatgaagaaactcattataacttgctgagatcattcagatttgcctgtgattatattagttaggcagtgtctgttttcctcccaggggtataaacctggaaaattacaacaaaaacaaaagcaaaaaccaaactcctcctttccttaaatttccacttctcaagtacagtgttttatctaacaagatctgctgcttagccacatccttgtttggctttcactgtctctctccaccctctactttccaccttcatttattaacattgtaaggaagcctggagcatacatgtgtggaatagtctccatggcaactcagcttggaactaataaggttatgggagagcttccatccctgcacctgccagtgtcacaagcagaagccatgttcctgtagcaaagattgtactctactgctaggcagctgtccccttgagccacccagccaggcacatgggatacagagagtatatggctcagcacgcactagtcactactattagaaaagctcaaagctgagtcactggtgccttcttcagaagggatgaacagctctctcacttgaatgccagaaaattatcttgcaaagcagacctatctgatagacatatttgcatcagagtagggcttgttatcagcaaggctgtaaggtgccctccccagtcttctgcaggataatccagggagccagattatagagaaagggcagccctctgttcctactcatctggctcagtattgaggatctccattcaccctttcctacccctgttcacctatccatcccctgtagttcctgacctgcaaagctattatgtggtcatagttgttatttatttccatgctaatcctgggcactgtttctctgaaaaatggagatttaagaataaggctgtcaggatacatctcagaagtattaggcgttgtattagtgtggctgctataaccacttgccacaaattcagtggcttaaaacaacaccaatttatgactttacacttctggaggtcagaagtctaaaataggtctcagtgggctcatatcaagtgtcaggagggctgtgttccttctagaggctccaaaggatgatctgttttcttgcttgtggccccttcctccattttcaaaaccagcagtggctggttgagtctttctcacactgcatttgctgatactcttctccctccttcttcttcagttaagagcccttatgattacattagtttcaccaagataattcaggataatcttattttacagtcagctgattagcaaccttacatctactactttagtttctttttgccatgtaacataacacattcacaggatccagggattaggacacagatgtcttgtgggagagggaacattattctgcctaccacatgcatacatcagaaaccatggttgaaacacaggaaacatgacagttcctcaaggcatacaattatgaccttgttgggttaaccttcactatccaaattttaatcacacaaacttttccttaatctcacagtaacttggcagtttcagtgtaagaaataatgatgttaattgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatn

SEQ ID NO: 10 - DNA, 20 bp, Homo sapiens
  misc_feature (7)..(7): n may be any nucleotide
gaagggnctgaacctgaagg

SEQ ID NO: 11 - DNA, 18 bp, Homo sapiens
  misc_feature (7)..(7): n may be any nucleotide
gaaggtnctgggaagatc

SEQ ID NO: 12 - DNA, 507 bp, Homo sapiens
  misc_feature (507)..(507): n may be any nucleotide
gtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatn

SEQ ID NO: 13 - DNA, 1442 bp, Homo sapiens
  misc_feature (1)..(1): n may be any nucleotide
nctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtn

SEQ ID NO: 14 - DNA, 759 bp, Homo sapiens
  misc_feature (1)..(1): n may be any nucleotide
nctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatn

SEQ ID NO: 15 - DNA, 2200 bp, Homo sapiens
  misc_feature (1)..(1): n may be any nucleotide
nctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtnctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatn

SEQ ID NO: 16 - protein, 1278 aa, Homo sapiens
  MISC_FEATURE (893)..(893): Xaa may be any amino acid
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGXGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPWDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 17 - DNA, 1103 bp, Homo sapiens
  misc_feature (810)..(810): n may be any nucleotide
ctgcagtgaccactgccccatcattgctggctgaggtggttggggtccatctggctatctgggcagctgttctcttctctcctttctctcctgtttccagacatgcagtatttccagagagaaggggccactctttggcaaagaacctgtctaacttgctatctatggcaggacctttgaagggttcacaggaagcagcacaaattgatactattccaccaagccatcagctccatctcatccatgccctgtctctcctttaggggtccccttgccaacagaatcacagaggaccagcctgaaagtgcagagacagcagctgaggcacagccaagagctctggctgtattaatgacctaagaagtcaccagaaagtcagaaggatgcatagcagaggcccagcaatctcagctaagtcaactccaccagcctttctagttgcccactgtgtgtacagcaccctggtagggaccagagccatgacagggaataagactagactatgcccttgaggagctcacctctgttcagggaaacaggcgtggaaacacaatggtggtaaagaggaaagaggacaataggattgcatgaaggggatggaaagtgcccaggggaggaaatggttacatctgtgtgaggagtttggtgaggaaagactctaagagaaggctctgtctgtctgggtttggaaggatgtgtaggagtcttctagggggcacaggcacactccaggcataggtaaagatctgtaggtgtggcttgttgggatgaatttcaagtattttggaatgaggacagccatagagacangggcaagagagaggcgatttaatagattttatgccaatggctccacttgagtttctgataagaacccagaacccttggactccccagtaacattgattgagttgtttatgatacctcatagaatatgaactcaaaggaggtcagtgagtggtgtgtgtgtgattctttgccaacttccaaggtggagaagcctcttccaactgcaggcagagcacaggtggccctgctactggctgcagctccagccctgcctccttctctagcatataaacaatccaacagcctcactgaat

SEQ ID NO: 18 - DNA, 27196 bp, Homo sapiens
cactgctgtgcagggcaggaaagctccatgcacatagcccagcaaagagcaacacagagctgaaaggaagactcagaggagagagataagtaaggaaagtagtgatggctctcatcccagacttggccatggaaacctggcttctcctggctgtcagcctggtgctcctctatctgtgagtaactgtccaggctcctcttctctgtttccttggacttggggtgctaatcaggcctctctttcccttatctgttttgaagatcaaaaaagatgttcaggccgggcgtggtggcttacacctgtaatcccagcactttgggaggctaaggcaagtggactgcctgaggtcaggagttcaagaccagcctggctaacatggtgaaactctgtctctactaaaaatacaaaaattagctgggcatggtggtgcacgcctgtattcccagctacttgggaggctgaggcaggagaattgcttgaacccggcaggcggaggttgcagtgagctgagatcatgccagtgcacttcagcctgggtgacagagtgaggctgtctcaaaaaaaaaaaaaaaaaaaaaaagatgttcaaggagcagtagcttaagtgttggatgctacaaacatatagaggttattgtagatcttatgcagctctataaaggaataaataagcatcttccccatccatctttagtggcaagaagggttttgggatagcattgattgaggatgatctacttgacaatagtttggacccaaggaggataaggaaggaaagtagtgacggatctcattccaaacttggctgtggaaacctggcttctccttactaaactagaatttggattttacattttcccctttatgttgcagtagaagaggatgaatcctctcactggtgggatcctgccatcctagagcaggtagagagaagagtcactccccactgtgggtagtggaggcttctcacatgtcacatttcacttctacctcaatttcactcttactaagatttgggaatcataatgacaggaaaatagaaaatataaacctcattttaattctttcacagaaaggttagaaattcagtgagttgtggcaacatattttccatcttctgaccttttaacactaattgatatggcttaaattcattctattttaaaccagatttttttggagatagtctatttccaacatgttccttctaggtgacaaatgagggctgttagttcagtatttgttacaataaatgtgtgtaaaataacctcacctttccagaatcatgtcaggaatatgaatctaatgcacaaatgtataactctatgacaagattgcatatatcttttaaaatataccttcccaacgttcattttaatacccctatttcaaacaaacctgcttagcaggttatgttaaacgctcagggcagaggagtaagcaagactgtgagccagtgatgacagcaaaagcatccaggtaggatcaaaatggagtaagaaaatattcctcatccctcagggtagaactccaaagagatattcatgggtcctggccccgtagtggaggtcactcaaaggacaaacatgtttgcatctcatctgcttgaagcctggacacagaggcaccatctgtgtcactctgtgtgtggtctgccatgttgtggggtggtcactacagactcaggcagctgggcagacaataccttagccttagatgatgctgatgcagcccaggagtcagaaactgtagtgcagacaatgccctccttaggccaacacaattaagtgcaatagatgactggcttttctgttagcctcttcattggaaccaaaagcagcattactctaccaaacagaggggagctggaaagaaactacacagtttgcccagcctagcctctgccttgacacggaaccatgtgagtctagacattcacctagatcattccttggggaccaatgctgctgacacattaactcaatagtttgtcctggcctgagaggtcatgtaacttgtagaaagtttagaagcagagattagtgtcatttatttgccatggctgtgacaacaaaggaaggaacaggagtgggaaaacccaaggccaccctggttttggtagatggtgcacacgcttccactaactgttctggggcaaagatccaaatgcactattgggcctggctatgctgcttctgctgggtcccccaaacatgagcctccacgccatttctcagttgtattttaccacatattatcacagtcaccggatttgtacagaatatttggaacctatactgtcttaagggctaccctttaaagaagagaaaacaaggttttaattcaactgtctggaacattttatgtttacttatgtggaatactacatcttttgttataaacaggagggaatgtggacattcgaaggcccctaccttttagctaaaagcccatatgaagcatatggatccatttatacacaccatgcttttcagctacattttcctaatttgcctctctggggccaaccttgtgggactagcagattcatggttgagttgaaggatggtgacctcttccatcagcttttcttcttcctccagtcttccaaccctcagtaacatcagactgggaaggtcttcagacatccagaaaccccagttcggggagttcatacatgacccatcaaagatgagttgcaagcaggcctgccttagggagcaccagccttaatgggttttcctacagagatagttgatgggcagatgcaataaactgactgctttgtgattgaccaccttgagaaaaattagcatgtctggctatgttagtcttttcttgcattgttataaagaaataccagagtctgggtatttataaagaaaagggatttaattgtctcatggttctgcaggctgtacagaaagcatagtgacttcttcctctagggaggtctcaggaaatttacaatcatggcagaaggtgaaggagaagcagacagatcttacatggctggagcagaagcaagagaggctggggagaaggtgccacacattcaaaccaccagatctcataaaaactcattgtcacgaggacaacacctaaggcgggatggtgtgaaacgatgagaaactgcccctaggatccaatcatctcctaccaggccccacctccagtattggggattgcatttcaacatgagattttggtaggggcacagattcagaccatatcactggcactgtgctaatcagatgaatatcaccagttggaaggctagattccacaagaggaggaatgacctggaaattggttctttagttgtgattcttctgcacactgtcattcagggaaatatgagtcaatcatccttcccaataggtcaaatcaaccagatcatctgatcacagagactgaggtgtagctgaaagctgctcacatttctatgaggtcaatggaagccttgagcacagttgtcaatctgtagaaataaggactctgtgactcctccaagacctctctgtgaatgacggtttaagaagaaccagatcctaaaacagggtcagagcttagagggaagggaaagcataaaagcctctgagcaaattctaaagacagggtcaccataggctctcagtgaccctctgtgactgagtggctgcagtgatgcaaaatctcatcatcactgcggaagacaaaaaaatgtcaccctttctacctaggatgagaatccccaaatttggggagaggccacttactaaatagacgtaaaggaacaaagtgacctggaagaattcctgcctgaacctctcaggatcattcacatttgagaacatttatcaaatattcattccaggactgggaccatgaagacttcagctgctttgagctaatcattgtaactttttggtgtctcatggtggaggcaggaaaggacctgatgaacaaatataatcattgccgtcagagttactgttattatttcttgccttaatgttacctcgttctcttgagcattccagttcctcagtcaatgactcatcaacccctatatctataaagtcacaatcgctgtgacttgatttctgtttcactttgtagatatggaacccattcacatggactttttaagaagcttggaattccagggcccacacctctgccttttttgggaaatattttgtcctaccataaggtgagtgtttttgagctcactcttttgcttcttatgattgccaagagcagcttagttccatcagtaaaaatgcttctcctcagggggaaggtctgaagttttacactttcagaaacagtgtgtaggcatcacccagaacatggcaatgtttacccaagggctctcttgctaactctcaggaacctcaggtttgcctcagttgaacagcccaaatctcaggtagatcagcaacctgatgctcagaacttgatgtgcaaactttgtgagcgccataaagaaggttttctttttgctctatgcaggttcccaggaaggtacagtcatacttagttagtattaaaagtaggaaaaggaccccctgattgtgacttgttatctgtgtacatgagagtcaaactttcctctctgtagcaagagctcttgagtagccttttttcccttccttctggacagctttgaaactcaatttattagggagcccaggtatttacctttgggtcatttgcacctgcttcaatcctctccgaaaaatgctaaattccctgagggtatctccatggctctaccccagctcttactccccatgacatcttttgcagctttgagagacagaacaaaggggttctgctttatttcttttccttcaaaggctgcaccttatgggtgagactgatcacagactggatataactgagatgagacagtggtggtcaatcaaactcagatatttaagcacaaaatgagtttgtggggttttttttttacacaaaggcggaatcacatgcaaaatcactagaaaggggatgatttgatgaaattattaaatatttaattttgctgaatagaatataatatgtgccacgtggaccgtgaactttggccgagcctttgtgcctaacctagtctgatgctctgcccagggtctgggccctggatggaaaattaggagcccatgtccacatggccagccgcagcagtcagctctttggcttacatcatctttcccacatacctgaggcttgtttctcagattctaattctctcaggtgagggctttgttgtctaattactatccaggaatttcatattttttccctgtgcaaaagcaataatttccccgccaccttttccaggtcaactctttagtagatgtacccccaagatgcacattcctgggacctttgtttgcacagttaaaatgtcacccctgaaatgtcgatacaggaaggtttgtttttaagtttcagtgaaaactctgagcaagtgttgtaatttgctgtgtccgatgtgtagaggggacattttctcagaacttccatgttaagctggaaaactggaaagtgagttcactttgtcattctgtcactcgttcattttctcactcaacaacatgcctcatacttacctaaatctgctagactaaaggagttccctggtgtctgtaactttccaattctgctagaactctagagcgagctcatgaaataaatgaaaaggatgacaaagagataaaacactgtgcattctcttctgatgctaattcactttcccttggcctcagtttccccatgtgcccctggaggtgatcattcagggattcatgagattttcaagacaacacatgaaaaagcaaaaagacatcagaaagacaaagaggtacttagtatttatacacaaggataagtcattcagtatccacaacacttggagagaattcaagagtgattttaaatttcccttttcaaatacctcctctgttttctcttatttcctttatgacgtctccaaataagcttcctctaactgccagcaagtctgatttcattggcttcgactgttttcatcccaattagaggcagggttaagtacattaaaaataataatcaaatattattttgtttctcctcccagggcttttgtatgtttgacatggaatgtcataaaaagtatggaaaagtgtgggggtgagtattctggaaacttccattggatagacttgtttctatgatgagtttaccccactgcacagaggacagtctcagcccaaagcctcttgggatgaagctcttgtcaacctaactacaaacagagagaagttctctgaaagaagaagatatttatttgggtgtagagtattgcaatgggaatctgcatgcctttataaactatgtgcaaattcagggaagtaaagcaagacaaagaggctccaaggaaaatatgaggaggatttcttatcagttttgaaataattatccttcgctacaaagatcagtaacaagggtgacgcctcaccaaggttggacaggcagttgctgggcaggtgtccttgcagaaatattttttttaatgttgggatggcctttgtgcaagcttgtagttttgcggagtcttttgtgatagttttgttatcaggcacacaagcatgagaatcctctcttcatagccttctttgatttatttgtcagggtttttacacacacacacacacacacacacacacacacaactagtgacatcattttggttctaacaacattcacactggttattgtaaaacttttcgaaggttgtcctaccaaggatcccatgtgtcaccaggtgtcaagttctacagtctgaactaggctgggagcattgtgattacttttctccagactttggtggcccagggactcacagcatcatgctctgtccagtgtctgcctattcccctcttctttttttttttccttaggtgcccttttattacatgtgttgtctcagacccttctaatatgtgctcataaatacatcatatcatctccttcccacatcaattcactttcaattaaaagccaaaactctttcatttagactttggatttaaagtgcttttgaatgaagggttgagagataatagagaaatagattggcaaaccatttatactctgctgttgttgttttttaattttatctgcaagtgtggaacttttcattctgttttgttattaaatttaagccaagactttttaatagaagggtatataagcatttctttgtctataccttcctgctgaatttgaagaaatgctgaatattcttaaccactggcgggctgatggactgtgattttattttattttttatttttagttttttaaattatactttaagttctgggttacatgcatagaatgtgtagttttgttacataggtatacacgtgccatggtggtttgctgcacccatcaacctgtcacctacattaggtatttctcctaatgctatctctcccctagccccccacccaacaacaggccccagtgtgtgatgtttccctccccgtgtccatgtgttctcattgttcaactcctacttaggagtgagaacatgtggtgttgagttttctgatcttgtgatagtttgctgagaatgatggtttccagcttcatccttgtccctgcaaaggacatgaactcattgtttttttatggctgcatagtattccatggggtatacgtggcacattttctttatccagtctgtcactgatggacatttgggttggttccaagtctttggtattgtgaatagttctgcaataaacatatgtgtgcatgtgtctttatcatagaatgatttatgctttgggtatatgcccagtaatgggattgctgggtcaaatggtatttctagttctagatccttgaggaatcaccacactgtcttccacaatggttgaactaatttacactcccaccaacagtgtaaaagtgttcctatttctccacatcctctccagcatctgttgtttcctgactttttaatgatcaccatactacctggcatgagatggtatctcattgtggttttgatttgcatttctctaatgaccagtgatgatgagcattttttcacatgtctgttggctgcatagatgtcttcttttgagaagtgtctgttcatatcctttgcctattttttgatggggttgtttgctttttttcttgtaaatttgtttaagttctttgtagattctggatgttagcccttcgtcagatggatagattgcaaaaattttctcccattctgtaggttgcctgtttgctctgatgatagtttcttttgctgtgtagaagctctttagtttaatcatatcccatttgtcaattttggcttttgttgccattgcttttggtgttatatttatgaagcctttgcccatgcctgtgtcctgaatggtattgcccaggttttcttctaggatttttatggtcctaggtcttacatttaagtctttaatccatcttgagttaatttttgtataaggtgtaaggaaggggtccagtttcaattttctgcatatggctaggcagtttcaccaacaccatttattaaataggaaatcttttccccattgcttttgtgtgtcaggtttgtcaaacatcagatggtagtagatgcatggtgttatttctgaggcctctgttctgttccattgatctatatttctgttttggtacctgtaccatgctgttttggttactgtagccttttagtataatttgaagtcaggtagcgtgatgcctccagttttgttctttttgcttaggattgtcttgtctatgtgggctcttttttggttccatatgaactttaaagtagttttttccaattctatgaggaaagtcagtggtagcttgatggaaatagcattgaatctataaattaccttgggcagtatggccattttcatgatatggagtcttcctacccatgagcatggaatgttcttccatttgtttgtgtcctcttttatttcattgagcagcggtttgtagttctccttgaagaggagaacttcacatcccttgtaagctggattcctagatattttattctctttgtagtaattgtgaatgggagttcactcatgatttggctctctgtttgtctattattggtgtgtaggaatgcttgtgatttttgtaaattgattttgtatcctgagactttcctgaagttgcttatcagcttaaggagttttggggctgagacgatggggttttctaaatatacaatcatgtcatctgcaaacagagacaatttgacttcctcttttcctaattgaatatccatttctttctcttgcctgattgccctattcagaacttccgacactatgttgaataggagtggtgagagaggacatccttgtcttgtgccggttttcaaagggaatgcttctagtttttgcccattcagtatgatattggctgtgggtttgtcataaatagctcttactattttgagatacgttccattgatacctagtttattcagagtttttagcatgaaaggctgttgaattttgtcaaaggccttttctgcatctattgaggtaattatgtggtttttgtcattggttctgtttatgtgatggattacatttattgatttggtattttgaacccagccttgcatcccagggatgatgctgacttgatcctggtggataagctttttgatgttttgctggatttgatttgccagtattttattgaggattttcgcatcgatattcattagggatattggactaaaattctctttttttgttgtgtctctgtcaggctttggtatcaggagatactggcctcataaaatgagttagggaggattccctccttttctattgttcagaaaaatttcagaaggaatgataacagctcctctttgtatctctggtagaattcagctgtgaatccatttggtcctggactttttttggttggtagcctattaataattgcctcaattcaaaacctattattggtctattcagagattcaacttcttcctggtttagtcttgggagggggcatgtgtccaggaatttatctatttcttctagattttctagtttatttgcatagaggtgtttatagtattctctgatggtaatttgcatttctgtgggatcagtggtgatatcctttttatcattatttattgcatctatttgattcttcttttttctttattagtcttgctagcagtctatctattttgttgacctttttcaaaaaaccagctcctggattcactgattttttgaagggtttgttgtgtctctatctccttctgttctgctctgatcttagttatttcttgtctcctgctagcttttgaatttgtttactcttgcttctctagttctgttaattgtgatgttactgtgtcaattttagatctttcctgctttctctagtgggcatttaatgctataaatttccctctacacactgctttaaatgtgtcccagagattctggtacattgtgcctttgttctcattggtttcatagaacatctttatttctgccttcacttccttatttacccagtagtcattcaggagcaagttgttcagtttccatgtagttgtgtgattttgagtcactttcttaatcatgagttgtaatttgatttcactgtggtctgagagacagtttgttgtgatttcttttcttttacatttgctgaggagtatattacttccaattatgtggtcaattttagaataagtgcgatgtggtgctgagaagaatgtatattctgttaatttggggtggagatttctgtagatgtctattaggtcctcttggtccagagctgagttcaagttctgaatatccttgttaattttctgtcttgttgatctgtctaatattgacagtggggtgttaaaatctcccattattattgtgtggaaatcttagtctctttgtaggtctctaagaacttgctttatgaatctgggtgctcctgtatttgggtgcatatatatttaggatatttagctcttcctgttgaattgatcccatcaccattatgtaatgcccatctttgtctcttttgatctttgttggtttaatattctttttaaaataaaatattttaaacatatgaaacattatggagaatggcatgggaaatatccacgtatgcaccacccagcttaacaaatgctctactgtcatttctaaccatggtctctttgaagagctcttttgtctttcaatgtctcttccttgtttggcccacattatccttcatcatatgaagacttgggtggctcctgtgtcagactcttgctgtgtgtcatacctaatgaactagaacctaagattactgtgtattgtacaactaagggattatgtaaagtcaggatcaaagtctggcttcctgggttgggctccagctgtagaataaggctgttgatgtttaatcaactctgtttttttcacacagcttttatgatggtcaacagcctgtgctggctatcacagatcctgacatgatcaaaacagtgctagtgaaagaatgttattctgtcttcacaaaccggagggtaagcattcatgtgttgaaattaaaatactgattgattaaatttatattttgaaattcttatatattcatagacagttgcctaaaaaatgtccaggaaggttccacgtccacttcatcctgtcccccccgaatggtaacatcttgcaatcttgcataactataaatacagtatattcatgttactatatagtacaggaaattgacattgatacagttcacagagcttattcagatttcaccagctttatgtgccctcatttctgttctatgcaaatttatcacaatcatagatttgtgcaatgaccagcatgatcaaaacgcagaacccatttgtgttctatacaaatttatcacatcattagtagatttgtgcaatgaccagcatgatcaagatgcagatccactctgtctccatgtggctccctcctgctatcctacagtcacaagtctttttctctgacagggtccatatcagagcaaactattttttattttgaggtgatcaatgtattaatatttccttttctggattgtacttttggtgccatgtttgaaagttctttgcctagccctccttcctatattgctgtttttttctaaaagtcataaagtttaatgctttactaaatgtagctctattatcaattttgtgttattttttgtataaggtatgagatttagatcaaggttcattttttttgtggcttatggctgtccaattcctccaacgccatttttggaaagatgggtatattgttaaaaatcaactgggcatacttctttgcatctctcacgtactactgtgtcccattgatcactgtgtttattattccaccaataccacactgtgttgaccctagtagctgtacactaactcttaacctcgtgtagagtgattcttcccacttttattgacttattttttcagatttgttttaactcttcctggtcctctgcttttccataaaaattcagaatgagtttctcagtgtctacaaataaacgtgctgttattttgacaagaattgaattaaatatatagaccagtttatggaaaatgtgtatctttactgtttgatctttcaattcatgaacatagtatgcctctccatttccttagattttctttgattgcttttaacatcttgtagttttcagcatagagatcctgtacatgttttgttagatttacagctaaattatttcatttttgttggaatgactgtaaatgatattatgtttttcttgtattttcagatattgattgttgttacatagaaatgtgctttgattttgtgtgttgatctggtatcctagaaccttgcagaactgacatagtagttctagaagtctctttgtatatgccttgagattttacacattgtcagttatgtcacttgaaaatagagacaattcttttatttcctttccaatctgtatgccttttatttcttttcttttgtctattgcactaggacttcccagtataatgttgagtaaatgtggtgaatttttgaattgttcctaatcttaggagggaagccttcagttctttcatcattaagcatcatttagatgaagtgggctgtttttttgtagattctctttatcaaattgaggaattttctctccctagtttgctgagtttttatcataaatgaatgctggaagtactcattataaaaaaaatggctatggaagatgaaagaaagtttcaagcatgttggcttgataggccagttccaagttggcaaaaataattatctcttttttctttctatccatgaaataaaaaattaagagacaagaatgtttatggaattgcattatttcttcaaaatatgttcctagttttaaaggtattacctactattttttttaaaaccatcacattgaggcacttctttttcacgttgcccatgctgcaggagaacataaagacagcttgtctgaggcaacatacaatccaccaaagtcacctgcttgtctgtctccactccgtctctacactgcagaagtgctaggtcttgattctgtttattgtactggaagaacacattctctaccacgtggataatttgcatgtaaaagaagactgggatatagaggctggagacgacatcagggtctcctgaacactgccgccacccccccgccccgccccacacaaatacatcccaggacactgggcatctgggataaatctctattgagcatctagcatagggcccatcacccagtagacagtcactaaatagttgttgaataagtgttcctgtttaacacattttctacaaccatggagacctccacaactgatgtaggacaaaatgtttctgctttgaactctagccttttggtccagtgggatttatgaaaagtgccatctctatagctgaggatgaagaatggaagagattacgatcattgctgtctccaaccttcaccagtggaaaactcaaggaggtatgaaaataacatgagttttaataagaaacttaaagaatgaatctggtggggacaggtataaaataagatcacagtccctttccaaggggtagtccactgaatttgagctgcctaaaaatggtcttttatctttatgtacagaaaacacatcacaaaattcattataaaatgtcacttactgctccatgctggggaaagccatgtccttctgggactagagtctgcacatttaactatgggtggtgttgtgttttgtgcttagatggtccctatcattgcccagtatggagatgtgttggtgagaaatctgaggcgggaagcagagacaggcaagcctgtcaccttgaaagagtaagtagaagcgcagccatggggttctgagctgtcatgaacccctccagctgcctgccatggagctgatattcctgctgttgggttattccagtgaccagacaaaaggagggctgtggtaatgcaacttcaatgggtctcccaagatggggcagctccgatgaggaggtggggcagctggaggaaaaggatcttctcccctgtgcacaggggccagggtttacatatccattaaattgtcaccttggatattctagaagactaaatatatcctttagggggaaaaagtgtgattgtaccaaagttttaagcatggagtgtatgggatggtggaaggggaaggcacttggtatctgttggttggcagtgagtaggttgggagagttataatggagaacttagaataactttgatcatttcatgtttttttctgaggatatcagtagaatactaaatattaaaattcctaccatttctttttcctccagtctcaaagagagagggtggtaaaaacactataggtagggcaagcctattatttgctatctacacttatgcagtaaaaacaggtgtaatctgagtttgtcctgggcagaccagggatatgtggtcactcactatagaaatttccaaatcaaattttgagagatttttttttaaccaggacattattggtcattatattttacaaaaataattctgctgtcagggcaacctcagctcaccacagctggggatagtggaattttccaaagcttgagcagggagtatagagaataaggatgatatttctaggagctcagaacagggtactgttgctttgtaaagtgctgaagaggaatcggctctgggcatagagtctgcagtcaggcaatatcacctgtcttgagccccttaggaagagttaattattctactcttgttctgctgaagcacagtgcttacccatcttgtatcatccacaatcaatacatgctactgtagttgtctgatagtgggtctctgtcttcctatgatgggctccttgatctcagaggtaggtctaattcagttcagtgtctccatcacacccagcgtagggccagctgcatcactggcacctgataacaccttctgatggagtgtgatagaaggtgatctagtagatctgaaagtctgtggctgtttgtctgtcttgactggacatgtgggtttcctgttgcatgcatagaggaaggatggtaaaaaggtgctgattttaattttccacatctttctccactcagcgtctttggggcctacagcatggatgtgatcactagcacatcatttggagtgaacatcgactctctcaacaatccacaagacccctttgtggaaaacaccaagaagcttttaagatttgattttttggatccattctttctctcaataagtatgtggactactatttccttttatttatctttctctcttaaaaataactgctttattgagatataaatcaccatgtaattcatccacttaaaatatacagttcagtgatttgtagtacatttgaagatatgtgtgaccatcatcattttaaactttaaaactttttttgtcaatctagagacctcatacatttttagctatcagccccctgtcacaaaccctgtcatcatatgcaaccactaatcaactttctgcctctatggatttgcctattctggacacttcatagaaatgatattaattcatcagggttttttattctctagttcatgaatttgtactttagtctgtatcattttctttcttctgctggcttcaggcttagtttgcccttcttcgtttactatgttgtggcatgaacatagattactgatttgtgatttttttgttcctctaaatttagacattacagctgtaactttccctctgagcacttcctttgctaaatcccatgagattgtggcctatcacatcttagtttttgttcacctcaaaacagtttctatttgcccttttggtttctactttgactcattggttacttaaatgtttattatttaacttccacatatgtgtgagtttctcaattttctttcccttattgattttatctttattccatgataggtgacagagatatgctgtgttatttctatcttgactacctactatttcttgaacagcaagattaattttgagcttcagattatgatttgggttattctaggagactgtagtccaatagataaaggcaaagagattagggcattgaattttgttccttttatccttcaaaagatgcacaaggggctgctgatctcactgctgtagcggtgctccttatgcatagacctgcccttgctcagccactggcctgaaagaggggcaaaagtcatagaaggaatggcttccagttgagaaccttgatgtcttttactcttctggttggtagagaaaactagaattgctccaggtaaattttgcacattcacaatgaatttctttttctgtttttgttttgtttttcctacagcagtctttccattcctcatcccaattcttgaagtattaaatatctgtgtgtttccaagagaagttacaaattttttaagaaaatctgtaaaaaggatgaaagaaagtcgcctcgaagatacacaaaaggtaaaatgtggtggtagttataggaggatgtttagtttttcataattttttagataatatacatatgatcagtgcagttacctgtatgtttttaaagaatgcttttaacatgaagactgctcatgtttagagcaagagaattcatttggtagaaatgcagaaagtggggtttggggaaggagatatgagaatgagtcagagacagcacatgaaatttgatatcagacacaacaattagtatgccacggcataaattttattgagataaaactttaccactttacttcccttcaataaattgtcagaggataaacattactgtttggaaatatattttactgatattatgctttccccagatcataaattggtaaagactcatttcaagtacaaacctgttattttacacattctcaaatgaaagtccctatcaggccacctgctgaagtgtagcgtgtgttaaatttgagccatctcacatgatagccagatttgtttcaggaaaacatcctgctttccaaggatttagaagggtatgtttttcactggtgattcaggcaacatgcatcagatttctggtcttcaaggagccatattctcagaagggagatcaaggaccacgcttgtgatttacttctgacttcaggagccactttctgtcagtgaaatttctctttttgcttctagcaccgagtggatttccttcagctgatgattgactctcagaattcaaaagaaactgagtcccacaaaggtaaccagagtgtttctgagggctacttgtggggcactcagagggaaggccttgttctgaaaatgtgcaggaagtattccaggatgatgagaatttctgccacatagcagaacgacacatgtttgaatgttataagtggtagttggaggcactttctagaggcatgcaggcatagatagccatgttctaagagtaaagggcaaccctaagcaaacctggcatgctagaaagtcagtctgcggtctgtggatcacctacatcagatcaaatgccaattctcagcctccttcagatccactgaataaaaatttctgcctagaaatttattaggttgctgcaaaattaattgtgggtttttccattacttttaattgcaaaaaaatgcaattaaaagtaatggcaaaaaccacaataacttttgcagctacctaatacatctaacatccaatcaggagccacgctgttccaaaaggggtgattttaactggcagtacttgttgaaagtgtgttcaccaggttaatctactgcaaagttattttccactttggaatggattagtaacttgtggggaaaacctctgagaccgtgtaaatatcctctttgtcttcaatgtttcacctacgagttttacaacccatgtatgttttttactgaagtcactattactatgacagtggcaaaatgatgaccatggtcaattacaagccaccaagacttggcaaccatctcacaaaattcctgaatatttaactattggttctagagagcaggactgggcttactccagcatactgctttaaatatatccatgtctacatccacttttgtctgtatgtctatgtatctatctatgtatctatctagctatgtatctatctatctatctatcatctatctatctatcatctatccatctatcatctatcatttatccatctatcatctatctacttatatattatctatctatcatctaactattcatctatctatcccaatataacttgctgtgataaaggaaatagtctattgttttactgtttcatatagaaatcactagacacatatggctattgagcactggatatgtggctagtgccattgaagatcaattttaaatggtatttcagtttaatttaataaaatttgattttaaatagccactagtgggtagtggctaccatattggacagcagagctctaaactttgagattatagttcaatttcacatcagtatcatcaggttcatgataactgaatactatgattaggtagttgaatttacttataactgcatcacagaagtcttcactggtaaatcacagctctgtcgacctttctcacactcctttcatattggttttggttgtgaattacatggttggagcaggcattatatttatttctatgttccaggtctctaaaggtcctaatccagtcctgatcaaacagaccagtgatggaccatcctgagcttctctcaggagaaaaatcaagaggggcccaacttgtaatcataggagcttatgctattttaatgccatccatcagactacaatcaattaccactcatctagctttttgtccatctctcattcttgtacatcctgagatagtcaattctgagaactgtagcctagatctatcacctgatgcctctcaaagatataatccgtgcttctcaagctaggctatgcacacaaatcactgcatcttgtgaaagttcagattttgaatcagtagttcaagggtggggtttgagattttgcatttctaatgagctctcagatgcttctgacccatggaccacactttgaataccaagaagtggtctgtagaccaatattggtcccttaagttccctcaaacatatcttcgggaaacgtcctttgattttccctacatttaaccattagtgttgcaaattctctcaaagtttgtcaagatatattgtagctaaaataaattacatttttcttgggggagagtactacctcatattaacttacaataaagtacttttaggatcattcaaggaacacacccataacactgagtatgttatgcggaaatgctctctctggaaattacacagctgtgcaggtggcgggggtggcatgaggaggagtggatggcccacattctcgaagaccttggggaaaactggattaaaatgatttgccttattctggttctgtaagatacacatcagaatgaaaccacccccagtgtacctctgaattgcttttctattcttttcccttagggatttgagggcttcacttagatttctcttcatctaaactgtgatgccctacattgatctgatttacctaaaatgtctttcctctcctttcagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggtgagtggatggtacatggagaaggagggaggaggtgaaaccttagcaaaaatgcctcctcaccacttcccaggagaatttttataaaaagcataatcactgattctttcactgactctatgtaggaaggctctgaaaagaaaaagaaagaaacatagcaaatggttgctactggcagaagcgtaagatctttgtaaaacgtgctggctctggttcatctgctttctattactacaataatgctaagtaaaaaacctccaaaaacctcagtggcatctaacaataagcatttgttgctcacactcatttcacattggttttggttgtgaattacatgtttgcagcaggcaccatagtggtgtgtgatgtccccttagctgtatccacatatggacacaggaatttgctctttttatctctttttattttcttggttacagacatgtgacttttttttttgaaaggtaacaatcactttctcatatgttatttgatgctagtggtcatagcctattagtcacatttgtttcaatgagaaagaaaaaccagtacacggttatgctaaggatttcagtccctggggtgagagccgtctcgaatgtctccccacttcataactcctccacacatcatagttggatagtgagctctgctgatattggcaggacttgctctggtctggctgtagtctgacggagcctggccctgggtgtgctgtgcaggctgactcagctctccccacacctatctcatgttccagtcaggcagtaactggtgaagaagccaagctaggaaccaggatatctggctcctgagctaaagtcttaaaacactatcatattgccttccaaatataacaccaaatactaggtgcatatcacccacactgttttcagacctctgccaaaattgggattctttgtggtatgaagagacacggctttggggctggcccggctgtggcagtgaggtgaacacaaagggatgttcttcagagattacagtccagccctgaagcaacaactaggagactgtttcagcaagtgaggacagggctgtgtggggttctatcctctttataacttccactagcactgaaatcgtgttctctggttacatccaaccagaaccttctttgtcatttttgggatagaaagggactagtttatcctcaaattatttatggagattttatataatatagtgtttctctccacattttctgtatataacaaaagtcctccttttagtgtgtgtatacacatatatatacacatatatatgtgtgtgtgtatgtgtgtgtgtatatatatatacacatatatacacggttatgctaaggatttcagtccctggggtgagagccttcccgaatgcttcccaccttcataactcctccacacatctcagtgggccactgagcacagcaatgggcatgacagttattaaaacactttataaatgcttcgatcctttaccagtatgagttagtctctggagctcctaatacttcattagtactgcatggactgagttaaaagttaattcaaaatctcaatttatccaaatctgtttcgttctttccaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaaggtacaaggcccctgggaagggagccctccctgaaccagcctggttcaagcatattctgcctctcttaatctacaggacagtcatgtggttgtataattatttgcttgtatttttatatttagagatttttttaatcatcaaattgattattgtcacactttacaaaccatagactagaaaaaagaaaactacagtcatccacaattccaacaacttacgatgaaggtcatcagttatgtccttatgggtcatcagtgtccaaaatgtaaggactcttttaaaaacacatgatcacaatgctattattatgtcccccaaatgaatatttttttcataaatataatcaaatatttaggaataacattttaataaaaaacatgcatctaatcttcaaagaattttatagcttactggaacagatagacaaagaaagcagtgatgatactgcattacatcggtacagtagcatcatatgcctgtgtaaattatctgacttcaactattctatggaggtgtgggggagaaagagggagagatggagagaagaagaaggaggagaaggagggagaagacaaggtaaggaggagaaggaggagaattagaaaaacaagagaggagatgagaaggaaagtgcaaaataacaattttgaaatagtacaagacaatttccccttctccttcctcatgaccaatgtaagtgtgacttgaggcaggaacctacttttccatcagtcagtcccatcacttatgtgccttttatagtgtggacacatcaccaccctgaatataatttcagtgtttagaaataagtattctttgcaacactatttatctcatctcaacaagactgaaagctcctatagtgtcaggagagtagaaaggatctgtagcttacaattctcatagcaaaataagcatagcaggatttcaatgaccagcccacaaaagtatcctgtgtactactagttgaggggtggcccctaagtaagaaaccctaacatgtaactcttaggggtattatgtcattaactttttaaaaatctaccaacgtggaaccagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacaggttagtcaattttctataaaaataatgttgtattaataattcttttaactgagtggtctgtattttttaaaaagaatatgcttgtttaatcttttactaatttgttctctgggccaaagaatcaattaggcccatctgtgatctttaagggtgcttcagttctggagttcaaaagctgtagcatcaaaaacatcatgtaaagcccatgtagattagcatggcataattatctgcagtctccttgaacttgagcaaagtttacattcagtttcaagtcgattggaaagatggggaagtattttgcacagtcatgaagtgtaatgattaccttgttgtgacttttgaatgctgctcttcccaaccagaacttggagaagctttctcatgagagtggctcccaaccactagctgtacattggaatcaccagggagctttaaaaattcatgatgtctgggatatcacagaaattctaaactaatttgcccagagtgtggctttaaaagcttccccattgcttctcatgtgaagccaaggttgagaatgactaatttaaggcatttctggtggatataaaggactaccacagtccaaggccatcctgactgacctcaccttccaggtgcctagctccatccagctgggctccttttcaacccaattataactctattaatgttgttcccagccaggcatggtggctcatgcctgtaatcccagcactttgggaggccgaagcaggcggatcatgaggtcaggagatcgagaccatcctggctaacacggtgaaaccccgtctctactaaaaatacaaaaaattaccctggagaggtggcaggcacctgtagtcccagctactctggaggctgaggcaggagaatggcatgaaccccagggagcggagcctgcagtgagccgagattgggccactgcactccagcctgggtgacagcgagactctgtctcaaaaaaaaaaaaatgttgttccctttctcctcattttgttcttatctttcaagtcctagttcaatccccaagcccctccaaagtgtcttctcctcctagtccagggcccatttacttctctgctctgttattggatactggaggctattatcataaatttgacaatttgccattaaatcattgagttttattctctatattttctttgtatctaaaatgtcttcccccctccattaacaatatcctctcattttattcctttttaaaatatcccagtggtgccttgcaagggactgtatctaatgcaagcatttggtaaatgtttaaggagtgatgtgcagttgatggcttgcatacatatattaagctatttaatgtgaacctttaaacaaatgccattcgtgcatatgcatgtgtgtgtgtgtgtgtgcacatgtgggcatgcatgtctgtctgcagtgaaaatatattcagggtttttgaaaatttttaaataataaggtattatatttatagaaagatttgaaatattttctctgaagaagttaaagaacagacgtcattgattcatattaaacaataccctataaatcttatttctaggtctcatgtatttattattcaattccaccccttaagtaggctttctatataggagaggaagaagacagaaatagtttccatattatttccatattccatattatttgtggctttaggccagcagtgtagctgtattatatgtgcccagacaggggactcagccctgaataaaagtggtcctctggcacacctgggatggggaaggtactccttggtaagctcccaacctggcacttcttgatctccctggcaattttcttgcccattactccatggagatcagaatatcactctgttgtgtcccctcaacacggaaggagtgtctcaataagaatggggctaaaagttgagtccaaacactgtaggaattgagaggttccccacttgcactacccttggaagccaagagaagatgttaaaaaataaaatggtaatgcttcctgaaggtgtcttcccatctttacactggatgggttcaattgggaggaattactggactctggaagttgaagactgtccatataattaaaatgtacaataactacccaggtttaccttgcaagtttcaacatacacaaaattaactttatatgactcttcaaaaacagtttgccatcatacctaataatctggtttaaattttaaaaactcatccattttacttaaaatttaaatcaaaaaagaacacaggtttccatgaatttgtctcaggcctggcacagaatagtactccataaatattttgttaaatgatagatgatgaatgctctcactgtccaatcttcacacatcttatagactaagtataaagaatccaagatttatagtgctgaaagtagtttttatatgtttacaaagcattattgtcattactgcattttttttgcccattactccatagagatcagaatatcactctgttgtgtcccctcaacactgaaggagtgtctcactcactttgatgctatactttctacttttgtttatttaatgcttctcaatatgcttgtttaactgttgcagatccccctgaaattaagcttaggaggacttcttcaaccagaaaaacccgttgttctaaaggttgagtcaagggatggcaccgtaagtggagcctgaattttcctaaggacttctgctttgctcttcaagaaatctgtgcctgagaacaccagagacctcaaattactttgtgaatagaactctgaaatgaagatgggcttcatccaatggactgcataaataaccggggattctgtacatgcattgagctctctcattgtctgtgtagagtgttatacttgggaatataaaggaggtgaccaaatcagtgtgaggaggtagatttggctcctctgcttctcacgggactatttccaccacccccagttagcaccattaactcctcctgagctctgataagagaatcaacatttctcaataatttcctccacaaattattaatgaaaataagaattattttgatggctctaacaatgacatttatatcacatgttttctctggagtattctataagttttatgttaaatcaataaagaccactttacaaaagtattatcagatgctttcctgcacattaaggagaaatctatagaactgaatgagaaccaacaagtaaatatttttggtcattgtaatcactgttggcgtggggcctttgtcagaactagaatttgattattaacataggtgaaagttaatccactgtgactttgcccattgtttagaaagaatattcatagtttaattatgccttttttgatcaggcacagtggctcacgcctgtaatcctagcagtttgggaggctgagccgggtggatcgcctgaggtcaggagttcaagacaagcctggcctacatggttgaaaccccatctctactaaaaatacacaaattagctaggcatggtggactcgcctgtaatctcactacacaggaggctgaggcaggagaatcacttgaacctgggaggcggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatatttctgggagacagaaaacatgtttccctacacctcttgcattccatcctcaacacccaactgtctcgatgcaatgaacacttaataaaaaacagtcgattggtc

SEQ ID NO: 19 - DNA, 2059 bp, Homo sapiens
  CDS (91)..(1602)
gcaggaaagctccatgcacatagcccagcaaagagcaacacagagctgaaaggaagactcagaggagagagataagtaaggaaagtagtgatggctctcatcccagacttggccatggaaacctggcttctcctggctgtcagcctggtgctcctctatctatatggaacccattcacatggactttttaagaagcttggaattccagggcccacacctctgccttttttgggaaatattttgtcctaccataagggcttttgtatgtttgacatggaatgtcataaaaagtatggaaaagtgtggggcttttatgatggtcaacagcctgtgctggctatcacagatcctgacatgatcaaaacagtgctagtgaaagaatgttattctgtcttcacaaaccggaggccttttggtccagtgggatttatgaaaagtgccatctctatagctgaggatgaagaatggaagagattacgatcattgctgtctccaaccttcaccagtggaaaactcaaggagatggtccctatcattgcccagtatggagatgtgttggtgagaaatctgaggcgggaagcagagacaggcaagcctgtcaccttgaaagacgtctttggggcctacagcatggatgtgatcactagcacatcatttggagtgaacatcgactctctcaacaatccacaagacccctttgtggaaaacaccaagaagcttttaagatttgattttttggatccattctttctctcaataacagtctttccattcctcatcccaattcttgaagtattaaatatctgtgtgtttccaagagaagttacaaattttttaagaaaatctgtaaaaaggatgaaagaaagtcgcctcgaagatacacaaaagcaccgagtggatttccttcagctgatgattgactctcagaattcaaaagaaactgagtcccacaaagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacagatccccctgaaattaagcttaggaggacttcttcaaccagaaaaacccgttgttctaaaggttgagtcaagggatggcaccgtaagtggagcctgaattttcctaaggacttctgctttgctcttcaagaaatctgtgcctgagaacaccagagacctcaaattactttgtgaatagaactctgaaatgaagatgggcttcatccaatggactgcataaataaccggggattctgtacatgcattgagctctctcattgtctgtgtagagtgttatacttgggaatataaaggaggtgaccaaatcagtgtgaggaggtagatttggctcctttgcttctcacgggactatttccaccacccccagttagcaccattaactcctcctgagctctgataagagaatcaacatttctcaataatttcctccacaaattattaatgaaaataagaattattttgatggctctaacaatgacatttatatcacatgttttctctggagtattctataagttttatgttaaatcaataaagaccactttacaaaagta

SEQ ID NO: 20 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 21 - protein, 503 aa, Homo sapiens; identical to SEQ ID NO: 20

SEQ ID NO: 22 - DNA, 35769 bp, Homo sapiens
  misc_feature (7087)..(7087): n may be any nucleotide
ctgctgtgcagggcagggaagctccaggcaaacagcccagcaaacagcagcactcagctaaaaggaagactcacagaacacagttgaagaaggaaagtggcgatggacctcatcccaaatttggcggtggaaacctggcttctcctggctgtcagcctggtgctcctctatctgtgagtaactgtccaaactcctctctttgtttccttggacttggggtgctaatcgggccccttttcccttatctgttttgaagatcaaaagagatgttcaaggagaagtagctgaagtgttggacgctacaaacgcatagaagttattattatcttatgcagatctatgaatgaataaataagcatttctcccatccaccttctaattttggtgactaggagggtttagggacagcatttggtagtgggaatgatttgattagcttagatctgacgaagactaatcaatgaaaacatggcagcggcagattacaaactgctgatcatgatggacagtgtgatcctcatccccttcccaggctctggggattctgggtacaggaaggagtggcttgcatttttgtctcattaattcgctttctgggttctgtgtctgctggaagggatgtgtagctgtattgcccctgtagacctggttcctgctcccccgccttccaacccaggatatcatttacataacgcaccaggggacaccaagacttcatgggaagctgtcccctggctcttccctctttcctgtgccatgcccctgaaaatcccctccctcctatgagtcactcctccaccctgtcatacacaggatggtttatcttgcaatgattaacctctagagcaaaggagacctggaggaagtttcgaggatttattctttgctttaatctttttcctcccgtctctgggaggctaggattaatatagagctttgtttctcacctaatgggaatctactagcagcctgaaaaggcaggagccatgaaagccaatttggattttacatatttttcccctttatgttacagtacaggagggcaaaccctctcactggtgggattcctggcatcctagagcaggtggagagaagagttactttccactgtgggtagtggaggctccacctgtcccattaacttctacctcaatttgacttttattaagagcagggaaccacaatgacatgaaaatagacactataaacctcattttaattctttcacagaaagcttaggaattcagtgagttgtggcaacatggtttccattgtctaacatttttaaatgaattgatatggtttaaattcattcatttttaaaccagaattttttggagatagactatttccagcatgttccttctggatggtaaaacagggctgttagttcagtatttgtgacaataagtgtgtgtaaaataatgtcacctttcctgaatgtcaggaatatgagtctaatgcacaaatgtatacctctaagacaagactgcacgtcttttcaaatatacctgtccggccatttattttaataactccttttcgaatatacctgcttagcagattgtcttaaactctcaggacaggggagtaagcaagactgtgagccagtgacgatagcaaaggcttccaggtaggatccatatgaagtgagaaaatattcctcagctctcagggtagaactccaaagagatattcatgggtcctggccccaccgtggaggtcactcaaagggcaaacaggttggcatctcatctgcttcaagcctggacacaggggcaccatctgtgtcactctgtgtgtggtctgccatgttgtgggccggtcactacagactcgggcagccaggcagacaatgccttagccttagacaatgctggtgcagcccaggagtcagaaaatgcagtgtagaccaggccctccttaggccaacacaattacatgcaatagatgactggcttttctgttagtctcttcactggacccaaaggctgcattactctaccagaggggagctggaaagaaactaaagagttcgcccagcacagcatctgccttgacatggtaccatgtgaatctagacactcaccaagatctttccttgggggccaatgctgctgacacattaactcaatagcttgtcctcacctgagaggtcaggtaatgtgtttaaagttcaggagcagagattagtgtcattgatttgacatggctgtgacaacaaaggagggaactgaagtgggaatacccaaggccaccctggctttggcaggtggtgcacgcacttccactaactgttctggggcagggaaccaaatgtatgactgggcctgctcatgctgcccctgctgagtcctccaaaccctgcccttcatgtaatttctcagttttattttatcacattttataagtcactggatgtttacaaaatgtttggaacctatactgccttgaaggctaacctctaaagaggagtaaacaaggtcttaatacaactctccgggacgttttatcattacttatcttatatgccatactgcaccatttgctatcaacaggaaagtacctggactttggaaggtccctctgtgtcttttagctgaaagtacatatgaggcatgtggattcttttatgcacatcatctttttcagccacatttttgtagtttgcctctctggagccaactgtgtggggctagcagcttcacagctgaatcagtgtctggcaacctcttccttcagcctctcttcttcctccagttttccatccctcagtcacaccggagggggaaggtctgcaaggatccagaaccatcagttggaggagtttgcacatgactcatgaaagatgagttccaggcaggcctgccatagtgaacaccaggcttaatgggtttttcctcagagatacttcacgtacagaggcagtgaactgactgctttctggttgaccaccttgaaaaagatgagtgtgcctggcactgtgcttctcaggtgagtatgacctgagaagtattagttgctggttcttctgcacacaatcattcaaggacatatggatcaaccatcctcctcaacagctcaaatcaaccagatcatctgaccacagagactgaggtgtacctgaaagctgcccacatttctataaggccaatagaagccatgaacacagttgtcaatctgtagaaataaggactccatgactcctccaaggcctctctgtgaatgaacgtttaagaagggctagatcctaaaacagggtcagagcttagagggaagaaaaagcataaacatttctgagcaaattgtaagggcagtgtcaccataggctcccagtgaccctctgtgattgagtgcatacagtgatgcaaaatctcatcatcagtgcaaaagacaaaaaaaatcttactctttctacctaggatgagagtccccaaatcagcgaagagtccacttactaaacagacataaggaaatgaagtgtcctggaagaattcctgcctgaacctctcaggagcatttgaggacatttatcaagtattcactccaggattgggactatgaagacttcagctgctttcagctaatcattgagacttttcaggggtctcagaatagtcaggaaaggacctgatgagtgaatgcaattactgatgttggagttgctgttattatttatcgtgtacatattacctccctctcttgaccattccagttcctgagtaactcaccagccctctgatctataaagtcacaatccctgtgacctgatttctgtttcactttgtagatatgggacccgtacacatggactttttaagagactgggaattccagggcccacacctctgcctttgttgggaaatgttttgtcctatcgtcaggtgagttgcttgagcttcctcttttgcttcttatggttgcaaacatcagcttagttccatcagtaaaaatgcccctccttgggagggagttctgaggtttcacattttcagaaatggtgggactgggtgcagtggatcatgcctgtaatctcagcctctgtgaggccaagactggcaaattgcttgagcccaggagtttgagaacagcctgggcaacacagtgagacacctgtctctagaaagaaaaaattacctgtgcatgatatggtagcccatgcctgtagtcccagctactctgaatgttaaggtgggaggattgtatgaacccaggaagtcaaggctgtattgagctgtgatcgcaccactgcactccagcttggtcaacagaacaagacagaaaggaagaaagaaagagagagagagaaagaaagagagaggaaggagaggggaggggaggggagaggagtggggaggagaggagaggagaggagagaaaaggagaggagagaggagaggagaggaaaaggtgtgtaggctccacccaaagcatggccaggtttacccctggagggaaagtcacaagctcatgtccagaaggccagtagcagcaagctgctctccagcccagatttcctatcctgtgtacctggagcttgtttctcagattctaactctcacaactgaagcctctgttgtctgattactatctgagaattctacacaattttaccctcgataaaagcagtaatttcttcttcatctttcccagatcaactcttgtagtagatcaacatttctgggaccttcttttgcatggttaaaacatcacagctgaatcttagcaacaggaaggtttgtttttatgtttcagaagtgaaagctcagagcacgcattgtaatttgctgggtgtgatgtgtagaggtggcatttctccatcttttctgtgttaagctagaaaactggaaaggaagtctactttctcattcactcactcactttctcactcaacaacatgccttagacttatctaaatctgcaagactaaaagaggttcctggtttctttaactttctaattctgctagagttctagagagagcacatgagataaatgaaaaggatactgatggaggagattaaaaaattgtgcattccctgcagacactcacttttcctcacctcagtttcacccctgcccttgcaggtgatcattcacggggttaggagactttagagagaataaaagaaaaagcaaaaatacatcagaaagacaaggaattacttactggtcatagacaagggtgagtccttcagtacttagagaaaattcaagagtgactttaaattccccacttcaaatatattctctgttttcttgtctttcccttaagacatctctgaatagcttccttcaactgccagtgaaagatagcaggcctgatttcattggacgcaactgttttcagccccaattagaggtagggtttattctatttaaaataataatcaacttgtattttgtttcctctcccagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggataattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaataacaagggtgatgccacaccaaggttggacaggcagttgctggacaggtgtccttgcagaaatatttttgtgtaaagttgaaatagcctttgtgcaaagttgtggtttttgtagacacttttgtaatagttttgtttccaggaacacaagcataagaatcctctcttcatagccttcttgggatttatttgtcagggttaaaaaacaattagtgacatcactttggttctgataaagttcacactcgctattgtaaaacttttcgaggcttgtcctaccaaggatcccatgtgtcaccaggtatcgaggtcttcagtctgaactaggctaggagcattgtggttaccacttttctgcaggttttggtggcccagggactcccagcatcgccttctgtccagtgtctgcctattcccctcttctttttttcttccttaggtgcccttttatcacatgcattgtctcagacccttctaatatgtgctcataaatgcatggcatcatctccttcccacattgattcactttcaattaaaagccaaaactccttcatttagactgaatttaacatgtgcttttgaaagaagggttgagagataatagagaaacagattgggaaaccacttatgctccacttttttaaactttctctgcaagtatggaattttttgttctgctttgttgtttaaatttaagccaaaacttcttaatagaaggatatacaaatatttattggtttataccattgcacttactttgaagaagagatgctgaatattattaaaccattgtgttccctggtgggctgatggactgtgattttataaggtggtctcagccaattgcagcagctgttccctgtcagaggggctagaggtttggtgagagcagtggatgaggtgcagtggtgtgtttgttcactagaagcaagtgggagaaagctttgcctctttgtacttcttcatcttctcccctcaagtcctcagaatccacagcgctgactgtggagtgctgtggagctggcatggcccatacaggcaacatgacttagtagacagatgacacgctctagatgtccatgggccccacaccaactgcccttgcagcatttagtccttgtgagcacttgatgatttacctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcantatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctgtaatcccagcactttgggaggccgaggcaggtggatcacgaggtcaggagttcgagatcagcctgaccaacatggtgaaaccccatctctactaaaaatacaaaaattagctgcgtgtggtggcatgcgcctgtagtcccagctactcagtagtctgagacaggagaatcgcttgaacctgggaggcggaggttgcagtgagccgagatcacgccattatactccagtctgggcaacccaatgagactccatctcaaacaacaacaacaacaacaacaacaaaaaccggcaaactgcaataacttttgcaccaacctaatactatagtacaggaaattgactttgatatagtttacagagcttttcagatttcaccagttttacatgcccttgtttgtgtgtgtttatgtgtgtgggtagttctaagcaatttttcacattcgtagatttgtgcaacgaccagcaccatcaagatgcagacccattccgtcaccatgtggctccctcctgctgtcctacagtcacaacatggagtttgtctttttctctgacaggttctatatcagagcaaacttttatttatttgaggaggccaatgtattaatatttccttttatggattgttcttttggtgttaagtctgaaaatcctttgcttagccctccttcctacattgctttttctaagagttatatagtttaacactttacaaaatgtaactctattacccattttgtgttaatatttgcataagttatgagatttagatcaaggttcattttctgtggactatggctgtccaaatgttccaacaccattttggaaaggtaggcatattgtcaaaactcagctgagtatattttgtgaatctatttcttattgtttactcctccactaataccacactgtggtgactctagtagctgtacagtaactcttaacatcatatagggcaattctttccactttattgatttatattttcagaatggctttagcttttcttgtcccttgcctttccataaaaattcagaataagcttgtaagtgtctacaaacaaacctgccataattttgataagaattaaagcagaggtgtccaatcttttggcttccctgggccacagtggaagaagaagtgtcgtgggccacacataaaatacacacacacacacacacacacacacacacacacacacacacacacaaatggtctgtgtatagttttcattatatatctaccaccacagataagcaaaaatgtccttgcataataatcctaattatgcactgccccattcagagggtctttcaaaatcattgaacaggttccaagtttgcaatcactgatacagaaaatgtacatatctagctaaacttcactacttttttgatattttttattataaaagaaaagagaacaacataaaactagtggggtacttgacattgtttttgagaaactaatccatcagtatctggcttgatggaagtagttgcaattctcagtgagttctcaaggtgctcatcagatattttggttctaattttactcttcgtgttcttcatccttgaaaatagtagctcacaaatgtaagtgctgccaaaaagcaatgacatgaacaaggtgtgattgtgaagcaagggatatttgtcattgggaagacaggtcttacaaaagtccagtaaagaggcaaaatcaaatttttctataagttgaacatcagattgcagctctaggcattccatttcaaaattgccaggtaacatatatatgtcgactgaaaatggagttgcaaatataccaaaatattgatgattttttcagaaatcttgaaatacctgttttcaaattcctgtatcaaattgaaaagcaaggctgcgtatttttggctgttcacaggaccatgtttagccaacatgtcgaaatgcataaaattgtttgccttaatttgagcttgccataatttcagtttcatatggaatgctgttatggtttgaaacattgtattgttaagttggttttcaacttgaagacacaggtttaactcacttaaatgggccgtcaaacccactaaaaatgctaaatctgtaagccagttttcattgtcaagttctggcaccaattttgtttgataccataaacagcttgatttcacatcacaaagcataaaatctttacattttgccttgacttaaccatcttacttctaaaaagtgaatgacttgctagagtcagcatccatacttttaaggaattcctgaaactagcgatgattcaattcctgggcccttgtgaaatttacagccttgatgacaatttgcatgacgttatctacttttaaagcttgtgcacatggattttcttgatgtattatgcaataatacttcatcaaatgtgagttttgtgtggcaactgcatcatctattaattgtacaagtccctctcttttacctaccatcgccagggcagcatctgtagctatatcacatatgtttacaaaggacaaagaaaattgctttaacatatttttcactgcttcatataaatctcttgatttagttgtgtcttttaatagcatggtgacatttcgatttcttcagtgacattatattcatcatcaatacctctaataaaaatagcaagttgtgccgtatctgtagtgtcagtgccttcatccatcaccaaagcataaaattttaaattagcagttttactctccaaacgtctttcaatagatttcccaatttctccaattctcctggctatagtctggtgagacaaactgattttagaaatatcagtttctcaaggcaaataatatctaccacatcttccagacattgcttaataaactcaccatcagtaaatggttttgatttttttgctattaaatttgctaccacataactaggttttaccttacgattgagtccgagttgtaactttttaaaaaatcttttttgttgaaaagacagactttttttcagttctgctattttgtccttacaacacatacacaccaaatttgtcagcacgtttttgcatataatgcctcttcaaattgtagtctttgaaaactggcacaaattccgtggaaattaagcagagtgcttcgctatttgcctcaacaagaaaaagtcatttgtccacttttcattgaacaatcttccttcatccataatttttgttttttagggttttctttttaagacattgtggaagccattctggaattaaaagcattataatagataagcaactatatttacttttattatggaaattaacagataggaaaatagaacagaaagcaaggtttaataatcaaataagaatacttacatgtcttctaaataatattaaacacctatcatctacaaaggtaggttgaaatattattgataattgctgggttttacttgccaaattgccacaaacacacctaatacctgacagtgtcaattcaactgtccgtgattagaagataacacactggaagtcgcacaccaccataaaactgaagccacacatgcgtacaaatggcgacagtgtctggtgtacagcagcgctctgccttgtccagaatacacacttgaattctttgtcacaattcacttcacgtggcactgcaatagcgtcctctcgctctttgttagttaattttaatggcttttaatttcttcttgctgaactgtttgcaattataatgcaaattatggatactagtccattatttgtggatgtgacatactctgattacccctttccattccattgttgtctacgaagttcacacttgagaatcacatagtcaaattacaaaattacaaaaaaaattgcaaaaaaactcaaaatgttttaagaaagtttccacatttgtattgggatacattcaaagccatcctggactgcatgaggcctgcaggccacaagttggacaagcttgaattaaaccaatagaacaatttgggtataatctatatctttactatgttcagcctttcatcccgtgaatatagtatgcctctccatttctttagcttttattactttcctcaacattttatagttttcagcatagaggtcctgtacatcttttgttagatttacaccagaaatatttcatttttgttggagtaactgtaaatgatactgtttttcttgtattttcagatattgattattgttacatagaaatgtgaataattttgtttgttgatcttgtatcctatagccttgcagaacttacctattcgttctagaaatttttttgtatattccttgacattttatacattgacaattatgtcacctgaaaatagagacaattctattatttcctttccaatctgtatgccttttatttctttttcttgtctagtgtattaagacatcaggtatgctctttagtaagaatgttgagagtgggcattttttagttcttcttgatcttggaaaaaccattcagtccttcatcattaaatgtgatttaactgaatgatttttttacagattgtctttatcaaatgaaggaactgtctctctcttcctagtttattgagattttatcatgacagctggaagtacacattttaaaacaaaacatagttgtggaagataagagaaagttccaagcatgctggcttgatagtccagccccaagttgggaaaagtaattatccctttctttttccttctatttatggaataaaaaattaagagaaaagaattttcaaggaaattgcattattccttcaaaacaggtttctagtctttaagtattacctacttttcaaaaaaaaatcaccacatcatggcatccctttttcaagttgcccatgctgtaggtgtattaaagacagagctggtctgaggcaacatacagtctgcccatctgtcaccaatccttttctactctgcacactcctggggaagggctaggtcttgttcctgtctattccactggaagaacagttccctaccacgtggagcatttgcaattaaaaggagactgagatatagaggcaggagaccacaccagatggctgggtctccccactcccacccccgccccacatacactcagaagaggctaggcatctaggatctccattgagcatcttgaatatggcttgccataatatcatatacagtcaataaatatttgttaaataaggatgcctcttcaatatattttgtgcaaccatgaagatcaccacaactaatgtgagaaaaaatgtttctgttgaactctagtctttaggcccagtgggatttatgaaaagtgccatctctttagctgaggatgaagaatggaagagaatacggtcattgctgtctccaaccttcaccagcggaaaactcaaggaggtatgaaaataagatgagtcttaattagaaatgtaaagaatgaatctggggacaggtagaaagtaagatcacagtccgtttccaaggggtagtccactgagttcgagcttcctaaaaatggtcttttatctttatgtacagaaaagacatcacaaaattcattacaaaatgtcacttactgctccatgctggagaaagccatatccttctgggacttgagtctgcacatttaactacaggtactgatctgttttgtgcttagatgttccccatcattgcccagtatggagatgtattggtgagaaacttgaggcgggaagcagagaaaggcaagcctgtcaccttgaaagagtaagtaggagcacagccatggggttctgagctgtcatgagcccttccagctgcctgccatggagtcgacagtcgcactgttgggttactccagtgaccagacaaaagcagggcagcgctgcaactccaaagagccacctaagagggagtggctcccatgaggcggcaagtcagcaagggaaaagggccttctctcctgtgcacaggagccaggatttacttatctgttaacttgtcaccataaatattctgggagattaaatacatactttagaaattaaaaaaacatgattgtatcaaagttttgagtgtagtggatatggaactgtgggtaagcaagcatttggtacttgttgccttgcattgggtaagatgggaaagttacaatggggaacttggaacaatttcaatcccttcatggtttttctgagaatatcagcaaactatgaactattaaaccttcccactacttccttttcctccaatctcaaaaaagaaagggtgctagaaatgctatgtgtagagcaagcctattatttgctgtctacaatggtatgtgcttcaattatgcaggaacgacaggtgtaatctgagcctgtcctgttcagacttgggacatgtggtcactcagttttgggttctccaaatcaatgttggagagatctattttttttaaccagaacattcttgattgtcacatcttacaaaaatgactctgctctcagcgcaacttcaggtcagaggagctggggatagtggggttttccagagcattagcagggagtgtagagaataaaggatgatatttctaggaactcagaacagggtgttactgttttgtaaagtgttgaagaggaattggctctgggcatagagtctgtagtcagacaacgccacctttcttgaatccactaggaagagttaattattctactcttgttctgctgaagcacagagcttacatatcttatatcatccacactcaacacatgctactgtagttgtctgataatgggtctctgtcttcctatgactgggctccttgacctcagaggtgagtctaactcagcttggtgtctccatcacccccagcatagggccagctccatcactggcaccagataaccaccttctgagggagtagatggaagatgattcagcagatagttctgaaagtctgtggctctttatgtgtcttgactggatatgtgggtttcttgctgcatgtatagtggaaggacggtaagaggtgctgattttaattttccatatctttctccactcagcatctttggggcctacagcatggatgtgattactggcacatcatttggagtgaacatcgactctctcaacaatccacaagacccctttgtggagagcactaagaagttcctaaaatttggtttcttagatccattatttctctcaataagtatgtgggctattatttctttctctctttttaaaaataactgctttcttgacatataattcacatatcgtataattcatccacttaaaaggtacaattccattgtttttaagataatcaaaaatatgtatgaccattactattgtaaactaaaatgtttttgtcaatctagagccctcacacactttagctgtcaacaccccaccacaaaccccactgccctaagcatccaataatcaactttctgcctctatagatttgcctattctggacacttcatagaaataatatcattgatttttctctgttgttttttattctctatttcatgagtttattttagtctgttattttctttcttttgctggctttaggtttcatttgctcttcttcttttagtgttttgtggtgtaaataattataatcaatttgagatattttcttcttttaaatttagatattacagctataaatttccctctgagcactggtttggctacatcctgtgttttggtacatcatgccttctttttgttcatctcaaaacaatttcttgttgcccttttgatttctgctttgactcactggtcacttaaaactgtattgtttaacttccacaaatgtatgagtttcccaaatttctttcccttattgatttctagttttattccatggaagttgatgtacatatgctgtgttaattctatcttgactatcatttcctgaacagcatgattaagttaagcagcagattatggtctacattaatccaaaaactctagtccaatagataaaggctaagaggtcagggaatttaattctattactttggtcactccaaagactcagaaggtgccattgatctcactgctgtagtggtgtttcctatgtatagacctgcccttgctcagtcgccggcctgaaagaagggcaaacatgataaaaggaatgggttccagttgagaatcatgatgttcttattcttattactggtagagaaaattataattgctccaggtaaagtttgcattttcaatgatttccttttgtttgttttgtttttcccacagtactctttccattccttaccccagtttttgaagcattaaatgtctctctgtttccaaaagataccataaattttttaagtaaatctgtaaacagaatgaagaaaagtcgcctcaacgacaaacaaaaggtaaaatctgatggtggttaaatgacgatgtttaggttttgataaatttagattttatacacatgatagagcatgtatctgtatttttaaaaataaagacagagaacttatgtttagaacaagagaagccatttggtagaaataaagaaggagattggggaaggagatgagaatgagtcagagagatagcatttaaaacttgaaatcaggcacaacaattagtatgtcatgatataaacagtattgagataaaattttaccacttctcttccctttaataaattgtcaaaggataaagtttcctgtttgaaaatatattttactggtattgtgctttcctcatatcacagattggtaaagaatcattttaagtccaagactcttattttacatattctgcaattaaaggtcctatgaggctacctgccgactgctgacatgtagtgtgtggtaaatgtgagtgtttcacagcctggagtgaacaggggtcttctctgagaattgaggttgcaaggctggctaactcagctttgccttcacgagccctagaggccagccgaaggatgtctgcaggtcagggagacaggaccaggtaacccagctgtcactgaagattatatagagtttgagaatgttggaatatttgaaaatgctcccccaaaaaagctgctgatgagttctggaaatgtcaggagattaatctatacggacactgctgaagaaaaaggtagaagaataaaagatccagtacttcttcctgggtaagcagttatgaccagagatggaaccggcaactctttggccagaaagctgtatccaaaagacagagaagatgagaaacagggagggcaaaggcgaaaaagcaattggacatgatagctagatttgtttcaggaaaacatcctgctttccaaggatttagatgaatgtttttgttcactggtgactcaggtaacacgtcttcaagaagccatagggaggttgagggagggaagtcaagaagggaggttgaggactgcacttttgatttacttctgacttcacgagtcactttctgccaaagaaatctctccttttgcttctagcaccgactagatttccttcagctgatgattgactcccagaattcgaaagaaactgagtcccacaaaggtaaccaaggagtgcttctgagggctactggcggggacactaagagggagggccttgttctgaaaatgtgcaggaagtattccaggaagatgagaatttttgccacatagcagaacaacacacatttagatgttataaatggtagctggaggcactttccagaagcccacaggtatagccatgttccaggctgaaagggcaaccctaagcaaacctagaatgcttggaggacagtcagtggtttgtggatcacctacatgagatcaaatgccagttctcagcctcctccagatccaccaagtgagaacctctacttggaaatttatatcaaacataccgatcaggaagcacactatcccagtaagggtgattttaactggcagtacttgaaagtgtgttcgcaaggttaatctactgcaaagttttatttttccctttgaaatgcataagtaactaatgggggacacctctgataccatgtaaatctacttcaatcttcagtcttgtatctactagttttatgacccatggatggttttaaccaaaaccattattactaagacagtggcaaaatgataaccatggtcaatttcaagctaccaagatttggcaaccatctcacaaaatttttgaatatttaacaattggttctagagagcaggactcagcagactccagtataccactttaaacatgtccatgtctacatctacttctgtctgtctatctatctgtcaatcatctatctgcctataatttatcaattaatcatctatctatctcaacaaaacttgctgtgataaagaaaatagtctatcatttcactgtttcatatagaaatcactagacacatatggctattgagtactggacatgtggccaatgccactgaagaacaatttttaagagtatttatttttaattgaataaaatttgaatttaaatagccacatgtggatagtggctaccagattggacagcagagctcccaactttaaaattacagttcaatttcaactcagtataatggggttcaatgtaactgagtaaaataattggatggttgaatttacccacagcagcatacagaaatattcactgataaatcagaactctgtagacctttctcacactcattttatattgtgtttggttgtgagttacatgattgctgcaggcaccatatttatttctgtgctccaggtctctaaaggtcctaatccagtcctgaccaaacagactagtgatggaccatcgtgagcttctctcaggagaaatatcaagagggaggccaacctgtaatcataagaacttctgctattttaatgccattcatcagactacagtcaatcaccatgcttctggctttttgtctatctctgctgtcttgtacatcctgagatagtccattctgagaactgtaccctagatcttgtattgcctgatgcctgtcaaagatgtaatccatgctgcttaagtgaggttgtgcacacaaatcaccatatctcctgcaagtttggattttgattcagtagttcgatggtggggtttgagattctgcatttctaataagctcccagatgtggctggtgctgctggtccatgaaacacactttgagtagcaagaggtgatctgtagctcagtattggtcctttaagttccctcaaacatatatagagaaaaggtcctaaatattgcaaattctctcaaagtttgtcaagctatattggaattctctcaaagtctgtcaagctctattgtagaaaatcaaatttttattgggaaaaagcctaccccatatttacttacagataaagtacttttaggatcattcaaggcacacacccataacactgagtatgtaagacagaaatgctctctctggaaattacagcagtgctggtgctgggatgccatgatgaggagtgtgtggcccacaatcatgtagaccttgggaaaacctggattaaaatgattttgcgtcatcctggccctgtataagatacatatcagaatgaaaaccactcccagtgtgactttgaattgcttttccattttttcttcttgggattagagagcttcacttagatttcatctaagctgtgatgttgtacgttgacctgatttacctaaaatgtctttcctctcctttcagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaataatgctaagtaaaaaaaaaaaaaaaaaaaaaaaaaaaaaaaggagtgtggcgagaagatggccaaacaggaacagctccagtctacagctcccagcgtgagcaacacagaagacgaatgatttctgcatttccaactgaggtaccgggtgcatctcaatggggattgttggagagtgggtgcaggacagtgggtgcagtgcacccagcctgagccaaagcagggcgaggcatcacctcacctgggaagtgcaaggggtcagggaattccctttcctaggggtgacggacagcacctggaaaatcaggtcactcccaccctaatactgcgcttttctgatggtcttagcaaacggcacaccaggagattatatcccgcgcatggctcggagggtcctacgcccatggagcctcgctcattgctagcacagcagtctgagatcgaactgcaaggcagcagcaaggctgggggaggggcgcccgccattgctaaggcttgagtaggtaaacaaagctgccaggaagctcaaactgggtgaagcccaccgcagctcaaggaggtctgcctgcctctgtagactccacctctaggggcagagcatagccaaccaaaaggcagcagaaacctctgcagacttaaatgtccctgtctgacagctttgaagagagtagtggttctcccagcacacagctggagatctgagaacagacagactgcctcctcaagtgggtccctgacccccgagcagcctaactgggaggcaccccccagtaggggcagactgacacctcacacggccgggtactcctctgagacaaaacttccagaggaatgatcaggcagcagcatttgcgggtcaccaataccgctgttctgcagcctccactcctgatacccaggcaaacagggtctggagtggacctccggcaaactccaacagacctgcagctgaggatcctgactgtcagaaggaaaactaacaaacagaaaggacatccacaccaaaacccatctgtacatcaccatcatcaaagatcaaaggtagataaaaacacaaagatgggggaaaaacagcagaaaaactgaaaaatctaaaaatcagagcacctctcctcctccaaaggaacgcagctccgcaccagcaacggaaagctggatggagaatgactttgacgagttgagagaagaaggcttcagacgatcaaactactccgagctaaaggaggaagttcgaacccatggcaaagaagttaaaaaccttgaaaaaagattagacaaatggctaactagaataatcaatgcagagaagtccttaaaggacctgatggagctgaagaccatggcacgagaactacgtgatgaatgcacaagcctcagtagccaattcaatcaactggaagaaagggtatcagtgatggaagatcaaatgaatgaaatgaagaaagaagagaagtttagaagaaaaagaataaaaagaaaggaacaaagcctccaagaaatatgggactatgtgaaaagaccaaatctacgtctgattggtgtacctgaaagtgacggggagaatagaacgaagttggaaaacactctgcaggatattatccaggagaacttccccaatctagcaaggcaggccaacattcaaattcaggaaatacagagaacgccacaaagatactcctcgagaagagcaactccaagacacataattgtcagattcaccaaagttgaaatgaaggaaaaaatgttaagggcagccagagagaaaggtcgggttacccacaaacacaaacccatcagactaacagtggatctctcggcagaaactctacaagccagtagagagtgggggccaatattcaacattcttaaagaaaagaattttcaacccagaatttcatttccagccaaactaagcttcataagtgaaggagaaataaaatactttacagacaagcaaatgctgagagattttgtcaccaccaggcctgccctaaaagagctcttgaaggaagcactaaacatggaaaggaacaactggtaccagccactgcaaaaacatgccaaattgtaaagaccatcgaggctaaggagaaactgcatcaactaacgagcaaaataatcagctaacatcataatgacaggatcaaattcacatataaaaatattaaccttaaatgtaaacgggctaaatgctccaattaaaagacacagactggcaaactggatagagtcaagacccatcggtgtgctgtattcaggaaacccatctcacgtgcaaagtaacacataggctcaaaataaagggatggaggaagatctaccaagcaaatggacaacaaaaaaaggcaggggttgcaatcctactctctgataaaacaggctttaaaccaacaaagatcaaaagagacaaagaaggccattacataatggtaaagggatcaattcaacaagaagagctaactatcctaaatatatatgcacccaatacaggagcacccagattcatgaagcaagtctttagagacttacaaagagagttagactcccacacaataataatggaagactttaacaccacactgtcaacactagacagatcaacaggacagaaagttaagaaggatatccaggaattgaactcagctctgcacaaagtggacataatagacatctacagaactctccaccccaaatcaacagaatatacattcttttcagcaccacaccacacctattccaaaattaaccacatagttggaagtaaagcactcctcagcaaatgtaaaagaacagacattataacaaactgtctctcagaccacagtgcaatcaaactagaactcaggattcagaaactcactcaaaaccgctcaactacatggaaactgaacaacctgctcctgaatgactactgggtacataacgaaatgaaggcagaaataaagatgttctttgaaaccaacaagaacaaagacacaacataccagaatctctgggccacattcaaagcaatgtgtagagggaaatttatagcactaaatgcctacaagagaaagcaggaaagatctaacattgacaccctaacatcacaatgaaaagaactagagaagcaggagcaaacacattcaaaagatagcagaaggcaagaaataactaagatcagagcagaactgaaggaaacagagacacaaaaaaacccttcaaaaaaatcaatgaatccaggagctggttttttgaaaagatcaacaaaattgatagaatgctagcaagactaataaagaagaaaagagagaagaatcaaatagatgcaataaaaatgataaaggggatatcaccacccatcccacagaaatacaaactaccatcagagaatactataaacacctctatgcaaataaactagaaaatctagaagaaatggataaattcctcgacacatacactctcccaagactaaaccaggaagaagttgaaactctgaatagaccaataacaggttctgaaattgaggcaataattaatagcttaccaaccaaaaaaagtccaggaccagatggattcaccgccgaattctaccagaggtacaaggaggacctggtaccattctttctgaaactattccaatcaatagaaaaagagggaatcctccctaactcattttatgaggccagcatcatcctgataccaaagcctggcagagacacaaccaaaaaagagaattttagaccaatatccctgatgaacagtgatacaaaaatcctcaataaaatactggcaaaccgaatccagcagcacatcaaaaagcttatccaccatgatcaagtgggcttcatccctgggatgcaaggctggttcaacatacgcaaatcaataaacataatccagcatataaacagaaccaacgacaaaacccacatgattatctcaatagatgcagaaaaggcctttaacaaaattcaacagcccttcatgctaaaaactctgaataaattaggtattgatggaacctatctcaaaataataagagcaaatttatgacaaacccacagccaatatcatactgaatggacaaaaactggaatcattccctttgaaaactggcacaagacagggatgccctctctcaccactcctattcaacatagtgttggaagttctggccagggcaatcaggcaagagaaagaaataaagggtattcaattaggaaaagaggaagtcaaattgtccctgtttgcagatgacatgattgtatatctagaaaaccccatcgtctcagcccaaaatctccttaagctgataaacaacttcagcaaagtatcaggatacaaaatcaatgtgcaaaaatcacaaatattcttatacaccaataacagacaaacagagagccaaatcatgagtgaactcccattcacaattgcttcaaagacaataaaatacctaggaattcaacttacaagggatgtgaaggacctcttcaaggagaattacaaaccactgctcaatgaaataaaagaagatacaaacaaatggaacaacattccatgctcatgggtaggaagaatcaatatcatgaaaatggccatactgcccaaggtaatttatagattcagtgccatcgccatcaagctaccaatgactttcttcacagaactggaaaaaactactttaaagttcatatggaaccaaaaaagagcccgcattgccaagtcaatcctaagccaaaagaacaaagccggaggcatcatgctacctgacttcaaactatactacaaggctacagtaaccaaaacagcatggtactggtaccaaaacagagatattgatcaatggagcagaacagagccctgagaaagaatgccacatatctacaaccatctgatctttgacaaacctgacaaaaacaagcagtggggaaaggattccctatttaataaatggtgctgggaaaactggctagccatatatagaaagctgaaactggatcccttccttacaccttatacaaaaattaattcaagatggattaaagacttacatgttagacctaaaaccataaaaaccctagaagaaaacctaggcaatatcattcaatacagaggcatgggcaaggacttcatgtctaaaacaccaaaagcaatggcaacaaaagccaaaattgacaaatgggatctaatgaaactaaagagcttctgcacagcaaaagaaactaccatcagagtgaacaggcaaccgacagaatgggagaaaatttttgcaacctactcatctgacaaagggctaatatccagaatctacaatgatctcaaacaaatttacaagaaaaaaacacaaccccatcaacaagtgggggaaggatatgaacagacacttctcaaaagacatttatgcagccaatagacacatgaaaaaatgttcatcatcactggccatcaaagaaatgcaaatcaaaaccacaatgagataccatctcacgccagttagaatggcgatcattaaaaagtcaggaaacaacaggtgctggagaggatgtggagaaaacaggaacacttttacactgttggtgggactgtaaactagttcaaccattgtggaagtcagtgtggtgattcctcagggatctagaactagaaataccatttgacccagccatcccattactgggtatatacccaaaggattataaatcatcctgctataaacacacatgcacacttatgtttattgcagcactattcacaatagcaaagacttggaaccaacccaaatgtccaataatgatagactggattaagaaaatgtggcacatatacaccatggaatgctatgcagccataaaaaatgatgagttcatgtcctttgtagagacatggatgaagctggaaaccatcattctcagcaaactatggcaaggacaaaaaaccaaacactgtatgttctcactcgtaggtgggaattgaacaatgagaacacatggacacaggaaggggaatatcacacactggggcctgttttggggtgggaggagtggggagggatagcattaggagatataccgaatgttaaatgacgagttaatgggtgcagcacaccaacatggcataggtatacatatgtaacaaacctgcacgttgtgtacatgtaccctaaaacttaaagtataaaaaaaaaaattcaaaaacctcagtggcatctaatgagaagcatttattgctcacaagactggatagtgagttctgctgatactgactggactcactctggtctggctatggtctgaggtagcctggccctgggggcgcgatggaggctgactcagctctccccacacctgtctcatgttccagtcaggtagccactggccaagaagccaagctaggaaccagggtatctgactcctgagctaaactctaaccctctacaatactgcctcccaaatataacaccaagtgctaggtacatatcatccacagttttcagacttctgcccaaactgggattctttttagtgtgaagagacctggcctgtggggctgaccctggtgtggctgtgaggcagacacaaagggacatttacatccagtcctgaagattacagtccagccctgaagcaacaactaggaaactattccaaaaggaggggatggggctgagtgtggggttctattctcttcataactttaactagaactcaaattgtgtaccttggtagcatccaatcataaatttattttgtcgtatttgtgatagaaaggaacaagtttatccacaaatttatttatttatttatttatttatttatttatttgagacagggtctgactctacgacccaagctggagggcagtggtgcaatctcagctcactgcaaactctgcctcccaggctcaagccatcctcccgcctctgtctcctgagtagctggaactacaggcacacgccaccacacccagctagtttttgtattttttgtagagatgggttttcaccatgtttcccaagctggtctcaaactcctcaaaagagttaccaagcaggactctgcaaccaataatccttgtgtgaagaggatatttgctcttttccctgtttttctttcttggtacagatgtgtgacctctttttgaaaggtgatagtgactttggtgtattttatttggtggtaatggtcatagccccattaatcacatttcttcccatgagaaagaaaaaccactacatggtcatgctaaggatttcagtccctggggtgaggatggtcttgaatatctcctacattcataactcctccacacatctcagtaggtcactgagcacatcaatggacatgccagttattaaaatacttcacgaatactatgatcatttaccagtatgagttattctctggagcttctaatacttcaatagtactgcatggactcagttgagagttaattcaaaatctcagattatccaattctgtttctttccttccaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggtacaagtctccagggaaatggagctcaccctgacccaggctggttcaagcatattctgcctctcttaatctacatgacaatcgtgtggttgtacaatcatttgcttgtaagtctttttatcacaaaaaagtgataattatcaaactttacaaaccacagactagaaaaaacgaaactacatccatccacagtcccagcacaagacaaagataatcaattatgtccctgtgggcatttttctacgcctatatagatttttaaaaattagaatggtatcactttttatttggtttgaattgctgcttacttgatttaacaggaaactatccactgacctatattactataaatatacatatatatgtatatatataaatatatatatatgtatatattgcatatgccataaaccatttaaccatgatgttatttcaggtgtataggctttttattcctttctgttttttctatgctgtgccctttagctctctgaatttaacagaaactttaaaacatgcttccacattccatttgctttcaacgttacttgctatttcctctgtagtaattataagagtgcaggctgaggtcctgagaagtcctcatccctaatggtttaagccacttcactgaagacacaagacagcacaggtcctcctggtcctatctgtggctgcagtcctgtgccagctcccttatactctcagtagacatctcacacactcctccttggaggtgtcttgagcatgctcttctgggaattcagggacaaggtcaggccttaggcacagttcgcactctggatatagttggtgttttcccattactgtattattaagcaaaatttagaatgaaatttttagggtactggctggtgattcaggatgcttgggatctagactttcattagcccctacctgcaagtttgctgatgggaggaaccttgtcttgttggtcatggtgtccctagtgctagcatggagtctgcacataatacttgttcacagagtaagtcagagctgaccaagttctctgttttctggagtagaggacttctatgtttcctgcaagctcagcacttccacctcctgtggctgcactaatacgaaatcagagaccactcgctgtacttcactttgaatcactcagtcaccaaaaagatagtgcttgccatgtgtcaggaacttggctaggcagggagaaattcatatgatttatataaatccataaatccatatgatttacataaatccataaattcatgtgatatatacgtatatgtgtgtgtatatatatattagagaatgtttgacatatacacaagtacatgttaccgacaccagcctatagaatagttttcgtgcatctccatatatctatcactggttccaacagccatcaatccatgttagctgccccatccaaatgccaccatcaccctcctcctgactatcatgttattttgaagcaatagcctgtaaatatttcagaatgctctccaaaatataaagactcctgtaaaaacatatgacaacaatgccattattactttctttgaatcaacattttttccttaatataatcaaatatttagaaatcaaatttgaataaaacatgggtcaatcttcaaagaatttatagcttaatggaacagatcaaggaaagcagggatgacactacagtagggtagcatcatatgcccatgtaacttatgtgacttaaactatcctgtaagggtgtgggggagaaagagaggaagagatggagagaagaaaaaggaagagaaggaggaggagaaggaggcagaggagaaggtggacggggaaggtagagaggaggaggaggggaattagaaaaaaagagatgacaggagaaggaaagggaaaaataacaacttgaaatagcacaagacgttttctccttctcctttctcaatgagcatgtgaccaacacaagtgtgagttgaggcaggaatccacttttccatccatcagtcttatcatttatgtgccttttatagtgtgaacacatcaccaccctgaatataattttagtgtttagagataaatattatttgcaacaatattcatctcatctcaagaaacgctcctatagggtatggagaatttaaaggacctgtaggttatgatgattataacgaaataaccaaagcaggatttcaatgaccagcccacaaaagtatcctgtgtactactggttgggaggtggaggggggttgttcttaagtaagaacccctaacatgtaactctgtggtttttatgtttcattaactatttaatctaccaatatggaactaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacaggtcagtacactttctgtatgttttattaagaatttttttaactgaagggtatatattttttaaaagaatatgcatgtttatcttttaataattcattctatgggccaaagaacctacttggatccatctttgatcattaaggatgcttcagttctggacttcaaaacctgtagcattaagaacatcatgtaaagtccacacagattagcatgacatgattatgtgtagtctctttgaacctgagtaagtttaaattcagtttcaagtcaattggaaagaagtgttttgcacaatcatgaagtgcaatgattacctggctgtgacttaaatggtgttctccatcaccagaacctgcagaagctctctcatgacagtggttctcaaccactagctgtatattggaatcaccagggagcttcaaaaattcatgatgcctgtgacatctcagaaattctaaactaattaacccagagcgtgactaggttctgtcatgctgtcgggtgaacccctgattagttctcacgtgaagccaaggtggagaatgactaatttcaggcatttctggtggatatgaaggactaccatagagcagggctatccttactccttgaccttatgttccaggtgatacatttaaagaaagatttagaatcttttctctgaagaagttaaagaacagatgtcattgattcatattaagcaatagcctataagtcttatttccaggaccggtgtatttaatatgcaactctaccccttaagtacactttgtgcttgggagaggaggaggatggagatggttgccatcttatctatggcttcagggcagctgtgtagctttcctatgtgtgtattcaggcagggggctcagccctgagagaaagtgggcctctggcacacctgggacagggaagatattccctggcaagctctcaggcatctcaggctggcacttctttgtatccatggcaatttgctttcccctcactgaactgagatcagaatgttactctgttggtggctcccccaacagtgaaggggtgactcagtgacaatagtgctagaagtatgagtcaaaacactgtacaacttgagaaattccccgtttgcactacgcttggaagccaagaggagatgttaaaaagaaaagaataattctttctgaagacatttcccatcattgcacttgatgggttcaactgggaagggttactagactctggaagttgaaaactgcccacataattaaactgtacaacagctactcaggattaccttgcaagttttaacctataaaaatttaactttatatagcacttccaaaatagtttgccataatacctactaatctggatttaatttttaaaactcatccttttaacttaagatttaaataaaaaaaaaaaaacacgagtccacaagaatttgtctcaggcctggcacagagtcagtgctccataaatattttgttaaacgatggatggtgagtgcttttactatccagtatttacccagcttatagattaagtatgaagagttcaagatacatggtgttaagagtcgtttttatatgcttgcaaagcatttttgtcatattttttctactttgcttccatcttttcttctttcacttcatttattaattctccatatgcttgtttaactattgtagatccccttgaaattagacacgcaaggacttcttcaaccagaaaaacccattgttctaaaggtggattcaagagatggaaccctaagtggagaatgagttattctaaggatttctactttggtcttcaagaaagctgtgccccagaacaccagagatttcaacttagtcaataaaaccttgaaataaagatgggcttaatctaatgtactgcatgagtagttggtgattttgtacattcattgagctctcccagagtctgtgtagagtgttgtgcattatgtagtataaaggaggtgaccaggtaagtgacagataggtagactcagcttctctgcttctcataggactacctctacccacctctagttagcattatcaactcctcctgagctctcatcagagaataaatatttctcaacaatttgatccataacttttaagaaaaataagaattatcatgatgactctaatagtgacatttatatcacgttttatttgtaatattctataagttttatattaagcgaagtgataaaatcccctttacaaaaatattatctgatgccatcctgcacactaaagagaaatctatagaactgaatgactgaaaaccagcaaataaacattttttatcattgtaatcactgttggtgtggggcctttgtcagaattccaatttgattattaacataggtgagagttaatctgctgtgactttgcccattgtttggagaaaatattcatagtttcattctgccttctttgaagaacatattttttgtaacactcaacgaagcacttatcatattattagttatgatttattatttttaccacatctcccctgacatttctggaacacaggaaacatgttttcttatacgtcttgcattccatcttcacctcccaattgtcttaatgcaatgaacactgaataaaaaattgtcaattcgtcagttgattgggcagcatgtctaaaagcactattcattttccttttttattctttcattttccctccttttctgaatactaaagccattaggtgggttgcagccatgtggtagccacacattaaggtggacaagagagtcatggtggctccaagtcagattccaagtgtgctggggaaggcatccacatggaggggcagcctgacctggaagcgggagcccaagcaatcagagaaggggtccacacagaggtgtggccttcaagagcagccagagcctaaatagggcctggagaacccacgtgaggtgaggagggtatccctgagtgggaagggatgggtgagagttggctacatagaagggattgatcacataagtaaataaagtatactggaagctaggtgtgtcacttttgcagaaaagagtcatagattcagaaagggagaaagctagcattaatcctatggtgttagattggaatggatgtatcagtgtacattcatacttttctagatagatagatggatagatagacagatgatagatagataatagatagttgatagataattagatgtaaatatatgtgtttgtatgtgtgagcatgcatgtgtgtgtgtgtgtgtgtgtgtgtgtgtgtgtgtgtgtgtataaaacatatattccctacttcactgatagggctagataacaatgacatttcaatagcaatgagtatacttagtgcctagatcttggcttatgaatatcattttccactgaagggaaccagagctcgttagagaaatagctgatgccagggctaggactaaaaatgttcaagatgagcccaggacactttttgtgccaggaagtaagaaaacactcaaatggtttctaaatggtatttggaaatgatttctaaatgatatttccatatgacttccaaatgatattttaaaaacctaaagactccaccaaagaactattagaactgataaacaaattcagtaaatttgcaggatacaaaatcaacgtacaaaattggtagcacttctatatgccagcaaggaacaatctgaaataaaatcaatagccacaaataaaattaaatacctaggaattaacttaatcaaagaagtgaaagatctctacaatgaaaactataaaacaccgatgaaagaaattgaagaggacacaaaaacatagaaagatattccatgtttatatatttcaagaatcaatattgttaaaaatgcccacaatacccaaagcaatatatggattcaatgcaatccctatcaaaataccaatgacattcttcacagaaatatttttttaatcctaaaatttgtatggattcacaaaagaccaagaagagccaaagctaacatcagcaaaaagaacaaaactggaggaaacacatttacctgacttcaaagtatactacagagctatagtaaccaaaacagcctggtactggtataaaaacagacacatagaccaatggaacagaatagagaccccagtaacaaatctacacatctacagtgaactcatttttgacaaaggtgccaagaacacagactggggaaaagaaatgcaaactatacatccaacaaaggtctaatagccaacatctataaagaacttaaatgtacaagaaaaaaaaattaaaaaatgggcaatggacatgaacagacatttttcaaaagaagacatacatgtggacgacaatcatataaaaaaaaagcttaacatcactgattactagagaagtgcaaatcaaaaccacaataagataccatctaacaccagtcagaatggctattaataaagagtcaaaaaataacaggtgtggcaaggttgtggagaaaaataaacacttatacactgtcggtgggagtgtgaattagttcaaccattatggaaaatagtgtggcaattcctcaaagatctaaagacagaaataccatttggcccagcaatcccattactgggtatatatccagaggaatataaatcataaagacacatgcacacatatgttcactgcagtactactcacaatagcaaagatatgaaattaacctaaacacccatcaattacggactgaataaagaaaatgtggtacatatacaccatgggatactatgcagccataaaaaggaacgagatcagctgagcatagtggctcacgcctgtaatcccagcactttcagaagccgaggtgggcagataacgaggtcaggagatcgagactatcctggccaacatggtgaaaccccatctctactaaaaatacaaaaattagctgcatgtggtggcactcacctgtaatctcagctactcgggaggctgaggcacgagaatcgcttgaacccaggaggcggaggttgcagtgagccaagatcgcgccattgcactccagcctggcaacagagtgagaccatccccacattctgaagtgaacctagctctggggccaggcttgatcgtcctcctcaaagccatcctgctcctgctgggtgggcttctgtggctccaggcacttgcatcaggagtgtctgcagtgaaggtctcacgtctgaccctcctgacctcctgagctgacctcaacctgtcccactatgtgttctcttttttcaaaaaaagaaaggtctgttacatacatggaaaggaaagaaaggagaccagatctggcattgcttttctgtgagtgaagaacatgcaggaaatcggctatcataatccctaggaatgtgcttctccactcattgacttgggaaaggaaacaaatgaagaaaggcctcataaaaatgagttcatgtgggaggaattatatgcagcttcctatcaattacagtaataggctgcaaagggcgtggcggaggaaaaagaattactcagagggtcacaacaactgccttctagtccaagtccctctcatttaccaaagacaccatgcttttagtccccctcatgctgttcaggcagcccaacttgcccatttctatctacaaaattccacttgtccttcaaggctcagatcaaatatcatgttcttcatgaagcctccagtgccctaggccaggagagggtcttctttcccctccagagttctccagaatgcaaattctgtgacagtccttttcatgctctgcttggcatcagtcaagtgcaccccaaatgctaccccaaacaggactgtttgttcttaagttgtataaaaga

SEQ ID NO: 23 - DNA, 1760 bp, Homo sapiens
  CDS (90)..(1598)
cagggaagctccaggcaaagagcccagcaaacagcagcactcagctaaaaggaagactcacagaacacagttgaagaaggaaagtggcgatggacctcatcccaaatttggcggtggaaacctggcttctcctggctgtcagcctggtgctcctctatctatatgggacccgtacacatggactttttaagagactgggaattccagggcccacacctctgcctttgttgggaaatgttttgtcctatcgtcagggtctctggaaatttgacacagagtgctataaaaagtatggaaaaatgtggggaacgtatgaaggtcaactccctgtgctggccatcacagatcccgacgtgatcagaacagtgctagtgaaagaatgttattctgtcttcacaaatcgaaggtctttaggcccagtgggatttatgaaaagtgccatctctttagctgaggatgaagaatggaagagaatacggtcattgctgtctccaaccttcaccagcggaaaactcaaggagatgttccccatcattgcccagtatggagatgtattggtgagaaacttgaggcgggaagcagagaaaggcaagcctgtcaccttgaaagacatctttggggcctacagcatggatgtgattactggcacatcatttggagtgaacatcgactctctcaacaatccacaagacccctttgtggagagcactaagaagttcctaaaatttggtttcttagatccattatttctctcaataatactctttccattccttaccccagtttttgaagcattaaatgtctctctgtttccaaaagataccataaattttttaagtaaatctgtaaacagaatgaagaaaagtcgcctcaacgacaaacaaaagcaccgactagatttccttcagctgatgattgactcccagaattcgaaagaaactgagtcccacaaagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacagatccccttgaaattagacacgcaaggacttcttcaaccagaaaaacccattgttctaaaggtggattcaagagatggaaccctaagtggagaatgagttattctaaggacttctactttggtcttcaagaaagctgtgccccagaacaccagagatttcaacttagtcaataaaaccttgaaataaagatgggcttaatctaatgtaaaaaaaaaaaaaaaaaaaaaaaaaaaaaaaaaaaaaaaaaaaaaaaaaaaa

SEQ ID NO: 24 - protein, 502 aa, Homo sapiens
MDLIPNLAVETWLLLAVSLVLLYLYGTRTHGLFKRLGIPGPTPLPLLGNVLSYRQGLWKFDTECYKKYGKMWGTYEGQLPVLAITDPDVIRTVLVKECYSVFTNRRSLGPVGFMKSAISLAEDEEWKRIRSLLSPTFTSGKLKEMFPIIAQYGDVLVRNLRREAEKGKPVTLKDIFGAYSMDVITGTSFGVNIDSLNNPQDPFVESTKKFLKFGFLDPLFLSIILFPFLTPVFEALNVSLFPKDTINFLSKSVNRMKKSRLNDKQKHRLDFLQLMIDSQNSKETESHKALSDLELAAQSIIFIFAGYETTSSVLSFTLYELATHPDVQQKLQKEIDAVLPNKAPPTYDAVVQMEYLDMVVNETLRLFPVAIRLERTCKKDVEINGVFIPKGSMVVIPTYALHHDPKYWTEPEEFRPERFSKKKDSIDPYIYTPFGTGPRNCIGMRFALMNMKLALIRVLQNFSFKPCKETQIPLKLDTQGLLQPEKPIVLKVDSRDGTLSGE

SEQ ID NO: 25 - DNA, 3612 bp, Homo sapiens
  misc_feature (1923)..(1923): n may be any nucleotide
cagggaagctccaggcaaagagcccagcaaacagcagcactcagctaaaaggaagactcacagaacacagttgaagaaggaaagtggcgatggacctcatcccaaatttggcggtggaaacctggcttctcctggctgtcagcctggtgctcctctatctatatgggacccgtacacatggactttttaagagactgggaattccagggcccacacctctgcctttgttgggaaatgttttgtcctatcgtcagggtctctggaaatttgacacagagtgctataaaaagtatggaaaaatgtgggggtgagtattctgaaaacctccattggatagacctgctactgtgaggaggttaccccactgcaggatagtctctgcccaggtcttcatgggatgaagctcttgtcaacctaaatacaaacagagagaggttctctgaaagaagaggataattacttgggagtagaatattgcaatgggaatctgcttgccgttataaactatgtgcaaattcagggaggtaaacaagacaaagatgctccatagaaaatatgagaagaatctcataactgttttgagataattattgttagctacaaagatcaataacaagggtgatgccacaccaaggttggacaggcagttgctggacaggtgtccttgcagaaatatttttgtgtaaagttgaaatagcctttgtgcaaagttgtggtttttgtagacacttttgtaatagttttgtttccaggaacacaagcataagaatcctctcttcatagccttcttgggatttatttgtcagggttaaaaaacaattagtgacatcactttggttctgataaagttcacactcgctattgtaaaacttttcgaggcttgtcctaccaaggatcccatgtgtcaccaggtatcgaggtcttcagtctgaactaggctaggagcattgtggttaccacttttctgcaggttttggtggcccagggactcccagcatcgccttctgtccagtgtctgcctattcccctcttctttttttcttccttaggtgcccttttatcacatgcattgtctcagacccttctaatatgtgctcataaatgcatggcatcatctccttcccacattgattcactttcaattaaaagccaaaactccttcatttagactgaatttaacatgtgcttttgaaagaagggttgagagataatagagaaacagattgggaaaccacttatgctccacttttttaaactttctctgcaagtatggaattttttgttctgctttgttgtttaaatttaagccaaaacttcttaatagaaggatatacaaatatttattggtttataccattgcacttactttgaagaagagatgctgaatattattaaaccattgtgttccctggtgggctgatggactgtgattttataaggtggtctcagccaattgcagcagctgttccctgtcagaggggctagaggtttggtgagagcagtggatgaggtgcagtggtgtgtttgttcactagaagcaagtgggagaaagctttgcctctttgtacttcttcatcttctcccctcaagtcctcagaatccacagcgctgactgtggagtgctgtggagctggcatggcccatacaggcaacatgacttagtagacagatgacacgctctagatgtccatgggccccacaccaactgcccttgcagcatttagtccttgtgagcacttgatgatttacctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcantatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaaggtctttaggcccagtgggatttatgaaaagtgccatctctttagctgaggatgaagaatggaagagaatacggtcattgctgtctccaaccttcaccagcggaaaactcaaggagatgttccccatcattgcccagtatggagatgtattggtgagaaacttgaggcgggaagcagagaaaggcaagcctgtcaccttgaaagacatctttggggcctacagcatggatgtgattactggcacatcatttggagtgaacatcgactctctcaacaatccacaagacccctttgtggagagcactaagaagttcctaaaatttggtttcttagatccattatttctctcaataatactctttccattccttaccccagtttttgaagcattaaatgtctctctgtttccaaaagataccataaattttttaagtaaatctgtaaacagaatgaagaaaagtcgcctcaacgacaaacaaaagcaccgactagatttccttcagctgatgattgactcccagaattcgaaagaaactgagtcccacaaagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaataaggcaccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacagatccccttgaaattagacacgcaaggacttcttcaaccagaaaaacccattgttctaaaggtggattcaagagatggaaccctaagtggagaatgagttattctaaggacttctactttggtcttcaagaaagctgtgccccagaacaccagagatttcaacttagtcaataaaaccttgaaataaagatgggcttaatctaatgtaaaaaaaaaaaaaaaaaaaaaaaaaaaaaaaaaaaaaaaaaaaaaaaaaaaa

SEQ ID NO: 26 - protein, 502 aa, Homo sapiens; identical to SEQ ID NO: 24

SEQ ID NO: 27 - protein, 102 aa, Homo sapiens
MDLIPNLAVETWLLLAVSLVLLYLYGTRTHGLFKRLGIPGPTPLPLLGNVLSYRQGLWKFDTECYKKYGKMWGTYEGQLPVLAITDPDVIRTVLVKECYSVF

SEQ ID NO: 28 - DNA, 23 bp, Artificial
  PCR Primer
gttcacttcagttacccatctcg

SEQ ID NO: 29 - DNA, 23 bp, Artificial
  PCT Primer
tatcctgtccatcaacactgacc

SEQ ID NO: 30 - DNA, 22 bp, Artificial
  PCR Primer
aggctataggttccaggcttgc

SEQ ID NO: 31 - DNA, 23 bp, Artificial
  PCT Primer
agaacagtgtgaagacaatggcc

SEQ ID NO: 32 - DNA, 24 bp, Artificial
  PCR primer
atctcacagtaacttggcagtttc

SEQ ID NO: 33 - DNA, 22 bp, Artificial
  PCR primer
aacccaaacaggaagtgtggcc

SEQ ID NO: 34 - DNA, 24 bp, Artificial
  Sequencing primer
gtcagttcctatatcctgtgtctg

SEQ ID NO: 35 - DNA, 23 bp, Artificial
  Sequencing primer
tcctgtccatcaacactgacctg

SEQ ID NO: 36 - DNA, 22 bp, Artificial
  Sequencing primer
cccatcattgcaatagcaggag

SEQ ID NO: 37 - DNA, 23 bp, Artificial
  Sequencing primer
gaacagtgtgaagacaatggcct

SEQ ID NO: 38 - DNA, 23 bp, Artificial
  Sequencing primer
gctggtcctgaagttgatctgtg

SEQ ID NO: 39 - DNA, 23 bp, Artificial
  Sequencing primer
aaacaggaagtgtggccagatgc